(19) United States
(12) Patent Application Publication
(10) Pub. No.: US 2004/0033502 A1
(43) Pub. Date: Feb. 19, 2004

(54) Gene expression profiles in esophageal tissue

(76) Inventors: Amanda Williams, Gaithersburg, MD (US); Joseph F. Boland, Gaithersburg, MD (US); Reginald V. Lord, Gaithersburg, MD (US); Christopher Alvares, Gaithersburg, MD (US); Jon C. Wetzel, Gaithersburg, MD (US); Uwe Scherf, Gaithersburg, MD (US); Joseph G. Vockley, Gaithersburg, MD (US)

Correspondence Address: MORGAN LEWIS & BOCKIUS LLP, 1111 PENNSYLVANIA AVENUE NW, WASHINGTON, DC, 20004, US

(21) Appl. No.: 10/240,425

(22) PCT Filed: Mar. 28, 2001
(86) PCT No.: PCT/US01/09847

(51) Int. Cl. C12Q 1/68; A61K 31/00

(52) U.S. Cl. 435/6; 514/1

(57) ABSTRACT

The invention concerns a novel nucleic acid fragment of the genome of rice pathogenic fungus Magnaporthe grisea comprising a gene coding for a protein (hereafter referred to as gene 763) whereof the presence and integrity are indispensable for pathogenesis of said fungus with respect to rice and barley. The invention also concerns the promoter of said gene, the gene coding for protein 763, protein 763 and uses thereof for identifying potential biological targets for novel fungicide molecules and for isolating genes coding for proteins controlling biochemical functions essential to the pathogenesis of the fungus Magnaporthe grisea with respect to rice and barley. The invention further concerns compounds inhibiting pathogenesis of fungi related to the expression of gene 763.

### BACKGROUND OF THE INVENTION

There are two main types of esophageal cancer; squamous cell carcinoma (SCC) and adenocarcinoma. The worldwide incidence of esophageal SCC is higher than that of adenocarcinoma; however, in the last few decades, the incidence of adenocarcinoma in Western countries has been increasing at a dramatic rate. As a result, esophageal adenocarcinoma is the most common cancer type among Caucasian patients in some populations (Blot & McLaughlin, Semin. Oncol. (1999) 26,2-8).

The main risk factor for development of esophageal adenocarcinoma is the presence of Barrett's esophagus, a disease in which the normal squamous epithelium of the lower esophagus is replaced by columnar mucosa in response to injury caused by chronic gastroesophageal reflux (Lagergren et al., N. Engl. J. Med. (1999) 340, 825-831; Barrett et al., Nat. Genet. (1999) 22, 106-109; Reid & Weinstein, Annu. Rev. Med. (1987) 38, 477-492). Barrett's esophagus is a disorder in which the lining of the esophagus undergoes cellular changes in response to chronic irritation and inflammation of reflux esophagitis. This condition is more common in men than women. The patient with Barrett's esophagus is at an increased risk of developing cancer of the esophagus. Symptoms are similar to those of reflux esophagitis and include heartburn, difficulty swallowing and pain relief with antiacid use or eating. The diagnosis of Barrett's is made by a biopsy of the esophageal mucosa through an endoscope. Treatment includes control of reflux disease, weight reduction and avoidance of alcohol, tobacco, fatty foods and lying flat after eating. Close follow-up is recommended to be certain the individual does not develop cancer of the esophagus.

The precursor cell for Barrett's epithelium has not been identified, leaving the origin of Barrett's esophagus open to speculation. One theory suggests that denudation of the squamous epithelium layer by reflux acid allows gastric columnar cells to move into the site and take over (Bremner et al., Surgery (1970) 68, 209-16). More recently, cytokeratin expression data has been used to suggest that Barrett's epithelium evolves from a basal cell in the esophageal squamous epithelium (Boch et al., Gastroenterology (1997) 112, 760-765; Salo et al., Arm. Med. (1996) 28, 305-309).

The advent of cDNA and oligonucleotide arrays has enabled researchers to map tissue-specific expression levels for thousands of genes (Alon et al., Proc. Natl. Acad. Sci. USA (1999) 96, 6745-6750; Iyer et al., (1999) Science 283, 83-87; Khan et al., Cancer Res. (1998) 58, 5009-13; Lee et al., Science (1999) 285, 1390-1393; Wang et al. Gene (1999) 229,101-108; Whitney et al., Ann. Neurol. (1999)46,425-428). Instead of assigning individual genes to a disease phenotype, expression profiles can be created which identify changes in total gene expression in the diseased tissue in relationship to normal adjacent tissue. Present day cancer research, particularly research in the field of adenocarcinoma, has focused on the determining the expression levels of individual genes with little effort expended on determining the global changes in gene expression that are correlated with the development and progression of adenocarcinoma.

There remains a need in the art for materials and methods that permit a more accurate diagnosis of esophageal cancer and, in particular, esophageal adenocarcinoma. In addition, there remains a need in the art for methods to treat and methods to identify agent that can effectively treat esophageal cancer. The present invention meets these and other needs.

### SUMMARY OF THE INVENTION

The present invention is based in part on the global changes in gene expression associated with esophageal cancer identified by examining gene expression in tissue from normal and diseased esophagus. The present invention also includes expression profiles which serve as useful diagnostic markers as well as markers that can be used to monitor disease states, disease progression, drug toxicity, drug efficacy and drug metabolism.

The invention includes methods of diagnosing esophageal cancer in a patient comprising the step of detecting the level of expression in a tissue sample of two or more genes from Tables 2-8; wherein differential expression of the genes in Tables 2-8 is indicative of esophageal cancer. In some preferred embodiments, the method may include detecting the expression level of one or more genes selected from a group consisting of apolipoprotein C-1, galectin 4, keratin 18, annexin A10, cathepsin E, homeobox C10, MPP1, transglutaminase 1, aquaporin 3, trefoil peptide1, trefoil peptide 2 or mucin 5J.

The invention also includes methods of detecting the progression of esophageal cancer. For instance, methods of the invention include detecting the progression of esophageal cancer in a patient comprising the step of detecting the level of expression in a tissue sample of two or more genes from Tables 2-8; wherein differential expression of the genes in Tables 2-8 is indicative of esophageal cancer progression. In some preferred embodiments, the progression may be the progression of Barrett's esophagus to esophageal cancer. In some preferred embodiments, the method may include detecting the expression level of one or more genes selected from a group consisting of apolipoprotein C-1, galectin 4, keratin 18, annexin A10, cathepsin E, homeobox C10, MPP1, transglutaminase 1, aquaporin 3, trefoil peptide1, trefoil peptide 2 or mucin 5B.

In some aspects, the present invention provides a method of monitoring the treatment of a patient with esophageal cancer, comprising administering a pharmaceutical composition to the patient and preparing a gene expression profile from a cell or tissue sample from the patient and comparing the patient gene expression profile to a gene expression from a cell population comprising normal esophageal cells or to a gene expression profile from a cell population comprising esophageal cancer cells or to both. In some preferred embodiments, the gene profile will include the expression level of one or more genes in Tables 2-8. In other preferred embodiments, one or more genes may be selected from a group consisting of apolipoprotein C-1, galectin 4, keratin 18, annexin A10, cathepsin E, homeobox C10, MPP1, transglutaminase 1, aquaporin 3, trefoil peptide1, trefoil peptide 2 or mucin 5B.

In another aspect, the present invention provides a method of treating a patient with esophageal cancer, comprising administering to the patient a pharmaceutical composition, wherein the composition alters the expression of at least one gene in Tables 2-8, preparing a gene expression profile from a cell or tissue sample from the patient comprising tumor cells and comparing the patient expression profile to a gene expression profile from an untreated cell population comprising esophageal cancer cells.

In one aspect, the present invention provides a method of diagnosing esophageal adenocarcinoma in a patient, comprising detecting the level of expression in a tissue sample of two or more genes from Tables 2-8, wherein differential expression of the genes in Tables 2-8 is indicative of esophageal adenocarcinoma.

In another aspect, the present invention provides a method of detecting the progression of esophageal adenocarcinoma in a patient, comprising detecting the level of expression in a tissue sample of two or more genes from Tables 2-8; wherein differential expression of the genes in Tables 2-8 is indicative of esophageal adenocarcinoma progression.

The present invention also provides materials and methods for monitoring the treatment of a patient with a esophageal adenocarcinoma. The present invention provides a method of monitoring the treatment of a patient with esophageal adenocarcinoma, comprising administering a pharmaceutical composition to the patient, preparing a gene expression profile from a cell or tissue sample from the patient and comparing the patient gene expression profile to a gene expression from a cell population comprising normal esophageal cells or to a gene expression profile from a cell population comprising esophageal adenocarcinoma cells or to both. In some preferred embodiments, the method may include detecting the level of expression of one or more genes selected from a group consisting of apolipoprotein C-1, galectin 4, keratin 18, annexin A10, cathepsin E, homeobox C10, MPP1, transglutaminase 1, aquaporin 3, trefoil peptide1, trefoil peptide 2 or mucin 5B.

In a related aspect, the present invention provides a method of treating a patient with esophageal adenocarcinoma, comprising administering to the patient a pharmaceutical composition, wherein the composition alters the expression of at least one gene in Tables 2-8, preparing a gene expression profile from a cell or tissue sample from the patient comprising esophageal adenocarcinoma cells and comparing the patient expression profile to a gene expression profile from an untreated cell population comprising esophageal adenocarcinoma cells. In some preferred embodiments, one or more genes may be selected from a group consisting of apolipoprotein C-1, galectin 4, keratin 18, annexin A10, cathepsin E, homeobox C10, MPP1, transglutaminase 1, aquaporin 3, trefoil peptide1, trefoil peptide 2 or mucin SB.

The invention further includes methods of screening for an agent capable of modulating the onset or progression of esophageal cancer, comprising the steps of exposing a cell to the agent; and detecting the expression level of two or more genes from Tables 2-8. In some embodiments, the esophageal cancer may be an esophageal adenocarcinoma. In some preferred embodiments, one or more genes may be selected from a group consisting of apolipoprotein C-1, galectin 4, keratin 18, annexin A10, cathepsin E, homeobox C10, MPP1, transglutaminase 1, aquaporin 3, trefoil peptide1, trefoil peptide 2 or mucin 5B. Preferred methods may detect all or nearly all of the genes in the tables.

The invention further includes compositions comprising at least two oligonucleotides, wherein each of the oligonucleotides comprises a sequence that specifically hybridizes to a gene in Tables 2-8 as well as solid supports comprising at least two probes, wherein each of the probes comprises a sequence that specifically hybridizes to a gene in Tables 2-8. In some preferred embodiments, one or more genes may be selected from a group consisting of apolipoprotein C-1, galectin 4, keratin 18, annexin A10, cathepsin E, homeobox C10, MPP1, transglutaminase 1, aquaporin 3, trefoil peptide1, trefoil peptide 2 or mucin 5B.

The invention further includes computer systems comprising a database containing information identifying the expression level in esophageal tissue of a set of genes comprising at least two genes in Tables 2-8 and a user interface to view the information. In some preferred embodiments, one or more genes may be selected from a group consisting of apolipoprotein C-1, galectin 4, keratin 18, annexin A10, cathepsin E, homeobox C10, MPP1, transglutaminase 1, aquaporin 3, trefoil peptide1, trefoil peptide 2 or mucin 5B. The database may further include sequence information for the genes, information identifying the expression level for the set of genes in normal esophageal tissue and cancerous tissue and may contain links to external databases such as GenBank.

Lastly, the invention includes methods of using the databases, such as methods of using the disclosed computer systems to present information identifying the expression level in a tissue or cell of at least one gene in Tables 2-8, comprising the step of comparing the expression level of at least one gene in Tables 2-8 in the tissue or cell to the level of expression of the gene in the database. In some preferred embodiments, one or more genes may be selected from a group consisting of apolipoprotein C-1, galectin 4, keratin 18, annexin A10, cathepsin E, homeobox C10, MPP1, transglutaminase 1, aquaporin 3, trefoil peptide1, trefoil peptide 2 or mucin 5B.

### BRIEF DESCRIPTION OF THE DRAWINGS

FIG. 1 shows the results of a cluster analysis. FIG. 1a shows genes under expressed in BA while FIGS. 1b, 1c and 1d show genes overexpressed in BA.

FIG. 2 shows the results of a cluster analysis. FIG. 2a shows genes identified as markers for squamous epithelial cells. FIG. 2b shows genes involved in extracellular matrix (ECM) modification. FIG. 2c shows genes involved in cell adhesion, migration, proliferation and differentiation.

### DETAILED DESCRIPTION

Many biological functions are accomplished by altering the expression of various genes through transcriptional (e.g., through control of initiation, provision of RNA precursors, RNA processing, etc.) and/or translational control. For example, fundamental biological processes such as cell cycle, cell differentiation and cell death, are often characterized by the variations in the expression levels of groups of genes.

Changes in gene expression also are associated with pathogenesis. For example, the lack of sufficient expression of functional tumor suppressor genes and/or the over expression of oncogene/protooncogenes could lead to tumorgenesis or hyperplastic growth of cells Marshall, (1991) Cell, 64, 313-326; Weinberg, (1991) Science, 254, 1138-1146). Thus, changes in the expression levels of particular genes (e.g., oncogenes or tumor suppressors) serve as signposts for the presence and progression of various diseases.

Monitoring changes in gene expression may also provide certain advantages during drug screening development. Often drugs are screened and prescreened for the ability to interact with a major target without regard to other effects the drugs have on cells. Often such other effects cause toxicity in the whole animal, which prevent the development and use of the potential drug.

Applicants have examined tissue from normal esophageal tissue and tissue from esophageal tumors to identify global changes in gene expression between tumor biopsies and normal tissue. These global changes in gene expression, also referred to as expression profiles, provide useful markers for diagnostic uses as well as markers that can be used to monitor disease states, disease progression, drug toxicity, drug efficacy and drug metabolism.

Expression profiles of genes in particular, tissues, disease states or disease progression stages provide molecular tools for evaluating toxicity, drug efficacy, drug metabolism, development, and disease monitoring. Changes in the expression profile from a baseline profile can be used as an indication of such effects. Those skilled in the art can use any of a variety of known techniques to evaluate the expression of one or more of the genes and/or ESTs identified in the instant application in order to observe changes in the expression profile.

The present application has identified differences in gene expression between normal esophageal tissue and esophageal adenocarcinoma. Barrett's epithelium was identified adjacent to many of the cancers. In some cases, the tumor involved an extensive area of esophageal mucosa suggesting that it had overgrown the Barrett's epithelium from which it derived. Genes and ESTs have been found whose expression significantly varies (>3 fold change up or down) between normal and malignant tissue. In preferred embodiments, the expression level of one or more of these genes and/or ESTs can be determined using as interrogators probes specific to one or more of these genes and/or ESTs. This permits the determination of the expression pattern in unknown cells or samples and their identification as benign or malignant. The expression patterns of the genes and ESTs which were examined are listed in Tables 2-8. The complete sequences of the genes and ESTs are available from GenBank using the Accession numbers shown in each table.

Definitions

In the description that follows, numerous terms and phrases known to those skilled in the art are used. In the interest of clarity and consistency of interpretation, the definitions of certain terms and phrases are provided.

The present invention provides compositions and methods to detect the level of expression of genes that may be differentially expressed dependent upon the state of the cell, i.e., normal versus cancerous. As used herein, the phrase “detecting the level expression” includes methods that quantify expression levels as well as methods that determine whether a gene of interest is expressed at all. Thus, an assay which provides a yes or no result without necessarily providing quantification of an amount of expression is an assay that requires “detecting the level of expression” as that phrase is used herein.

As used herein, oligonucleotide sequences that are complementary to one or more of the genes described herein, refers to oligonucleotides that are capable of hybridizing under stringent conditions to at least part of the nucleotide sequence of said genes. Such hybridizable oligonucleotides will typically exhibit at least about 75% sequence identity at the nucleotide level to said genes, preferably about 80% or 85% sequence identity or more preferably about 90% or 95% or more sequence identity to said genes.

“Bind(s) substantially” refers to complementary hybridization between a probe nucleic acid and a target nucleic acid and embraces minor mismatches that can be accommodated by reducing the stringency of the hybridization media to achieve the desired detection of the target polynucleotide sequence.

The terms “background” or “background signal intensity” refer to hybridization signals resulting from non-specific binding, or other interactions, between the labeled target nucleic acids and components of the oligonucleotide array (e.g. the oligonucleotide probes, control probes, the array substrate, etc.). Background signals may also be produced by intrinsic fluorescence of the array components themselves. A single background signal can be calculated for the entire array, or a different background signal may be calculated for each target nucleic acid. In a preferred embodiment, background is calculated as the average hybridization signal intensity for the lowest 5% to 10% of the probes in the array, or, where a different background signal is calculated for each target gene, for the lowest 5% to 10% of the probes for each gene. Of course, one of skill in the art will appreciate that where the probes to a particular gene hybridize well and thus appear to be specifically binding to a target sequence, they should not be used in a background signal calculation. Alternatively, background may be calculated as the average hybridization signal intensity produced by hybridization to probes that are not complementary to any sequence found in the sample (e.g., probes directed to nucleic acids of the opposite sense or to genes not found in the sample such as bacterial genes where the sample is mammalian nucleic acids). Background can also be calculated as the average signal intensity produced by regions of the array that lack any probes at all.

The phrase “hybridizing specifically to” refers to the binding, duplexing or hybridizing of a molecule substantially to or only to a particular nucleotide sequence or sequences under stringent conditions when that sequence is present in a complex mixture (e.g., total cellular) DNA or RNA.

Assays and methods of the invention may utilize available formats to simultaneously screen at least about 100, preferably about 1000, more preferably about 10,000 and most preferably about 1,000,000 or more different nucleic acid hybridizations.

The terms “mismatch control” or “mismatch probe” refer to a probe whose sequence is deliberately selected not to be perfectly complementary to a particular target sequence. For each mismatch (MM) control in a high-density array there typically exists a corresponding perfect match (PM) probe that is perfectly complementary to the same particular target sequence. The mismatch may comprise one or more bases.

While the mismatch(s) may be located anywhere in the mismatch probe, terminal mismatches are less desirable as a terminal mismatch is less likely to prevent hybridization of the target sequence. In a particularly preferred embodiment, the mismatch is located at or near the center of the probe such that the mismatch is most likely to destabilize the duplex with the target sequence under the test hybridization conditions.

The term “perfect match probe” refers to a probe that has a sequence that is perfectly complementary to a particular target sequence. The test probe is typically perfectly complementary to a portion (subsequence) of the target sequence. The perfect match (PM) probe can be a “test probe”, a “normalization control” probe, an expression level control probe and the like. A perfect match control or perfect match probe is, however, distinguished from a “mismatch control” or “mismatch probe.”

As used herein a “probe” is defined as a nucleic acid, preferably an oligonucleotide, capable of binding to a target nucleic acid of complementary sequence through one or more types of chemical bonds, usually through complementary base pairing, usually through hydrogen bond formation. As used herein, a probe may include natural (i.e., A, G, U, C or T) or modified bases (7-deazaguanosine, inosine, etc.). In addition, the bases in probes may be joined by a linkage other than a phosphodiester bond, so long as it does not interfere with hybridization. Thus, probes may be peptide nucleic acids in which the constituent bases are joined by peptide bonds rather than phosphodiester linkages.

The term “stringent conditions” refers to conditions under which a probe will hybridize to its target subsequence, but with only insubstantial hybridization to other sequences or to other sequences such that the difference may be identified. Stringent conditions are sequence-dependent and will be different in different circumstances. Longer sequences hybridize specifically at higher temperatures. Generally, stringent conditions are selected to be about 5° C. lower than the thermal melting point (Tₘ) for the specific sequence at a defined ionic strength and pH.

Typically, stringent conditions will be those in which the salt concentration is at least about 0.01 to 1.0 M sodium ion concentration (or other salts) at pH 7.0 to 8.3 and the temperature is at least about 30° C. for short probes (e.g., 10 to 50 nucleotide). Stringent conditions may also be achieved with the addition of destabilizing agents such as formamide.

The “percentage of sequence identity” or “sequence identity” is determined by comparing two optimally aligned sequences or subsequences over a comparison window or span, wherein the portion of the polynucleotide sequence in the comparison window may optionally comprise additions or deletions (i.e., gaps) as compared to the reference sequence (which does not comprise additions or deletions) for optimal alignment of the two sequences. The percentage is calculated by determining the number of positions at which the identical subunit (e.g., nucleic acid base or amino acid residue) occurs in both sequences to yield the number of matched positions, dividing the number of matched positions by the total number of positions in the window of comparison and multiplying the result by 100 to yield the percentage of sequence identity. Percentage sequence identity when calculated using the programs GAP or BESTFIT (see below) is calculated using default gap weights.

Homology or identity may be determined by BLAST (Basic Local Alignment Search Tool) analysis using the algorithm employed by the programs blastp, blastn, blastx, tblastn and tblastx (Karlin et al., (1990) Proc. Natl. Acad. Sci. USA 87, 2264-2268 and Altschul, (1993) J. Mol. Evol. 36,290-300, fully incorporated by reference) which are tailored for sequence similarity searching. The approach used by the BLAST program is to first consider similar segments between a query sequence and a database sequence, then to evaluate the statistical significance of all matches that are identified and finally to summarize only those matches which satisfy a preselected threshold of significance. For a discussion of basic issues in similarity searching of sequence databases, see Altschul et al., ((1994) Nature Genet. 6, 119-129) which is fully incorporated by reference. The search parameters for histogram, descriptions, alignments, expect (i.e., the statistical significance threshold for reporting matches against database sequences), cutoff, matrix and filter are at the default settings. The default scoring matrix used by blastp, blastx, tblasta, and tblastx is the BLOSUM62 matrix (Henikoff et al., (1992) Proc. Natl. Acad. Sci. USA 89, 10915-10919, fully incorporated by reference). Four blastn parameters were adjusted as follows Q=10 (gap creation penalty); R=10 (gap extension penalty); wink=1 (generates word hits at every winks position along the query); and gapw=16 (sets the window width within which gapped alignments are generated). The equivalent Blastp parameter settings were Q=9; R=2; wink=1; and gapw=32. A Bestfit comparison between sequences, available in the GCG package version 10.0, uses DNA parameters GAP=50 (gap creation penalty) and LENz=3 (gap extension penalty) and the equivalent settings in protein comparisons are GAP=8 and LEN=2.

Uses of Differentially Expressed Genes

The present invention identifies those genes differentially expressed between normal esophageal tissue and cancerous esophageal tissue. One of skill in the art can select one or more of the genes identified as being differentially expressed and use the information and methods provided herein to interrogate or test a particular sample. For a particular interrogation of two conditions or sources, it is desirable to select those genes that display a great difference in the expression pattern between the two conditions or sources. At least a two-fold difference is desirable, but a three, five-fold or ten-fold difference may be preferred. Interrogations of the genes or proteins can be performed to yield information on gene expression as well as on the levels of the encoded proteins.

Diagnostic Uses for the Esophageal Cancer Markers

As described herein, the genes and gene expression information provided in Tables 2-8 may be used as diagnostic markers for the prediction or identification of the malignant state of the esophageal tissue. For instance, an esophageal tissue sample or other sample from a patient may be assayed by any of the methods known to those skilled in the art, and the expression levels from one or more genes from Tables 2-8, may be compared to the expression levels found in normal esophageal tissue, tissue from esophageal adenocarinoma or both. Expression profiles generated from the tissue or other sample that substantially resemble an expression profile from normal or diseased esophageal tissue may be used, for instance, to aid in disease diagnosis. Comparison of the expression data, as well as available sequence or other information may be done by researcher or diagnostician or may be done with the aid of a computer and databases as described herein.

Use of the Esophageal Cancer Markers for Monitoring Disease Progression

Molecular expression markers for esophageal cancer can be used to confirm the type and progression of the cancer made on the basis of morphological criteria. For example, squamous cell carcinoma could be distinguished from adenocarcinoma based on the level and type of genes expressed in a tissue sample. In some situations, identifications of cell type or source is ambiguous based on classical criteria. In these situations the molecular expression markers of the present invention are useful.

In addition, progression of esophageal squamous cell carcinoma to adenocarcinoma can be monitored by following the expression patterns of the involved genes using the molecular expression markers of the present invention. Perturbed expression can be observed in the diseased state. Monitoring of the efficacy of certain drug regimens can also be accomplished by following the expression patterns of the molecular expression markers.

Although only a few different disease progression time points have been observed, as shown in the examples below, other developmental stages can be studied using these same molecular expression markers. The importance of these markers in development has been shown here, however, variations in their expression may occur at other times. For example, one could study the expression of these markers at benign stages for comparison to expression at malignant states.

As described above, the genes and gene expression information provided in Tables 28 may also be used as markers for the monitoring of disease progression, for instance, the development of esophageal cancer. For instance, an esophageal tissue sample or other sample from a patient may be assayed by any of the methods known to those of skill in the art, and the expression levels in the sample from a gene or genes from Tables 2-8 may be compared to the expression levels found in normal esophageal tissue, tissue from esophageal cancer, in particular, Barrett's-associated esophageal adenocarcinoma (BA), or both. Comparison of the expression data, as well as available sequence or other information may be done by researcher or diagnostician or may be done with the aid of a computer and databases as described herein.

Use of the Esophageal Cancer Markers for Drug Screening

Potential drugs can be screened to determine if application of the drug alters the expression of one or more of the genes identified herein. This may be useful, for example, in determining whether a particular drug is effective in treating a particular patient or patient population with esophageal cancer. In the case where the expression of a gene is affected by the potential drug such that its level of expression returns to normal, the drug is indicated in the treatment of esophageal cancer. Similarly, a drug that causes expression of a gene which is not normally expressed by epithelial cells in the esophagus, may be contraindicated in the treatment of esophageal cancer.

According to the present invention, the genes identified in Tables 2-8 may be used as markers to evaluate the effects of a candidate drug or agent on a cell, particularly a cell undergoing malignant transformation, for instance, an esophageal cancer cell or tissue sample. A candidate drug or agent can be screened for the ability to stimulate the transcription or expression of a given marker or markers (drug targets) or to down-regulate or inhibit the transcription or expression of a marker or markers. According to the present invention, one can also compare the specificity of the effects of a drug by looking at the number of markers affected by the drug and comparing them to the number of markers affected by a different drug. A more specific drug will affect fewer transcriptional targets. Similar sets of markers identified for two drugs indicates a similarity of effects.

Assays to monitor the expression of a marker or markers as defined in Tables 2-8 may utilize any available means of monitoring for changes in the expression level of the nucleic acids of the invention. As used herein, an agent is said to modulate the expression of a nucleic acid of the invention if it is capable of up- or down-regulating expression of the nucleic acid in a cell.

Agents that are assayed in the above methods can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

As used herein, an agent is said to be rationally selected or designed when the agent is chosen on a nonrandom basis which takes into account the sequence of the target site and/or its conformation in connection with the agent's action. Agents can be rationally selected or rationally designed by utilizing the peptide sequences that make up these sites. For example, a rationally selected peptide agent can be a peptide whose amino acid sequence is identical to or a derivative of any functional consensus site.

The agents of the present invention can be, as examples, peptides, small molecules, vitamin derivatives, as well as carbohydrates, lipids, oligonucleotides and covalent and non-covalent combinations thereof. Dominant negative proteins, DNA encoding these proteins, antibodies to these proteins, peptide fragments of these proteins or mimics of these proteins may be introduced into cells to affect function. “Mimic” as used herein refers to the modification of a region or several regions of a peptide molecule to provide a structure chemically different from the parent peptide but topographically and functionally similar to the parent peptide (see Grant, (1995) in Molecular Biology and Biotechnology Meyers (editor) VCH Publishers). A skilled artisan can readily recognize that there is no limit as to the structural nature of the agents of the present invention.

Assay Formats

The genes identified as being differentially expressed in esophageal cancer may be used in a variety of nucleic acid detection assays to detect or quantify the expression level of a gene or multiple genes in a given sample. For example, traditional Northern blotting, nuclease protection, RT-PCR and differential display methods may be used for detecting gene expression levels.

The protein products of the genes identified herein can also be assayed to determine the amount of expression. Methods for assaying for a protein include Western blot, immunoprecipitation, radioimmunoassay. It is preferred, however, that the mRNA be assayed as an indication of expression. Methods for assaying for mRNA include Northern blots, slot blots, dot blots, and hybridization to an ordered array of oligonucleotides. Any method for specifically and quantitatively measuring a specific protein or mRNA or DNA product can be used. However, methods and assays of the invention are most efficiently designed with array or chip hybridization-based methods for detecting the expression of a large number of genes.

Any hybridization assay format may be used, including solution-based and solid support-based assay formats. A preferred solid support is a high density array also known as a DNA chip or a gene chip. In one assay format, gene chips containing probes to at least two genes from Tables 2-8 may be used to directly monitor or detect changes in gene expression in the treated or exposed cell as described herein.

Additional assay formats may be used to monitor the ability of the agent to modulate the expression of a gene identified in Tables 2-8. For instance, as described above, mRNA expression may be monitored directly by hybridization of probes to the nucleic acids of the invention. Cell lines are exposed to an agent to be tested under appropriate conditions and time and total RNA or mRNA is isolated by standard procedures such those disclosed in

In another format, cell lines that contain reporter gene fusions between the open reading frame and/or the 3′ or 5′ regulatory regions of a gene in Tables 2-8 and any assayable fusion partner may be prepared. Numerous assayable fusion partners are known and readily available including the firefly luciferase gene and the gene encoding chloramphenicol acetyltransferase (Alam et al., (1990) Anal. Biochem 188, 245-254). Cell lines containing the reporter gene fusions are then exposed to the agent to be tested under appropriate conditions and time. Differential expression of the reporter gene between samples exposed to the agent and control samples identifies agents which modulate the expression of the nucleic acid.

In another assay format, cells or cell lines are first identified which express one or more of the gene products of the invention physiologically. Cells and/or cell lines so identified would preferably comprise the necessary cellular machinery to ensure that the transcriptional and/or translational apparatus of the cells would faithfully mimic the response of normal or cancerous esophageal tissue to an exogenous agent. Such machinery would likely include appropriate surface transduction mechanisms and/or cytosolic factors. Such cell lines may be, but are not required to be, derived from esophageal tissue. The cells and/or cell lines may then be contacted with an agent and the expression of one or more of the genes of interest may then be assayed. The genes may be assayed at the mRNA level and/or at the protein level.

In some embodiments, such cells or cell lines may be transduced or transfected with an expression vehicle (e.g., a plasmid or viral vector) containing an expression construct comprising an operable 5′-promoter containing end of a gene of interest identified in Tables 2-8 fused to one or more nucleic acid sequences encoding one or more antigenic fragments. The construct may comprise all or a portion of the coding sequence of the gene of interest which may be positioned 5′- or 3′- to a sequence encoding an antigenic fragment. The coding sequence of the gene of interest may be translated or un-translated after transcription of the gene fusion. At least one antigenic fragment may be translated. The antigenic fragments are selected so that the fragments are under the transcriptional control of the promoter of the gene of interest and are expressed in a fashion substantially similar to the expression pattern of the gene of interest. The antigenic fragments may be expressed as polypeptides whose molecular weight can be distinguished from the naturally occurring polypeptides. In some embodiments, gene products of the invention may further comprise an immunologically distinct tag. Such a process is well known in the art (see Sambrook et al., (1989) Molecular Cloning—A Laboratory Manual, Cold Spring Harbor Laboratory Press).

Cells or cell lines transduced or transfected as outlined above are then contacted with agents under appropriate conditions; for example, the agent comprises a pharmaceutically acceptable excipient and is contacted with cells comprised in an aqueous physiological buffer such as phosphate buffered saline (PBS) at physiological pH, Eagles balanced salt solution (BSS) at physiological pH, PBS or BSS comprising serum or conditioned media comprising PBS or BSS and serum incubated at 37° C. Said conditions may be modulated as deemed necessary by one of skill in the art. Subsequent to contacting the cells with the agent, said cells will be disrupted and the polypeptides of the lysate are fractionated such that a polypeptide fraction is pooled and contacted with an antibody to be farther processed by immunological assay (e.g., ELISA, immunoprecipitation or Western blot). The pool of proteins isolated from the “agent-contacted” sample will be compared with a control sample where only the excipient is contacted with the cells and an increase or decrease in the immunologically generated signal from the “agent-contacted” sample compared to the control will be used to distinguish the effectiveness of the agent.

Another embodiment of the present invention provides methods for identifying agents that modulate the levels, concentration or at least one activity of a protein(s) encoded by the genes in Tables 2-8. Such methods or assays may utilize any means of monitoring or detecting the desired activity.

In one format, the relative amounts of a protein of the invention produced in a cell population that has been exposed to the agent to be tested may be compared to the amount produced in an unexposed control cell population. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe, such as a specific antibody.

The genes and ESTs of the present invention may be assayed in any convenient form. For example, they may be assayed in the form mRNA or reverse transcribed mRNA. The genes may be cloned or not and the genes may be amplified or not. The cloning itself does not appear to bias the representation of genes within a population. However, it may be preferable to use polyA+ RNA as a source, as it can be used with less processing steps. In some embodiments, it may be preferable to assay the protein or peptide encoded by the gene.

The sequences of the expression marker genes are in the public databases. Tables 2-8 provide the Accession numbers and name for each of the sequences. In Tables 2-6, the number following the notation gb=is the GenBank accession number. The sequences of the genes in GenBank are expressly incorporated by reference and are publicly available at, for example, www.ncbi.nih.gov. IMAGE gives the clone number from the IMAGE consortium.

Probe Design

Probes based on the sequences of the genes described herein may be prepared by any commonly available method. Oligonucleotide probes for assaying the tissue or cell sample are preferably of sufficient length to specifically hybridize only to appropriate, complementary genes or transcripts. Typically the oligonucleotide probes will be at least 10, 12, 14, 16, 18, 20 or 25 nucleotides in length. In some cases longer probes of at least 30, 40, or 50 nucleotides will be desirable.

One of skill in the art will appreciate that an enormous number of array designs are suitable for the practice of this invention. The high density array will typically include a number of probes that specifically hybridize to the sequences of interest. See WO 99/32660 for methods of producing probes for a given gene or genes. In addition, in a preferred embodiment, the array will include one or more control probes.

High density array chips of the invention include “test probes.” Test probes may be oligonucleotides that range from about 5 to about 500 or about 5 to about 50 nucleotides, more preferably from about 10 to about 40 nucleotides and most preferably from about 15 to about 40 nucleotides in length. In other particularly preferred embodiments, the probes are about 20 or 25 nucleotides in length. In another preferred embodiment, test probes are double or single strand DNA sequences. DNA sequences may be isolated or cloned from natural sources or amplified from natural sources using natural nucleic acid as templates. These probes have sequences complementary to particular subsequences of the genes whose expression they are designed to detect. Thus, the test probes are capable of specifically hybridizing to the target nucleic acid they are to detect.

In addition to test probes that bind the target nucleic acid(s) of interest, the high density array can contain a number of control probes. The control probes fall into three categories referred to herein as (1) normalization controls; (2) expression level controls; and (3) mismatch controls.

Normalization controls are oligonucleotide or other nucleic acid probes that are complementary to labeled reference oligonucleotides or other nucleic acid sequences that are added to the nucleic acid sample. The signals obtained from the normalization controls after hybridization provide a control for variations in hybridization conditions, label intensity, “reading” efficiency and other factors that may cause the signal of a perfect hybridization to vary between arrays. In a preferred embodiment, signals (e.g. fluorescence intensity) read from all other probes in the array are divided by the signal (, fluorescence intensity) from the control probes thereby normalizing the measurements.

Virtually any probe may serve as a normalization control. However, it is recognized that hybridization efficiency varies with base composition and probe length. Preferred normalization probes are selected to reflect the average length of the other probes present in the array, however, they can be selected to cover a range of lengths. The normalization control(s) can also be selected to reflect the (average) base composition of the other probes in the array, however in a preferred embodiment, only one or a few probes are used and they are selected such that they hybridize well (i.e., no secondary structure) and do not match any target-specific probes.

Expression level controls are probes that hybridize specifically with constitutively expressed genes in the biological sample. Virtually any constitutively expressed gene provides a suitable target for expression level controls. Typical expression level control probes have sequences complementary to subsequences of constitutively expressed “housekeeping genes” including, but not limited to the β-actin gene, the transferrin receptor gene, the GAPDH gene, and the like.

Mismatch controls may also be provided for the probes to the target genes, for expression level controls or for normalization controls. Mismatch controls are oligonucleotide probes or other nucleic acid probes identical to their corresponding test or control probes except for the presence of one or more mismatched bases. A mismatched base is a base selected so that it is not complementary to the corresponding base in the target sequence to which the probe would otherwise specifically hybridize. One or more mismatches are selected such that under appropriate hybridization conditions (e.g., stringent conditions) the test or control probe would be expected to hybridize with its target sequence, but the mismatch probe would not hybridize (or would hybridize to a significantly lesser extent). Preferred mismatch probes contain a central mismatch. Thus, for example, where a probe is a twenty-mer, a corresponding mismatch probe may have the identical sequence except for a single base mismatch (e.g., substituting a G, a C or a T for an A) at any of positions 6 through 14 (the central mismatch).

Mismatch probes thus provide a control for non-specific binding or cross hybridization to a nucleic acid in the sample other than the target to which the probe is directed. Mismatch probes also indicate whether a hybridization is specific or not. For example, if the target is present the perfect match probes should be consistently brighter than the mismatch probes. In addition, if all central mismatches are present, the mismatch probes can be used to detect a mutation. The difference in intensity between the perfect match and the mismatch probe (I_{(PM)}-I_{(M-M)}) provides a good measure of the concentration of the hybridized material.

Nucleic Acid Samples

As is apparent to one of ordinary skill in the art, nucleic acid samples used in the methods and assays of the invention may be prepared by any available method or process. Methods of isolating total mRNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I Theory and Nucleic Acid Preparation, Tijssen, (1993) (editor) Elsevier Press. Such samples include RNA samples, but also include cDNA synthesized from a mRNA sample isolated from a cell or tissue of interest. Such samples also include DNA amplified from the cDNA, and an RNA transcribed from the amplified DNA. One of skill in the art would appreciate that it may be desirable to inhibit or destroy RNase present in homogenates before homogenates can be used.

Biological samples may be of any biological tissue or fluid or cells from any organism as well as cells raised in vitro, such as cell lines and tissue culture cells. Frequently the sample will be a “clinical sample” which is a sample derived from a patient. Typical clinical samples include, but are not limited to, sputum, blood, blood-cells (e.g., white cells), tissue or fine needle biopsy samples, urine, peritoneal fluid, and pleural fluid, or cells therefrom.

Biological samples may also include sections of tissues, such as frozen sections or formalin fixed sections taken for histological purposes.

Solid Supports

Solid supports containing oligonucleotide probes for differentially expressed genes can be any solid or semisolid support material known to those skilled in the art. Suitable examples include, but are not limited to, membranes, filters, tissue culture dishes, polyvinyl chloride dishes, beads, test strips, silicon or glass based chips and the like. Suitable glass wafers and hybridization methods are widely available, for example, those disclosed by Beattie (WO 95/11755). Any solid surface to which oligonucleotides can be bound, either directly or indirectly, either covalently or non-covalently, can be used. In some embodiments, it may be desirable to attach some oligonucleotides covalently and others non-covalently to the same solid support.

A preferred solid support is a high density array or DNA chip. These contain a particular oligonucleotide probe in a predetermined location on the array. Each predetermined location may contain more than one molecule of the probe, but each molecule within the predetermined location has an identical sequence. Such predetermined locations are termed features. There may be, for example, from 2, 10, 100, 1000 to 10,000, 100,000 or 400,000 of such features on a single solid support. The solid support, or the area within which the probes are attached may be on the order of a square centimeter.

Oligonucleotide probe arrays for expression monitoring can be made and used according to any techniques known in the art (see for example, Lockhart et al., Nat. Biotechnol. (1996) 14, 1675-1680; McGall et al., Proc. Nat. Acad. Sci. USA (1996) 93, 13555-13460). Such probe arrays may contain at least two or more oligonucleotides that are complementary to or hybridize to two or more of the genes described herein. Such arrays my also contain oligonucleotides that are complementary or hybridize to at least 3, 4, 5, 6, 7, 8, 9, 10, 20, 30, 50, 70 or more the genes described herein.

Oligonucleotide arrays are particularly useful for creating gene expression profiles comparing cancer tissue to adjacent normal tissue.

The use of available oligonucleotide arrays enabled the determination of the expression levels of numerous genes and ESTs simultaneously. From this mass of expression data, differentially expressed genes were identified using Fold Change and Gene Signature Differential analysis.

Gene Signature Differential analysis is a method designed to detect genes present in one sample set, and absent in another. Genes with differential expression in cancer tissue versus normal tissue are better diagnostic and therapeutic targets than genes that do not change in expression.

Methods of forming high density arrays of oligonucleotides with a minimal number of synthetic steps are known. The oligonucleotide analogue array can be synthesized on a solid substrate by a variety of methods, including, but not limited to, light-directed chemical coupling, and mechanically directed coupling (see Pirrung et al., (1992) U.S. Pat. No. 5,143, 854; Fodor et al., (1998) U.S. Pat. No. 5,800,992; Chee et al., (1998) 5,837,832

In brief, the light-directed combinatorial synthesis of oligonucleotide arrays on a glass surface proceeds using automated phosphoramidite chemistry and chip masking techniques. In one specific implementation, a glass surface is derivatized with a silane reagent containing a functional group, e.g., a hydroxyl or amine group blocked by a photolabile protecting group. Photolysis through a photolithogaphic mask is used selectively to expose functional groups which are then ready to react with incoming 5′ photoprotected nucleoside phosphoramidites. The phosphoramidites react only with those sites which are illuminated (and thus exposed by removal of the photolabile blocking group). Thus, the phosphoramidites only add to those areas selectively exposed from the preceding step. These steps are repeated until the desired array of sequences have been synthesized on the solid surface. Combinatorial synthesis of different oligonucleotide analogues at different locations on the array is determined by the pattern of illumination during synthesis and the order of addition of coupling reagents.

In addition to the foregoing, additional methods which can be used to generate an array of oligonucleotides on a single substrate are described in Fodor et al., (1993). WO 93/09668. High density nucleic acid arrays can also be fabricated by depositing premade or natural nucleic acids in predetermined positions. Synthesized or natural nucleic acids are deposited on specific locations of a substrate by light directed targeting and oligonucleotide directed targeting. Another embodiment uses a dispenser that moves from region to region to deposit nucleic acids in specific spots.

Hybridization

Nucleic acid hybridization simply involves contacting a probe and target nucleic acid under conditions where the probe and its complementary target can form stable hybrid duplexes through complementary base pairing (see Lockhart et al., (1999) WO 99/32660). The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA-DNA, RNA-RNA or RNA-DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions may be selected to provide any degree of stringency. In a preferred embodiment, hybridization is performed at low stringency, in this case in 6×SSPE-T at 37° C. (0.005% Triton x-100) to ensure hybridization and then subsequent washes are performed at higher stringency (e.g., 1×SSPE-T at 37° C.) to eliminate mismatched hybrid duplexes. Successive washes may be performed at increasingly higher stringency (e.g. down to as low as 0.25×SSPE-T at 37° C. to 50° C. until a desired level of hybridization specificity is obtained. Stringency can also be increased by addition of agents such as formamide. Hybridization specificity may be evaluated by comparison of hybridization to the test probes with hybridization to the various controls that can be present (e.g., expression level control, normalization control, mismatch controls, etc.).

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest.

Signal Detection

The hybridized nucleic acids are typically detected by detecting one or more labels attached to the sample nucleic acids. The labels may be incorporated by any of a number of means well known to those of skill in the art (see Lockhart et al., (1999) WO 99/32660).

Databases

The present invention includes relational databases containing sequence information, for instance for the genes of Tables 2-8, as well as gene expression information in various esophageal tissue samples. Databases may also contain information associated with a given sequence or tissue sample such as descriptive information about the gene associated with the sequence information, or descriptive information concerning the clinical status of the tissue sample, or the patient from which the sample was derived. The database may be designed to include different parts, for instance a sequences database and a gene expression database. Methods for the configuration and construction of such databases are widely available, for instance, see Akerblom et al., (1999) U.S. Pat. No. 5,953,727, which is specifically incorporated herein by reference in its entirety.

The databases of the invention may be linked to an outside or external database. In a preferred embodiment, as described in Tables 2-8, the external database is GenBank and the associated databases maintained by the National Center for Biotechnology Information (NCBI).

Any appropriate computer platform may be used to perform the necessary comparisons between sequence information, gene expression information and any other information in the database or provided as an input. For example, a large number of computer workstations are available from a variety of manufacturers, such has those available from Silicon Graphics. Client-server environments, database servers and networks are also widely available and appropriate platforms for the databases of the invention.

The databases of the invention may be used to produce, among other things, electronic Northerns to allow the user to determine the cell type or tissue in which a given gene is expressed and to allow determination of the abundance or expression level of a given gene in a particular tissue or cell.

The databases of the invention may also be used to present information identifying the expression level in a tissue or cell of a set of genes comprising at least one gene in Tables 2-8 comprising the step of comparing the expression level of at least one gene in Tables 2-8 in the tissue to the level of expression of the gene in the database. Such methods may be used to predict the physiological state of a given tissue by comparing the level of expression of a gene or genes in Tables 2-8 from a sample to the expression levels found in tissue from normal esophageal tissue, tissue from esophageal adenocarcinoma or both. Such methods may also be used in the drug or agent screening assays as described herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### EXAMPLES

### Example 1

### Tissue Sample Acquisition and Preparation

For tissue specimens, nine normal esophagus samples and eight BA tissue samples, which included seven matched tumor-normal sets, were used. Six of the eight BA samples were lymph node invasive.

With minor modifications, the sample preparation protocol followed the Affymetrix GeneChip Expression Analysis Manual. Frozen tissue was first ground to powder using the Spex Certiprep 6800 Freezer Mill. Total RNA was then extracted using Trizol (Life Technologies). The total RNA yield for each sample (average tissue weight of 300 mg) was 200-500 μg. Next, mRNA was isolated using the Oligotex mRNA Midi kit (Qiagen). Since the mRNA was eluted in a final volume of 400 air, an ethanol precipitation step was required to bring the concentration to 1 μg/μl. Using 1-5 μg of mRNA, double stranded cDNA was created using the SuperScript Choice system (Gibco-BRL). First strand cDNA synthesis was primed with a T7-(dT₂₄) oligonucleotide. The cDNA was then phenol-chloroform extracted and ethanol precipitated to a final concentration of 1 μg/μl.

From 2 μg of cDNA, cRNA was synthesized according to standard procedures. To biotin label the cRNA, nucleotides Bio-11-CTP and Bio-16-UTP (Enzo Diagnostics) were added to the reaction. After a 37° C. incubation for six hours, the labeled cRNA was cleaned up according to the RNeasy Mini kit protocol (Qiagen). The cRNA was then fragmented (5× fragmentation buffer: 200 mM Tris-Acetate (pH 8.1), 500 mM KOAc, 150 mM MgOAc) for thirty-five minutes at 94° C.

55 μg of fragmented cRNA was hybridized on the human and the Human Genome U95 set of arrays for twenty-four hours at 60 rpm in a 45° C. hybridization oven. The chips were washed and stained with Streptavidin Phycoerythrin (SAPE) (Molecular Probes) in Affymetrix fluidics stations. To amplify staining, SAPE solution was added twice with an anti-streptavidin biotinylated antibody (Vector Laboratories) staining step in between. Hybridization to the probe arrays was detected by fluorometric scanning Hewlett Packard Gene Array Scanner). Following hybridization and scanning, the microarray images were analyzed for quality control, looking for major chip defects or abnormalities in hybridization signal. After all chips passed QC, the data was analyzed using Affymetrix GeneChip software (v3.0), and Experimental Data Mining Tool (EDMT) software (v1.0).

### Example 2

### Gene Expression Analysis

All samples were prepared as described and hybridized onto the Affymetrix Human Genome U95 array set.

Each chip contains 16-20 oligonucleotide probe pairs per gene or cDNA clone. These probe pairs include perfectly matched sets and mismatched sets, both of which are necessary for the calculation of the average difference. The average difference is a measure of the intensity difference for each probe pair, calculated by subtracting the intensity of the mismatch from the intensity of the perfect match. This takes into consideration variability in hybridization among probe pairs and other hybridization artifacts that could affect the fluorescence intensities. Using the average difference value that has been calculated, the GeneChip software then makes an absolute call for each gene or EST.

The absolute call of present, absent or marginal is used to generate a Gene Signature, a tool used to identify those genes that are commonly present or commonly absent in a given sample set, according to the absolute call. For each set of samples, a median average difference was figured using the average differences of each individual sample within the set. The Gene Signature for one set of samples is compared to the Gene Signature of another set of samples to determine the Gene Signature Differential. This comparison identifies the genes that are consistently present in one set of samples and consistently absent in the second set of samples.

The Gene Signature Curve is a graphic view of the number of genes consistently present in a given set of samples as the sample size increases, taking into account the genes commonly expressed among a particular set of samples, and discounting those genes whose expression is variable among those samples. The curve is also indicative of the number of samples necessary to generate an accurate Gene Signature. As the sample number increases, the number of genes common to the sample set decreases. The curve is generated using the positive Gene Signatures of the samples in question, determined by adding one sample at a time to the Gene Signature, beginning with the sample with the smallest number of present genes and adding samples in ascending order. The curve displays the sample size required for the most consistency and the least amount of expression variability from sample to sample. The point where this curve begins to level off represents the minimum number of samples required for the Gene Signature. Graphed on the x-axis is the number of samples in the set, and on the y-axis is the number of genes in the positive Gene Signature. As a general rule, the acceptable percent of variability in the number of positive genes between two sample sets should be less than 5%.

### Example 3

### Expression Profiles

Using the above described methods, genes that were predominantly over-expressed in BA, or predominantly under-expressed in BA were identified. The revealed genes were used to identify gene clusters generated by hierarchical clustering that exhibited a consistent fold change and/or dominant expression pattern between the normal and diseased sample sets. Genes with consistent differential expression patterns provide potential targets for broad range diagnostics and therapeutics.

First, the expression profiles of the nine normal esophagus samples were pooled and used to determine the genes that are commonly expressed or commonly not expressed. To find the expression pattern consistent to disease, the same procedure was followed with the eight samples from patients with BA. Table 1 lists, by array type, the number of genes with expression patterns common to the majority of normal or diseased samples.

Next, the unique pattern of genes over-expressed in the disease was identified by determining those genes that were commonly expressed in BA, but commonly NOT expressed in normal esophagus. Similarly, the unique pattern of genes under-expressed in disease was found by identifying genes that were expressed in the majority of normal esophagus samples, but NOT expressed in the majority of BA samples. Table 1 lists the number of genes uniquely under-expressed and over-expressed in. BA by array type. With this method 423 genes were identified to be unique for BA.

### Example 4

### Fold Change Analysis

The data was first filtered to exclude all genes that showed no expression in any of the samples. The ratio (tumor/normal) was calculated by comparing the mean expression value for each gene in the BA sample set against the mean expression value of that gene in the normal esophagus sample set. Genes were included in the analysis if they had a fold change ≧3 in either direction, and a P value <0.05 as determined by a two-tail unequal variance t-test. Out of the 60,000 genes surveyed by the Human Genome U95 set, 1584 genes were present in the overall fold change analysis, 701 were over-expressed in BA and 883 were under-expressed in BA. Out of the 423 unique genes for BA (244 under-expressed and 179 over-expressed) previously identified, 170 were also present in the fold change analysis. Determining these 170 genes independently by both methods overcomes the limitations of accuracy inherent in either method. These 170 key disease-related genes have both significant overall fold changes, and 87 are not detectable in BA while the remaining 83 are not detectable in normal esophagus.

The genes identified in the fold change analysis are listed in Tables 2-6. Table 2 lists those genes identified using the Human Genome U95A chip, Table 3 lists those genes identified using the Human Genome U95B chip, Table 4 lists those genes identified using the Human Genome U95C chip, Table 5 lists those genes identified using the Human Genome U95D chip and Table 6 lists those genes identified using the Human Genome U9SE chip.

### Example 5

### Cluster Analysis

The data was first filtered to exclude all genes that showed no expression in any of the samples. To normalize the data, fold change values for the samples were calculated by dividing each gene expression value by the mean of the expression values for all samples, both normal esophagus and BA, for that gene. Genes were included in the cluster analysis if they had at least one instance of a fold change ≧3 in either direction, and a P value of <0.05 as assessed by a two-tail unequal variance t-test. Using a hierarchical clustering algorithm, genes were grouped according to their expression pattern similarities across all samples (Eisen, et al., Cluster analysis and display of genome-wide expression patterns. Proc. Natl. Acad. Sci. USA 95, 14863-14868 (1998)).

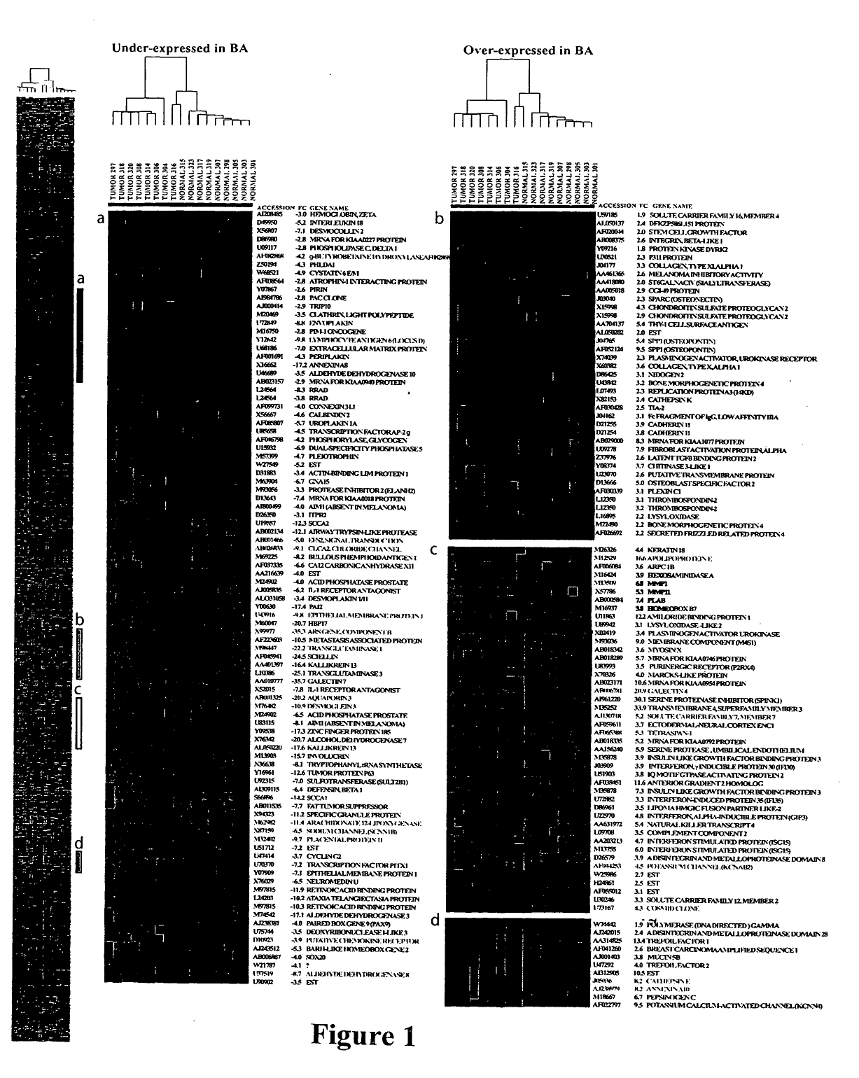

For the Human Genome U95A array, 1100 full-length known genes or ESTs (8.7% of the genes present on the array) were included in the cluster analysis. The resulting dendrogram (FIG. 1) grouped all nine normal esophagus samples and seven of the eight BA samples into separate trees. BA sample 316 clustered in a branch with its matched normal esophagus sample (315) rather than with the other tumors. A number of genes on the Human Genome U95A array are present in duplicate. In most cases the duplicate genes cluster next to each other or in close proximity of each other, verifying internal microarray reproducibility. Four clusters were chosen for in-depth analyses, based on the presence of a portion of the 170 key disease-related genes previously identified by our fingerprinting and fold change analysis methods. FIG. 1 shows the results obtained using a hierarchical clustering to measure expression variation for 1100 full-length genes present on the Affymetrix Human Genome U95A oligonucleotide array. Four clusters (a-d) are presented that include genes from the 170 gene list identified by both our analysis methods. Those genes are labeled in red. Cluster (a) contains genes under-expressed in Barrett's-associated esophageal adenocarcinoma (BA), while clusters (b-d) contain genes over-expressed in BA. The dendrogram summarizes the expression similarities between samples. Each gene is represented by a single row, and each sample by a single column Relative to the mean expression level of all samples, red squares represent an over-expression, green squares represent an under-expression, black squares represent no expression change, and grey squares denote a missing sample. The overall fold change (FC), the fold change calculated between the two groups of samples, for each gene is also listed.

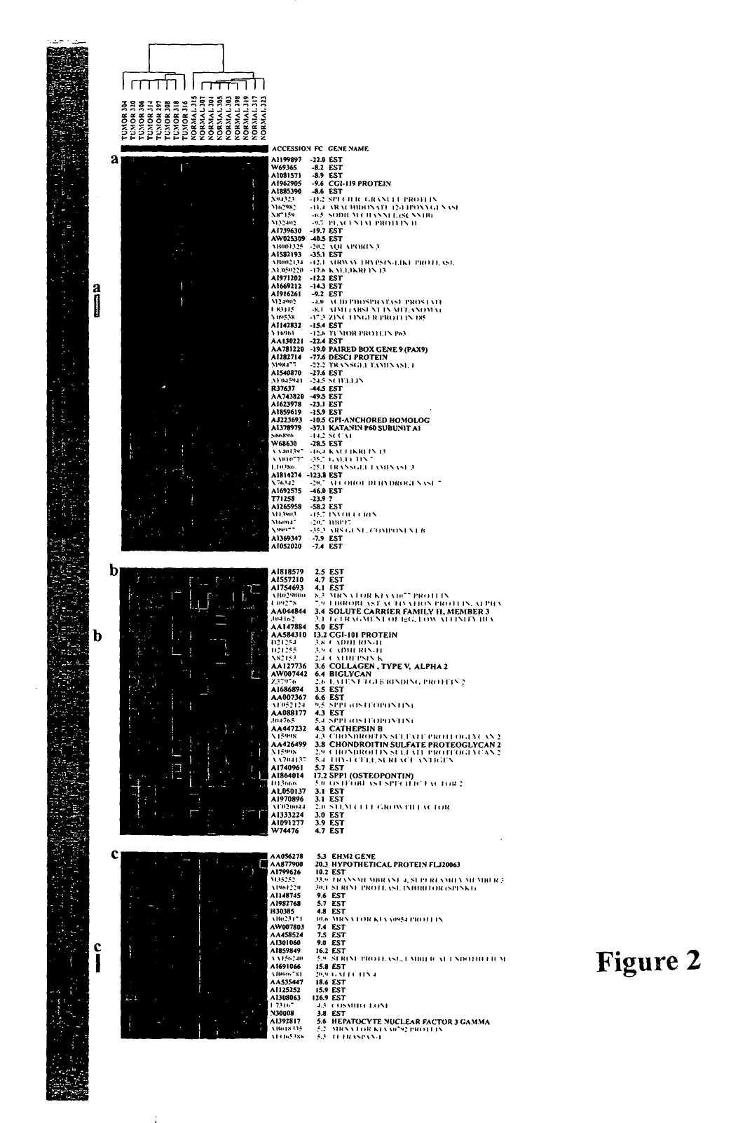

FIG. 2 shows the results obtained from a clustering analysis performed for 4,521 genes from the Human Genome U95 array set. A representative cluster was chosen that contained a number of genes from the U95A FIG. 1 cluster d). Genes in common between clusters are labeled in green. Based on expression similarities to known genes, the biological function of ESTs can be determined. The genes thus identified are listed in Table 8.

The cluster analysis also identified genes not identified in the fold change analysis. Table 7 provides a list of those genes identified as present in the U95A chip cluster analysis but not identified as present in the fold change analysis.

The clusters of genes thus identified contain genes that exhibit a consistent fold change between the normal and diseased sample sets, providing targets for broad range diagnostics and therapeutics.

### Example 6

### Tissue Markers

As the progression from normal esophagus to BA occurs, squamous epithelial cells are replaced with a heterogeneous population of columnar cells that exhibit both intestinal and gastric-like characteristics. The methods of the present invention were used to identify clusters containing genes differentially expressed in all normal or diseased samples. The genes thus identified were screened for the presence of marker genes corresponding to gross morphological changes.

The stratified squamous epithelial terminal differentiation markers, transglutaminase 1, transglutaminase-3, involucrin, envoplakin, periplakin and sciellin were all present in the cluster of genes under-expressed in BA (FIG. 1a). A distinct cluster (FIG. 1c) was also identified that included over-expressed genes associated with the Barrett's esophagus phenotype (see Labouvie, et al., Differential expression of mucins and trefoil peptides in native epithelium, Barrett's metaplasia and squamous cell carcinoma of the oesophagus. J. Cancer Res. Clin. Oncol. 125, 71-6 (1999) and Westerveld, et al., Gastric proteases in Barrett's esophagus. Gastroenterology 93, 774-8 (1987)). The genes trefoil peptide 1(TFF-1), trefoil peptide 2 (TFF-2), mucin 5B, and pepsinogen C were present in this cluster.

### Example 7

### Metastasis-Related Genes

The majority of BA tumors in this study (6 out of 8) displayed regional lymph node invasion. Genes with expression changes that correlate highly with the metastatic phenotype are very valuable diagnostic markers. The first step in metastasis is the loss of cell adhesion at the primary site. Desmosomes are multi-component structures involved in epithelial cell to cell adhesion and intracellular anchoring of intermediate filaments. The desmosomal components, desmoglein 3, desmocollin 2, and desmoplakin, are all present in the cluster of genes under-expressed in BA (FIG. 1a).

Once cell to cell adhesion is broken, the extracellular matrix (ECM) must be breached to enable movement into metastatic sites. A number of proteases, including metalloproteinase 1 (MMP-1), metalloproteinase 11 (MMP-11), cathepsin E, cathepsin K, and urokinase plasminogen activator (u-PA), that are involved in basement membrane and ECM degradation are spread throughout the clusters containing genes over-expressed in BA (FIGS. 1b-1d). MMP-1, MMP-11, and u-PA expression has previously been correlated with metastasis and/or poor prognosis in esophageal carcinoma (see Murray, et al., Matrix metalloproteinase-1 is associated with poor prognosis in oesophageal cancer. J Pathol. 185, 256-61 (1998), Porte, et al., Overexpression of stromelysin-3, BM-40/SPARC, and MET genes in human esophageal carcinoma: implications for prognosis. Clin. Cancer Res. 4, 1375-82 (1998) and Hewin, et al., Plasminogen activators in oesophageal carcinoma. Br. J. Surg. 83, 1152-5 (1996)).

In parallel with the expression increase in ECM proteinases, an expression decrease was seen in a number of proteinase inhibitors, including squamous cell carcinoma antigen 1 (SCCA1), squamous cell carcinoma antigen 2 (SCCA2), cystatin 6, and ELANH2 (FIG. 3A, i). The loss of inhibitory proteinases may allow metastatic tumor progression to occur more rapidly.

As the tumor moves through the stromal compartment into secondary sites, a balance must be reached between ECM degradation and renewal. The tumor requires the break down of ECM components to enable invasion, but the stromal environment must also be altered to create an environment with which the tumor can adhere and migrate. SPARC/osteonectin, SPP1/osteopontin, and thrombospondin-2 are secreted proteins involved in mediating cell to matrix interactions. These genes cluster together (FIG. 1d), and are over-expressed in BA. SPARC, SPP-1, and thrombospondin-1 have previously been linked to oesophageal carcinoma (see Porte, et al., supra, Casson, et al., Ras mutation and expression of the ras-regulated genes osteopontin and cathepsin L in human esophageal cancer. Int. J. Cancer 72, 739-45 (1997) and Oshiba, et al., Stromal thrombospondin-1 expression is correlated with progression of esophageal squamous cell carcinoma Anticancer Res. 19, 4375-8 (1999)).

Further denoting the changes in the stromal environment, the ECM components, chondroitin sulfate proteoglycan 2, collagen type X alpha 1, and collagen type X alpha 1, are also present in this cluster (FIG. 1d).

### Example 8

### Other Gene Clusters

A number of additional clusters, besides those directly related to the metastatic process, have been identified. Reflecting a change in the tumor's transcriptional program, one distinct cluster under-expressed in BA contained the homeobox genes, PITX1, PAX9 and BARX2 (FIG. 1a). The homeobox gene, HOXB7, was over-expressed in BA (FIG. 1b). Homeobox genes are nuclear transcription factors that regulate development. Another cluster contained genes elicited by the body's anti-tumoral immune response FIG. 1b). Two genes induced by interferon alpha and beta, IFI35 and IFI30, two genes induced by interferon gamma, ISG15 and GIP3, and interferon-induced complement component C2 were present in a cluster over-expressed in BA (FIG. 1b). Natural killer transcript 4 (NK4) also clusters with these genes.

### Example 9

### EST Clustering

Clustering was performed for the full Human Genome U95 set. After filtering, 4521 genes (7.5% of the genes present on all 5 arrays) were analyzed via hierarchical clustering and the results are shown in FIG. 2. A list of the genes thus identified is provided in Table 8. A hierarchical clustering was used to measure expression variation for 4,521 known genes or ESTs from the Affymetrix HG-U95 array set. Three clusters are shown that include genes from the HG-U95A analysis (see FIG. 1). Genes in common between clusters are labeled in green. The dendrogram summarizes expression similarities between samples. Each gene and sample presentation is the same as in FIG. 1. The overall fold change (FC), fold change between the groups of tissue samples, are also listed for each gene. Based on expression similarities to known genes, the biological function of some EST's can be assigned. Cluster A represents a number of marker genes for squamous epithelial cells. ESTs grouped around these genes are novel diagnostic markers whose expression loss follows BA progression. Cluster B represents a number of genes involved in ECM modification. Cluster C represents genes involved in cell adhesion, migration, proliferation and differentiation. Interestingly, EST AA877900 clusters around the cell surface protein encoded by tetraspanins and shows homology to the mouse cell surface antigen 114/A10 precursor. The resulting dendrogram grouped all nine normal oesophagus and all eight BA samples into separate trees. FIG. 2 shows the incorporation of these additional genes, consisting primarily of ESTs, into the Human Genome U95A cluster (FIG. 1). The U95A cluster contained a number of proteins involved in extracellular matrix modification and structure. Based on expression similarities to known genes, the biological function of surrounding ESTs can be postulated. Supporting this theory, the extracellular matrix proteins, collagen type V alpha 2, biglycan, and SPP1 (osteopontin) are represented in the new cluster (FIG. 2).

The present invention provides methods to identify genes and ESTs that are differentially expressed in normal and cancerous esophageal tissue. The method entails using several tissues of the same disease type to identify the gene expression patterns that are unique to normal and diseased tissues, comparing these patterns to determine the expression patterns that uniquely identify the disease, and performing fold change analysis to discover which genes are the most important determinants of disease. Applying the method, Applicants have identified key disease-related genes, and furthermore demonstrate that these weighted genes, can be used to identify significant clusters generated by hierarchical clustering algorithms. This overall approach, can potentially determine novel targets for diagnostic and therapeutic intervention in a wide variety of tissues, as demonstrated here with BA.

Although the present invention has been described in detail with reference to examples above, it is understood that various modifications can be made without departing from the spirit of the invention. Accordingly, the invention is limited only by the following claims. All cited patents and publications referred to in this application are herein incorporated by reference in their entirety.

TABLE 1

| | HG-U95A | HG-U95B | HG-U95C | HG-U95D | HG-U95E | Totals |
|---|---|---|---|---|---|---|
| A) | | | | | | |
| No. of genes whose expression was detected in normal esophagus | 5652 | 4394 | 2743 | 699 | 1266 | 14754 |
| No. of genes whose expression was not detected in BA | 5123 | 6158 | 8162 | 10397 | 8017 | 37857 |
| No. of genes uniquely under-expressed in BA | 101 | 62 | 46 | 20 | 15 | 244 |
| B) | | | | | | |
| No. of genes whose expression was detected in BA | 5433 | 4659 | 2772 | 934 | 1667 | 15465 |
| No. of genes whose expression was not detected in normal esophagus | 4170 | 5347 | 7184 | 9621 | 8163 | 34485 |
| No. of genes uniquely over-expressed in BA | 47 | 51 | 19 | 25 | 37 | 179 |
| Sum of genes unique for BA | | | | | | 423 |

A) Affymetrix probe array data was used to determine the number of genes expressed in normal esophagus and the number of genes NOT expressed in Barrett's-associated esophageal adenocarcinoma (BA). Relative to gene expression in normal esophagus, 244 genes were uniquely under-expressed in BA.
B) The number of genes expressed in BA was compared against the number of normal esophagus genes whose expression was NOT detectable. 179 genes were uniquely over-expressed in BA.

TABLE 2
U95_A Fold Change Genes (>3 over-expressed in Barrett's associated
esophageal adenocarcinoma (BA), <0.33 under-expressed in BA)

| Affy ID | Gene Name | Fold Change | P-value |
|---|---|---|---|
| 38469_at | Cluster Incl M35252: Human CO-029 /cds = (137,850) | 33.86361231 | 0.000002 |
| | /gb = M35252 /gi = 180925 /ug = Hs.84072 | | |
| | /len = 1083 | | |
| 38582_at | Cluster Incl AI961220: wt15b04.x1 Homo sapiens cDNA, | 30.1318728 | 0.000014 |
| | 3 end /clone = IMAGE-2507503 /clone_end = 3″ | | |
| | /gb = AI961220 /gi = 5753933 /ug = Hs.181286 /len = 510″ | | |
| 765_s_at | AB006781 /FEATURE = /DEFINITION = AB006781 | 20.91494437 | 0.000002 |
| | Homo sapiens mRNA for galectin-4, complete cds | | |
| 608_at | M12529 /FEATURE = mRNA /DEFINITION = | 16.55132436 | 0 |
| | HUMAPOE Human apolipoprotein E mRNA, complete cds | | |
| 31798_at | Cluster Incl AA314825: EST186646 Homo sapiens cDNA, | 13.40389513 | 0.003038 |
| | 5 end /clone = ATCC-111986 /clone_end = 5″ | | |
| | /gb = AA314825 /gi = 1967154 /ug = Hs.1406 /len = 574″ | | |
| 37186_s_{—} | Cluster Incl U11863: Human clone HP-DAO2 diamine oxidase, | 12.17002487 | 0.000007 |
| | copper/topa quinone containing mRNA, complete cds /cds = (73,2385) | | |
| | /gb = U11863 /gi = 533537 /ug = Hs.75741 /len = 2473 | | |
| 38827_at | Cluster Incl AF038451: Homo sapiens secreted cement gland protein | 11.64190393 | 0.000049 |
| | XAG-2 homolog (hAG-2/R) mRNA, complete cds /cds = (58,585) | | |
| | /gb = AF038451 /gi = 3779225 /ug = Hs.91011 /len = 1059 | | |
| 33327_at | Cluster Incl AB023171: Homo sapiens mRNA for KIAA0954 protein, | 10.57914803 | 0.000175 |
| | partial cds /cds = (0,2682) /gb = AB023171 /gi = 4589551 | | |
| | /ug = Hs.184640 /len = 5057 | | |
| 38239_at | Cluster Incl AI312905: qp84d03.x1 Homo sapiens cDNA, 3 end | 10.52282698 | 0.004857 |
| | /clone = IMAGE-1929701 /clone_end = 3″ /gb = | | |
| | AI312905 /gi = 4018510 /ug = Hs.16762 /len = 493″ | | |
| 41106_at | Cluster Incl AF022797: Homo sapiens intermediate conductance | 9.590462962 | 0.005712 |
| | calcium-activated potassium channel (hKCa4) mRNA, complete cds /cds = | | |
| | (396,1679) /gb = AF022797 /gi = 2674355 /ug = Hs.10082 /len = 2238 | | |
| 34342_s_{—} | Cluster Incl AF052124: Homo sapiens clone 23810 osteopontin mRNA, | 9.533960576 | 0.000045 |
| | complete cds /cds = (87,989) /gb = AF052124 /gi = | | |
| | 3360431 /ug = Hs.313 /len = 1504 | | |
| 575_s_at | M93036 /FEATURE = mRNA /DEFINITION = HUMGA7A08 | 8.991837359 | 0.000002 |
| | Human (clone 21726) carcinoma-associated antigen GA733-2 (GA733-2) | | |
| | mRNA, exon 9 and complete cds | | |
| 35832_at | Cluster Incl AB029000: Homo sapiens mRNA for KIAA1077 protein, | 8.290812823 | 0.000042 |
| | partial cds /cds = (0,2456) /gb = AB029000 /gi = | | |
| | 5689490 /ug = Hs.70823 /len = 4834 | | |
| 32349_at | Cluster Incl AJ238979: Homo sapiens mRNA for annexin A10 protein | 8.241398197 | 0.004318 |
| | (ANXA10 gene) /cds = (117,1091) /gb = AJ238979 /gi = | | |
| | 5689749 /ug = Hs.188401 /len = 1408 | | |
| 271_s_at | J05036 /FEATURE = mRNA /DEFINITION = HUMCTSE | 8.235638183 | 0.000008 |
| | Human cathepsin E mRNA, complete cds | | |
| 39945_at | Cluster Incl U09278: Human fibroblast activation protein mRNA, | 7.883016283 | 0.000057 |
| | complete cds /cds = (208,2490) /gb = U09278 /gi = | | |
| | 1888315 /ug = Hs.418 /len = 2814 | | |
| 1890_at | AB000584 /FEATURE = /DEFINITION = AB000584 Homo sapiens mRNA for | 7.410761415 | 0.000062 |
| | TGF-beta superfamily protein, complete cds | | |
| 37319_at | Cluster Incl M35878: Human insulin-like growth factor-binding | 7.315749117 | 0.00202 |
| | protein-3 gene, complete cds, clone HL1006d /cds = (132,1007) /gb = | | |
| | M35878 /gi = 184522 /ug = Hs.77326 /len = 3446 | | |
| 38124_at | Cluster Incl X55110: Human mRNA for neurite outgrowth-promoting | 7.314122689 | 0.0002 |
| | protein /cds = (25,456) /gb = X55110 /gi = 35086 /ug = | | |
| | Hs.82045 /len = 786 | | |
| 36781_at | Cluster Incl X01683: Human mRNA for alpha 1-antitrypsin /cds = (38,1294) | 7.157108509 | 0.000155 |
| | /gb = X01683 /gi = 28965 /ug = Hs.75621 /len = 1371 | | |
| 38428_at | Cluster Incl M13509: Human skin collagenase mRNA, complete cds /cds = | 6.752025797 | 0.000234 |
| | (68,1477) /gb = M13509 | | |
| | /gi = 180664 /ug = Hs.83169 /len = 1970 | | |
| 33699_at | Cluster Incl M18667: Human pepsinogen C gene /cds = (73,1230) /gb = | 6.70507087 | 0.007673 |
| | M18667 /gi = 189820 /ug = Hs.1867 /len = 1359 | | |
| 41470_at | Cluster Incl AF027208: Homo sapiens AC133 antigen mRNA, complete cds | 6.236624008 | 0.000202 |
| | /cds = (37,2634) /gb = AF027208 /gi = 2688948 /ug = | | |
| | Hs.112360 /len = 3794 | | |
| 1107_s_at | M13755 /FEATURE = mRNA /DEFINITION = HUMIFN15K Human | 6.024908182 | 0.000042 |
| | interferon-induced 17-kDa/15-kDa protein mRNA, complete cds | | |
| 33702_f_a | Cluster Incl L05144: Homo sapiens (clone lamda-hPEC-3) phosphoenolpyruvate | 5.944698576 | 0.000527 |
| | carboxykinase (PCK1) mRNA, complete cds /cds = (121,1989) /gb = | | |
| | L05144 /gi = 189944 /ug = Hs.1872 /len = 2657 | | |
| 38261_at | Cluster Incl AF085692: Homo sapiens multidrug resistance-associated | 5.920947513 | 0.001318 |
| | protein 3B (MRP3) mRNA, complete cds /cds = (36,1568) /gb = | | |
| | AF085692 /gi = 4106443 /ug = Hs.90786 /len = 5327 | | |
| 40079_at | Cluster Incl AA156240: zI50c12.s1 Homo sapiens cDNA, 3 end /clone = | 5.897828308 | 0.000012 |
| | IMAGE-505366 /clone_end = 3″ /gb = AA156240 /gi = | | |
| | 1727858 /ug = Hs.154737 /len = 668″ | | |
| 41585_at | Cluster Incl AB018289: Homo sapiens mRNA for KIAA0746 protein, partial | 5.679597385 | 0.00001 |
| | cds /cds = (0,3091) /gb = AB018289 /gi = 3882212 /ug = | | |
| | Hs.49500 /len = 4086 | | |
| 40201_at | Cluster Incl M76180: Human aromatic amino acid decarboxylase (ddc) mRNA, | 5.655152421 | 0.00268 |
| | complete cds /cds = (69,1511) /gb = M76180 /gi = 181520 | | |
| | /ug = Hs.150403 /len = 1930 | | |
| 39395_at | Cluster Incl AA704137: ag47g01.s1 Homo sapiens cDNA, 3 end /clone = | 5.386956374 | 0.0002 |
| | IMAGE-1119984 /clone_end = 3″ /gb = AA704137 /gi = | | |
| | 2714055 /ug = Hs.125359 /len = 923″ | | |
| 2092_s_a | J04765 /FEATURE = /DEFINITION = HUMOSTRO Human osteopontin | 5.386687312 | 0.008537 |
| | mRNA, complete cds | | |
| 39119_s_{—} | Cluster Incl AA631972: fmfc39 Homo sapiens cDNA /clone = CR7-5 | 5.364323204 | 0.000015 |
| | /gb = AA631972 /gi = 2554583 /ug = Hs.943 /len = 896 | | |
| 34775_at | Cluster Incl AF065388: Homo sapiens tetraspan NET-1 mRNA, complete cds | 5.286352347 | 0.000004 |
| | /cds = (121,846) /gb = AF065388 /gi = 3152700 /ug = | | |
| | Hs.38972 /len = 1278 | | |
| 38181_at | Cluster Incl X57766: Human stromelysin-3 mRNA /cds = (9,1475) | 5.282204273 | 0.001357 |
| | /gb = X57766 /gi = 456256 /ug = Hs.155324 /len = 2247 | | |
| 38615_at | Cluster Incl AF097021: Homo sapiens GW112 protein (GW112) mRNA, complete | 5.282142721 | 0.01537 |
| | cds /cds = (508,1071) /gb = AF097021 /gi = 3860076 | | |
| | /ug = Hs.100347 /len = 2830 | | |
| 33731_at | Cluster Incl AJ130718: Homo sapiens mRNA for glycoprotein-associated | 5.179186526 | 0.001054 |
| | amino acid transporter y + LAT1 /cds = (293,1828) /gb = | | |
| | AJ130718 /gi = 3970724 /ug = Hs.194693 /len = 2214 | | |
| 37117_at | Cluster Incl Z83838: Human DNA sequence from PAC 127B20 on chromosome | 5.160630589 | 0.006643 |
| | 22q11.2-qter, contains gene for GTPase-activating protein similar to | | |
| | rhoGAP protein, ribosomal protein L6 pseudogene, ESTs and CA repeat | | |
| | /cds = (0,926) /gb = Z83838 /gi = 2276307 /ug = | | |
| | Hs.102336 /len = 927 | | |
| 39339_at | Cluster Incl AB018335: Homo sapiens mRNA for KIAA0792 protein, complete | 5.158180582 | 0.000121 |
| | cds /cds = (250,2673) /gb = AB018335 /gi = 3882304 | | |
| | /ug = Hs.119387 /len = 4074 | | |
| 41764_at | Cluster Incl AA976838: oq35c12.s1 Homo sapiens cDNA, 3 end /clone = | 5.012716912 | 0.002917 |
| | IMAGE-1588342 /clone_end = 3″ /gb = AA976838 /gi = | | |
| | 3154284 /ug = Hs.182778 /len = 468″ | | |
| 451_s_at | D13666 /FEATURE = /DEFINITION = HUMOSF2OS Homo sapiens osf-2 | 4.951150996 | 0.000646 |
| | mRNA for osteoblast specific actor 2 (OSF-2os), complete cds | | |
| 36883_at | Cluster Incl X14640: Human mRNA for keratin 13 /cds = (42,1418) | 4.916594286 | 0.000477 |
| | /gb = X14640 /gi = 34032 /ug = Hs.74070 /len = 1691 | | |
| 35703_at | Cluster Incl X06374: Human mRNA for platelet-derived growth factor | 4.875862208 | 0.003643 |
| | PDGF-A /cds = (403,993) /gb = X06374 /gi = 35363 /ug = | | |
| | Hs.37040 /len = 2305 | | |
| 1358_s_a | U22970 /FEATURE = mRNA#1 /DEFINITION = HSU22970 Human | 4.830291781 | 0.000138 |
| | interferon-inducible peptide (6-16) gene, complete cds | | |
| 35822_at | Cluster Incl L15702: Human complement factor B mRNA, complete cds | 4.749292589 | 0.0006 |
| | /cds = (40,2334) /gb = L15702 /gi = 291921 /ug = | | |
| | Hs.69771 /len = 2388 | | |
| 38432_at | Cluster Incl AA203213: zx57e04.r1 Homo sapiens cDNA, 5 end /clone = | 4.746919578 | 0.00033 |
| | IMAGE-446622 /clone_end = 5″ /gb = AA203213 /gi = | | |
| | 1798923 /ug = Hs.833 /len = 879″ | | |
| 40462_at | Cluster Incl AF055022: Homo sapiens clone 24684 mRNA sequence /cds = | 4.654507719 | 0.002269 |
| | UNKNOWN /gb = AF055022 /gi = 3005750 /ug = Hs.168073 | | |
| | /len = 1864 | | |
| 35313_at | Cluster Incl AB002308: Human mRNA for KIAA0310 gene, complete cds | 4.633034993 | 0.000025 |
| | /cds = (1655,4300) /gb = AB002308 /gi = 2224560 | | |
| | /ug = Hs.5716 /len = 5955 | | |
| 40762_g_{—} | Cluster Incl AA705628: zf40a01.s1 Homo sapiens cDNA, 3 end /clone = | 4.598316089 | 0.005112 |
| | 379368 /clone_end = 3″ /gb = AA705628 /gi = 2715546 | | |
| | /ug = Hs.90911 /len = 514″ | | |
| 32306_g_{—} | Cluster Incl J03464: Human collagen alpha-2 type I mRNA, complete cds, | 4.574917305 | 0.000593 |
| | clone pHCOL2A1 /cds = (469,4569) /gb = J03464 /gi = 179595 | | |
| | /ug = Hs.179573 /len = 5416 | | |
| 31901_at | Cluster Incl AF044253: Homo sapiens potassium channel beta 2 subunit | 4.485703929 | 0.000413 |
| | (HKvbeta2.2) mRNA, alternatively spliced, complete cds /cds = | | |
| | (0,1061) /gb = AF044253 /gi = 2827465 /ug = Hs.154417 | | |
| | /len = 1062 | | |
| 35766_at | Cluster Incl M26326: Human keratin 18 mRNA, complete cds /cds = | 4.434133847 | 0.000464 |
| | (51,1343) /gb = M26326 /gi = 186690 /ug = Hs.65114 | | |
| | /len = 1412 | | |
| 36495_at | Cluster Incl U21931: Human fructose-1,6-biphosphatase (FBP1) gene | 4.359840196 | 0.000517 |
| | /cds = (211,1227) /gb = U21931 /gi = 1000077 /ug = | | |
| | Hs.574 /len = 1476 | | |
| 33118_at | Cluster Incl U73167: Homo sapiens cosmid clone LUCA14 from 3p21.3 | 4.325421706 | 0.001122 |
| | /cds = (0,2252) /gb = U73167 /gi = 2880032 /ug = | | |
| | Hs.82222 /len = 2253 | | |
| 38111_at | Cluster Incl X15998: H. sapiens mRNA for the chondroitin sulphate | 4.314531945 | 0.005225 |
| | proteoglycan versican, V1 splice-variant; precursor peptide /cds = | | |
| | (266,7495) /gb = X15998 /gi = 37662 /ug = Hs.81800 | | |
| | /len = 8224 | | |
| 34712_at | Cluster Incl AB023227: Homo sapiens mRNA for KIAA1010 protein, partial | 4.249345789 | 0.006337 |
| | cds /cds = (0,3949) /gb = AB023227 /gi = 4589669 /ug = | | |
| | Hs.23860 /len = 5524 | | |
| 36482_s_{—} | Cluster Incl Y15724: Homo sapiens SERCA3 gene, exons 1-7 (and joined | 4.242412448 | 0.000245 |
| | CDS) /cds = (152,3241) /gb = Y15724 /gi = 3021395 /ug = | | |
| | Hs.5541 /len = 4800 | | |
| 39801_at | Cluster Incl AF046889: Homo sapiens lysyl hydroxylase isoform 3 (PLOD3) | 4.237391894 | 0.001831 |
| | mRNA, complete cds /cds = (216,2432) /gb = AF046889 /gi = | | |
| | 3153234 /ug = Hs.153357 /len = 2735 | | |
| 39959_at | Cluster Incl AL031983: dJ271M21.6 (Diubiquitin) /cds = (18,515) | 4.224484244 | 0.013448 |
| | /gb = AL031983 /gi = 4160199 /ug = Hs.44532 /len = 777 | | |
| 38671_at | Cluster Incl AB014520: Homo sapiens mRNA for KIAA0620 protein, partial cds | 4.204467196 | 0.000003 |
| | /cds = (0,5957) /gb = AB014520 /gi = 3327053 /ug = | | |
| | Hs.105958 /len = 6754 | | |
| 34022_at | Cluster Incl M36821: Human cytokine (GRO-gamma) mRNA, complete cds | 4.186691813 | 0.003842 |
| | /cds = (77,397) /gb = M36821 /gi = 183632 /ug = | | |
| | Hs.89690 /len = 1064 | | |
| 33904_at | Cluster Incl AB000714: Homo sapiens hRVP1 mRNA for RVP1, complete cds | 4.160312065 | 0.009967 |
| | /cds = (198,860) /gb = AB000714 /gi = 2570128 /ug = | | |
| | Hs.25640 /len = 1250 | | |
| 1930_at | U83659 /FEATURE = /DEFINITION = HSU83659 Human multidrug | 4.103745262 | 0.001081 |
| | resistance-associated protein homolog (MRP3) mRNA, partial cds | | |
| 38646_s_{—} | Cluster Incl AI763065: wi64h03.x1 Homo sapiens cDNA, 3 end /clone = | 4.034467285 | 0.045602 |
| | IMAGE-2398133 /clone_end = 3″ /gb = AI763065 /gi = | | |
| | 5178732 /ug = Hs.1032 /len = 670″ | | |
| 35937_at | Cluster Incl U65416: Human MHC class I molecule (MICB) gene, complete cds | 4.031356856 | 0.004886 |
| | /cds = (5,1156) /gb = U65416 /gi = 1815636 /ug = | | |
| | Hs.211580 /len = 2367 | | |
| 36174_at | Cluster Incl X70326: H. sapiens MacMarcks mRNA /cds = (13,600) | 4.021774238 | 0.006155 |
| | /gb = X70326 /gi = 38434 /ug = Hs.75061 /len = 1334 | | |
| 34433_at | Cluster Incl AF035299: Homo sapiens clone 23863 mRNA, partial cds | 3.999547716 | 0.000374 |
| | /cds = (0,1531) /gb = AF035299 /gi = 2661055 /ug = | | |
| | Hs.103854 /len = 1953 | | |
| 34251_at | Cluster Incl M92299: Human homeobox 2.1 protein (HOX2A) mRNA, complete | 3.989655556 | 0.002597 |
| | cds /cds = (275,1084) /gb = M92299 /gi = 184292 /ug = | | |
| | Hs.22554 /len = 2037 | | |
| 1230_g_at | U78556 /FEATURE = /DEFINITION = HSU78556 Human cisplatin | 3.986593947 | 0.007319 |
| | resistance associated alpha protein (hCRA alpha) mRNA, complete cds | | |
| 41812_s_{—} | Cluster Incl AB020713: Homo sapiens mRNA for KIAA0906 protein, partial cds | 3.955302442 | 0.001398 |
| | /cds = (0,2772) /gb = AB020713 /gi = 4240300 /ug = | | |
| | Hs.56966 /len = 4217 | | |
| 33489_at | Cluster Incl U47292: Human spasmolytic polypeptide (SP) gene, 5 region | 3.951274898 | 0.000058 |
| | and /cds = (0,389) /gb = U47292 /gi = 1477544 /ug = | | |
| | Hs.2979 /len = 965″ | | |
| 40712_at | Cluster Incl D26579: Homo sapiens mRNA for transmembrane protein, complete | 3.926351461 | 0.000403 |
| | cds /cds = (9,2483) /gb = D26579 /gi = 1864004 /ug = | | |
| | Hs.86947 /len = 3236 | | |
| 33322_i_a | Cluster Incl X57348: H. sapiens mRNA (clone 9112) /cds = (165,911) | 3.918698039 | 0.000123 |
| | /gb = X57348 /gi = 23939 /ug = Hs.184510 /len = 1407 | | |
| 36976_at | Cluster Incl D21255: Human mRNA for OB-cadherin-2, complete cds /cds = | 3.912909005 | 0.000009 |
| | (476,2557) /gb = D21255 /gi = 575578 /ug = Hs.75929 | | |
| | /len = 3867 | | |
| 39340_at | Cluster Incl M16424: Human beta-hexosaminidase alpha chain (HEXA) gene | 3.895118855 | 0 |
| | /cds = (0,1589) /gb = M16424 /gi = 179457 /ug = | | |
| | Hs.119403 /len = 1590 | | |
| 1586_at | M35878 /FEATURE = expanded_cds /DEFINITION = HUMIBP3 | 3.855842022 | 0.010079 |
| | Human insulin-like growth factor-binding protein-3 gene, complete cds, | | |
| | clone HL1006d | | |
| 925_at | J03909 /FEATURE = /DEFINITION = HUMIIP Human gamma- | 3.854414845 | 0.000001 |
| | interferon-inducible protein (IP-30) mRNA, complete cds | | |
| 37276_at | Cluster Incl U51903: Human RasGAP-related protein (IQGAP2) mRNA, complete | 3.836460946 | 0.004826 |
| | cds /cds = (222,4949) /gb = U51903 /gi = 1262925 /ug = | | |
| | Hs.78993 /len = 5767 | | |
| 37618_at | Cluster Incl M16937: Human homeo box c1 protein, mRNA, complete cds | 3.827480523 | 0.000352 |
| | /cds = (99,752) /gb = M16937 /gi = 184300 /ug = | | |
| | Hs.819 /len = 1359 | | |
| 32609_at | Cluster Incl AI885852: wl62d08.x1 Homo sapiens cDNA, 3 end /clone = | 3.790091828 | 0.000766 |
| | IMAGE-2429487 /clone_end = 3″ /gb = AI885852 /gi = | | |
| | 5591016 /ug = Hs.795 /len = 580″ | | |
| 40118_at | Cluster Incl X07290: Human HF.12 gene mRNA /cds = (0,589) /gb = | 3.770379359 | 0.000033 |
| | X07290 /gi = 32072 /ug = Hs.155470 /len = 1801 | | |
| 2087_s_at | D21254 /FEATURE = /DEFINITION = HUMOSF4A Human mRNA for | 3.756489413 | 0.001994 |
| | OB-cadherin-1, complete cds | | |
| 1482_g_at | L23808 /FEATURE = /DEFINITION = HUMHME Human metalloproteinase | 3.724668585 | 0.001197 |
| | (HME) mRNA, complete cds | | |
| 182_at | U01062 /FEATURE = mRNA /DEFINITION = HUMIP3R3 Human type 3 | 3.723090192 | 0.000948 |
| | inositol 1,4,5-trisphosphate receptor (ITPR3) mRNA, complete cds | | |
| 39542_at | Cluster Incl AF059611: Homo sapiens nuclear matrix protein NRP/B (NRPB) | 3.700905422 | 0.000005 |
| | mRNA, complete cds /cds = (399,2168) /gb = AF059611 /gi = | | |
| | 3309572 /ug = Hs.104925 /len = 4784 | | |
| 32072_at | Cluster Incl U40434: Human mesothelin or CAK1 antigen precursor mRNA, | 3.700537751 | 0.019363 |
| | complete cds /cds = (99,1985) /gb = U40434 /gi = 1145723 | | |
| | /ug = Hs.155981 /len = 2114 | | |
| 36197_at | Cluster Incl Y08374: H. sapiens gene encoding cartilage GP-39 protein, | 3.695365733 | 0.000483 |
| | exon 1 and 2 (and joined CDS) /cds = (126,1277) /gb = Y08374 | | |
| | /gi = 2121309 /ug = Hs.75184 /len = 1925 | | |
| 36184_at | Cluster Incl L06419: Homo sapiens lysyl hydroxylase (PLOD) mRNA, complete | 3.684437007 | 0.000536 |
| | cds /cds = (200,2383) /gb = L06419 /gi = 190073 /ug = | | |
| | Hs.75093 /len = 3115 | | |
| 35362_at | Cluster Incl AB018342: Homo sapiens mRNA for KIAA0799 protein, partial | 3.622214911 | 0.000505 |
| | cds /cds = (0,2138) /gb = AB018342 /gi = 3882318 /ug = | | |
| | Hs.61638 /len = 5613 | | |
| 39649_at | Cluster Incl X78817: H. sapiens partial C1 mRNA /cds = (42,2882) | 3.589352648 | 0.006513 |
| | /gb = X78817 /gi = 840785 /ug = Hs.3109 /len = 3236 | | |
| 1693_s_at | D11139 /FEATURE = exons #1-4 /DEFINITION = | 3.581791533 | 0.001006 |
| | HUMTIMP Human gene for tissue inhibitor of metalloproteinases, partial sequence | | |
| 38566_at | Cluster Incl X60382: H. sapiens COL10A1 gene for collagen (alpha-1 type X) | 3.568272096 | 0.023539 |
| | /cds = (0,2042) /gb = X60382 /gi = 30094 /ug = | | |
| | Hs.179729 /len = 3198 | | |
| 39043_at | Cluster Incl AF006084: Homo sapiens Arp2/3 protein complex subunit | 3.563560014 | 0.000113 |
| | p41-Arc (ARC41) mRNA, complete cds /cds = (80,1198) /gb = | | |
| | AF006084 /gi = 2282033 /ug = Hs.11538 /len = 1408 | | |
| 35926_s_{—} | Cluster Incl AF004230: Homo sapiens monocyte/macrophage Ig-related | 3.560518932 | 0.019359 |
| | receptor MIR-7 (MIR cl-7) mRNA, complete cds /cds = (170,2125) | | |
| | /gb = AF004230 /gi = 2343108 /ug = Hs.204040 /len = 2882 | | |
| 38332_at | Cluster Incl U83993: Human P2X4 purinoreceptor mRNA, complete cds | 3.540548694 | 0.000001 |
| | /cds = (309,1475) /gb = U83993 /gi = 4099120 /ug = | | |
| | Hs.9610 /len = 2031 | | |
| 37542_at | Cluster Incl D86961: Human mRNA for KIAA0206 gene, partial cds /cds = | 3.531781728 | 0.00014 |
| | (0,581) /gb = D86961 /gi = 1503995 /ug = Hs.79299 | | |
| | /len = 4249 | | |
| 33374_at | Cluster Incl L09708: Human complement component 2 (C2) gene allele b | 3.50128085 | 0.043555 |
| | /cds = (271,2529) /gb = L09708 /gi = 2804581 /ug = | | |
| | Hs.2253 /len = 2844 | | |
| 35372_r_a | Cluster Incl M17017: Human beta-thromboglobulin-like protein mRNA, | 3.492697198 | 0.004805 |
| | complete cds /cds = (90,389) /gb = M17017 /gi = 179579 | | |
| | /ug = Hs.624 /len = 1639 | | |
| 38464_at | Cluster Incl X87237: H. sapiens mRNA for processing a-glucosidase I | 3.488460577 | 0.000501 |
| | /cds = (132,2642) /gb = X87237 /gi = 2344809 /ug = | | |
| | Hs.83919 /len = 2854 | | |
| 33730_at | Cluster Incl AF095448: Homo sapiens putative G protein-coupled receptor | 3.420612984 | 0.000001 |
| | (RAIG1) mRNA, complete cds /cds = (99,1172) /gb = AF095448 | | |
| | /gi = 4063889 /ug = Hs.194691 /len = 2288 | | |
| 37310_at | Cluster Incl X02419: H. sapiens uPA gene /cds = (119,1414) /gb = | 3.389908873 | 0.000181 |
| | X02419 /gi = 37601 /ug = Hs.77274 /len = 2344 | | |
| 32305_at | Cluster Incl J03464: Human collagen alpha-2 type I mRNA, complete cds, | 3.385860182 | 0.00189 |
| | clone pHCOL2A1 /cds = (469,4569) /gb = J03464 /gi = 179595 | | |
| | /ug = Hs.179573 /len = 5416 | | |
| 38388_at | Cluster Incl M11810: Human (2-5″) oligo A synthetase E gene | 3.354436847 | 0.00674 |
| | /cds = (0,1202) /gb = M11810 /gi = 189323 /ug = | | |
| | Hs.82396 /len = 1552″ | | |
| 37423_at | Cluster Incl U30246: Human bumetanide-sensitive Na—K—Cl | 3.349808467 | 0.003674 |
| | cotransporter (NKCC1) mRNA, complete cds /cds = (164,3802) /gb = | | |
| | U30246 /gi = 903681 /ug = Hs.110736 /len = 4098 | | |
| 464_s_at | U72882 /FEATURE = /DEFINITION = HSU72882 Human Interferon-induced | 3.328892723 | 0.000109 |
| | leucine zipper protein (IFP35) mRNA, partial cds | | |
| 36732_at | Cluster Incl AI004207: ot94g05.x1 Homo sapiens cDNA, 3 end /clone = | 3.3206594 | 0.004758 |
| | IMAGE-1624472 /clone_end = 3″ /gb = AI004207 /gi = | | |
| | 3213717 /ug = Hs.55879 /len = 517″ | | |
| 32629_f_a | Cluster Incl U90552: Human butyrophilin (BTF5) mRNA, complete cds | 3.282634677 | 0.015555 |
| | /cds = (359,1900) /gb = U90552 /gi = 2062705 /ug = | | |
| | Hs.167740 /len = 3416 | | |
| 38607_at | Cluster Incl AF027204: Homo sapiens putative tetraspan transmembrane | 3.279897454 | 0.001734 |
| | protein L6H (TM4SF5) mRNA, complete cds /cds = (32,625) /gb = | | |
| | AF027204 /gi = 2587053 /ug = Hs.184194 /len = 708 | | |
| 35355_at | Cluster Incl AB020697: Homo sapiens mRNA for KIAA0890 protein, complete | 3.279064388 | 0.00002 |
| | cds /cds = (143,3727) /gb = AB020697 /gi = 4240268 | | |
| | /ug = Hs.6141 /len = 3800 | | |
| 39230_at | Cluster Incl AL022318: bK150C2.2 (Phorbolin 3) /cds = (29,1177) | 3.24749805 | 0.000479 |
| | /gb = AL022318 /gi = 4826439 /ug = Hs.226307 /len = 1512 | | |
| 33813_at | Cluster Incl AI813532: wj83a09.x1 Homo sapiens cDNA, 3 end /clone = | 3.24556325 | 0.001423 |
| | IMAGE-2409400 /clone_end = 3″ /gb = AI813532 /gi = | | |
| | 5424738 /ug = Hs.204298 /len = 733″ | | |
| 40333_at | Cluster Incl U43842: Homo sapiens bone morphogenetic protein-4 (hBMP-4) | 3.244861857 | 0.003231 |
| | gene, complete cds /cds = (435,1661) /gb = U43842 /gi = | | |
| | 3850194 /ug = Hs.68879 /len = 1946 | | |
| 38064_at | Cluster Incl X79882: H. sapiens Irp mRNA /cds = (105,2795) | 3.242458638 | 0.000285 |
| | /gb = X79882 /gi = 895839 /ug = Hs.80680 /len = 2816 | | |
| 36642_at | Cluster Incl J00287: Human pepsinogen gene /cds = (55,1221) | 3.238347007 | 0.004957 |
| | /gb = J00287 /gi = 189798 /ug = Hs.75558 /len = 1381 | | |
| 658_at | L12350 /FEATURE = mRNA /DEFINITION = HUMTHRSPO Human | 3.207772873 | 0.013942 |
| | thrombospondin 2 (THBS2) mRNA, complete cds | | |
| 35345_at | Cluster Incl X83618: H. sapiens mRNA for 3-hydroxy-3-methylglutaryl | 3.189979043 | 0.000053 |
| | coenzyme A synthase /cds = (51,1577) /gb = X83618 /gi = | | |
| | 619876 /ug = Hs.59889 /len = 2043 | | |
| 36194_at | Cluster Incl M63959: Human alpha-2-macroglobulin receptor-associated | 3.189185993 | 0.004792 |
| | protein mRNA, complete cds /cds = (13,1086) /gb = M63959 | | |
| | /gi = 177873 /ug = Hs.75140 /len = 1493 | | |
| 37637_at | Cluster Incl U27655: Human RGP3 mRNA, complete cds /cds = (287,1846) | 3.161911203 | 0.001763 |
| | /gb = U27655 /gi = 1216368 /ug = Hs.82294 /len = 2638 | | |
| 37200_at | Cluster Incl J04162: Human leukocyte IgG receptor (Fc-gamma-R) mRNA, | 3.134424959 | 0.00002 |
| | complete cds /cds = (17,718) /gb = J04162 /gi = 183036 | | |
| | /ug = Hs.763 /len = 1977 | | |
| 33127_at | Cluster Incl U89942: Human lysyl oxidase-related protein (WS9-14) mRNA, | 3.13386346 | 0.020488 |
| | complete cds /cds = (247,2571) /gb = U89942 /gi = 1890107 | | |
| | /ug = Hs.83354 /len = 3432 | | |
| 33890_at | Cluster Incl AB008109: Homo sapiens mRNA for RGS5, complete cds | 3.122107461 | 0.000677 |
| | /cds = (81,626) /gb = AB008109 /gi = 2554613 /ug = | | |
| | Hs.24950 /len = 2076 | | |
| 38363_at | Cluster Incl W60864: zd27g05.s1 Homo sapiens cDNA, 3 end /clone = | 3.115921272 | 0.0026 |
| | IMAGE-341912 /clone_end = 3″ /gb = W60864 /gi = | | |
| | 1367661 /ug = Hs.9963 /len = 541″ | | |
| 39167_r_a | Cluster Incl D83174: Human mRNA for collagen binding protein 2, complete | 3.112282703 | 0.000802 |
| | cds /cds = (87,1343) /gb = D83174 /gi = 1199486 /ug = | | |
| | Hs.9930 /len = 2047 | | |
| 31856_at | Cluster Incl Z24680: H. sapiens garp gene mRNA, complete CDS /cds = | 3.10615755 | 0.000566 |
| | (94,2082) /gb = Z24680 /gi = 439295 /ug = Hs.151641 | | |
| | /len = 4153 | | |
| 1717_s_at | U45878 /FEATURE = /DEFINITION = HSU45878 Human inhibitor of | 3.096213742 | 0.001867 |
| | apoptosis protein 1 mRNA, complete cds | | |
| 659_g_at | L12350 /FEATURE = mRNA /DEFINITION = HUMTHRSPO Human | 3.089235008 | 0.007846 |
| | thrombospondin 2 (THBS2) mRNA, complete cds | | |
| 753_at | D86425 /FEATURE = /DEFINITION = D86425 Homo sapiens mRNA | 3.077550233 | 0.001355 |
| | for osteonidogen, complete cds | | |
| 32193_at | Cluster Incl AF030339: Homo sapiens receptor for viral semaphorin | 3.076341896 | 0.001147 |
| | protein (VESPR) mRNA, complete cds /cds = (249,4955) /gb = | | |
| | AF030339 /gi = 3176761 /ug = Hs.184697 /len = 5121 | | |
| 38612_at | Cluster Incl M69023: Human globin gene /cds = UNKNOWN /gb = | 3.067377921 | 0.000648 |
| | M69023 /gi = 183127 /ug = Hs.100090 /len = 1119 | | |
| 32634_s_{—} | Cluster Incl U38260: Human islet cell autoantigen ICAp69 mRNA, complete | 3.064088746 | 0.002374 |
| | cds /cds = (169,942) /gb = U38260 /gi = 1675205 /ug = | | |
| | Hs.167927 /len = 1415 | | |
| 38311_at | Cluster Incl AF055012: Homo sapiens clone 24615 mRNA sequence /cds = | 3.057506025 | 0.000007 |
| | UNKNOWN /gb = AF055012 /gi = 3005735 /ug = Hs.94785 | | |
| | /len = 1757 | | |
| 34871_at | Cluster Incl W30677: zb75h10.r1 Homo sapiens cDNA, 5 end /clone = | 3.046742301 | 0.00934 |
| | IMAGE-309475 /clone_end = 5″ /gb = W30677 /gi = 1311730 | | |
| | /ug = Hs.5019 /len = 614″ | | |
| 32128_at | Cluster Incl Y13710: Homo sapiens mRNA for alternative activated | 3.044427861 | 0.001745 |
| | macrophage specific CC chemokine 1 /cds = (70,339) /gb = | | |
| | Y13710 /gi = 2326515 /ug = Hs.16530 /len = 779 | | |
| 39421_at | Cluster Incl D43969: Human AML1 mRNA for AML1c protein (alternatively | 3.036973884 | 0.001109 |
| | spliced product), complete cds /cds = (444,1886) /gb = D43969 | | |
| | /gi = 966998 /ug = Hs.129914 /len = 6212 | | |
| 41354_at | Cluster Incl U25997: Homo sapiens stanniocalcin precursor (STC) mRNA, | 3.032061085 | 0.001245 |
| | complete cds /cds = (284,1027) /gb = U25997 /gi = 3006202 | | |
| | /ug = Hs.25590 /len = 3881 | | |
| 35064_at | Cluster Incl X81006: H. sapiens HCG I mRNA /cds = UNKNOWN /gb = | 3.024314786 | 0.005685 |
| | X81006 /gi = 531406 /ug = Hs.104114 /len = 1224 | | |
| 35127_at | Cluster Incl AI039144: ox31b09.s1 Homo sapiens cDNA, 3 end /clone = | 3.011925912 | 0.024025 |
| | IMAGE-1657913 /clone_end = 3″ /gb = AI039144 /gi = 3278338 | | |
| | /ug = Hs.121017 /len = 527″ | | |
| 31720_s_{—} | Cluster Incl M10905: Human cellular fibronectin mRNA /cds = (0,2383) | 0.329485955 | 0.002116 |
| | /gb = M10905 /gi = 182696 /ug = Hs.118162 /len = 2384 | | |
| 32618_at | Cluster Incl X93086: H. sapiens mRNA for biliverdin IX alpha reductase | 0.329108511 | 0.00447 |
| | /cds = (60,950) /gb = X93086 /gi = 1246748 /ug = Hs.81029 | | |
| | /len = 1053 | | |
| 36684_at | Cluster Incl M21154: Human S-adenosylmethionine decarboxylase mRNA, complete cds | 0.328867318 | 0.001013 |
| | /cds = (248,1252) /gb = M21154 /gi = 178517 /ug = Hs.75744 | | |
| | /len = 1805 | | |
| 32668_at | Cluster Incl AL080076: Homo sapiens mRNA; cDNA DKFZp564C0362 (from clone | 0.32860867 | 0.000001 |
| | DKFZp564C0362) /cds = (59,1144) /gb = AL080076 /gi = 5262486 | | |
| | /ug = Hs.169833 /len = 1711 | | |
| 38158_at | Cluster Incl D79987: Human mRNA for KIAA0165 gene, complete cds /cds = | 0.328288405 | 0.000035 |
| | (1113,6500) /gb = D79987 /gi = 1136391 /ug = Hs.153479 | | |
| | /len = 6662 | | |
| 31525_s_{—} | Cluster Incl J00153: Human alpha globin gene cluster on chromosome 16- zeta | 0.328281737 | 0.008516 |
| | gene /cds = (0,428) /gb = J00153 /gi = 183794 /ug = | | |
| | Hs.182374 /len = 429 | | |
| 38010_at | Cluster Incl AF002697: Homo sapiens E1B 19K/Bcl-2-binding protein Nip3 mRNA, | 0.328241183 | 0.005897 |
| | nuclear gene encoding mitochondrial protein, complete cds /cds = (126,710) | | |
| | /gb = AF002697 /gi = 2511528 /ug = Hs.79428 /len = 1518 | | |
| 33559_at | Cluster Incl U61412: Human non-receptor type protein tyrosine kinase (PTK6) | 0.328077412 | 0.003401 |
| | gene /cds = (813,2168) /gb = U61412 /gi = 3551752 /ug = | | |
| | Hs.51133 /len = 3527 | | |
| 41320_s_{—} | Cluster Incl U69609: Human transcriptional represser (GCF2) mRNA, complete cds | 0.327287446 | 0.0018 |
| | /cds = (124,2382) /gb = U69609 /gi = 3421044 /ug = Hs.239894 | | |
| | /len = 3505 | | |
| 39120_at | Cluster Incl AA224832: nc33b06.s1 Homo sapiens cDNA/clone = IMAGE-1009907 | 0.327269934 | 0.000091 |
| | /gb = AA224832 /gi = 1846120 /ug = Hs.94360 /len = 447 | | |
| 36214_at | Cluster Incl U70663: Human zinc finger transcription factor hEZF (EZF) mRNA, | 0.326147693 | 0.003789 |
| | complete cds /cds = (503,1915) /gb = U70663 /gi = 1857160 | | |
| | /ug = Hs.236377 /len = 1953 | | |
| 38508_s_{—} | Cluster Incl U89337: Human HLA class III region containing cAMP response | 0.325801777 | 0.03159 |
| | element binding protein-related protein (CREB-RP) and tenascin X (tenascin-X) | | |
| | genes, complete cds /cds = (0,12869) /gb = U89337 /gi = 1841544 | | |
| | /ug = Hs.169886 /len = 12870 | | |
| 41257_at | Cluster Incl D16217: Human mRNA for calpastatin, complete cds /cds = | 0.32559376 | 0.014175 |
| | (162,2288) /gb = D16217 /gi = 303598 /ug = Hs.226067 | | |
| | /len = 2493 | | |
| 38407_r_a | Cluster Incl AI207842: ao89h09.x1 Homo sapiens cDNA, 3 end /clone = | 0.325385636 | 0.038443 |
| | IMAGE-1953089 /clone_end = 3″ /gb = AI207842 /gi = 3769784 | | |
| | /ug = Hs.8272 /len = 771″ | | |
| 39159_at | Cluster Incl X99656: H. sapiens mRNA for protein containing SH3 domain, SH3GL1 | 0.324435334 | 0.000015 |
| | /cds = (15,1121) /gb = X99656 /gi = 1869809 /ug = Hs.97616 | | |
| | /len = 2349 | | |
| 853_at | S74017 /FEATURE = /DEFINITION = S74017 Nrf2 = NF-E2-like basic | 0.323486825 | 0.002917 |
| | leucine zipper transcriptional activator [human, hemin-induced K562 cells, | | |
| | mRNA, 2304 nt] | | |
| 33839_at | Cluster Incl D26350: Human mRNA for type 2 inositol 1,4,5-trisphosphate receptor, | 0.322681628 | 0.014546 |
| | complete cds /cds = (418,8523) /gb = D26350 /gi = 450468 | | |
| | /ug = Hs.238272 /len = 10524 | | |
| 39693_at | Cluster Incl N53547: yv43b12.s1 Homo sapiens cDNA, 3 end /clone = | 0.320728331 | 0.014542 |
| | IMAGE-245471 /clone_end = 3″ /gb = N53547 /gi = 1194713 | | |
| | /ug = Hs.13662 /len = 665″ | | |
| 40508_at | Cluster Incl AF025887: Homo sapiens glutathione S-transferase A4-4 (GSTA4) mRNA, | 0.320203001 | 0.011333 |
| | complete cds /cds = (73,741) /gb = AF025887 /gi = 3046391 | | |
| | /ug = Hs.169907 /len = 1260 | | |
| 1590_s_at | J00277 /FEATURE = cds /DEFINITION = HUMRASH Human (genomic clones | 0.319460667 | 0.001811 |
| | lambda-[SK2-T2, HS578T]; cDNA clones RS-[3,4,6]) c-Ha-ras1 | | |
| | proto-oncogene, complete coding sequence | | |
| 351_f_at | D28423 /FEATURE = /DEFINITION = HUMPSF82 Human mRNA for pre-mRNA | 0.318411751 | 0.000636 |
| | splicing factor SRp20, 5″ UTR (sequence from the 5″ cap to the start | | |
| | codon) | | |
| 871_s_at | M95585 /FEATURE = mRNA /DEFINITION = HUMHLF Human hepatic leukemia | 0.317171499 | 0.001365 |
| | factor (HLF) mRNA, complete cds | | |
| 33113_at | Cluster Incl U65093: Human msg1-related gene 1 (mrg1) mRNA, complete cds | 0.316986667 | 0.00001 |
| | /cds = (199,840) /gb = U65093 /gi = 1853998 /ug = Hs.82071 | | |
| | /len = 899 | | |
| 32380_at | Cluster Incl Z34974: H. sapiens mRNA for plakophilin (partial) /cds = | 0.316462158 | 0.01096 |
| | (252,2432) /gb = Z34974 /gi = 550114 /ug = Hs.198382 | | |
| | /len = 2680 | | |
| 863_g_at | U04313 /FEATURE = /DEFINITION = HSU04313 Human maspin mRNA, | 0.316356159 | 0.000203 |
| | complete cds | | |
| 32666_at | Cluster Incl U19495: Human intercrine-alpha (hIRH) mRNA, complete cds | 0.316156704 | 0.000461 |
| | /cds = (473,742) /gb = U19495 /gi = 1754834 /ug = Hs.169672 | | |
| | /len = 2244 | | |
| 34197_at | Cluster Incl X80907: H. sapiens mRNA for p85 beta subunit of phosphatidyl- | 0.314829826 | 0.004387 |
| | inositol-3-kinase /cds = (241,2427) /gb = X80907 /gi = 2160047 | | |
| | /ug = Hs.211586 /len = 3201 | | |
| 39908_at | Cluster Incl AF069735: Homo sapiens PCAF associated factor 65 alpha mRNA, | 0.314036765 | 0.001203 |
| | complete cds /cds = (0,1868) /gb = AF069735 /gi = 3335558 | | |
| | /ug = Hs.131846 /len = 1869 | | |
| 33421_s_{—} | Cluster Incl AB016247: Homo sapiens mRNA for sterol-C5-desaturase, complete | 0.313717219 | 0.000009 |
| | cds /cds = (81,980) /gb = AB016247 /gi = 3721881 /ug = | | |
| | Hs.227947 /len = 2104 | | |
| 39669_at | Cluster Incl AJ009985: Homo sapiens mRNA for annexin 31 /cds = (436,1452) | 0.312453495 | 0.002723 |
| | /gb = AJ009985 /gi = 3688369 /ug = Hs.3346 /len = 1762 | | |
| 36496_at | Cluster Incl AF014398: Homo sapiens myo-inositol monophosphatase 2 mRNA, complete | 0.310618357 | 0.002251 |
| | cds /cds = (141,1007) /gb = AF014398 /gi = 2406665 /ug = | | |
| | Hs.5753 /len = 1428 | | |
| 40684_at | Cluster Incl U78190: Human GTP cyclohydrolase I feedback regulatory protein gene, | 0.310587584 | 0.000134 |
| | complete cds /cds = (94,348) /gb = U78190 /gi = 1698996 | | |
| | /ug = Hs.83081 /len = 712 | | |
| 32363_at | Cluster Incl AF059214: Homo sapiens cholesterol 25-hydroxylase mRNA, complete cds | 0.30897539 | 0.000255 |
| | /cds = (10,828) /gb = AF059214 /gi = 4038307 /ug = Hs.194687 | | |
| | /len = 1360 | | |
| 39364_s_{—} | Cluster Incl Y18207: Homo sapiens mRNA for protein phosphatase 1 (PPP1R5) | 0.308335729 | 0.000446 |
| | /cds = (91,1044) /gb = Y18207 /gi = 3805818 /ug = Hs.12112 | | |
| | /len = 1158 | | |
| 33305_at | Cluster Incl M93056: Human mononcyte/neutrophil elastase inhibitor mRNA sequence | 0.307283402 | 0.000768 |
| | /cds = UNKNOWN /gb = M93056 /gi = 188621 /ug = Hs.183583 | | |
| | /len = 1298 | | |
| 37662_at | Cluster Incl AI701164: we10g07.x1 Homo sapiens cDNA, 3 end /clone = | 0.306979084 | 0.000015 |
| | IMAGE-2340732 /clone_end = 3″ /gb = AI701164 /gi = 4989064 | | |
| | /ug = Hs.78563 /len = 777″ | | |
| 1490_at | M19720 /FEATURE = mRNA#2 /DEFINITION = HUMMYC3L Human L-myc | 0.306261149 | 0.000001 |
| | protein gene, complete cds | | |
| 34986_at | Cluster Incl AF030455: Homo sapiens epithelial V-like antigen precursor (EVA) | 0.305646698 | 0 |
| | mRNA, complete cds /cds = (119,766) /gb = AF030455 /gi = 3169829 | | |
| | /ug = Hs.151602 /len = 1342 | | |
| 41359_at | Cluster Incl Z98265: Homo sapiens mRNA for plakophilin 3 /cds = (74,2467) | 0.304122892 | 0.014199 |
| | /gb = Z98265 /gi = 4995640 /ug = Hs.26557 /len = 2808 | | |
| 31737_at | Cluster Incl J00068: Human adult skeletal muscle alpha-actin mRNA /cds = | 0.304080909 | 0.002586 |
| | (103,1236) /gb = J00068 /gi = 178028 /ug = Hs.1288 | | |
| | /len = 1374 | | |
| 33678_i_a | Cluster Incl X02344: Homo sapiens beta 2 gene /cds = (0,1337) /gb = | 0.303098003 | 0.003362 |
| | X02344 /gi = 37493 /ug = Hs.184582 /len = 1338 | | |
| 38086_at | Cluster Incl AB007935: Homo sapiens mRNA for KIAA0466 protein, partial cds | 0.303085926 | 0.000129 |
| | /cds = (0,2104) /gb = AB007935 /gi = 3413893 /ug = | | |
| | Hs.81234 /len = 4974 | | |
| 36040_at | Cluster Incl AI337192: qx88h10.x1 Homo sapiens cDNA, 3 end /clone = | 0.302572056 | 0.000013 |
| | IMAGE-2009635 /clone_end = 3″ /gb = AI337192 /gi = 4074119 | | |
| | /ug = Hs.47438 /len = 925″ | | |
| 35773_i_a | Cluster Incl AA527880: nh86h10.s1 Homo sapiens cDNA, 3 end /clone = | 0.301976692 | 0.000118 |
| | IMAGE-965443 /clone_end = 3″ /gb = AA527880 /gi = 2269949 | | |
| | /ug = Hs.661 /len = 568″ | | |
| 33369_at | Cluster Incl AI535653: P9-C4.T3.P9.D4 Homo sapiens cDNA, 3 end /clone_end = | 0.301863032 | 0.000092 |
| | 3″ /gb = AI535653 | | |
| | /gi = 4449788 /ug = Hs.223018 /len = 590″ | | |
| 40403_at | Cluster Incl Z97171: Homo sapiens GLC1A (trabecular meshwork induced glucocortcoid | 0.301281577 | 0.000355 |
| | response) gene, exon I, joined CDS /cds = (77,1591) /gb = Z97171 | | |
| | /gi = 2425156 /ug = Hs.78454 /len = 2262 | | |
| 36916_at | Cluster Incl X74570: H. sapiens mRNA for Gal-beta(1-3/1-4)GlcNAc | 0.301200654 | 0 |
| | alpha-2.3-sialyltransferase /cds = (162,1151) /gb = X74570 /gi = | | |
| | 414890 /ug = Hs.75268 /len = 1741 | | |
| 32570_at | Cluster Incl L76465: Homo sapiens NAD+-dependent 15 hydroxyprostaglandin | 0.300349654 | 0.003846 |
| | dehydrogenase (PGDH) mRNA, complete cds /cds = (17,817) /gb = L76465 | | |
| | /gi = 1203983 /ug = Hs.77348 /len = 2518 | | |
| 31932_f_a | Cluster Incl M90357: Human basic transcription factor 3a (BTF3a) gene /cds = | 0.299724858 | 0.000004 |
| | (0,476) /gb = M90357 /gi = 457435 /ug = Hs.166033 /len = 487 | | |
| 37225_at | Cluster Incl D79994: Human mRNA for KIAA0172 gene, partial cds /cds = | 0.297788145 | 0.000001 |
| | (0,3923) /gb = D79994 /gi = 1136403 /ug = Hs.77546 | | |
| | /len = 4792 | | |
| 40155_at | Cluster Incl D31883: Human mRNA for KIAA0059 gene, complete cds /cds = | 0.297603964 | 0.000298 |
| | (221,1609) /gb = D31883 /gi = 505093 /ug = Hs.158203 | | |
| | /len = 6754 | | |
| 31605_at | Cluster Incl U72518: Human destrin-2 pseudogene mRNA, complete cds /cds = | 0.297321107 | 0.000296 |
| | (268,798) /gb = U72518 /gi = 1673523 /ug = Hs.199299 | | |
| | /len = 1057 | | |
| 38057_at | Cluster Incl AL049798: Human DNA sequence from clone 797M17 on chromosome | 0.296747826 | 0.04127 |
| | 1q22-24.3. Contains the DPT gene for Dermatopontin, ESTs, an STS and GSSs | | |
| | /cds = (9,614) /gb = AL049798 /gi = 4995638 | | |
| | /ug = Hs.80552 /len = 1705 | | |
| 35730_at | Cluster Incl X03350: Human mRNA for alcohol dehydrogenase beta-1-subunit | 0.295479632 | 0.001241 |
| | (ADH1-2 allele) /cds = (72,1199) /gb = X03350 /gi = 28415 | | |
| | /ug = Hs.4 /len = 2532 | | |
| 34642_at | Cluster Incl U28964: Homo sapiens 14-3-3 protein mRNA, complete cds | 0.295377674 | 0.00066 |
| | /cds = (126,863) /gb = U28964 /gi = 899458 /ug = Hs.75103 | | |
| | /len = 1030 | | |
| 36133_at | Cluster Incl AL031058: Human DNA sequence from clone 512B11 on chromosome | 0.295336914 | 0.00182 |
| | 6p24-25. Contains the Desmoplakin I (DPI) gene, ESTs, STSs and GSSs /cds = | | |
| | (279,8894) /gb = AL031058 /gi = 3395507 /ug = Hs.74316 | | |
| | /len = 9591 | | |
| 41770_at | Cluster Incl AA420624: nc61c12.r1 Homo sapiens cDNA /clone = IMAGE-745750 | 0.293839135 | 0.005885 |
| | /gb = AA420624 /gi = 2094502 /ug = Hs.183109 /len = 533 | | |
| 31831_at | Cluster Incl AI888563: wn33a05.x1 Homo sapiens cDNA, 3 end /clone = | 0.293100784 | 0.000087 |
| | IMAGE-2447216 /clone_end = 3″ /gb = AI888563 /gi = 5593727 | | |
| | /ug = Hs.149098 /len = 678″ | | |
| 32242_at | Cluster Incl AL038340: DKFZp566K192_s1 Homo sapiens cDNA, 3 end /clone = | 0.293076261 | 0.000031 |
| | DKFZp566K192 /clone end = 3″/gb = AL038340 /gi = 5407591 | | |
| | /ug = Hs.1940 /len = 746″ | | |
| 1629_s_at | Tyrosine Phosphatase 1, Non-Receptor, Alt. Splice 3 | 0.292518104 | 0.020967 |
| 757_at | D28364 /FEATURE = /DEFINITION = HUMAI23 Human mRNA for annexin II, | 0.2917755 | 0.007341 |
| | 5″UTR (sequence from the 5″ cap to the start codon) | | |
| 36900_at | Cluster Incl U52426: Homo sapiens GOK (STIM1) mRNA, complete cds /cds = | 0.291126081 | 0.003618 |
| | (565,2622) /gb = U52426 /gi = 2264345 /ug = Hs.74597 | | |
| | /len = 4040 | | |
| 37159_at | Cluster Incl U79259: Human clone 23945 mRNA, complete cds /cds = (636,1403) | 0.2910031 | 0.008274 |
| | /gb = U79259 /gi = 1710213 /ug = Hs.10700 /len = 1683 | | |
| 36105_at | Cluster Incl M18728: Human nonspecific crossreacting antigen mRNA, complete cds | 0.290919286 | 0.000145 |
| | /cds = UNKNOWN /gb = M18728 /gi = 189084 /ug = Hs.73848 | | |
| | /len = 2533 | | |
| 32331_at | Cluster Incl X60673: Human AK3 mRNA for adenylate kinase 3 /cds = UNKNOWN | 0.29044214 | 0.000343 |
| | /gb = X60673 /gi = 28576 /ug = Hs.182740 /len = 1692 | | |
| 37131_at | Cluster Incl AB008390: Homo sapiens mRNA for neuropsin type1, complete cds | 0.290436248 | 0.000002 |
| | /cds = (91,873) /gb = AB008390 /gi = 5672472 /ug = | | |
| | Hs.104570 /len = 922 | | |
| 38627_at | Cluster Incl M95585: Human hepatic leukemia factor (HLF) mRNA, complete cds | 0.288939128 | 0.001437 |
| | /cds = (322,1209) /gb = M95585 /gi = 184223 /ug = Hs.101047 | | |
| | /len = 3865 | | |
| 40409_at | Cluster Incl U46689: Human microsomal aldehyde dehydrogenase (ALD10) mRNA, | 0.288681109 | 0.000525 |
| | complete cds /cds = (449,1906) /gb = U46689 /gi = 1870243 | | |
| | /ug = Hs.159608 /len = 3918 | | |
| 41401_at | Cluster Incl U57646: Homo sapiens cysteine and glycine-rich protein 2 (CSRP2) | 0.287838122 | 0.003583 |
| | mRNA, complete cds /cds = (56,637) /gb = U57646 /gi = 1373337 | | |
| | /ug = Hs.10526 /len = 883 | | |
| 36955_at | Cluster Incl U10362: Human GP36b glycoprotein mRNA, complete cds /cds = | 0.287754466 | 0.003797 |
| | (0,1070) /gb = U10362 /gi = 505651 /ug = Hs.75864 | | |
| | /len = 1407 | | |
| 34738_at | Cluster Incl L11931: Human cytosolic serine hydroxymethyltransferase (SHMT) | 0.285933018 | 0.000091 |
| | mRNA, complete cds /cds = (24,1475) /gb = L11931 /gi = 307421 | | |
| | /ug = Hs.239644 /len = 1669 | | |
| 37972_at | Cluster Incl U75744: Homo sapiens DNase gamma mRNA, complete cds /cds = | 0.285701166 | 0.000029 |
| | (92,1009) /gb = U75744 /gi = 3236319 /ug = Hs.88646 | | |
| | /len = 1093 | | |
| 32522_f_a | Cluster Incl M20469: Human brain-type clathrin light-chain b mRNA, complete cds | 0.285155024 | 0.001194 |
| | /cds = (172,861) /gb = M20469 /gi = 179398 /ug = Hs.73919 | | |
| | /len = 1134 | | |
| 38370_at | Cluster Incl U90902: Human clone 23612 mRNA sequence /cds = UNKNOWN | 0.284903455 | 0.000865 |
| | /gb = U90902 /gi = 1913880 /ug = Hs.82141 /len = 1528 | | |
| 36079_at | Cluster Incl AF010309: Homo sapiens Pig3 (PIG3) mRNA, complete cds /cds = | 0.28144582 | 0.000001 |
| | (527,1495) /gb = AF010309 /gi = 2754811 /ug = Hs.50649 | | |
| | /len = 1670 | | |
| 2049_s_at | M29039 /FEATURE = cds /DEFINITION = HUMJUNCAA Human transactivator | 0.278175501 | 0.009389 |
| | (jun-B) gene, complete cds | | |
| 41365_at | Cluster Incl Y09788: H. sapiens MUC5B gene /cds = (0,2538) /gb = | 0.275992902 | 0.007022 |
| | Y09788 /gi = 2370132 /ug = Hs.102482 /len = 3100 | | |
| 31410_at | Cluster Incl AF023614: Homo sapiens transmembrane activator and CAML interactor | 0.275927919 | 0.001766 |
| | (TACI) mRNA, complete cds /cds = (13,894) /gb = AF023614 /gi = | | |
| | 2554947 /ug = Hs.158341 /len = 1357 | | |
| 39631_at | Cluster Incl U52100: Human XMP mRNA, complete cds /cds = (63,566) /gb = | 0.275453313 | 0.000281 |
| | U52100 /gi = 2474095 /ug = Hs.29191 /len = 690 | | |
| 32275_at | Cluster Incl X04470: Human mRNA for antileukoprotease (ALP) from cervix uterus | 0.273498414 | 0.003954 |
| | /cds = (18,416) /gb = X04470 /gi = 28638 /ug = Hs.169793 | | |
| | /len = 594 | | |
| 37723_at | Cluster Incl U47414: Human cyclin G2 mRNA, complete cds /cds = (135,1169) | 0.273304688 | 0.015963 |
| | /gb = U47414 /gi = 1335886 /ug = Hs.79069 /len = 2044 | | |
| 35330_at | Cluster Incl AJ012737: Homo sapiens mRNA for filamin, muscle isoform /cds = | 0.273256372 | 0.006517 |
| | (18,8096) /gb = AJ012737 /gi = 5419654 /ug = Hs.58414 | | |
| | /len = 8842 | | |
| 179_at | U38980 /FEATURE = /DEFINITION = U38980 Human PMS2 related (hPMSR6) mRNA, | 0.271809973 | 0.001563 |
| | complete cds | | |
| 37926_at | Cluster Incl D14520: Human mRNA for GC-Box binding protein BTEB2, complete cds | 0.270544367 | 0.000116 |
| | /cds = (558,1217) /gb = D14520 /gi = 303596 /ug = Hs.84728 | | |
| | /len = 1301 | | |
| 32847_at | Cluster Incl U48959: Homo sapiens myosin light chain kinase (MLCK) mRNA, complete | 0.270328396 | 0.006524 |
| | cds /cds = (119,5863) /gb = U48959 /gi = 1377819 /ug = | | |
| | Hs.211582 /len = 5926 | | |
| 2094_s_at | K00650 /FEATURE = cds /DEFINITION = HUMFOS Human fos proto-oncogene | 0.270175289 | 0.000004 |
| | (c-fos), complete cds | | |
| 467_at | U63717 /FEATURE = /DEFINITION = HSU63717 Homo sapiens osteoclast | 0.27004121 | 0.000962 |
| | stimulating factor mRNA, complete cds | | |
| 36524_at | Cluster Incl AB029035: Homo sapiens mRNA for KIAA1112 protein, partial cds | 0.262861548 | 0.00005 |
| | /cds = (0,2086) /gb = AB029035 /gi = 5689560 /ug = Hs.6066 | | |
| | /len = 3800 | | |
| 38971_r_a | Cluster Incl AJ011896: Homo sapiens mRNA for HIV-1, Nef-associated factor 1 | 0.261633057 | 0.029949 |
| | beta (Naf1 beta) /cds = (110,2017) /gb = AJ011896 /gi = 3758820 | | |
| | /ug = Hs.109281 /len = 2710 | | |
| AFFX-HU | M33197 Human glyceraldehyde-3-phosphate dehydrogenase (GAPDH) mRNA, complete | 0.261185115 | 0.015471 |
| | cds (_5, _M, _3 represent transcript regions 5 prime, Middle, and 3 prime | | |
| | respectively) | | |
| 39528_at | Cluster Incl L24564: Human Rad mRNA, complete cds /cds = (123,1049) | 0.260631274 | 0.000003 |
| | /gb = L24564 /gi = 439602 /ug = Hs.1027 /len = 1443 | | |
| 36734_at | Cluster Incl M21302: Human small proline rich protein (sprll) mRNA, clone 174N | 0.257892687 | 0 |
| | /cds = (60,278) /gb = M21302 /gi = 338424 /ug = Hs.56306 | | |
| | /len = 683 | | |
| 33849_at | Cluster Incl U02020: Human pre-B cell enhancing factor (PBEF) mRNA, complete cds | 0.256441757 | 0.010246 |
| | /cds = (27,1502) /gb = U02020 /gi = 404012 /ug = Hs.239138 | | |
| | /len = 2376 | | |
| 34951_at | Cluster Incl D10923: Human mRNA for HM74 /cds = (60,1223) /gb = D10923 | 0.254262997 | 0.000014 |
| | /gi = 219866 /ug = Hs.137555 /len = 2041 | | |
| 41198_at | Cluster Incl AF055008: Homo sapiens clone 24720 epithelin 1 and 2 mRNA, complete | 0.254025212 | 0.000352 |
| | cds /cds = (62,1843) /gb = AF055008 /gi = 3005729 /ug = | | |
| | Hs.180577 /len = 2148 | | |
| 34281_at | Cluster Incl AF039555: Homo sapiens visinin-like protein 1 (VSNL1) mRNA, complete | 0.25338608 | 0.015165 |
| | cds /cds = (192,767) /gb = AF039555 /gi = 4104813 /ug = | | |
| | Hs.2288 /len = 1559 | | |
| 34198_at | Cluster Incl U12128: Human protein tyrosine phosphatase 1E (PTP1E) mRNA, complete | 0.252855276 | 0.004144 |
| | cds /cds = (217,7689) /gb = U12128 /gi = 557287 /ug = | | |
| | Hs.211595 /len = 8287 | | |
| 41043_at | Cluster Incl AB006867: Homo sapiens mRNA for hSOX20 protein, complete cds | 0.2523451 | 0.000279 |
| | /cds = (494,1195) /gb = AB006867 /gi = 3061309 /ug = | | |
| | Hs.95582 /len = 1394 | | |
| 36873_at | Cluster Incl D16532: Human gene for very low density lipoprotein receptor, | 0.252075639 | 0.011366 |
| | 5flanking and /cds = (615,3236) /gb = D16532 /gi = 407220 | | |
| | /ug = Hs.73729 /len = 3853″ | | |
| 34933_at | Cluster Incl AJ238381: Homo sapiens pax9 gene, exons 1-2 and joined CDS | 0.251978856 | 0.043656 |
| | /cds = (391,1416) /gb = AJ238381 /gi = 4699895 /ug = | | |
| | Hs.132576 /len = 1630 | | |
| 34651_at | Cluster Incl M58525: Homo sapiens catechol-O-methyltransferase (COMT) mRNA, | 0.250728594 | 0.000702 |
| | complete cds /cds = (204,1019) /gb = M58525 /gi = 179954 | | |
| | /ug = Hs.78534 /len = 1206 | | |
| 32527_at | Cluster Incl AI381790: te41h10.x1 Homo sapiens cDNA, 3 end /clone = | 0.250563902 | 0.014061 |
| | IMAGE-2089315 /clone_end = 3″ /gb = AI381790 /gi = 4194571 | | |
| | /ug = Hs.74120 /len = 544″ | | |
| 38903_at | Cluster Incl AF099731: Homo sapiens connexin 31.1 (GJB5) gene, complete cds | 0.249028452 | 0.000008 |
| | /cds = (27,848) /gb = AF099731 /gi = 4009521 /ug = Hs.198249 | | |
| | /len = 1370 | | |
| 32112_s_{—} | Cluster Incl AI800499: tc11f11.x1 Homo sapiens cDNA, 3 end /clone = | 0.248564076 | 0.000001 |
| | IMAGE-2063565 /clone_end = 3″ /gb = AI800499 /gi = 5365971 | | |
| | /ug = Hs.161002 /len = 403″ | | |
| 36838_at | Cluster Incl AF055481: Homo sapiens normal epithelial cell-specific 1 (NES1) | 0.248424399 | 0.001939 |
| | gene, complete cds /cds = (82,912) /gb = AF055481 /gi = 3065710 | | |
| | /ug = Hs.69423 /len = 1442 | | |
| 32163_f_a | Cluster Incl AA216639: zq95f07.s1 Homo sapiens cDNA, 3 end /clone = | 0.247712958 | 0.010541 |
| | IMAGE-649765 /clone_end = 3″ /gb = AA216639 /gi = 1817337 | | |
| | /ug = Hs.184093 /len = 569″ | | |
| 32200_at | Cluster Incl M24902: Human prostatic acid phosphatase mRNA, complete cds | 0.247567897 | 0.009707 |
| | /cds = (23,1183) /gb = M24902 /gi = 189618 /ug = Hs.1852 | | |
| | /len = 3097 | | |
| 38881_i_a | Cluster Incl AF096870: Homo sapiens estrogen-responsive B box protein (EBBP) | 0.245430421 | 0.000297 |
| | mRNA, complete cds /cds = (227,1921) /gb = AF096870 /gi = 3916726 | | |
| | /ug = Hs.194540 /len = 2568 | | |
| 39171_at | Cluster Incl W21787: 58b10 Homo sapiens cDNA /clone = (not-directional) | 0.24193833 | 0.000959 |
| | /gb = W21787 /gi = 1298717 /ug = Hs.99816 /len = 775 | | |
| 31655_at | Cluster Incl AL031737: Human DNA sequence from clone 8B22 on chromosome | 0.240900298 | 0.027265 |
| | 1p35.1-36.21 Contains gene similar to cytoplasmic dynein light chain 1, GSSs | | |
| | /cds = (92,361) /gb = AL031737 /gi = 4464258 /ug = Hs.225980 | | |
| | /len = 371 | | |
| 37215_at | Cluster Incl AF046798: untitled /cds = (113,2656) /gb = AF046798 | 0.240324439 | 0.000027 |
| | /gi = 3170406 /ug = Hs.771 /len = 2828 | | |
| 38339_at | Cluster Incl AF082868: Homo sapiens gamma butyrobetaine hydroxylase (BBH) mRNA, | 0.238959561 | 0.000097 |
| | complete cds /cds = (66,1229) /gb = AF082868 /gi = 3746804 | | |
| | /ug = Hs.9667 /len = 1584 | | |
| 35803_at | Cluster Incl S82240: RhoE = 26 kda GTPase homolog [human, HeLa cell | 0.238923582 | 0.005486 |
| | line, mRNA, 833 nt] /cds = (24,713) /gb = S82240 /gi = | | |
| | 1839516 /ug = Hs.6838 /len = 833 | | |
| 40096_at | Cluster Incl D14710: Human mRNA for ATP synthase alpha subunit, complete cds | 0.234166294 | 0.016171 |
| | /cds = (63,1724) /gb = D14710 /gi = 559324 /ug = Hs.155101 | | |
| | /len = 1857 | | |
| 36890_at | Cluster Incl AF001691: Homo sapiens 195 kDa cornified envelope precursor mRNA, | 0.233555948 | 0.020797 |
| | complete cds | | |
| | /cds = (90,5360) /gb = AF001691 /gi = 3168845 /ug = Hs.74304 | | |
| | /len = 6227 | | |
| 39052_at | Cluster Incl J00124: Homo sapiens 50 kDa type I epidermal keratin gene, complete | 0.231924773 | 0.03245 |
| | cds /cds = (61,1479) /gb = J00124 /gi = 186704 /ug = | | |
| | Hs.117729 /len = 1634 | | |
| 32010_at | Cluster Incl Z82180: Human DNA sequence from clone E81G9 on chromosome 22 Contains | 0.231601307 | 0.000002 |
| | novel gene EAN57, ESTs and GSS /cds = (0,181) /gb = Z82180 /gi = | | |
| | 5051358 /ug = Hs.225954 /len = 285 | | |
| 41469_at | Cluster Incl L10343: Huma elafin gene, complete cds /cds = (516,869) | 0.231452377 | 0.00149 |
| | /gb = L10343 /gi = 190337 /ug = Hs.112341 /len = 871 | | |
| 35909_at | Cluster Incl Z50194: H. sapiens mRNA for PQ-rich protein /cds = (159,1361) | 0.231034761 | 0.003707 |
| | /gb = Z50194 /gi = 929659 /ug = Hs.198179 /len = 1412 | | |
| 36555_at | Cluster Incl AF044311: Homo sapiens gamma-synuclein gene, complete cds | 0.230334156 | 0.013692 |
| | /cds = (52,435) /gb = AF044311 /gi = 3347841 /ug = Hs.63236 | | |
| | /len = 708 | | |
| 1582_at | M29540 /FEATURE = /DEFINITION = HUMCEAF Human carcinoembryonic antigen | 0.228698047 | 0.000018 |
| | mRNA (CEA), complete cds | | |
| 39248_at | Cluster Incl N74607: za55a01.s1 Homo sapiens cDNA, 3 end /clone = IMAGE-296424 | 0.227986593 | 0.00066 |
| | /clone_end = 3″ /gb = N74607 /gi = 1231892 /ug = | | |
| | Hs.234642 /len = 487″ | | |
| 33370_r_a | Cluster Incl U60205: Human methyl sterol oxidase (ERG25) mRNA, complete cds | 0.22574526 | 0.000114 |
| | /cds = (26,907) /gb = U60205 /gi = 1408205 /ug = Hs.223018 | | |
| | /len = 1751 | | |
| 32392_s_{—} | Cluster Incl M57951: Human bilirubin UDP-glucuronosyltransferase isozyme 2 mRNA, | 0.221926907 | 0.005613 |
| | complete cds /cds = (29,1633) /gb = M57951 /gi = 184474 | | |
| | /ug = Hs.233441 /len = 2368 | | |
| 40303_at | Cluster Incl U85658: Human transcription factor ERF-1 mRNA, complete cds | 0.220714707 | 0.000106 |
| | /cds = (166,1518) /gb = U85658 /gi = 2058552 /ug = Hs.61796 | | |
| | /len = 2785 | | |
| 37157_at | Cluster Incl X56667: Human mRNA for calretinin /cds = (43,858) /gb = | 0.219711115 | 0.000083 |
| | X56667 /gi = 29635 /ug = Hs.106857 /len = 1426 | | |
| 234_s_at | M57399 /FEATURE = /DEFINITION = HUMHBNF1 Human nerve growth factor | 0.213743207 | 0.002063 |
| | (HBNF-1) mRNA, complete cds | | |
| 33792_at | Cluster Incl AF043498: Homo sapiens prostate stem cell antigen (PSCA) mRNA, | 0.212093464 | 0 |
| | complete cds /cds = (17,388) /gb = AF043498 /gi = 2909843 | | |
| | /ug = Hs.20166 /len = 990 | | |
| 37765_at | Cluster Incl X54162: Human mRNA for a 64 Kd autoantigen expressed in thyroid | 0.21133549 | 0.027383 |
| | and extra-ocular muscle /cds = (212,1930) /gb = X54162 /gi = | | |
| | 28968 /ug = Hs.79386 /len = 3849 | | |
| 31542_at | Cluster Incl M60502: Human profilaggrin mRNA, 3 end /cds = (0,2393) | 0.209204984 | 0.000756 |
| | /gb = M60502 /gi = 190403 /ug = Hs.73995 /len = 2930″ | | |
| 33128_s_{—} | Cluster Incl W68521: zd36f07.r1 Homo sapiens cDNA, 5 end /clone = IMAGE-342757 | 0.205685908 | 0.000172 |
| | /clone_end = 5″ /gb = W68521 /gi = 1377410 /ug = | | |
| | Hs.83393 /len = 579″ | | |
| 36242_at | Cluster Incl M21539: Human small proline rich protein (sprll) mRNA, clone 1292 | 0.202617237 | 0.000204 |
| | /cds = (86,304) /gb = M21539 /gi = 338426 /ug = Hs.2421 | | |
| | /len = 678 | | |
| 33883_at | Cluster Incl AB001466: Homo sapiens mRNA for Efs1, complete cds /cds = | 0.201124632 | 0.001354 |
| | (608,2293) /gb = AB001466 /gi = 2829301 /ug = Hs.24587 | | |
| | /len = 3114 | | |
| 32582_at | Cluster Incl AF001548: Human Chromosome 16 BAC clone CIT987SK-A-815A9 /cds = | 0.198342888 | 0.001142 |
| | (0,5573) /gb = AF001548 /gi = 2104552 /ug = Hs.78344 | | |
| | /len = 6428 | | |
| 913_at | U47414 /FEATURE = /DEFINITION = HSU47414 Human cyclin G2 mRNA, | 0.197140492 | 0.000035 |
| | complete cds | | |
| 34203_at | Cluster Incl D17408: Homo sapiens mRNA for calponin, complete cds /cds = | 0.195771996 | 0.000225 |
| | (92,985) /gb = D17408 /gi = 1783204 /ug = Hs.21223 | | |
| | /len = 1517 | | |
| 32963_s_{—} | Cluster Incl W27549: 32d11 Homo sapiens cDNA /gb = W27549 /gi = | 0.193598948 | 0.000017 |
| | 1307353 /ug = Hs.235634 /len = 912 | | |
| 1165_at | D49950 /FEATURE = /DEFINITION = D49950 Homo sapiens mRNA for | 0.191184898 | 0.000062 |
| | interferon-gamma inducing factor(IGIF), complete cds | | |
| 1005_at | X68277 /FEATURE = cds /DEFINITION = HSCL100 H. sapiens CL 100 mRNA | 0.190308395 | 0.008438 |
| | for protein tyrosine phosphatase | | |
| 32523_at | Cluster Incl M20470: Human lymphocyte clathrin light-chain B mRNA, complete cds | 0.189040488 | 0.005713 |
| | /cds = (139,774) /gb = M20470 /gi = 187056 /ug = Hs.73919 | | |
| | /len = 1051 | | |
| 40016_g_{—} | Cluster Incl AB002301: Human mRNA for KIAA0303 gene, partial cds /cds = | 0.188340227 | 0.000827 |
| | (0,6415) /gb = AB002301 /gi = 2224546 /ug = Hs.54985 | | |
| | /len = 6629 | | |
| 35425_at | Cluster Incl AJ243512: Homo sapiens mRNA for Barx2 protein (Barx2 gene) | 0.187189147 | 0.000087 |
| | /cds = (16,855) /gb = AJ243512 /gi = 5459378 /ug = | | |
| | Hs.167218 /len = 1374 | | |
| 2027_at | M87068 /FEATURE = /DEFINITION = HUMCAN H. sapiens CaN19 mRNA sequence | 0.187103745 | 0.000726 |
| 36925_at | Cluster Incl L26336: Human heat shock protein HSPA2 gene, complete cds | 0.182654009 | 0.000097 |
| | /cds = (0,1919) /gb = L26336 /gi = 476704 /ug = Hs.75452 | | |
| | /len = 1920 | | |
| 39015_f_a | Cluster Incl L42611: Homo sapiens keratin 6 isoform K6e (KRT6E) mRNA, complete | 0.181632277 | 0.020188 |
| | cds /cds = (40,1734) /gb = L42611 /gi = 908802 /ug = | | |
| | Hs.111758 /len = 1986 | | |
| 613_at | M21389 /FEATURE = mRNA /DEFINITION = HUMKER2A Human keratin type II | 0.175195643 | 0.035399 |
| | (58 kD) mRNA, complete cds | | |
| 36378_at | Cluster Incl AF085807: Homo sapiens uroplakin la mRNA, partial cds /cds = | 0.174269851 | 0.000023 |
| | (0,771) /gb = AF085807 /gi = 3550409 /ug = Hs.159309 | | |
| | /len = 1218 | | |
| 31846_at | Cluster Incl AW003733: ws16b04.x1 Homo sapiens cDNA, 3 end /clone = | 0.167432167 | 0.010423 |
| | IMAGE-2497327 /clone_end = 3″ /gb = AW003733 /gi = 5850649 | | |
| | /ug = Hs.15114 /len = 839″ | | |
| 1197_at | D00654 /FEATURE = cds /DEFINITION = HUMACTSG7 Homo sapiens gene for | 0.163684848 | 0.009945 |
| | enteric smooth muscle gamma-actin, exon 9, complete cds | | |
| 31343_at | Cluster Incl AJ005835: Homo sapiens mRNA for interleukin 1 receptor antagonist, | 0.16202207 | 0.000012 |
| | type III /cds = (0,10) /gb = AJ005835 /gi = 3115205 /ug = | | |
| | Hs.131958 /len = 171 | | |
| 37576_at | Cluster Incl U52969: Human PEP19 (PCP4) mRNA, complete cds /cds = (81,269) | 0.160690395 | 0.000535 |
| | /gb = U52969 /gi = 1568614 /ug = Hs.80296 /len = 554 | | |
| 2090_i_at | H12458 /FEATURE = /DEFINITION = H12458 yj12d03.s1 Soares placenta | 0.159035469 | 0.01138 |
| | Nb2HP Homo sapiens cDNA clone IMAGE: 148517 3″ similar to SP: WNT6_MOUSE | | |
| | P22727 WNT-6 PROTEIN;, mRNA sequence | | |
| 33331_at | Cluster Incl U17077: Human BENE mRNA, partial cds /cds = (0,446) /gb = | 0.156306005 | 0.000286 |
| | U17077 /gi = 1000711 /ug = Hs.185055 /len = 2269 | | |
| 40035_at | Cluster Incl AB012917: Homo sapiens mRNA for serine protease (TLSP), complete cds | 0.15559402 | 0.002195 |
| | /cds = (25,873) /gb = AB012917 /gi = 3649790 /ug = Hs.57771 | | |
| | /len = 1186 | | |
| 39660_at | Cluster Incl AI309115: qo71a09.x1 Homo sapiens cDNA, 3 end /clone = | 0.155477892 | 0.000234 |
| | IMAGE-1913944 /clone_end = 3″ /gb = AI309115 /gi = 4003986 | | |
| | /ug = Hs.32949 /len = 450″ | | |
| 33546_at | Cluster Incl AI923984: wn49d12.x1 Homo sapiens cDNA, 3 end /clone = | 0.155471597 | 0.009457 |
| | IMAGE-2448791 /clone_end = 3″ /gb = AI923984 /gi = 5659948 | | |
| | /ug = Hs.46320 /len = 440″ | | |
| 41471_at | Cluster Incl W72424: zd66a09.s1 Homo sapiens cDNA, 3 end /clone = | 0.15508022 | 0.018334 |
| | IMAGE-345592 /clone_end = 3″ /gb = W72424 /gi = 1382379 | | |
| | /ug = Hs.112405 /len = 604″ | | |
| 33483_at | Cluster Incl X76029: H. sapiens mRNA for neuromedin U /cds = (105,629) | 0.154692443 | 0.022939 |
| | /gb = X76029 /gi = 609012 /ug = Hs.2841 /len = 817 | | |
| 39682_at | Cluster Incl X87159: H. sapiens mRNA for beta subunit of epithelial amiloride- | 0.154579413 | 0.000005 |
| | sensitive sodium channel cds = (127,2049) /gb = X87159 /gi = | | |
| | 1004270 /ug = Hs.37129 /len = 2545 | | |
| 617_at | M24902 /FEATURE = mRNA /DEFINITION = HUMPAPA Human prostatic acid | 0.153293459 | 0 |
| | phosphatase mRNA, complete cds | | |
| 34820_at | Cluster Incl M57399: Human nervegrowth factor (HBNF-1) mRNA, complete cds | 0.152145671 | 0.000666 |
| | /cds = (395,901) /gb = M57399 /gi = 292072 /ug = Hs.44 | | |
| | /len = 1029 | | |
| 36454_at | Cluster Incl AF037335: Homo sapiens carbonic anhydrase precursor (CA 12) mRNA, | 0.151522134 | 0.001281 |
| | complete cds /cds = (115,1179) /gb = AF037335 /gi = 2708638 | | |
| | /ug = Hs.5338 /len = 2755 | | |
| 39544_at | Cluster Incl AB002351: Human mRNA for KIAA0353 gene, partial cds /cds = | 0.148964837 | 0.01262 |
| | (0,4125) /gb = AB002351 /gi = 2224646 /ug = Hs.10587 | | |
| | /len = 6651 | | |
| 40365_at | Cluster Incl M63904: Human G-alpha 16 protein mRNA, complete cds /cds = | 0.148225853 | 0.000153 |
| | (219,1343) /gb = M63904 /gi = 182891 /ug = Hs.73797 | | |
| | /len = 2060 | | |
| 37160_at | Cluster Incl M19888: Human small proline rich protein (sprl) mRNA, clone 128 | 0.145719365 | 0.000021 |
| | /cds = (68,337) /gb = M 19888 /gi = 338416 /ug = Hs.1076 | | |
| | /len = 623 | | |
| 529_at | U15932 /FEATURE = /DEFINITION = HSU15932 Human dual-specificity | 0.144995121 | 0.000002 |
| | protein phosphatase mRNA, complete cds | | |
| 770_at | D00632 /FEATURE = /DEFINITION = HUMGSHPXA Homo sapiens mRNA for | 0.143850665 | 0.002402 |
| | glutathione peroxidase, complete cds | | |
| 31805_at | Cluster Incl M64347: Human novel growth factor receptor mRNA, 3 cds /cds = | 0.14334791 | 0.000421 |
| | (0,2195) /gb = M64347 /gi = 182564 /ug = Hs.1420 | | |
| | /len = 3799″ | | |
| 41034_s_{—} | Cluster Incl U92315: Homo sapiens hydroxysteroid sulfotransferase SULT2B1b | 0.143222232 | 0.000049 |
| | (HSST2) mRNA, complete cds /cds = (81,1178) /gb = U92315 /gi = | | |
| | 1923292 /ug = Hs.94581 /len = 1199 | | |
| 37600_at | Cluster Incl U68186: Human extracellular matrix protein 1 mRNA, complete cds | 0.141941707 | 0.000773 |
| | /cds = (103,1725) /gb = U68186 /gi = 2660683 /ug = Hs.81071 | | |
| | /len = 1819 | | |
| 37762_at | Cluster Incl Y07909: H. sapiens mRNA for Progression Associated Protein | 0.141800128 | 0.001618 |
| | /cds = (218,691) /gb = Y07909 /gi = 1542882 /ug = Hs.79368 | | |
| | /len = 2774 | | |
| 35726_at | Cluster Incl AI539439: te51e07.x1 Homo sapiens cDNA, 3 end /clone = | 0.141239149 | 0.000072 |
| | IMAGE-2090244 /clone_end = 3″ /gb = AI539439 /gi = 4453574 | | |
| | /ug = Hs.38991 /len = 455″ | | |
| 39302_at | Cluster Incl X56807: Human DSC2 mRNA for desmocollins type 2a and 2b | 0.14069071 | 0.000006 |
| | /cds = (0,2583) /gb = X56807 /gi = 30507 /ug = Hs.239727 | | |
| | /len = 3212 | | |
| 37920_at | Cluster Incl U70370: Human hindlimb expressed homeobox protein backfoot (Bft) | 0.139179451 | 0.000061 |
| | mRNA, complete cds /cds = (111,1055) /gb = U70370 /gi = 1870670 | | |
| | /ug = Hs.84136 /len = 2049 | | |
| 39698_at | Cluster Incl U51712: HSU51712 Homo sapiens cDNA /gb = U51712 /gi = | 0.13905098 | 0.000053 |
| | 1255282 /ug = Hs.13775 /len = 1110 | | |
| 36497_at | Cluster Incl W28438: 47g10 Homo sapiens cDNA /gb = W28438 /gi = | 0.136810256 | 0.005909 |
| | 1308449 /ug = Hs.57548 /len = 845 | | |
| 601_s_at | M28439 /FEATURE = cds /DEFINITION = HUMKER16A8 Human keratin type | 0.136363596 | 0.003168 |
| | 16 gene, exon 8 | | |
| 36658_at | Cluster Incl D13643: Human mRNA for KIAA0018 gene, complete cds /cds = | 0.135965128 | 0.008011 |
| | (38,1210) /gb = D13643 /gi = 285996 /ug = Hs.75616 | | |
| | /len = 4186 | | |
| 38202_at | Cluster Incl AB011535: Homo sapiens mRNA for MEGF1, partial cds /cds = | 0.129294854 | 0.000014 |
| | (0,1721) /gb = AB011535 /gi = 3449295 /ug = Hs.158159 | | |
| | /len = 3193 | | |
| 37603_at | Cluster Incl X52015: H. sapiens mRNA for interleukin-1 receptor antagonist | 0.128827966 | 0.000242 |
| | /cds = (23,556) /gb = X52015 /gi = 32576 /ug = Hs.81134 | | |
| | /len = 1740 | | |
| 32113_at | Cluster Incl U83115: Human non-lens beta gamma-crystallin like protein (AIM1) | 0.124007546 | 0.000013 |
| | mRNA, partial cds /cds = (0,4913) /gb = U83115 /gi = 2072424 | | |
| | /ug = Hs.161002 /len = 6796 | | |
| 39366_at | Cluster Incl N36638: yx88f05.r1 Homo sapiens cDNA, 5 end /clone = | 0.123584314 | 0.000028 |
| | IMAGE-268833 /clone_end = 5″ /gb = N36638 /gi = 1157780 | | |
| | /ug = Hs.12112 /len = 543″ | | |
| 40304_at | Cluster Incl M69225: Human bullous pemphigoid antigen (BPAG1) mRNA, complete cds | 0.121641611 | 0 |
| | /cds = UNKNOWN /gb = M69225 /gi = 179522 /ug = Hs.620 | | |
| | /len = 8930 | | |
| 37473_at | Cluster Incl AF061812: Homo sapiens keratin 16 (KRT16A) mRNA, complete cds | 0.120176887 | 0.002993 |
| | /cds = (14,1435) /gb = AF061812 /gi = 4091878 /ug = | | |
| | Hs.115947 /len = 1588 | | |
| 1776_at | L24564 /FEATURE = /DEFINITION = HUMRAD Human Rad mRNA, complete cds | 0.119942667 | 0.000005 |
| 38051_at | Cluster Incl X76220: H. sapiens MAL gene exon 1 (and joined CDS) /cds = | 0.118545163 | 0.015366 |
| | (59,520) /gb = X76220 /gi = 433225 /ug = Hs.80395 | | |
| | /len = 1056 | | |
| 37956_at | Cluster Incl U37519: Human aldehyde dehydrogenase (ALDH8) mRNA, complete cds | 0.114940468 | 0.000183 |
| | /cds = (616,1773) /gb = U37519 /gi = 1051280 /ug = Hs.87539 | | |
| | /len = 2827 | | |
| 39569_at | Cluster Incl U72849: untitled /cds = (98,6199) /gb = U72849 | 0.113225316 | 0.000035 |
| | /gi = 4097997 /ug = Hs.25482 /len = 6457 | | |
| 40356_at | Cluster Incl AB026833: Homo sapiens mRNA for chloride channel protein, | 0.109385318 | 0.000001 |
| | complete cds /cds = (41,2872) /gb = AB026833 /gi = 4887600 | | |
| | /ug = Hs.72365 /len = 3604 | | |
| 37093_at | Cluster Incl M32402: Human placental protein (PP11) mRNA, complete cds | 0.103523453 | 0.000448 |
| | /cds = UNKNOWN /gb = M32402 /gi = 190210 /ug = Hs.997 | | |
| | /len = 2320 | | |
| 1321_s_at | U43916 /FEATURE = /DEFINITION = HSU43916 Human tumor-associated | 0.102454011 | 0 |
| | membrane protein homolog (TMP) mRNA, complete cds | | |
| 36284_at | Cluster Incl Y12642: H. sapiens E48 gene /cds = (24,410) /gb = Y12642 | 0.101688161 | 0.000003 |
| | /gi = 2739293 /ug = Hs.3185 /len = 748 | | |
| 774_g_at | D10667 /FEATURE = /DEFINITION = HUMMHCAAA Homo sapiens mRNA for | 0.098993518 | 0.004537 |
| | smooth muscle myosin heavy chain, partial cds | | |
| 1898_at | L24203 /FEATURE = /DEFINITION = HUMDK Homo sapiens ataxia- | 0.097775137 | 0.000124 |
| | telangiectasia group D-associated protein mRNA, complete cds | | |
| 41783_at | Cluster Incl M97815: Human retinoic acid-binding protein II (CRABP-II) gene | 0.097133202 | 0.000064 |
| | /cds = (137,553) /gb = M97815 /gi = 181029 /ug = Hs.183650 | | |
| | /len = 969 | | |
| 773_at | D10667 /FEATURE = /DEFINITION = HUMMHCAAA Homo sapiens mRNA for | 0.095787941 | 0.005341 |
| | smooth muscle myosin heavy chain, partial cds | | |
| 41641_at | Cluster Incl AJ223603: Homo sapiens mRNA encoding rat C4.4-like protein | 0.095093025 | 0.000087 |
| | /cds = (86,1126) /gb = AJ223603 /gi = 3821054 /ug = Hs.11950 | | |
| | /len = 1684 | | |
| 40776_at | Cluster Incl M63391: Human desmin gene, complete cds /cds = (80,1489) | 0.094099776 | 0.000018 |
| | /gb = M63391 /gi = 181539 /ug = Hs.171185 /len = 2220 | | |
| 862_at | U04313 /FEATURE = /DEFINITION = HSU04313 Human maspin mRNA, | 0.091970059 | 0.000527 |
| | complete cds | | |
| 33693_at | Cluster Incl M76482: Human 130-kD pemphigus vulgaris antigen mRNA, complete cds | 0.091756719 | 0.000006 |
| | /cds = (83,3082) /gb = M76482 /gi = 190751 /ug = Hs.1925 | | |
| | /len = 3336 | | |
| 36464_at | Cluster Incl X94323: H. sapiens mRNA for SGP28 protein /cds = (40,777) | 0.088981309 | 0.000056 |
| | /gb = X94323 /gi = 1213612 /ug = Hs.54431 /len = 2124 | | |
| 35124_at | Cluster Incl M62982: Human arachidonate 12-lipoxygenase mRNA, complete cds | 0.087650494 | 0.004249 |
| | /cds = (53,2044) /gb = M62982 /gi = 177106 /ug = Hs.1200 | | |
| | /len = 2348 | | |
| 1057_at | M97815 /FEATURE = expanded_cds /DEFINITION = HUMCRABP02 Human | 0.084318624 | 0.000101 |
| | retinoic acid-binding protein II (CRABP-II) gene exons 2-4, complete cds | | |
| 31345_at | Cluster Incl AB002134: Homo sapiens mRNA for airway trypsin-like protease, | 0.082579556 | 0.013315 |
| | complete cds/cds = (61,1317) /gb = AB002134 /gi = 3184183 | | |
| | /ug = Hs.132195 /len = 1500 | | |
| 1549_s_at | U19557 /FEATURE = /DEFINITION = HSU19557 Human Squamous cell | 0.08107938 | 0.000031 |
| | carcinoma antigen 2 (SCCA2) mRNA, complete cds | | |
| 31791_at | Cluster Incl Y16961: Homo sapiens mRNA for KET protein /cds = (27,2069) | 0.079103695 | 0.009833 |
| | /gb = Y16961 /gi = 3970716 /ug = Hs.137569 /len = 4849 | | |
| 1343_s_at | S66896 /FEATURE = /DEFINITION = S66896 Squamous cell carcinoma | 0.07050762 | 0.000109 |
| | antigen = serine protease inhibitor [human, mRNA, 1711 nt] | | |
| 36355_at | Cluster Incl M13903: Human involucrin mRNA /cds = (0,1757) /gb = | 0.063600753 | 0.000334 |
| | M13903 /gi = 186520 /ug = Hs.157091 /len = 1758 | | |
| 40315_at | Cluster Incl AJ228139: Homo sapiens mRNA for LETKI precursor /cds = (43,3237) | 0.063426329 | 0.001565 |
| | /gb = AJ228139 /gi = 4585698 /ug = Hs.64867 /len = 3528 | | |
| 37582_at | Cluster Incl X07696: Human mRNA for cytokeratin 15 /cds = (61,1431) | 0.063223853 | 0.00109 |
| | /gb = X07696 /gi = 34070 /ug = Hs.80342 /len = 1709 | | |
| 36406_at | Cluster Incl AA401397: zu68b01.s1 Homo sapiens cDNA, 3 end /clone = | 0.06095209 | 0.000001 |
| | IMAGE-743113 /clone_end = 3″ /gb = AA401397 /gi = 2053605 | | |
| | /ug = Hs.165296 /len = 519″ | | |
| 40031_at | Cluster Incl M74542: Human aldehyde dehydrogenase type III (ALDHIII) mRNA, | 0.058614393 | 0.000689 |
| | complete cds /cds = (42,1403) /gb = M74542 /gi = 178401 | | |
| | /ug = Hs.575 /len = 1636 | | |
| 37954_at | Cluster Incl X16662: Human mRNA for vascular anticoagulant-beta (VAC-beta) | 0.058021351 | 0.000785 |
| | /cds = (106,1089) /gb = X16662 /gi = 37638 /ug = Hs.87268 | | |
| | /len = 1940 | | |
| 32139_at | Cluster Incl Y09538: H. sapiens mRNA for ZNF185 gene /cds = (40,1398) | 0.057956613 | 0.000018 |
| | /gb = Y09538 /gi = 2370125 /ug = Hs.16622 /len = 3604 | | |
| 37185_at | Cluster Incl Y00630: Human mRNA for Arg-Serpin (plasminogen activator-inhibitor 2, | 0.057481589 | 0.005043 |
| | PAI-2) /cds = (72,1319) /gb = Y00630 /gi = 35267 /ug = | | |
| | Hs.75716 /len = 1900 | | |
| 39581_at | Cluster Incl AA570193: nf38c11.s1 Homo sapiens cDNA /clone = IMAGE-916052 | 0.057372976 | 0.003457 |
| | /gb = AA570193 /gi = 2344173 /ug = Hs.2621 /len = 450 | | |
| 36407_at | Cluster Incl AL050220: Homo sapiens mRNA; cDNA DKFZp586J1923 (from clone | 0.056802495 | 0.000001 |
| | DKFZp586J1923) /cds = (0,590) /gb = AL050220 /gi = 4884461 | | |
| | /ug = Hs.165296 /len = 733 | | |
| 39249_at | Cluster Incl AB001325: Human AQP3 gene for aquaporine 3 (water channel), partail | 0.049433675 | 0.000144 |
| | cds /cds = (60,938) /gb = AB001325 /gi = 1854373 /ug = | | |
| | Hs.234642 /len = 1442 | | |
| 38489_at | Cluster Incl M60047: Human heparin binding protein (HBp17) mRNA, complete cds | 0.048264708 | 0.000019 |
| | /cds = (97,801) /gb = M60047 /gi = 183950 /ug = Hs.1690 | | |
| | /len = 1146 | | |
| 33529_at | Cluster Incl X76342: H. sapiens ADH7 mRNA /cds = UNKNOWN /gb = X76342 | 0.048228074 | 0 |
| | /gi = 541674 /ug = Hs.389 /len = 2055 | | |
| 35947_at | Cluster Incl M98447: H. sapiens keratinocyte transglutaminase gene, complete cds | 0.04499404 | 0.045596 |
| | /cds = (90,2543) /gb = M98447 /gi = 186734 /ug = Hs.22 | | |
| | /len = 2719 | | |
| 35105_at | Cluster Incl AF045941: Homo sapiens sciellin (SCEL) mRNA, complete cds /cds = | 0.040865578 | 0.022763 |
| | (86,2092) /gb = AF045941 /gi = 3893854 /ug = Hs.115166 | | |
| | /len = 2327 | | |
| 32868_at | Cluster Incl L10386: Homo sapiens transglutaminase E3 (TGASE3) mRNA, complete cds | 0.039911624 | 0.000146 |
| | /cds = (41,2122) /gb = L10386 /gi = 307503 /ug = Hs.2022 | | |
| | /len = 2619 | | |
| 31705_at | Cluster Incl X99977: H. sapiens ARS gene, component B /cds = (26,337) | 0.028363962 | 0.000051 |
| | /gb = X99977 /gi = 1536901 /ug = Hs.103505 /len = 528 | | |
| 38608_at | Cluster Incl AA010777: ze22f06.r1 Homo sapiens cDNA, 5 end /clone = | 0.028030428 | 0.000245 |
| | IMAGE-359747 /clone_end = 5″ /gb = AA010777 /gi = 1471804 | | |
| | /ug = Hs.99923 /len = 521″ | | |

TABLE 3
U95_B Fold Change Genes (>3 over-expressed in Barrett's associated
esophageal adenocarcinoma (BA), <0.33 under-expressed in BA)

| Affy ID | Gene Name | Fold Change | P-value |
|---|---|---|---|
| 55805_g_a | Cluster Incl. AW007803:wt03d03.x1 Homo sapiens cDNA, 3 end | 51.27773413 | 0.000037 |
| | /clone = IMAGE-2506373 /clone_end = 3′ | | |
| | /gb = AW007803 /gi = 5856581 /ug = Hs.61311 /len = 544′ | | |
| 47862_at | Cluster Incl. AA158234:zo76b01.s1 Homo sapiens cDNA, | 22.47041512 | 0.00011 |
| | 3 end /clone = IMAGE-592777 /clone_end = 3′ | | |
| | /gb = AA158234 /gi = 1733029 /ug = Hs.72222 /len = 614′ | | |
| 51811_at | Cluster Incl. AA535447:nf84h07.s1 Homo sapiens cDNA, | 18.5502306 | 0.000002 |
| | 3 end /clone = IMAGE-926653 /clone_end = 3′ | | |
| | /gb = AA535447 /gi = 2279700 /ug = Hs.5366 /len = 524′ | | |
| 49252_at | Cluster Incl. R40393:yf71c01.s1 Homo sapiens cDNA, | 15.5072013 | 0.000108 |
| | 3 end /clone = IMAGE-27679 /clone_end = 3′ | | |
| | /gb = R40393 /gi = 822823 /ug = Hs.239147 /len = 485′ | | |
| 55610_at | Cluster Incl. AI742239:wg39e09.x1 Homo sapiens cDNA, | 14.88983284 | 0.000003 |
| | 3 end /clone = IMAGE-2367496 /clone_end = 3′ | | |
| | /gb = AI742239 /gi = 5110527 /ug = Hs.91109 /len = 493′ | | |
| 46682_at | Cluster Incl. AI985652:wt19b08.x1 Homo sapiens cDNA, | 14.5125698 | 0.000036 |
| | 3 end /clone = IMAGE-2507895 /clone_end = 3′ | | |
| | /gb = AI985652 /gi = 5812929 /ug = Hs.7362 /len = 539′ | | |
| 45204_s_at | Cluster Incl. AI378647:tc57a04.x1 Homo sapiens cDNA, | 12.18224933 | 0.000011 |
| | 3 end /clone = IMAGE-2068686 /clone_end = 3′ | | |
| | /gb = AI378647 /gi = 4188500 /ug = Hs.42502 /len = 481′ | | |
| 54001_at | Cluster Incl. AI092936:qa81b05.x1 Homo sapiens cDNA, | 11.81231717 | 0.000235 |
| | 3 end /clone = IMAGE-1693137 /clone_end = 3′ | | |
| | /gb = AI092936 /gi = 3431912 /ug = Hs.6459 /len = 516′ | | |
| 45294_at | Cluster Incl. AI697470:tq08h01.x1 Homo sapiens cDNA, | 10.79542287 | 0.002361 |
| | 3 end /clone = IMAGE-2208241 /clone_end = 3′ | | |
| | /gb = AI697470 /gi = 4985370 /ug = Hs.205126 /len = 494′ | | |
| 46165_at | Cluster Incl. AA001552:ze46c08.s1 Homo sapiens cDNA, | 9.689374943 | 0.000026 |
| | 3 end /clone = IMAGE-362030 /clone_end = 3′ | | |
| | /gb = AA001552 /gl = 1437017 /ug = Hs.59839 /len = 566′ | | |
| 44558_at | Cluster Incl. AI148745:qc69e03.x1 Homo sapiens cDNA, | 9.603193191 | 0.000001 |
| | 3 end /clone = IMAGE-1714876 /clone_end = 3′ | | |
| | /gb = AI148745 /gi = 3677214 /ug = Hs.30715 /len = 733′ | | |
| 56495_at | Cluster Incl. AI301060:qo16c05.x1 Homo sapiens cDNA, | 8.981853987 | 0.000031 |
| | 3 end /clone = IMAGE-1908680 /clone_end = 3′ | | |
| | /gb = AI301060 /gi = 3960406 /ug = Hs.11365 /len = 618′ | | |
| 47475_at | Cluster Incl. AI057608:oy31e08.x1 Homo sapiens cDNA, | 7.95757126 | 0.001394 |
| | 3 end /clone = IMAGE-1667462 /clone_end = 3′ | | |
| | /gb = AI057608 /gi = 3331474 /ug = Hs.127826 /len = 657′ | | |
| 53718_at | Cluster Incl. AI684645:wa84h12.x1 Homo sapiens cDNA, | 7.724496401 | 0.00059 |
| | 3 end /clone = IMAGE-2302919 /clone_end = 3′ | | |
| | /gb = AI684645 /gi = 4895939 /ug = Hs.12126 /len = 585′ | | |
| 42973_at | Cluster Incl. AA458524:zx96b11.r1 Homo sapiens cDNA, | 7.478781625 | 0.000217 |
| | 5 end /clone = IMAGE-811581 /clone_end = 5′ | | |
| | /gb = AA458524 /gi = 2183431 /ug = Hs.21835 /len = 470′ | | |
| 55802_at | Cluster Incl. AW007803:wt03d03.x1 Homo sapiens cDNA, | 7.379846073 | 0.016111 |
| | 3 end /clone = IMAGE-2506373 /clone_end = 3′ | | |
| | /gb = AW007803 /gi = 5856581 /ug = Hs.61311 /len = 544′ | | |
| 46652_at | Cluster Incl. AA524036:ng32f10.s1 Homo sapiens cDNA, | 7.07207438 | 0.001998 |
| | 3 end /clone = IMAGE-936523 /clone_end = 3′ | | |
| | /gb = AA524036 /gi = 2264964 /ug = Hs.71190 /len = 608′ | | |
| 52119_at | Cluster Incl. AA418063:zv97b02.r1 Homo sapiens cDNA, | 6.967128041 | 0.000278 |
| | 5 end /clone = IMAGE-767691 /clone_end = 5′ | | |
| | /gb = AA418063 /gi = 2079937 /ug = Hs.44278 /len = 585′ | | |
| 54150_at | Cluster Incl. AI669065:we70d12.x1 Homo sapiens cDNA, | 6.834403508 | 0.000058 |
| | 3 end /clone = IMAGE-2346455 /clone_end = 3′ | | |
| | /gb = AI669065 /gi = 4833839 /ug = Hs.71779 /len = 518′ | | |
| 50849_at | Cluster Incl. AI922323:wn90h03.x1 Homo sapiens cDNA, | 6.666123575 | 0.000285 |
| | 3 end /clone = IMAGE-2453141 /clone_end = 3′ | | |
| | /gb = AI922323 /gi = 5658287 /ug = Hs.122576 /len = 483′ | | |
| 56122_at | Cluster Incl. AI223817:qi33d01.x1 Homo sapiens cDNA, | 6.326746141 | 0.002608 |
| | 3 end /clone = IMAGE-1858273 /clone_end = 3′ | | |
| | /gb = AI223817 /gi = 3806530 /ug = Hs.100686 /len = 620′ | | |
| 46659_at | Cluster Incl. AI742057:wg38d12.x1 Homo sapiens cDNA, | 6.316573114 | 0.000494 |
| | 3 end /clone = IMAGE-2367383 /clone_end = 3′ | | |
| | /gb = AI742057 /gi = 5110345 /ug = Hs.7155 /len = 603′ | | |
| 56806_at | Cluster Incl. AI809891:wf59e01.x1 Homo sapiens cDNA, | 6.165253015 | 0.001843 |
| | 3 end /clone = IMAGE-2359896 /clone_end = 3′ | | |
| | /gb = AI809891 /gi = 5396457 /ug = Hs.104573 /len = 481′ | | |
| 57094_at | Cluster Incl. AI189381:qd05h06.x1 Homo sapiens cDNA, | 6.135568271 | 0.000033 |
| | 3 end /clone = IMAGE-1722875 /clone_end = 3′ | | |
| | /gb = AI189381 /gi = 3740590 /ug = Hs.203125 /len = 807′ | | |
| 47060_at | Cluster Incl. AI422335:tf65f01.x1 Homo sapiens cDNA, | 6.055112775 | 0.000721 |
| | 3 end /clone = IMAGE-2104153 /clone_end = 3′ | | |
| | /gb = AI422335 /gi = 4268266 /ug = Hs.78358 /len = 540′ | | |
| 46644_at | Cluster Incl. AA523925:ng24g06.s1 Homo sapiens cDNA, | 6.039307564 | 0.000053 |
| | 3 end /clone = IMAGE-935770 /clone_end = 3′ | | |
| | /gb = AA523925 /gi = 2264853 /ug = Hs.70732 /len = 662′ | | |
| 56904_g_a | Cluster Incl. AW004007:wq94g10.x1 Homo sapiens cDNA, | 6.027803262 | 0.000267 |
| | 3 end /clone = IMAGE-2479746 /clone end = 3′ | | |
| | /gb = AW004007 /gi = 5850923 /ug = Hs.109257 /len = 262′ | | |
| 47890_at | Cluster Incl. AI921465:wo25d05.x1 Homo sapiens cDNA, | 5.778899418 | 0.000053 |
| | 3 end /clone = IMAGE-2456361 /clone_end = 3′ | | |
| | /gb = AI921465 /gi = 5657429 /ug = Hs.146314 /len = 504′ | | |
| 57861_at | Cluster Incl. AI096493:qa03d12.x1 Homo sapiens cDNA, | 5.69536482 | 0.000123 |
| | 3 end /clone = IMAGE-1685687 /clone_end = 3′ | | |
| | /gb = AI096493 /gi = 3445987 /ug = Hs.172572 /len = 586′ | | |
| 45382_g_a | Cluster Incl. AI392817:tg10a06.x1 Homo sapiens cDNA, | 5.623492806 | 0.000069 |
| | 3 end /clone = IMAGE-2108338 /clone_end = 3′ | | |
| | /gb = AI392817 /gi = 4222364 /ug = Hs.239331 /len = 543′ | | |
| 59661_at | Cluster Incl. W81116:zh49d10.s1 Homo sapiens cDNA, | 5.495775955 | 0.000252 |
| | 3 end /clone = IMAGE-415411 /clone_end = 3′ | | |
| | /gb = W81116 /gi = 1391615 /ug = Hs.20524 /len = 622′ | | |
| 44140_at | Cluster Incl. AA056278:zf53h03.s1 Homo sapiens cDNA, | 5.287393752 | 0.000019 |
| | 3 end /clone = IMAGE-380693 /clone_end = 3′ | | |
| | /gb = AA056278 /gi = 1548682 /ug = Hs.28669 /len = 632′ | | |
| 48745_s_at | Cluster Incl. AI979240:wu03g12.x1 Homo sapiens cDNA, | 5.222721476 | 0.003423 |
| | 3 end /clone = IMAGE-2515942 /clone_end = 3′ | | |
| | /gb = AI979240 /gi = 5804361 /ug = Hs.233729 /len = 226′ | | |
| 45183_at | Cluster Incl. AL118812:DKFZp761G1111_r1 Homo sapiens cDNA, | 5.057329191 | 0.000756 |
| | 5 end /clone = DKFZp761G1111 /clone_end = 5′ | | |
| | /gb = AL118812 /gi = 5924711 /ug = Hs.41139 /len = 1642′ | | |
| 49052_at | Cluster Incl. AA147884:zl50b04.s1 Homo sapiens cDNA, | 4.985943579 | 0.001171 |
| | 3 end /clone = IMAGE-505327 /clone_end = 3′ | | |
| | /gb = AA147884 /gi = 1717300 /ug = Hs.9812 /len = 652′ | | |
| 47522_at | Cluster Incl. AW007479:ws52c07.x1 Homo sapiens cDNA, | 4.974197171 | 0.000006 |
| | 3 end /clone = IMAGE-2500812 /clone_end = 3′ | | |
| | /gb = AW007479 /gi = 5856257 /ug = Hs.143974 /len = 497′ | | |
| 48120_at | Cluster Incl. H30385:ym58d12.s1 Homo sapiens cDNA, | 4.798932814 | 0.000126 |
| | 3 end /clone = IMAGE-52891 /clone_end = 3′ | | |
| | /gb = H30385 /gi = 901295 /ug = Hs.11067 /len = 519′ | | |
| 46675_at | Cluster Incl. AA625199:af70e02.r1 Homo sapiens cDNA, | 4.790908464 | 0.004853 |
| | 5 end /clone = IMAGE-1047386 /clone_end = 5′ | | |
| | /gb = AA625199 /gi = 2537584 /ug = Hs.72289 /len = 566′ | | |
| 52054_at | Cluster Incl. AI434780:ti20c05.x1 Homo sapiens cDNA, | 4.774162147 | 0.000001 |
| | 3 end /clone = IMAGE-2131016 /clone_end = 3′ | | |
| | /gb = AI434780 /gi = 4298572 /ug = Hs.4248 /len = 933′ | | |
| 46219_at | Cluster Incl. Z99410:HSZ99410 Homo sapiens cDNA | 4.7581476 | 0.000006 |
| | /clone = DKFZphamy1_1c7 /gb = Z99410 /gi = 2415650 | | |
| | /ug = Hs.6314 /len = 669 | | |
| 47627_at | Cluster.Incl. AI445492:tj24c09.x1 Homo sapiens cDNA, | 4.705362783 | 0.006739 |
| | 3 end /clone = IMAGE-2142448 /clone_end = 3′ | | |
| | /gb = AI445492 /gi = 4288591 /ug = Hs.98370 /len = 545′ | | |
| 44770_s_at | Cluster Incl. AI341166:qx89h02.x1 Homo sapiens cDNA, | 4.699694547 | 0.000442 |
| | 3 end /clone = IMAGE-2009715 /clone_end = 3′ | | |
| | /gb = AI341166 /gi = 4078093 /ug = Hs.233977 /len = 257′ | | |
| 52019_at | Cluster Incl. AI557210:PT2.1_14_H10.r Homo sapiens cDNA, | 4.685097403 | 0.00302 |
| | 3 end /clone_end = 3′ /gb = AI557210 | | |
| | /gi = 4489573 /ug = Hs.41271 /len = 867′ | | |
| 53781_at | Cluster Incl. W74476:zd75a11.s1 Homo sapiens cDNA, | 4.663360742 | 0.003285 |
| | 3 end /clone = IMAGE-346460 /clone_end = 3′ | | |
| | /gb = W74476 /gi = 1384763 /ug = Hs.12680 /len = 675′ | | |
| 56172_at | Cluster Incl. AI979261:wr72g05.x1 Homo sapiens cDNA, | 4.605593005 | 0.001197 |
| | 3 end /clone = IMAGE-2493272 /clone_end = 3′ | | |
| | /gb = AI979261 /gi = 5804280 /ug = Hs.102720 /len = 809′ | | |
| 52117_at | Cluster Incl. AI670876:wa06c12.x1 Homo sapiens cDNA, | 4.577288892 | 0.013189 |
| | 3 end /clone = IMAGE-2297302 /clone_end = 3′ | | |
| | /gb = AI670876 /gi = 4850607 /ug = Hs.44276 /len = 798′ | | |
| 59614_g_a | Cluster Incl. AI669308:wb85b10.x1 Homo sapiens cDNA, | 4.508059219 | 0.001927 |
| | 3 end /clone = IMAGE-2312443 /clone_end = 3′ | | |
| | /gb = AI669308 /gi = 4834082 /ug = Hs.196337 /len = 632′ | | |
| 56688_at | Cluster Incl. AI631355:tz83d05.x1 Homo sapiens cDNA, | 4.461870453 | 0.001025 |
| | 3 end /clone = IMAGE-2295177 /clone_end = 3′ | | |
| | /gb = AI631355 /gi = 4682685 /ug = Hs.92096 /len = 390′ | | |
| 59709_at | Cluster Incl. AI675453:wb99f04.x1 Homo sapiens cDNA, | 4.443984215 | 0.004424 |
| | 3 end /clone = IMAGE-2313823 /clone_end = 3′ | | |
| | /gb = AI675453 /gi = 4875933 /ug = Hs.21432 /len = 562′ | | |
| 43963_at | Cluster Incl. AI703454:we24d09.x1 Homo sapiens cDNA, | 4.381967582 | 0.004589 |
| | 3 end /clone = IMAGE-2342033 /clone_end = 3′ | | |
| | /gb = AI703454 /gi = 4991354 /ug = Hs.26176 /len = 567′ | | |
| 56534_at | Cluster Incl. N20945:yx54f12.s1 Homo sapiens cDNA, | 4.354613332 | 0.000004 |
| | 3 end /clone = IMAGE-265583 /clone_end = 3′ | | |
| | /gb = N20945 /gi = 1126115 /ug = Hs.12210 /len = 621′ | | |
| 49162_f_at | Cluster Incl. AI610692:tp40f03.x1 Homo sapiens cDNA, | 4.340360862 | 0.00009 |
| | 3 end /clone = IMAGE-2190269 /clone_end = 3′ | | |
| | /gb = AI610692 /gi = 4619859 /ug = Hs.234412 /len = 474′ | | |
| 54850_at | Cluster Incl. AI860751:wl05b07.x1 Homo sapiens cDNA, | 4.319799905 | 0.004106 |
| | 3 end /clone = IMAGE-2423989 /clone_end = 3′ | | |
| | /gb = AI860751 /gi = 5514367 /ug = Hs.182476 /len = 637′ | | |
| 51244_at | Cluster Incl. AA447232:zw93a05.r1 Homo sapiens cDNA, | 4.254851949 | 0.008281 |
| | 5 end /clone = IMAGE-784496 /clone_end = 5′ | | |
| | /gb = AA447232 /gi = 2159897 /ug = Hs.34806 /len = 580′ | | |
| 47548_f_at | Cluster Incl. AA621124:af34f07.s1 Homo sapiens cDNA, | 4.238866373 | 0.00289 |
| | 3 end /clone = IMAGE-1033573 /clone_end = 3′ | | |
| | /gb = AA621124 /gi = 2525063 /ug = Hs.93135 /len = 639′ | | |
| 42988_at | Cluster Incl. AI344312:tc03e01.x1 Homo sapiens cDNA, | 4.225234676 | 0.001321 |
| | 3 end /clone = IMAGE-2062776 /clone_end = 3′ | | |
| | /gb = AI344312 /gi = 4081518 /ug = Hs.22011 /len = 578′ | | |
| 48431_at | Cluster Incl. AI970292:wr09b06.x1 Homo sapiens cDNA, | 4.223781701 | 0.000002 |
| | 3 end /clone = IMAGE-2481011 /clone_end = 3′ | | |
| | /gb = AI970292 /gi = 5767118 /ug = Hs.20279 /len = 605′ | | |
| 48077_at | Cluster Incl. AI741321:wg20c02.x1 Homo sapiens cDNA, | 4.208264213 | 0.000188 |
| | 3 end /clone = IMAGE-2365634 /clone_end = 3′ | | |
| | /gb = AI741321 /gi = 5109609 /ug = Hs.10760 /len = 525′ | | |
| 44112_at | Cluster Incl. AA224245:zr14h04.s1 Homo sapiens cDNA, | 4.200119211 | 0.000005 |
| | 3 end /clone = IMAGE-648823 /clone_end = 3′ | | |
| | /gb = AA224245 /gi = 1844770 /ug = Hs.26612 /len = 455′ | | |
| 45874_at | Cluster Incl. AI961994:wt41c03.x1 Homo sapiens cDNA, | 4.191359471 | 0.011876 |
| | 3 end /clone = IMAGE-2510020 /clone_end = 3′ | | |
| | /gb = AI961994 /gi = 5754696 /ug = Hs.30899 /len = 433′ | | |
| 52275_s_at | Cluster Incl. H11652:ym17g07.s1 Homo sapiens cDNA, | 4.123364243 | 0.000508 |
| | 3 end /clone = IMAGE-48178 /clone_end = 3′ | | |
| | /gb = H11652 /gi = 876472 /ug = Hs.236698 /len = 481′ | | |
| 55755_at | Cluster Incl. AI345945:qp47e12.x1 Homo sapiens cDNA, | 4.108035965 | 0.000005 |
| | 3 end /clone = IMAGE-1926190 /clone_end = 3′ | | |
| | /gb = AI345945 /gi = 4083151 /ug = Hs.10114 /len = 796′ | | |
| 48854_i_at | Cluster Incl. AW025683:wu07a09.x1 Homo sapiens cDNA, | 4.071776301 | 0.000025 |
| | 3 end /clone = IMAGE-2516248 /clone_end = 3′ | | |
| | /gb = AW025683 /gi = 5879213 /ug = Hs.237383 /len = 319′ | | |
| 53484_at | Cluster Incl. AI631585:wa99g06.x1 Homo sapiens cDNA, | 3.999414942 | 0.004362 |
| | 3 end /clone = IMAGE-2304346 /clone_end = 3′ | | |
| | /gb = AI631585 /gi = 4682915 /ug = Hs.33977 /len = 503′ | | |
| 57597_at | Cluster Incl. AW025904:wv71e11.x1 Homo sapiens cDNA, | 3.946689721 | 0.000247 |
| | 3 end /clone = IMAGE-991071 /clone_end = 3′ | | |
| | /gb = AW025904 /gi = 5879434 /ug = Hs.123661 /len = 674′ | | |
| 56940_g_a | Cluster Incl. AI963304:wt61d01.x1 Homo sapiens cDNA, | 3.934211473 | 0.037291 |
| | 3 end /clone = IMAGE-2511937 /clone_end = 3′ | | |
| | /gb = AI963304 /gi = 5756017 /ug = Hs.110373 /len = 660′ | | |
| 45144_at | Cluster Incl. AA641636:nr80c04.s1 Homo sapiens cDNA, | 3.920750642 | 0.008126 |
| | 3 end /clone = IMAGE-1174278 /clone_end = 3′ | | |
| | /gb = AA641636 /gi = 2566854 /ug = Hs.37477 /len = 569′ | | |
| 55636_at | Cluster Incl. W84893:zd88d09.s1 Homo sapiens cDNA, | 3.91767742 | 0.001305 |
| | 3 end /clone = IMAGE-347729 /clone_end = 3′ | | |
| | /gb = W84893 /gi = 1395457 /ug = Hs.9305 /len = 619′ | | |
| 52349_s_at | Cluster Incl. AA765843:oa25e09.s1 Homo sapiens cDNA | 3.908250964 | 0.000084 |
| | /clone = IMAGE-1306024 /gb = AA765843 /gi = 2817081 | | |
| | /ug = Hs.238961 /len = 496 | | |
| 44744_at | Cluster Incl. AI816843:wj34g08.x1 Homo sapiens cDNA, | 3.885817076 | 0.00012 |
| | 3 end /clone = IMAGE-2404766 /clone_end = 3′ | | |
| | /gb = AI816843 /gi = 5435922 /ug = Hs.172613 /len = 822′ | | |
| 45718_at | Cluster Incl. AA426499:zw02b06.r1 Homo sapiens cDNA, | 3.844537888 | 0.001962 |
| | 5 end /clone = IMAGE-768083 /clone_end = 5′ | | |
| | /gb = AA426499 /gi = 2106744 /ug = Hs.239900 /len = 553′ | | |
| 47598_at | Cluster Incl. AI767291:wh25h08.x1 Homo sapiens cDNA, | 3.797287151 | 0.004107 |
| | 3 end /clone = IMAGE-2381823 /clone_end = 3′ | | |
| | /gb = AI767291 /gi = 5233888 /ug = Hs.97101 /len = 601′ | | |
| 48546_s_at | Cluster Incl. N30008:yx82e08.s1 Homo sapiens cDNA, | 3.759130275 | 0.000038 |
| | 3 end /clone = IMAGE-268262 /clone_end = 3′ | | |
| | /gb = N30008 /gi = 1148528 /ug = Hs.17756 /len = 592′ | | |
| 51222_at | Cluster Incl. AW007811:wt03d11.x1 Homo sapiens cDNA, | 3.758109572 | 0.004507 |
| | 3 end /clone = IMAGE-2506389 /clone_end = 3′ | | |
| | /gb = AW007811 /gi = 5856589 /ug = Hs.32793 /len = 550′ | | |
| 52641_at | Cluster Incl. AI828579:wl42e03.x1 Homo sapiens cDNA, | 3.75632061 | 0.001209 |
| | 3 end /clone = IMAGE-2427580 /clone_end = 3′ | | |
| | /gb = AI828579 /gi = 5449250 /ug = Hs.239275 /len = 384′ | | |
| 52337_g_a | Cluster Incl. AI762208:wi54c10.x1 Homo sapiens cDNA, | 3.709836573 | 0.000216 |
| | 3 end /clone = IMAGE-2394066 /clone_end = 3′ | | |
| | /gb = AI762208 /gi = 5177875 /ug = Hs.238411 /len = 553′ | | |
| 52290_g_a | Cluster Incl. AA514342:nf56d01.s1 Homo sapiens cDNA, | 3.658373973 | 0.001941 |
| | 3 end /clone = IMAGE-923905 /clone_end = 3′ | | |
| | /gb = AA514342 /gi = 2253850 /ug = Hs.236950 /len = 606′ | | |
| 45331_at | Cluster Incl. AA102468:zm26a12.s1 Homo sapiens cDNA, | 3.627638605 | 0.00039 |
| | 3 end /clone = IMAGE-526750 /clone_end = 3′ | | |
| | /gb = AA102468 /gi = 1647486 /ug = Hs.234977 /len = 573′ | | |
| 52823_at | Cluster Incl. AI620209:tu54d10.x1 Homo sapiens cDNA, | 3.626992495 | 0.001038 |
| | 3 end /clone = IMAGE-2254867 /clone_end = 3′ | | |
| | /gb = AI620209 /gi = 4629335 /ug = Hs.37916 /len = 576′ | | |
| 47534_at | Cluster Incl. AI569980:tr90f02.x1 Homo sapiens cDNA, | 3.623794274 | 0.001497 |
| | 3 end /clone = IMAGE-2226363 /clone_end = 3′ | | |
| | /gb = AI569980 /gi = 4533354 /ug = Hs.9238 /len = 495′ | | |
| 56223_at | Cluster Incl. AI769689:wj25f12.x1 Homo sapiens cDNA, | 3.617285719 | 0.00013 |
| | 3 end /clone = IMAGE-2403887 /clone_end = 3′ | | |
| | /gb = AI769689 /gi = 5236198 /ug = Hs.192878 /len = 552′ | | |
| 56673_at | Cluster Incl. AA769578:nz42a04.s1 Homo sapiens cDNA | 3.598876589 | 0.001823 |
| | /clone = IMAGE-1290414 /gb = AA769578 /gi = 2820816 | | |
| | /ug = Hs.90488 /len = 575 | | |
| 48054_at | Cluster Incl. AA024984:ze80c07.s1 Homo sapiens cDNA, | 3.583829556 | 0.004289 |
| | 3 end /clone = IMAGE-365292 /clone_end = 3′ | | |
| | /gb = AA024984 /gi = 1489889 /ug = Hs.10574 /len = 474′ | | |
| 58322_at | Cluster Incl. AI765890:wh66g10.x1 Homo sapiens cDNA, | 3.5683257 | 0.000325 |
| | 3 end /clone = IMAGE-2385762 /clone_end = 3′ | | |
| | /gb = AI765890 /gi = 5232399 /ug = Hs.16341 /len = 511′ | | |
| 52826_at | Cluster Incl. AL037412:DKFZp564L0571_s1 Homo sapiens cDNA, | 3.568196085 | 0.000189 |
| | 3 end /clone = DKFZp564L0571 /clone_end = 3′ | | |
| | /gb = AL037412 /gi = 5406808 /ug = Hs.38039 /len = 804′ | | |
| 57195_at | Cluster Incl. AI935271:wp16c05.x1 Homo sapiens cDNA, | 3.544558558 | 0.000007 |
| | 3 end /clone = IMAGE-2465000 /clone_end = 3′ | | |
| | /gb = AI935271 /gi = 5674141 /ug = Hs.5663 /len = 562′ | | |
| 44040_at | Cluster Incl. AA524093:ng33f04.s1 Homo sapiens cDNA, | 3.544444641 | 0.002777 |
| | 3 end /clone = IMAGE-936607 /clone_end = 3′ | | |
| | /gb = AA524093 /gi = 2265021 /ug = Hs.23158 /len = 718′ | | |
| 48099_at | Cluster Incl. AA005023:zh96a11.s1 Homo sapiens cDNA, | 3.528226654 | 0.012069 |
| | 3 end /clone = IMAGE-429116 /clone_end = 3′ | | |
| | /gb = AA005023 /gi = 1448864 /ug = Hs.10888 /len = 593′ | | |
| 51270_at | Cluster Incl. AA005361:zh97b01.s1 Homo sapiens cDNA, | 3.504429429 | 0.00292 |
| | 3 end /clone = IMAGE-429193 /clone_end = 3′ | | |
| | /gb = AA005361 /gi = 1448394 /ug = Hs.36723 /len = 690′ | | |
| 49888_f_at | Cluster Incl. AI357616:qu20g08.x1 Homo sapiens cDNA, | 3.47870117 | 0.000114 |
| | 3 end /clone = IMAGE-1965374 /clone_end = 3′ | | |
| | /gb = AI357616 /gi = 4109237 /ug = Hs.101651 /len = 405′ | | |
| 51160_at | Cluster Incl. AA004208:zh97c02.s1 Homo sapiens cDNA, | 3.477061385 | 0.000445 |
| | 3 end /clone = IMAGE-429218 /clone_end = 3′ | | |
| | /gb = AA004208 /gi = 1448403 /ug = Hs.27437 /len = 665′ | | |
| 45826_at | Cluster Incl. AA044844:zk74f11.r1 Homo sapiens cDNA, | 3.434292964 | 0.000168 |
| | 5 end /clone = IMAGE-488589 /clone_end = 5′ | | |
| | /gb = AA044844 /gi = 1523066 /ug = Hs.5944 /len = 645′ | | |
| 52016_at | Cluster Incl. AI672414:ty64h06.x1 Homo sapiens cDNA, | 3.423366376 | 0.001144 |
| | 3 end /clone = IMAGE-2283899 /clone_end = 3′ | | |
| | /gb = AI672414 /gi = 4852145 /ug = Hs.41241 /len = 536′ | | |
| 46720_at | Cluster Incl. AI066598:ov47g09.x1 Homo sapiens cDNA, | 3.418349709 | 0.000205 |
| | 3 end /clone = IMAGE-1640512 /clone_end = 3′ | | |
| | /gb = AI066598 /gi = 3367300 /ug = Hs.9853 /len = 760′ | | |
| 48545_at | Cluster Incl. W87383:zh66f09.r1 Homo sapiens cDNA, | 3.403794238 | 0.000121 |
| | 5 end /clone = IMAGE-417065 /clone_end = 5′ | | |
| | /gb = W87383 /gi = 1401438 /ug = Hs.17731 /len = 625′ | | |
| 44630_at | Cluster Incl. AA521049:aa71c10.s1 Homo sapiens cDNA, | 3.356102325 | 0.003255 |
| | 3 end /clone = IMAGE-826386 /clone_end = 3′ | | |
| | /gb = AA521049 /gi = 2261592 /ug = Hs.34487 /len = 593′ | | |
| 54625_at | Cluster Incl. AA057543:zl93d01.s1 Homo sapiens cDNA, | 3.35563894 | 0.003164 |
| | 3 end /clone = IMAGE-512161 /clone_end = 3′ | | |
| | /gb = AA057543 /gi = 1550248 /ug = Hs.15911 /len = 655′ | | |
| 42831_at | Cluster Incl. AI201843:qs76f10.x1 Homo sapiens cDNA, | 3.352230883 | 0.000686 |
| | 3 end /clone = IMAGE-1944043 /clone_end = 3′ | | |
| | /gb = AI201843 /gi = 3754449 /ug = Hs.169133 /len = 358′ | | |
| 44778_g_a | Cluster Incl. AA553396:nk80b05.s1 Homo sapiens cDNA, | 3.349340084 | 0.001436 |
| | 3 end /clone = IMAGE-1019793 /clone_end = 3′ | | |
| | /gb = AA553396 /gi = 2323935 /ug = Hs.234190 /len = 454′ | | |
| 48540_at | Cluster Incl. R26843:yh52a03.s1 Homo sapiens cDNA, | 3.348102248 | 0.006734 |
| | 3 end /clone = IMAGE-133324 /clone_end = 3′ | | |
| | /gb = R26843 /gi = 782978 /ug = Hs.17558 /len = 490′ | | |
| 55752_at | Cluster Incl. AI198880:qf66c09.x1 Homo sapiens cDNA, | 3.344024791 | 0.004837 |
| | 3 end /clone = IMAGE-1754992 /clone_end = 3′ | | |
| | /gb = AI198880 /gi = 3751486 /ug = Hs.10098 /len = 786′ | | |
| 57171_at | Cluster Incl. X84716:HSEST92 Homo sapiens cDNA | 3.338417032 | 0.000053 |
| | /clone = MEC-92 /gb = X84716 /gi = 673403 | | |
| | /ug = Hs.29748 /len = 1214 | | |
| 48013_at | Cluster Incl. AI768116:wg81c10.x1 Homo sapiens cDNA, | 3.322252213 | 0.015046 |
| | 3 end /clone = IMAGE-2371506 /clone_end = 3′ | | |
| | /gb = AI768116 /gi = 5234625 /ug = Hs.10086 /len = 557′ | | |
| 51840_at | Cluster Incl. AI765436:wi80a08.x1 Homo sapiens cDNA, | 3.318140109 | 0.020706 |
| | 3 end /clone = IMAGE-2399606 /clone_end = 3′ | | |
| | /gb = AI765436 /gi = 5231945 /ug = Hs.5605 /len = 498′ | | |
| 45228_at | Cluster Incl. AI271425:qi19b04.x1 Homo sapiens cDNA, | 3.317124397 | 0.007521 |
| | 3 end /clone = IMAGE-1856911 /clone_end = 3′ | | |
| | /gb = AI271425 /gi = 3890592 /ug = Hs.43749 /len = 551′ | | |
| 52062_f_at | Cluster Incl. H98105:yx09b10.s1 Homo sapiens cDNA, | 3.313066937 | 0.002819 |
| | 3 end /clone = IMAGE-261211 /clone_end = 3′ | | |
| | /gb = H98105 /gi = 1118990 /ug = Hs.42491 /len = 584′ | | |
| 50795_at | Cluster Incl. AI693592:wd79a05.x1 Homo sapiens cDNA, | 3.31259622 | 0.000537 |
| | 3 end /clone = IMAGE-2337776 /clone_end = 3′ | | |
| | /gb = AI693592 /gi = 4970932 /ug = Hs.112712 /len = 508′ | | |
| 57022_at | Cluster Incl. AI742490:wg43e10.x1 Homo sapiens cDNA, | 3.310509474 | 0.000572 |
| | 3 end /clone = IMAGE-2367882 /clone_end = 3′ | | |
| | /gb = AI742490 /gi = 5110778 /ug = Hs.112885 /len = 550′ | | |
| 42999_at | Cluster Incl. AI758408:ty68f09.x1 Homo sapiens cDNA, | 3.305590563 | 0.007574 |
| | 3 end /clone = IMAGE-2284265 /clone_end = 3′ | | |
| | /gb = AI758408 /gi = 5152131 /ug = Hs.22247 /len = 633′ | | |
| 58695_at | Cluster Incl. AA461376:zx70g03.r1 Homo sapiens cDNA, | 3.290571553 | 0.000149 |
| | 5 end /clone = IMAGE-796852 /clone_end = 5′ | | |
| | /gb = AA461376 /gi = 2186496 /ug = Hs.9625 /len = 588′ | | |
| 47110_at | Cluster Incl. AA205598:zq68d01.s1 Homo sapiens cDNA, | 3.286952373 | 0.011612 |
| | 3 end /clone = IMAGE-646753 /clone_end = 3′ | | |
| | /gb = AA205598 /gi = 1803606 /ug = Hs.86366 /len = 618′ | | |
| 53922_at | Cluster Incl. AA195789:zp95b07.r1 Homo sapiens cDNA, | 3.282601218 | 0.015234 |
| | 5 end /clone = IMAGE-627925 /clone_end = 5′ | | |
| | /gb = AA195789 /gi = 1791380 /ug = Hs.60440 /len = 619′ | | |
| 47899_at | Cluster Incl. AA056755:zk81g02.s1 Homo sapiens cDNA, | 3.276090147 | 0.007052 |
| | 3 end /clone = IMAGE-489266 /clone_end = 3′ | | |
| | /gb = AA056755 /gi = 1549130 /ug = Hs.151714 /len = 496′ | | |
| 43053_g_a | Cluster Incl. AW025012:wu93a04.x1 Homo sapiens cDNA, | 3.25557678 | 0.00068 |
| | 3 end /clone = IMAGE-990913 /clone_end = 3′ | | |
| | /gb = AW025012 /gi = 5878542 /ug = Hs.23295 /len = 520′ | | |
| 48080_at | Cluster Incl. A1566481:tr60d03.x2 Homo sapiens cDNA, | 3.224686597 | 0.000089 |
| | 3 end /clone = IMAGE-2222693 /clone_end = 3′ | | |
| | /gb = AI566481 /gi = 4524933 /ug = Hs.10784 /len = 551′ | | |
| 50417_at | Cluster Incl. AA641023:nr72c02.s1 Homo sapiens cDNA, | 3.222451104 | 0.000231 |
| | 3 end /clone = IMAGE-1173506 /clone_end = 3′ | | |
| | /gb = AA641023 /gi = 2566273 /ug = Hs.25303 /len = 571′ | | |
| 55749_at | Cluster Incl. AA135720:zl11b08.s1 Homo sapiens cDNA, | 3.211891299 | 0.002311 |
| | 3 end /clone = IMAGE-501591 /clone_end = 3′ | | |
| | /gb = AA135720 /gi = 1696732 /ug = Hs.10083 /len = 563′ | | |
| 45390_at | Cluster Incl. AI769269:wg35h12.x1 Homo sapiens cDNA, | 3.203408013 | 0.001334 |
| | 3 end /clone = IMAGE-2367143 /clone_end = 3′ | | |
| | /gb = AI769269 /gi = 5235778 /ug = Hs.239541 /len = 443′ | | |
| 56035_at | Cluster Incl. AL041162:DKFZp434G1516_s1 Homo sapiens cDNA, | 3.199890488 | 0.000887 |
| | 3 end /clone = DKFZp434G1516 /clone_end = 3′ | | |
| | /gb = AL041162 /gi = 5410098 /ug = Hs.98587 /len = 775′ | | |
| 54594_at | Cluster Incl. AA009571:zi04h05.r1 Homo sapiens cDNA, | 3.199200539 | 0.042469 |
| | 5 end /clone = IMAGE-429849 /clone_end = 5′ | | |
| | /gb = AA009571 /gi = 1470709 /ug = Hs.82290 /len = 467′ | | |
| 56827_at | Cluster Incl. AI932306:wd26g11.x1 Homo sapiens cDNA, | 3.177892231 | 0.000647 |
| | 3 end /clone = IMAGE-2329316 /clone_end = 3′ | | |
| | /gb = A1932306 /gi = 5671043 /ug = Hs.105463 /len = 528′ | | |
| 45143_at | Cluster Incl. AA160973:zq40h10.s1 Homo sapiens cDNA, | 3.169675904 | 0.000021 |
| | 3 end /clone = IMAGE-632227 /clone_end = 3′ | | |
| | /gb = AA160973 /gi = 1735680 /ug = Hs.37443 /len = 544′ | | |
| 44974_at | Cluster Incl. AI970896:wr21b11.x1 Homo sapiens cDNA, | 3.140815237 | 0.001848 |
| | 3 end /clone = IMAGE-2488317 /clone_end = 3′ | | |
| | /gb = AI970896 /gi = 5767722 /ug = Hs.233634 /len = 514′ | | |
| 54049_at | Cluster Incl. AI652991:wb42b11.x1 Homo sapiens cDNA, | 3.138854596 | 0.014071 |
| | 3 end /clone = IMAGE-2308317 /clone_end = 3′ | | |
| | /gb = AI652991 /gi = 4736970 /ug = Hs.6682 /len = 528′ | | |
| 59346_at | Cluster Incl. W96205:ze10d04.s1 Homo sapiens cDNA, | 3.133106727 | 0.018285 |
| | 3 end /clone = IMAGE-358567 /clone_end = 3′ | | |
| | /gb = W96205 /gi = 1426131 /ug = Hs.163703 /len = 408′ | | |
| 45280_at | Cluster Incl. AA703316:zj11b07.s1 Homo sapiens cDNA, | 3.093960419 | 0.000016 |
| | 3 end /clone = 449941 /clone_end = 3′ | | |
| | /gb = AA703316 /gi = 2713234 /ug = Hs.19221 /len = 499′ | | |
| 50287_at | Cluster Incl. R61210:yh06e09.r1 Homo sapiens cDNA, | 3.083985493 | 0.023892 |
| | 5 end /clone = IMAGE-42341 /clone_end = 5′ | | |
| | /gb = R61210 /gi = 831905 /ug = Hs.239431 /len = 551′ | | |
| 58317_at | Cluster Incl. AI638611:tt31b11.x1 Homo sapiens cDNA, | 3.068722453 | 0.000172 |
| | 3 end /clone = IMAGE-2242365 /clone_end = 3′ | | |
| | /gb = AI638611 /gi = 4690845 /ug = Hs.16229 /len = 639′ | | |
| 51155_at | Cluster Incl. AL043669:DKFZp434J2027_s1 Homo sapiens cDNA, | 3.056662545 | 0.002265 |
| | 3 end /clone = DKFZp434J2027 /clone_end = 3′ | | |
| | /gb = AL043669 /gi = 5423056 /ug = Hs.27268 /len = 540′ | | |
| 43366_at | Cluster Incl. AI979079:wr70b09.x1 Homo sapiens cDNA, | 3.055354669 | 0.007779 |
| | 3 end /clone = IMAGE-2493017 /clone_end = 3′ | | |
| | /gb = AI979079 /gi = 5804109 /ug = Hs.233723 /len = 490′ | | |
| 52848_at | Cluster Incl. AI491983:to07e03.x1 Homo sapiens cDNA, | 3.008324353 | 0.003676 |
| | 3 end /clone = IMAGE-2178364 /clone_end = 3′ | | |
| | /gb = AI491983 /gi = 4392986 /ug = Hs.74911 /len = 655′ | | |
| 57596_at | Cluster Incl. T75313:yc89d05.r1 Homo sapiens cDNA, | 3.001649666 | 0.020524 |
| | 5 end /clone = IMAGE-23443 /clone_end = 5′ | | |
| | /gb = T75313 /gi = 692075 /ug = Hs.12365 /len = 530′ | | |
| 43994_at | Cluster Incl. AI333224:qq05h01.x1 Homo sapiens cDNA, | 3.000320561 | 0.004173 |
| | 3 end /clone = IMAGE-1931665 /clone_end = 3′ | | |
| | /gb = AI333224 /gi = 4069783 /ug = Hs.26955 /len = 504′ | | |
| 43993_at | Cluster Incl. AA223148:zr07g09.s1 Homo sapiens cDNA, | 0.328758769 | 0.000839 |
| | 3 end /clone = IMAGE-650848 /clone_end = 3′ | | |
| | /gb = AA223148 /gi = 1843707 /ug = Hs.26942 /len = 492′ | | |
| 42833_g_a | Cluster Incl. W61185:zd31c11.s1 Homo sapiens cDNA, | 0.328541123 | 0.000416 |
| | 3 end /clone = IMAGE-342260 /clone_end = 3′ | | |
| | /gb = W61185 /gi = 1368020 /ug = Hs.169345 /len = 400′ | | |
| 45878_at | Cluster Incl. W21966:59c7 Homo sapiens cDNA | 0.328253841 | 0.000258 |
| | /clone = (not-directional) /gb = W21966 /gi = 1298799 | | |
| | /ug = Hs.31097 /len = 767 | | |
| 44711_at | Cluster Incl. AI928869:au64d01.x1 Homo sapiens cDNA | 0.327580662 | 0.023675 |
| | /clone = IMAGE /gb = AI928869 /gi = 5664768 | | |
| | /ug = Hs.15977 /len = 630 | | |
| 50314_i_at | Cluster Incl. AI761506:wi61b11.x1 Homo sapiens cDNA, | 0.326479347 | 0.018768 |
| | 3 end /clone = IMAGE-2394717 /clone_end = 3′ | | |
| | /gb = AI761506 /gi = 5177173 /ug = Hs.239692 /len = 544′ | | |
| 42065_at | Cluster Incl. H28581:yl63e11.s1 Homo sapiens cDNA, | 0.324964383 | 0.000041 |
| | 3 end /clone = IMAGE-162956 /clone_end = 3′ | | |
| | /gb = H28581 /gi = 898934 /ug = Hs.92711 /len = 416′ | | |
| 46079_at | Cluster Incl. W63785:zd30g09.s1 Homo sapiens cDNA, | 0.319996048 | 0.000015 |
| | 3 end /clone = IMAGE-342208 /clone_end = 3′ | | |
| | /gb = W63785 /gi = 1371386 /ug = Hs.57843 /len = 495′ | | |
| 42987_at | Cluster Incl. AI003579:ai88d07.s1 Homo sapiens cDNA, | 0.318539582 | 0.000912 |
| | 3 end /clone = IMAGE-1387885 /clone_end = 3′ | | |
| | /gb = AI003579 /gi = 3203913 /ug = Hs.22003 /len = 600′ | | |
| 57915_at | Cluster Incl. AA613523:nq22d01.s1 Homo sapiens cDNA, | 0.315418617 | 0.001639 |
| | 3 end /clone = IMAGE-1144609 /clone_end = 3′ | | |
| | /gb = AA613523 /gi = 2464561 /ug = Hs.22824 /len = 507′ | | |
| 56272_at | Cluster Incl. AI827230:wl09e02.x1 Homo sapiens cDNA, | 0.311014472 | 0.001289 |
| | 3 end /clone = IMAGE-2424410 /clone_end = 3′ | | |
| | /gb = AI827230 /gi = 5447901 /ug = Hs.20665 /len = 748′ | | |
| 59466_at | Cluster Incl. AA447295:AC.F02R Homo sapiens cDNA, | 0.310739955 | 0.002774 |
| | 5 end /clone = AC.F02 /clone_end = 5′ /gb = AA447295 | | |
| | /gi = 3025381 /ug = Hs.168953 /len = 589′ | | |
| 42803_at | Cluster Incl. AI377305:te60a03.x1 Homo sapiens cDNA, | 0.307373662 | 0.00498 |
| | 3 end /clone = IMAGE-2091052 /clone_end = 3′ | | |
| | /gb = AI377305 /gi = 4187158 /ug = Hs.165694 /len = 474′ | | |
| 46200_at | Cluster Incl. AA742697:nx30g04.s1 Homo sapiens cDNA | 0.307203732 | 0.04226 |
| | /clone = IMAGE-1257654 /gb = AA742697 /gi = 2782203 | | |
| | /ug = Hs.62492 /len = 526 | | |
| 45635_at | Cluster Incl. AI801898:tx28h03.x1 Homo sapiens cDNA, | 0.306826026 | 0.001547 |
| | 3 end /clone = IMAGE-2270933 /clone_end = 3′ | | |
| | /gb = AI801898 /gi = 5367370 /ug = Hs.47166 /len = 604′ | | |
| 46095_at | Cluster Incl. AL044632:DKFZp434O082_r1 Homo sapiens cDNA, | 0.306352205 | 0.003349 |
| | 5 end /clone = DKFZp434O082 /clone_end = 5′ | | |
| | /gb = AL044632 /gi = 5432847 /ug = Hs.55016 /len = 554′ | | |
| 47653_r_at | Cluster Incl. AW023468:df55c11.y1 Homo sapiens cDNA, | 0.306297842 | 0.003547 |
| | 5 end /clone = IMAGE-2487188 /clone_end = 5′ | | |
| | /gb = AW023468 /gi = 5876998 /ug = Hs.165320 /len = 473′ | | |
| 43046_at | Cluster Incl. AI419030:tf53b01.x1 Homo sapiens cDNA, | 0.306182917 | 0.034114 |
| | 3 end /clone = IMAGE-2102953 /clone_end = 3′ | | |
| | /gb = AI419030 /gi = 4264961 /ug = Hs.23202 /len = 473′ | | |
| 49227_at | Cluster Incl. H09620:yl97f09.s1 Homo sapiens cDNA, | 0.303321471 | 0.001357 |
| | 3 end /clone = IMAGE-46287 /clone_end = 3′ | | |
| | /gb = H09620 /gi = 874442 /ug = Hs.23783 /len = 531′ | | |
| 52988_at | Cluster Incl. AI765981:wj23a06.x1 Homo sapiens cDNA, | 0.303206128 | 0.000057 |
| | 3 end /clone = IMAGE-2403634 /clone_end = 3′ | | |
| | /gb = AI765981 /gi = 5232490 /ug = Hs.49657 /len = 580′ | | |
| 58356_at | Cluster Incl. AA142978:zl49d03.s1 Homo sapiens cDNA, | 0.301800249 | 0.000424 |
| | 3 end /clone = IMAGE-505253 /clone_end = 3′ | | |
| | /gb = AA142978 /gi = 1712437 /ug = Hs.167951 /len = 654′ | | |
| 53275_at | Cluster Incl. AA829709:oe50e07.s1 Homo sapiens cDNA | 0.301386846 | 0.00009 |
| | /clone = IMAGE-1415076 /gb = AA829709 /gi = 2902808 | | |
| | /ug = Hs.28733 /len = 438 | | |
| 55107_at | Cluster Incl. AI916306:we29c09.x1 Homo sapiens cDNA, | 0.300526553 | 0.000003 |
| | 3 end /clone = IMAGE-2342512 /clone_end = 3′ | | |
| | /gb = AI916306 /gi = 5636251 /ug = Hs.87125 /len = 532′ | | |
| 42909_at | Cluster Incl. AA281967:zt11g07.s1 Homo sapiens cDNA, | 0.299343167 | 0.000852 |
| | 3 end /clone = IMAGE-712860 /clone_end = 3′ | | |
| | /gb = AA281967 /gi = 1924791 /ug = Hs.221168 /len = 454′ | | |
| 42832_at | Cluster Incl. W61185:zd31c11.s1 Homo sapiens cDNA, | 0.298246963 | 0.000023 |
| | 3 end /clone = IMAGE-342260 /clone_end = 3′ | | |
| | /gb = W61185 /gi = 1368020 /ug = Hs.169345 /len = 400′ | | |
| 44599_at | Cluster Incl. AA631908:np78a09.s1 Homo sapiens cDNA | 0.2973351 | 0.000806 |
| | /clone = IMAGE-1132408 /gb = AA631908 /gi = 2554519 | | |
| | /ug = Hs.32417 /len = 569 | | |
| 46169_at | Cluster Incl. AI745624:wc33h03.x1 Homo sapiens cDNA, | 0.297165449 | 0.00003 |
| | 3 end /clone = IMAGE-2317013 /clone_end = 3′ | | |
| | /gb = AI745624 /gi = 5113912 /ug = Hs.6065 /len = 574′ | | |
| 43063_at | Cluster Incl. AA584408:nn81b03.s1 Homo sapiens cDNA, | 0.296993211 | 0.009651 |
| | 3 end /clone = IMAGE-1090253 /clone_end = 3′ | | |
| | /gb = AA584408 /gi = 2369017 /ug = Hs.233852 /len = 620′ | | |
| 54831_r_at | Cluster Incl. AA147881:zl50a04.s1 Homo sapiens cDNA, | 0.295444255 | 0.000453 |
| | 3 end /clone = IMAGE-505326 /clone_end = 3′ | | |
| | /gb = AA147881 /gi = 1717297 /ug = Hs.180777 /len = 620′ | | |
| 50411_at | Cluster Incl. AI659533:tu12a12.x1 Homo sapiens cDNA, | 0.294474802 | 0.008774 |
| | 3 end /clone = IMAGE-2250814 /clone_end = 3′ | | |
| | /gb = AI659533 /gi = 4763103 /ug = Hs.25248 /len = 654′ | | |
| 46628_at | Cluster Incl. AI636631:ts92a04.x1 Homo sapiens cDNA, | 0.293823752 | 0.003684 |
| | 3 end /clone = IMAGE-2238702 /clone_end = 3′ | | |
| | /gb = AI636631 /gi = 4687961 /ug = Hs.6828 /len = 495′ | | |
| 56238_at | Cluster Incl. AI589776:tm74c04.x1 Homo sapiens cDNA, | 0.293416233 | 0.004513 |
| | 3 end /clone = IMAGE-2163846 /clone_end = 3′ | | |
| | /gb = AI589776 /gi = 4598824 /ug = Hs.19447 /len = 760′ | | |
| 47283_at | Cluster Incl. AA398628:zt74e03.s1 Homo sapiens cDNA, | 0.293232708 | 0.00032 |
| | 3 end /clone = IMAGE-728092 /clone_end = 3′ | | |
| | /gb = AA398628 /gi = 2051737 /ug = Hs.163845 /len = 467′ | | |
| 57143_r_at | Cluster Incl. AI740811:wg24e02.x1 Homo sapiens cDNA, | 0.292284773 | 0.002541 |
| | 3 end /clone = IMAGE-2366042 /clone_end = 3′ | | |
| | /gb = AI740811 /gi = 5109099 /ug = Hs.23579 /len = 572′ | | |
| 58104_at | Cluster Incl. AI161427:qc78f03.x1 Homo sapiens cDNA, | 0.291974611 | 0.000569 |
| | 3 end /clone = IMAGE-1715741 /clone_end = 3′ | | |
| | /gb = AI161427 /gi = 3693188 /ug = Hs.104871 /len = 436′ | | |
| 44680_at | Cluster Incl. AA464020:zx86a03.r1 Homo sapiens cDNA, | 0.291478075 | 0.000129 |
| | 5 end /clone = IMAGE-810604 /clone_end = 5′ | | |
| | /gb = AA464020 /gi = 2188904 /ug = Hs.32246 /len = 534′ | | |
| 58015_at | Cluster Incl. AA993222:ou02c09.s1 Homo sapiens cDNA, | 0.290171632 | 0.000436 |
| | 3 end /clone = IMAGE-1625104 /clone_end = 3′ | | |
| | /gb = AA993222 /gi = 3179767 /ug = Hs.101915 /len = 471′ | | |
| 47918_at | Cluster Incl. H17938:ym37a06.s1 Homo sapiens cDNA, | 0.289844258 | 0.000045 |
| | 3 end /clone = IMAGE-50084 /clone_end = 3′ | | |
| | /gb = H17938 /gi = 884178 /ug = Hs.16365 /len = 473′ | | |
| 44031_at | Cluster Incl. AI478812:tm52f03.x1 Homo sapiens cDNA, | 0.289231972 | 0.000021 |
| | 3 end /clone = IMAGE-2161757 /clone_end = 3′ | | |
| | /gb = AI478812 /gi = 4373625 /ug = Hs.28917 /len = 511′ | | |
| 45260_at | Cluster Incl. W22565:70E5 Homo sapiens cDNA | 0.288048543 | 0.010503 |
| | /clone = (not-directional) /gb = W22565 /gi = 1299447 | | |
| | /ug = Hs.182344 /len = 805 | | |
| 50190_at | Cluster Incl. AI697875:we18c06.x1 Homo sapiens cDNA, | 0.28704917 | 0.00027 |
| | 3 end /clone = IMAGE-2341450 /clone_end = 3′ | | |
| | /gb = AI697875 /gi = 4985775 /ug = Hs.31664 /len = 551′ | | |
| 52125_at | Cluster Incl. AI991041:wu36b09.x1 Homo sapiens cDNA, | 0.28697383 | 0.001537 |
| | 3 end /clone = IMAGE-990828 /clone_end = 3′ | | |
| | /gb = AI991041 /gi = 5837938 /ug = Hs.44579 /len = 534′ | | |
| 52480_at | Cluster Incl. AI334409:qq26b07.x1 Homo sapiens cDNA, | 0.286020382 | 0.002088 |
| | 3 end /clone = IMAGE-1933621 /clone_end = 3′ | | |
| | /gb = AI334409 /gi = 4070968 /ug = Hs.26192 /len = 439′ | | |
| 43601_g_a | Cluster Incl. AA100793:zm26c02.s1 Homo sapiens cDNA, | 0.285572376 | 0.017485 |
| | 3 end /clone = IMAGE-526754 /clone_end = 3′ | | |
| | /gb = AA100793 /gi = 1647210 /ug = Hs.193380 /len = 500′ | | |
| 42242_at | Cluster Incl. AA978128:op32e03.s1 Homo sapiens cDNA, | 0.284819299 | 0.000022 |
| | 3 end /clone = IMAGE-1578556 /clone_end = 3′ | | |
| | /gb = AA978128 /gi = 3155574 /ug = Hs.99513 /len = 382′ | | |
| 44256_g_a | Cluster Incl. AA480017:zv18e06.r1 Homo sapiens cDNA, | 0.284758761 | 0.037516 |
| | 5 end /clone = IMAGE-754018 /clone_end = 5′ | | |
| | /gb = AA480017 /gi = 2208168 /ug = Hs.226513 /len = 499′ | | |
| 49125_at | Cluster Incl. AI609604:tw91e09.x1 Homo sapiens cDNA, | 0.284325628 | 0.015787 |
| | 3 end /clone = IMAGE-2267080 /clone_end = 3′ | | |
| | /gb = AI609604 /gi = 4618771 /ug = Hs.227806 /len = 460′ | | |
| 49819_at | Cluster Incl. AI432375:tg54h06.x1 Homo sapiens cDNA, | 0.283787158 | 0.000007 |
| | 3 end /clone = IMAGE-2112635 /clone_end = 3′ | | |
| | /gb = AI432375 /gi = 4281193 /ug = Hs.99083 /len = 470′ | | |
| 53914_at | Cluster Incl. AI767222:wi94e09.x1 Homo sapiens cDNA, | 0.283775141 | 0.000275 |
| | 3 end /clone = IMAGE-2401000 /clone_end = 3′ | | |
| | /gb = AI767222 /gi = 5233731 /ug = Hs.15830 /len = 651′ | | |
| 47324_at | Cluster Incl. AA199881:zq52f10.s1 Homo sapiens cDNA, | 0.283572718 | 0 |
| | 3 end /clone = IMAGE-645259 /clone_end = 3′ | | |
| | /gb = AA199881 /gi = 1795588 /ug = Hs.58350 /len = 511′ | | |
| 53796_at | Cluster Incl. AI819282:wj05f11.x1 Homo sapiens cDNA, | 0.283060388 | 0.04394 |
| | 3 end /clone = IMAGE-2401965 /clone_end = 3′ | | |
| | /gb = AI819282 /gi = 5438361 /ug = Hs.12936 /len = 593′ | | |
| 47481_at | Cluster Incl. AA621478:af92e12.s1 Homo sapiens cDNA, | 0.282636217 | 0.016871 |
| | 3 end /clone = IMAGE-1055278 /clone_end = 3′ | | |
| | /gb = AA621478 /gi = 2525417 /ug = Hs.130699 /len = 398′ | | |
| 45333_at | Cluster Incl. AI525592:PT1.3_03_F03.r Homo sapiens cDNA, | 0.279658196 | 0.000052 |
| | 5 end /clone_end = 5′ /gb = AI525592 | | |
| | /gi = 4439727 /ug = Hs.23579 /len = 887′ | | |
| 59126_at | Cluster Incl. AI660548:we68a05.x1 Homo sapiens cDNA, | 0.278719933 | 0.0035 |
| | 3 end /clone = IMAGE-2346224 /clone_end = 3′ | | |
| | /gb = AI660548 /gi = 4764118 /ug = Hs.19165 /len = 501′ | | |
| 48822_s_at | Cluster Incl. AW015546:UI-H-BI0p-aau-d-11-0-UI.s1 | 0.27784195 | 0.004766 |
| | Homo sapiens cDNA, | | |
| | 3 end /clone = IMAGE-2710700 /clone_end = 3′ | | |
| | /gb = AW015546 /gi = 5864303 /ug = Hs.234250 /len = 629′ | | |
| 43395_s_at | Cluster Incl. AW006148:wz92h09.x1 Homo sapiens cDNA, | 0.277596215 | 0.030595 |
| | 3 end /clone = IMAGE-2566337 /clone_end = 3′ | | |
| | /gb = AW006148 /gi = 5854926 /ug = Hs.234072 /len = 502′ | | |
| 47071_at | Cluster Incl. AI279709:ql53e09.x1 Homo sapiens cDNA, | 0.276885721 | . 0 |
| | 3 end /clone = IMAGE-1876072 /clone_end = 3′ | | |
| | /gb = AI279709 /gi = 3917943 /ug = Hs.79741 /len = 601′ | | |
| 58454_at | Cluster Incl. AI738919:wi12a04.x1 Homo sapiens cDNA, | 0.274374246 | 0.000036 |
| | 3 end /clone = IMAGE-2389998 /clone_end = 3′ | | |
| | /gb = AI738919 /gi = 5100900 /ug = Hs.239355 /len = 648′ | | |
| 54369_at | Cluster Incl. AI242381:qi15f07.x1 Homo sapiens cDNA, | 0.274129957 | 0.000022 |
| | 3 end /clone = IMAGE-1856581 /clone_end = 3′ | | |
| | /gb = AI242381 /gi = 3837778 /ug = Hs.47282 /len = 263′ | | |
| 54310_at | Cluster Incl. AA975511:on30e03.s1 Homo sapiens cDNA, | 0.272632887 | 0.003 |
| | 3 end /clone = IMAGE-1558204 /clone_end = 3′ | | |
| | /gb = AA975511 /gi = 3151303 /ug = Hs.44565 /len = 497′ | | |
| 50151_at | Cluster Incl. AA403227:zv66h05.r1 Homo sapiens cDNA, | 0.271893984 | 0.000269 |
| | 5 end /clone = IMAGE-758649 /clone_end = 5′ | | |
| | /gb = AA403227 /gi = 2055725 /ug = Hs.29604 /len = 557′ | | |
| 46011_at | Cluster Incl. AI916225:wi49f07.x1 Homo sapiens cDNA, | 0.269822445 | 0.000412 |
| | 3 end /clone = IMAGE-2393605 /clone_end = 3′ | | |
| | /gb = AI916225 /gi = 5636080 /ug = Hs.58559 /len = 507′ | | |
| 56216_at | Cluster Incl. AA740543:ob28g10.s1 Homo sapiens cDNA | 0.269334271 | 0.001353 |
| | /clone = IMAGE-1325058 /gb = AA740543 /gi = 2779135 | | |
| | /ug = Hs.103913 /len = 486 | | |
| 57452_at | Cluster Incl. AA451798:zx46b07.r1 Homo sapiens cDNA, | 0.268116773 | 0.000009 |
| | 5 end /clone = IMAGE-795253 /clone_end = 5′ | | |
| | /gb = AA451798 /gi = 2165467 /ug = Hs.99249 /len = 407′ | | |
| 45456_at | Cluster Incl. R60584:yh14g05.s1 Homo sapiens cDNA, | 0.26781965 | 0.007247 |
| | 3 end /clone = IMAGE-37856 /clone_end = 3′ | | |
| | /gb = R60584 /gi = 831279 /ug = Hs.26789 /len = 492′ | | |
| 46105_at | Cluster Incl. AA149641:zl39c08.s1 Homo sapiens cDNA, | 0.267205089 | 0.000264 |
| | 3 end /clone = IMAGE-504302 /clone_end = 3′ | | |
| | /gb = AA149641 /gi = 1720442 /ug = Hs.55405 /len = 570′ | | |
| 58567_s_at | Cluster Incl. AI308197:tb25g11.x1 Homo sapiens cDNA, | 0.265573576 | 0.034363 |
| | 3 end /clone = IMAGE-2055428 /clone_end = 3′ | | |
| | /gb = AI308197 /gi = 4002832 /ug = Hs.40496 /len = 462′ | | |
| 58359_g_a | Cluster Incl. AA142978:zl49d03.s1 Homo sapiens cDNA, | 0.264528974 | 0.000057 |
| | 3 end /clone = IMAGE-505253 /clone_end = 3′ | | |
| | /gb = AA142978 /gi = 1712437 /ug = Hs.167951 /len = 654′ | | |
| 53166_at | Cluster Incl. W74591:zd77g09.s1 Homo sapiens cDNA, | 0.263493949 | 0.002128 |
| | 3 end /clone = IMAGE-346720 /clone end = 3′ | | |
| | /gb = W74591 /gi = 1384892 /ug = Hs.5771 /len = 663′ | | |
| 45256_at | Cluster Incl. AI271460:qi19e06.x1 Homo sapiens cDNA, | 0.259328936 | 0.001801 |
| | 3 end /clone = IMAGE-1856962 /clone_end = 3′ | | |
| | /gb = AI271460 /gi = 3890627 /ug = Hs.181368 /len = 559′ | | |
| 47771_at | Cluster Incl. AA234670:zs37f06.r1 Homo sapiens cDNA, | 0.257739498 | 0.000014 |
| | 5 end /clone = IMAGE-687395 /clone_end = 5′ | | |
| | /gb = AA234670 /gi = 1859377 /ug = Hs.62654 /len = 316′ | | |
| 59704_at | Cluster Incl. H15288:ym30g12.s1 Homo sapiens cDNA, | 0.256002125 | 0.000103 |
| | 3 end /clone = IMAGE-49810 /clone_end = 3′ | | |
| | /gb = H15288 /gi = 880108 /ug = Hs.21420 /len = 562′ | | |
| 48896_at | Cluster Incl. AI082244:ox79g07.x1 Homo sapiens cDNA, | 0.255967313 | 0.000028 |
| | 3 end /clone = IMAGE-1662588 /clone_end = 3′ | | |
| | /gb = AI082244 /gi = 3419036 /ug = Hs.93764 /len = 552′ | | |
| 45210_at | Cluster Incl. AA035736:ze25b11.r1 Homo sapiens cDNA, | 0.255599052 | 0.000002 |
| | 5 end /clone = IMAGE-359997 /clone_end = 5′ | | |
| | /gb = AA035736 /gi = 1507626 /ug = Hs.4285 /len = 470′ | | |
| AFFX-HUM | M33197 Human glyceraldehyde-3-phosphate dehydrogenase | 0.253971231 | 0.02209 |
| | (GAPDH) mRNA, complete cds (_5, _M, _3 represent transcript | | |
| | regions 5 prime, Middle, and 3 prime respectively) | | |
| 50223_at | Cluster Incl. AA020743:ze63c11.s1 Homo sapiens cDNA, | 0.253657497 | 0.048419 |
| | 3 end /clone = IMAGE-363668 /clone_end = 3′ | | |
| | /gb = AA020743 /gi = 1484525 /ug = Hs.234026 /len = 626′ | | |
| 47097_at | Cluster Incl. AI674565:wc03c02.x1 Homo sapiens cDNA, | 0.252742984 | 0.000395 |
| | 3 end /clone = IMAGE-2314082 /clone_end = 3′ | | |
| | /gb = AI674565 /gi = 4875045 /ug = Hs.8379 /len = 563′ | | |
| 50361_at | Cluster Incl. AI658662:tu22a11.x1 Homo sapiens cDNA, | 0.247649714 | 0.007879 |
| | 3 end /clone = IMAGE-2251772 /clone_end = 3′ | | |
| | /gb = AI658662 /gi = 4762232 /ug = Hs.24192 /len = 503′ | | |
| 58288_at | Cluster Incl. W63676:zd30b03.s1 Homo sapiens cDNA, | 0.246615101 | 0.000134 |
| | 3 end /clone = IMAGE-342125 /clone_end = 3′ | | |
| | /gb = W63676 /gi = 1371257 /ug = Hs.15641 /len = 627′ | | |
| 57772_g_a | Cluster Incl. AA886870:oj25g11.s1 Homo sapiens cDNA, | 0.246598396 | 0.00327 |
| | 3 end /clone = IMAGE-1493252 /clone_end = 3′ | | |
| | /gb = AA886870 /gi = 3001978 /ug = Hs.107381 /len = 572′ | | |
| 41955_r_at | Cluster Incl. Z41561:HSCZYA022 Homo sapiens cDNA, | 0.244644274 | 0.000008 |
| | 3 end /clone = c-zya02 /clone_end = 3′ /gb = Z41561 | | |
| | /gi = 567709 /ug = Hs.65739 /len = 251′ | | |
| 57141_f_at | Cluster Incl. AI740811:wg24e02.x1 Homo sapiens cDNA, | 0.244451584 | 0.000034 |
| | 3 end /clone = IMAGE-2366042 /clone_end = 3′ | | |
| | /gb = AI740811 /gi = 5109099 /ug = Hs.23579 /len = 572′ | | |
| 55525_at | Cluster Incl. AI972498:wr38c04.x1 Homo sapiens cDNA, | 0.240971633 | 0.00039 |
| | 3 end /clone = IMAGE-2489958 /clone_end = 3′ | | |
| | /gb = AI972498 /gi = 5769244 /ug = Hs.97469 /len = 546′ | | |
| 47124_at | Cluster Incl. AI672049:ty63b04.x1 Homo sapiens cDNA, | 0.239306276 | 0.022482 |
| | 3 end /clone = IMAGE-2283727 /clone_end = 3′ | | |
| | /gb = AI672049 /gi = 4851780 /ug = Hs.8734 /len = 574′ | | |
| 44606_at | Cluster Incl. AA205873:zq54c06.s1 Homo sapiens cDNA, | 0.238440601 | 0.000031 |
| | 3 end /clone = IMAGE-645418 /clone_end = 3′ | | |
| | /gb = AA205873 /gi = 1801444 /ug = Hs.32807 /len = 528′ | | |
| 51909_at | Cluster Incl. AI130027:qc36c08.x1 Homo sapiens cDNA, | 0.236891013 | 0.007448 |
| | 3 end /clone = IMAGE-1711694 /clone_end = 3′ | | |
| | /gb = AI130027 /gi = 3598541 /ug = Hs.65748 /len = 461′ | | |
| 46691_at | Cluster Incl. AI472143:tj86b02.x1 Homo sapiens cDNA, | 0.236351122 | 0.00165 |
| | 3 end /clone = IMAGE-2148363 /clone_end = 3′ | | |
| | /gb = AI472143 /gi = 4334233 /ug = Hs.92198 /len = 613′ | | |
| 47578_at | Cluster Incl. AA160156:zo81c09.s1 Homo sapiens cDNA, | 0.235200728 | 0.000029 |
| | 3 end /clone = IMAGE-593296 /clone_end = 3′ | | |
| | /gb = AA160156 /gi = 1734749 /ug = Hs.95612 /len = 528′ | | |
| 48476_at | Cluster Incl. AI768887:wh71f02.x1 Homo sapiens cDNA, | 0.23307379 | 0.000002 |
| | 3 end /clone = IMAGE-2386203 /clone_end = 3′ | | |
| | /gb = AI768887 /gi = 5235396 /ug = Hs.21958 /len = 539′ | | |
| 49709_at | Cluster Incl. AA127924:zl13h06.s1 Homo sapiens cDNA, | 0.231365733 | 0.000001 |
| | 3 end /clone = IMAGE-501851 /clone_end = 3′ | | |
| | /gb = AA127924 /gi = 1687231 /ug = Hs.71034 /len = 578′ | | |
| 43412 s_at | Cluster Incl. AA622152:nq56e11.s1 Homo sapiens cDNA, | 0.230949759 | 0.000001 |
| | 3 end /clone = IMAGE-1147916 /clone_end = 3′ | | |
| | /gb = AA622152 /gi = 2526028 /ug = Hs.234262 /len = 414′ | | |
| 56894_at | Cluster Incl. N29070:yx41d02.r1 Homo sapiens cDNA, | 0.229718565 | 0 |
| | 5 end /clone = IMAGE-264291 /clone_end = 5′ | | |
| | /gb = N29070 /gi = 1147306 /ug = Hs.108923 /len = 489′ | | |
| 46927_at | Cluster Incl. AA086045:zl84e03.s1 Homo sapiens cDNA, | 0.229016351 | 0.000019 |
| | 3 end /clone = IMAGE-511324 /clone_end = 3′ | | |
| | /gb = AA086045 /gi = 1629586 /ug = Hs.137313 /len = 518′ | | |
| 47474_at | Cluster Incl. H40296:yn86d03.s1 Homo sapiens cDNA, | 0.228996683 | 0.005309 |
| | 3 end /clone = IMAGE-175301 /clone_end = 3′ | | |
| | /gb = H40296 /gi = 916348 /ug = Hs.127588 /len = 398′ | | |
| 45386_at | Cluster Incl. AI924317:wn55c12.x1 Homo sapiens cDNA, | 0.228351474 | 0.000023 |
| | 3 end /clone = IMAGE-2449366 /clone_end = 3′ | | |
| | /gb = AI924317 /gi = 5660281 /ug = Hs.239375 /len = 490′ | | |
| 47650_at | Cluster Incl. AA397625:zt79d07.r1 Homo sapiens cDNA, | 0.228224396 | 0.001661 |
| | 5 end /clone = IMAGE-728557 /clone_end = 5′ | | |
| | /gb = AA397625 /gi = 2050727 /ug = Hs.163913 /len = 429′ | | |
| 46476_at | Cluster Incl. AA531287:nj09e04.s1 Homo sapiens cDNA, | 0.226386204 | 0.004254 |
| | 3 end /clone = IMAGE-985854 /clone_end = 3′ | | |
| | /gb = AA531287 /gi = 2273993 /ug = Hs.105805 /len = 507′ | | |
| 48050_at | Cluster Incl. AI817448:wk38e01.x1 Homo sapiens cDNA, | 0.225524389 | 0.000145 |
| | 3 end /clone = IMAGE-2417688 /clone end = 3′ | | |
| | /gb = AI817448 /gi = 5436527 /ug = Hs.105421 /len = 572′ | | |
| 49985_at | Cluster Incl. AI917602:wi35g02.x1 Homo sapiens cDNA, | 0.224810828 | 0.00025 |
| | 3 end /clone = IMAGE-2392274 /clone_end = 3′ | | |
| | /gb = AI917602 /gi = 5637457 /ug = Hs.106440 /len = 530′ | | |
| 45747_at | Cluster Incl. AA127810:zk88a10.r1 Homo sapiens cDNA, | 0.218922022 | 0.001011 |
| | 5 end /clone = IMAGE-489882 /clone_end = 5′ | | |
| | /gb = AA127810 /gi = 1687089 /ug = Hs.30376 /len = 619′ | | |
| 52999_at | Cluster Incl. AA126704:zl29b10.s1 Homo sapiens cDNA, | 0.215581442 | 0.007024 |
| | 3 end /clone = IMAGE-503323 /clone_end = 3′ | | |
| | /gb = AA126704 /gi = 1688008 /ug = Hs.50107 /len = 588′ | | |
| 46120_at | Cluster Incl. AA149250:zl26d06.s1 Homo sapiens cDNA, | 0.215193255 | 0.03997 |
| | 3 end /clone = IMAGE-503051 /clone_end = 3′ | | |
| | /gb = AA149250 /gi = 1719966 /ug = Hs.56105 /len = 575′ | | |
| 47928_at | Cluster Incl. AI858054:wj69g04.x1 Homo sapiens cDNA, | 0.215190727 | 0.002954 |
| | 3 end /clone = IMAGE-2408118 /clone_end = 3′ | | |
| | /gb = AI858054 /gi = 5511670 /ug = Hs.165169 /len = 746′ | | |
| 48039_at | Cluster Incl. AI634580:tz30b04.x1 Homo sapiens cDNA, | 0.21474006 | 0.017632 |
| | 3 end /clone = IMAGE-2290063 /clone_end = 3′ | | |
| | /gb = AI634580 /gi = 4685910 /ug = Hs.10339 /len = 686′ | | |
| 43600_at | Cluster Incl. AA100793:zm26c02.s1 Homo sapiens cDNA, | 0.211158214 | 0.001494 |
| | 3 end /clone = IMAGE-526754 /clone_end = 3′ | | |
| | /gb = AA100793 /gi = 1647210 /ug = Hs.193380 /len = 500′ | | |
| 46699_at | Cluster Incl. AI052110:oy30a11.x1 Homo sapiens cDNA, | 0.210882988 | 0.004296 |
| | 3 end /clone = IMAGE-1667324 /clone_end = 3′ | | |
| | /gb = AI052110 /gi = 3308101 /ug = Hs.93961 /len = 567′ | | |
| 46700_at | Cluster Incl. W55956:zc58g06.r1 Homo sapiens cDNA, | 0.210863305 | 0.004741 |
| | 5 end /clone = IMAGE-326554 /clone_end = 5′ | | |
| | /gb = W55956 /gi = 1358060 /ug = Hs.94030 /len = 622′ | | |
| 53011_at | Cluster Incl. AA036952:zk30h01.s1 Homo sapiens cDNA, | 0.210525571 | 0.003777 |
| | 3 end /clone = IMAGE-472081 /clone_end = 3′ | | |
| | /gb = AA036952 /gi = 1510009 /ug = Hs.50841 /len = 571′ | | |
| 45394_s_at | Cluster Incl. AA563933:nk23b02.s1 Homo sapiens cDNA, | 0.209901504 | 0.003068 |
| | 3 end /clone = IMAGE-1014315 /clone_end = 3′ | | |
| | /gb = AA563933 /gi = 2335572 /ug = Hs.239604 /len = 518′ | | |
| 46199_at | Cluster Incl. AI076809:oz01a06.x1 Homo sapiens cDNA, | 0.208199399 | 0.000172 |
| | 3 end /clone = IMAGE-1674034 /clone_end = 3′ | | |
| | /gb = AI076809 /gi = 3404638 /ug = Hs.62472 /len = 516′ | | |
| 59560_at | Cluster Incl. AI669751:tu12g01.x1 Homo sapiens cDNA, | 0.206207696 | 0.000002 |
| | 3 end /clone = IMAGE-2250864 /clone_end = 3′ | | |
| | /gb = AI669751 /gi = 4834525 /ug = Hs.175783 /len = 692′ | | |
| 45207_at | Cluster Incl. N42752:yy11g08.r1 Homo sapiens cDNA, | 0.205356842 | 0.000126 |
| | 5 end /clone = IMAGE-270974 /clone_end = 5′ | | |
| | /gb = N42752 /gi = 1167182 /ug = Hs.42645 /len = 535′ | | |
| 48436_at | Cluster Incl. AI922968:wn66h03.x1 Homo sapiens cDNA, | 0.203844179 | 0.000015 |
| | 3 end /clone = IMAGE-2450453 /clone_end = 3′ | | |
| | /gb = AI922968 /gi = 5659018 /ug = Hs.203485 /len = 498′ | | |
| 48421_at | Cluster Incl. AI990027:ws37c08.x1 Homo sapiens cDNA, | 0.201114358 | 0.000466 |
| | 3 end /clone = IMAGE-2499374 /clone_end = 3′ | | |
| | /gb = AI990027 /gi = 5836908 /ug = Hs.19813 /len = 554′ | | |
| 47982_at | Cluster Incl. AA630981:nq76c07.s1 Homo sapiens cDNA, | 0.1965858 | 0.001398 |
| | 3 end /clone = IMAGE-1158252 /clone_end = 3′ | | |
| | /gb = AA630981 /gi = 2553592 /ug = Hs.174024 /len = 475′ | | |
| 46188_at | Cluster Incl. AI422243:tf58h09.x1 Homo sapiens cDNA, | 0.196273084 | 0.001778 |
| | 3 end /clone = IMAGE-2103521 /clone_end = 3′ | | |
| | /gb = AI422243 /gi = 4268174. /ug = Hs.61762 /len = 593′ | | |
| 52080_at | Cluster Incl. AI864898:wj66d08.x1 Homo sapiens cDNA, | 0.194711313 | 0.003268 |
| | 3 end /clone = IMAGE-2407791 /clone_end = 3′ | | |
| | /gb = AI864898 /gi = 5529005 /ug = Hs.43125 /len = 523′ | | |
| 47123_at | Cluster Incl. AI247543:qh60c07.x1 Homo sapiens cDNA, | 0.194396588 | 0.000008 |
| | 3 end /clone = IMAGE-1849068 /clone_end = 3′ | | |
| | /gb = AI247543 /gi = 3842940 /ug = Hs.87310 /len = 699′ | | |
| 48367_at | Cluster Incl. H62656:yr45a06.r1 Homo sapiens cDNA, | 0.193760977 | 0.000029 |
| | 5 end /clone = IMAGE-208210 /clone_end = 5′ | | |
| | /gb = H62656 /gi = 1017002 /ug = Hs.182670 /len = 504′ | | |
| 43849_s_at | Cluster Incl. AA622570:np21f04.s1 Homo sapiens cDNA, | 0.190479348 | 0.00232 |
| | 3 end /clone = IMAGE-1116991 /clone_end = 3′ | | |
| | /gb = AA622570 /gi = 2526446 /ug = Hs.238528 /len = 461′ | | |
| 58902_r_at | Cluster Incl. R44987:yg33g06.s1 Homo sapiens cDNA, | 0.188238717 | 0.000376 |
| | 3 end /clone = IMAGE-34444 /clone_end = 3′ | | |
| | /gb = R44987 /gi = 824341 /ug = Hs.124207 /len = 481′ | | |
| 48981_at | Cluster Incl. AI954620:wq34c02.x1 Homo sapiens cDNA, | 0.188158824 | 0.000359 |
| | 3 end /clone = IMAGE-2473154 /clone_end = 3′ | | |
| | /gb = AI954620 /gi = 5746930 /ug = Hs.97499 /len = 545′ | | |
| 47605_at | Cluster Incl. AI022657:ox05h04.x1 Homo sapiens cDNA, | 0.187925204 | 0.000006 |
| | 3 end /clone = IMAGE-1655479 /clone_end = 3′ | | |
| | /gb = AI022657 /gi = 3237898 /ug = Hs.97345 /len = 710′ | | |
| 51610_at | Cluster Incl. AI808807:wf97e02.x1 Homo sapiens cDNA, | 0.187409743 | 0.000242 |
| | 3 end /clone = IMAGE-2363546 /clone_end = 3′ | | |
| | /gb = AI808807 /gi = 5395373 /ug = Hs.17404 /len = 538′ | | |
| 47329_at | Cluster Incl. AI092869:qa77c01.x1 Homo sapiens cDNA, | 0.186424395 | 0.000245 |
| | 3 end /clone = IMAGE-1692768 /clone_end = 3′ | | |
| | /gb = AI092869 /gi = 3431845 /ug = Hs.58611 /len = 430′ | | |
| 50648_at | Cluster Incl. AI763136:wi65a06.x1 Homo sapiens cDNA, | 0.184618742 | 0 |
| | 3 end /clone = IMAGE-2398162 /clone_end = 3′ | | |
| | /gb = AI763136 /gi = 5178803 /ug = Hs.165215 /len = 558′ | | |
| 58057_r_at | Cluster Incl. AA009593:ze82f11.s1 Homo sapiens cDNA, | 0.181735047 | 0.000014 |
| | 3 end /clone = IMAGE-365517 /clone_end = 3′ | | |
| | /gb = AA009593 /gi = 1470752 /ug = Hs.103296 /len = 511′ | | |
| 52140_at | Cluster Incl. AL046941:DKFZp586l0717_r1 Homo sapiens cDNA, | 0.181720222 | 0.01492 |
| | 5 end /clone = DKFZp586I0717 /clone_end = 5′ | | |
| | /gb = AL046941 /gi = 5435000 /ug = Hs.46531 /len = 704′ | | |
| 52634_at | Cluster Incl. AW025596:wu75d09.x1 Homo sapiens cDNA, | 0.180172622 | 0.000001 |
| | 3 end /clone = IMAGE-990756 /clone_end = 3′ | | |
| | /gb = AW025596 /gi = 5879126 /ug = Hs.238927 /len = 488′ | | |
| 52742_at | Cluster Incl. AI767756:wh39d09.x1 Homo sapiens cDNA, | 0.179940127 | 0.000009 |
| | 3 end /clone = IMAGE-2383121 /clone_end = 3′ | | |
| | /gb = AI767756 /gi = 5234265 /ug = Hs.82302 /len = 501′ | | |
| 51291_at | Cluster Incl. W91952:zh47f03.r1 Homo sapiens cDNA, | 0.179194305 | 0.001469 |
| | 5 end /clone = IMAGE-415229 /clone_end = 5′ | | |
| | /gb = W91952 /gi = 1424313 /ug = Hs.37331 /len = 594′ | | |
| 48116_at | Cluster Incl. AI096389:qb91f12.x1 Homo sapiens cDNA, | 0.17789649 | 0.000023 |
| | 3 end /clone = IMAGE-1707503 /clone_end = 3′ | | |
| | /gb = AI096389 /gi = 3446300 /ug = Hs.110341 /len = 566′ | | |
| 51122_at | Cluster Incl. AI689756:tx30h05.x1 Homo sapiens cDNA, | 0.177272252 | 0.000009 |
| | 3 end /clone = IMAGE-2271129 /clone_end = 3′ | | |
| | /gb = AI689756 /gi = 4901050 /ug = Hs.26244 /len = 542′ | | |
| 50729_at | Cluster Incl. N39954:yw69f12.r1 Homo sapiens cDNA, | 0.174253817 | 0.000343 |
| | 5 end /clone = IMAGE-257519 /clone_end = 5′ | | |
| | /gb = N39954 /gi = 1163499 /ug = Hs.109012 /len = 531′ | | |
| 53970_at | Cluster Incl. AF150266:AF150266 Homo sapiens cDNA | 0.17406147 | 0.000039 |
| | /clone = CBFBIG05 /gb = AF150266 /gi = 5133702 | | |
| | /ug = Hs.62643 /len = 1425 | | |
| 55697_at | Cluster Incl. AI653211:wb23h04.x1 Homo sapiens cDNA, | 0.173630297 | 0.009509 |
| | 3 end /clone = IMAGE-2306551 /clone_end = 3′ | | |
| | /gb = AI653211 /gi = 4737190 /ug = Hs.96657 /len = 620′ | | |
| 47597_at | Cluster Incl. AL040944:DKFZp434K2415_s1 Homo sapiens cDNA, | 0.173346375 | 0.01163 |
| | 3 end /clone = DKFZp434K2415 /clone_end = 3′ | | |
| | /gb = AL040944 /gi = 5409888 /ug = Hs.96937 /len = 768′ | | |
| 55772_at | Cluster Incl. AI765963:wh69g08.x1 Homo sapiens cDNA, | 0.171422781 | 0.000022 |
| | 3 end /clone = IMAGE-2386046 /clone_end = 3′ | | |
| | /gb = AI765963 /gi = 5232472 /ug = Hs.10299 /len = 518′ | | |
| 43961_s_at | Cluster Incl. AI627648:ty81b09.x1 Homo sapiens cDNA, | 0.171272429 | 0.000004 |
| | 3 end /clone = IMAGE-2285465 /clone_end = 3′ | | |
| | /gb = AI627648 /gi = 4664448 /ug = Hs.26168 /len = 754′ | | |
| 45757_at | Cluster Incl. W73855:zd52f10.r1 Homo sapiens cDNA, | 0.16034244 | 0.000246 |
| | 5 end /clone = IMAGE-344299 /clone_end = 5′ | | |
| | /gb = W73855 /gi = 1384028 /ug = Hs.32343 /len = 677′ | | |
| 48070_at | Cluster Incl. AI743780:wg53g09.x1 Homo sapiens cDNA, | 0.155837517 | 0.000073 |
| | 3 end /clone = IMAGE-2368864 /clone_end = 3′ | | |
| | /gb = AI743780 /gi = 5112068 /ug = Hs.107203 /len = 529′ | | |
| 44392_at | Cluster Incl. H07855:yl86a02.s1 Homo sapiens cDNA, | 0.150341872 | 0.000005 |
| | 3 end /clone = IMAGE-44907 /clone_end = 3′ | | |
| | /gb = H07855 /gi = 872677 /ug = Hs.30604 /len = 430′ | | |
| 50891_at | Cluster Incl. AI768853:wj12f11.x1 Homo sapiens cDNA, | 0.149604741 | 0.00915 |
| | 3 end /clone = IMAGE-2402637 /clone_end = 3′ | | |
| | /gb = AI768853 /gi = 5235362 /ug = Hs.134478 /len = 451′ | | |
| 48486_at | Cluster Incl. AW024960:wu70c08.x1 Homo sapiens cDNA, | 0.149120971 | 0.00082 |
| | 3 end /clone = IMAGE-991041 /clone_end = 3′ | | |
| | /gb = AW024960 /gi = 5878490 /ug = Hs.110945 /len = 806′ | | |
| 52955_at | Cluster Incl. T68813:yc29f09.r1 Homo sapiens cDNA, | 0.148934239 | 0.000064 |
| | 5 end /clone = IMAGE-82121 /clone_end = 5′ | | |
| | /gb = T68813 /gi = 679961 /ug = Hs.11896 /len = 583′ | | |
| 52637_g_a | Cluster Incl. AW025596:wu75d09.x1 Homo sapiens cDNA, | 0.14616299 | 0.00001 |
| | 3 end /clone = IMAGE-990756 /clone_end = 3′ | | |
| | /gb = AW025596 /gi = 5879126 /ug = Hs.238927 /len = 488′ | | |
| 46108_at | Cluster Incl. AI804914:tu43d04.x1 Homo sapiens cDNA, | 0.145421966 | 0.000387 |
| | 3 end /clone = IMAGE-2253799 /clone_end = 3′ | | |
| | /gb = AI804914 /gi = 5391504 /ug = Hs.55565 /len = 535′ | | |
| 54066_at | Cluster Incl. AI356291:qz25h11.x1 Homo sapiens cDNA, | 0.143940201 | 0.000279 |
| | 3 end /clone = IMAGE-2027973 /clone_end = 3′ | | |
| | /gb = AI356291 /gi = 4107912 /ug = Hs.6775 /len = 753′ | | |
| 44113_at | Cluster Incl. U55984:HSU55984 Homo sapiens cDNA | 0.143500649 | 0 |
| | /clone = 37941 /gb = U55984 /gi = 1354540 | | |
| | /ug = Hs.26662 /len = 1147 | | |
| 52049_at | Cluster Incl. AI742029:wg38b07.x1 Homo sapiens cDNA, | 0.141341539 | 0.000017 |
| | 3 end /clone = IMAGE-2367349 /clone_end = 3′ | | |
| | /gb = AI742029 /gi = 5110317 /ug = Hs.4232 /len = 769′ | | |
| 48293_at | Cluster Incl. AI128031:qc42h07.x1 Homo sapiens cDNA, | 0.140884301 | 0.010169 |
| | 3 end /clone = IMAGE-1712317 /clone_end = 3′ | | |
| | /gb = AI128031 /gi = 3596545 /ug = Hs.87383 /len = 503′ | | |
| 45220_at | Cluster Incl. AA150501:zl08g02.s1 Homo sapiens cDNA, | 0.136941145 | 0.000008 |
| | 3 end /clone = IMAGE-491762 /clone_end = 3′ | | |
| | /gb = AA150501 /gi = 1722015 /ug = Hs.43148 /len = 645′ | | |
| 44104_at | Cluster Incl. AL036582:DKFZp564L0462_s1 Homo sapiens cDNA, | 0.135894846 | 0.000009 |
| | 3 end /clone = DKFZp564L0462 /clone_end = 3′ | | |
| | /gb = AL036582 /gi = 5927810 /ug = Hs.25740 /len = 582′ | | |
| 51261_at | Cluster Incl. AI052020:oy29e06.x1 Homo sapiens cDNA, | 0.135142446 | 0.000016 |
| | 3 end /clone = IMAGE-1667266 /clone_end = 3′ | | |
| | /gb = AI052020 /gi = 3308011 /ug = Hs.3593 /len = 757′ | | |
| 52986_at | Cluster Incl. AI743925:wg54f04.x1 Homo sapiens cDNA, | 0.134644236 | 0.000003 |
| | 3 end /clone = IMAGE-2368927 /clone_end = 3′ | | |
| | /gb = AI743925 /gi = 5112213 /ug = Hs.4944 /len = 505′ | | |
| 47328_at | Cluster Incl. AA483389:ne74d10.s1 Homo sapiens cDNA | 0.134567862 | 0.000026 |
| | /clone = IMAGE-910003 /gb = AA483389 /gi = 2212202 | | |
| | /ug = Hs.58566 /len = 486 | | |
| 55924_at | Cluster Incl. AA085776:zk71h10.s1 Homo sapiens cDNA, | 0.130253189 | 0.000015 |
| | 3 end /clone = IMAGE-488323 /clone_end = 3′ | | |
| | /gb = AA085776 /gi = 1629238 /ug = Hs.68784 /len = 633′ | | |
| 56474_at | Cluster Incl. W23068:78H5 Homo sapiens cDNA | 0.128217729 | 0.002301 |
| | /clone = (not-directional) /gb = W23068 /gi = 1299950 | | |
| | /ug = Hs.111676 /len = 627 | | |
| 50607_at | Cluster Incl. R41725:yg11b02.s1 Homo sapiens cDNA, | 0.123861065 | 0.000016 |
| | 3 end /clone = IMAGE-31813 /clone_end = 3′ | | |
| | /gb = R41725 /gi = 817432 /ug = Hs.140852 /len = 437′ | | |
| 55249_at | Cluster Incl. W69365:zd46f09.s1 Homo sapiens cDNA, | 0.122494713 | 0 |
| | 3 end /clone = IMAGE-343721 /clone_end = 3′ | | |
| | /gb = W69365 /gi = 1378676 /ug = Hs.58006 /len = 446′ | | |
| 45684_at | Cluster Incl. AL040936:DKFZp434K1715_s1 Homo sapiens cDNA, | 0.122114371 | 0.000002 |
| | 3 end /clone = DKFZp434K1715 /clone_end = 3′ | | |
| | /gb = AL040936 /gi = 5409880 /ug = Hs.5027 /len = 742′ | | |
| 47385_at | Cluster Incl. AI147256:qb36h11.x1 Homo sapiens cDNA, | 0.121799678 | 0.000003 |
| | 3 end /clone = IMAGE-1698405 /clone_end = 3′ | | |
| | /gb = AI147256 /gi = 3674938 /ug = Hs.61460 /len = 436′ | | |
| 51096_at | Cluster Incl. AL048651:DKFZp564O2171_s1 Homo sapiens cDNA | 0.118284422 | 0.000009 |
| | /clone = DKFZp564O2171 /gb = AL048651 | | |
| | /gi = 4727712 /ug = Hs.25740 /len = 840 | | |
| 47032_at | Cluster Incl. AI885390:wl93c03.x1 Homo sapiens cDNA, | 0.11658742 | 0.000001 |
| | 3 end /clone = IMAGE-2432452 /clone_end = 3′ | | |
| | /gb = AI885390 /gi = 5590554 /ug = Hs.49031 /len = 575′ | | |
| 57685_at | Cluster Incl. Al669815:tu31f07.x1 Homo sapiens cDNA, | 0.114663319 | 0.002628 |
| | 3 end /clone = IMAGE-2252677 /clone_end = 3′ | | |
| | /gb = AI669815 /gi = 4834589 /ug = Hs.129911 /len = 646′ | | |
| 51289_at | Cluster Incl. C00414:HUMGS0005283 Homo sapiens cDNA | 0.114661173 | 0.001594 |
| | /gb = C00414 /gi = 1432644 /ug = Hs.37331 | | |
| | /len = 546 | | |
| 53593_at | Cluster Incl. AI494498:qz01a06.x1 Homo sapiens cDNA, | 0.112799017 | 0 |
| | 3 end /clone = IMAGE-2020210 /clone_end = 3′ | | |
| | /gb = AI494498 /gi = 4395501 /ug = Hs.44685 /len = 450′ | | |
| 55491_at | Cluster Incl. AI081571:ox59h10.s1 Homo sapiens cDNA, | 0.111799777 | 0.000002 |
| | 3 end /clone = IMAGE-1660675 /clone_end = 3′ | | |
| | /gb = AI081571 /gi = 3418363 /ug = Hs.96457 /len = 468′ | | |
| 51939_at | Cluster Incl. AA142913:zl40e07.s1 Homo sapiens cDNA, | 0.111488827 | 0.000898 |
| | 3 end /clone = IMAGE-504420 /clone_end = 3′ | | |
| | /gb = AA142913 /gi = 1712300 /ug = Hs.71721 /len = 572′ | | |
| 51837_at | Cluster Incl. AI916261:wg99d02.x1 Homo sapiens cDNA, | 0.109272217 | 0 |
| | 3 end /clone = IMAGE-2379363 /clone_end = 3′ | | |
| | /gb = AI916261 /gi = 5636116 /ug = Hs.55409 /len = 842′ | | |
| 55278_at | Cluster Incl. AI659657:tu25b10.x1 Homo sapiens cDNA, | 0.103460982 | 0.000001 |
| | 3 end /clone = IMAGE-2252059 /clone_end = 3′ | | |
| | /gb = AI659657 /gi = 4763227 /ug = Hs.58561 /len = 678′ | | |
| 43431_at | Cluster Incl. AW006202:wz93e02.x1 Homo sapiens cDNA, | 0.10274855 | 0.004097 |
| | 3 end /clone = IMAGE-2566394 /clone_end = 3′ | | |
| | /gb = AW006202 /gi = 5854980 /ug = Hs.235162 /len = 539′ | | |
| 46104_at | Cluster Incl. AA772055:ai41e03.s1 Homo sapiens cDNA, | 0.098385581 | 0.000009 |
| | 3 end /clone = 1359580 /clone_end = 3′ | | |
| | /gb = AA772055 /gi = 2823838 /ug = Hs.55314 /len = 504′ | | |
| 51071_at | Cluster Incl. N49836:yz08a11.s1 Homo sapiens cDNA, | 0.096194999 | 0 |
| | 3 end /clone = IMAGE-282428 /clone_end = 3′ | | |
| | /gb = N49836 /gi = 1191002 /ug = Hs.46969 /len = 638′ | | |
| 47929_at | Cluster Incl. AI097310:qb35a09.x1 Homo sapiens cDNA, | 0.083211562 | 0.000054 |
| | 3 end /clone = IMAGE-1698232 /clone_end = 3′ | | |
| | /gb = AI097310 /gi = 3446892 /ug = Hs.165240 /len = 476′ | | |
| 44613_at | Cluster Incl. AI681917:tx50f03.x1 Homo sapiens cDNA, | 0.082599471 | 0.000147 |
| | 3 end /clone = IMAGE-2273021 /clone_end = 3′ | | |
| | /gb = AI681917 /gi = 4892099 /ug = Hs.3321 /len = 711′ | | |
| 46924_at | Cluster Incl. AI824107:wj46c09.x1 Homo sapiens cDNA, | 0.081634712 | 0.000003 |
| | 3 end /clone = IMAGE-2405872 /clone_end = 3′ | | |
| | /gb = AI824107 /gi = 5444778 /ug = Hs.55408 /len = 589′ | | |
| 55436_at | Cluster Incl. AI669212:wc13c10.x1 Homo sapiens cDNA, | 0.069851384 | 0.000001 |
| | 3 end /clone = IMAGE-2315058 /clone_end = 3′ | | |
| | /gb = AI669212 /gi = 4833986 /ug = Hs.92127 /len = 596′ | | |
| 49925_at | Cluster Incl. AI971227:wr26g05.x1 Homo sapiens cDNA, | 0.066652473 | 0.000018 |
| | 3 end /clone = IMAGE-2488856 /clone_end = 3′ | | |
| | /gb = AI971227 /gi = 5768053 /ug = Hs.104266 /len = 545′ | | |
| 54445_at | Cluster Incl. AA531023:nj07e04.s1 Homo sapiens cDNA, | 0.065598325 | 0.000095 |
| | 3 end /clone = IMAGE-985662 /clone_end = 3′ | | |
| | /gb = AA531023 /gi = 2273729 /ug = Hs.59771 /len = 611′ | | |
| 46172_at | Cluster Incl. AI142832:qa26f12.s1 Homo sapiens cDNA, | 0.064763267 | 0 |
| | 3 end /clone = IMAGE-1687919 /clone_end = 3′ | | |
| | /gb = AI142832 /gi = 3659191 /ug = Hs.61232 /len = 571′ | | |
| 54005_at | Cluster Incl. AI082067:oz52f09.x1 Homo sapiens cDNA, | 0.064237546 | 0.000001 |
| | 3 end /clone = IMAGE-1678985 /clone_end = 3′ | | |
| | /gb = AI082067 /gi = 3418859 /ug = Hs.64746 /len = 629′ | | |
| 50877_at | Cluster Incl. AA351682:EST59783 Homo sapiens cDNA, | 0.059597786 | 0.000008 |
| | 3 end /clone = ATCC-152104 /clone_end = 3′ | | |
| | /gb = AA351682 /gi = 2004217 /ug = Hs.12714 /len = 415′ | | |
| 56607_at | Cluster Incl. AA195677:zr32h04.s1 Homo sapiens cDNA, | 0.058208329 | 0.000001 |
| | 3 end /clone = IMAGE-665143 /clone_end = 3′ | | |
| | /gb = AA195677 /gi = 1785355 /ug = Hs.86023 /len = 475′ | | |
| 47119_at | Cluster Incl. AA130221:zl37h09.r1 Homo sapiens cDNA, | 0.044591279 | 0.000022 |
| | 5 end /clone = IMAGE-504161 /clone_end = 5′ | | |
| | /gb = AA130221 /gi = 1691225 /ug = Hs.87013 /len = 595′ | | |
| 47941_at | Cluster Incl. AI540870:PEC1.2_07_G11.r Homo sapiens cDNA, | 0.036240181 | 0.00003 |
| | 5 end /clone_end = 5′ /gb = AI540870 | | |
| | /gi = 4458243 /ug = Hs.16740 /len = 862′ | | |
| 51669_r_at | Cluster Incl, AA583578:nn61c02.s1 Homo sapiens cDNA, | 0.035202429 | 0.000027 |
| | 3 end /clone = IMAGE-1088354 /clone_end = 3′ | | |
| | /gb = AA583578 /gi = 2368187 /ug = Hs.185158 /len = 306′ | | |
| 53747_at | Cluster Incl. AA422178:zv31g07.r1 Homo sapiens cDNA, | 0.034458051 | 0.000093 |
| | 5 end /clone = IMAGE-755292 /clone_end = 5′ | | |
| | /gb = AA422178 /gi = 2101029 /ug = Hs.124660 /len = 503′ | | |
| 51214_at | Cluster Incl. AI378979:tc40d07.x1 Homo sapiens cDNA, | 0.02696214 | 0.0002 |
| | 3 end /clone = IMAGE-2067085 /clone_end = 3′ | | |
| | /gb = AI378979 /gi = 4188832 /ug = Hs.32426 /len = 527′ | | |
| 47448_at | Cluster Incl. R37637:yf61b01.s1 Homo sapiens cDNA, | 0.022494655 | 0.00001 |
| | 3 end /clone = IMAGE-26665 /clone_end = 3′ | | |
| | /gb = R37637. /gi = 795093 /ug = Hs.12286 /len = 460′ | | |
| 46163_at | Cluster Incl. AI692575:wd73h12.x1 Homo sapiens cDNA, | 0.021738726 | 0.000026 |
| | 3 end /clone = IMAGE-2337287 /clone_end = 3′ | | |
| | /gb = AI692575 /gi = 4969915 /ug = Hs.59761 /len = 493′ | | |

TABLE 4
U95_C Fold Change Genes (>3 over-expressed in Barrett's associated
esophageal adenocarcinoma (BA), <0.33 under-expressed in BA)

| Affy ID | Gene Name | Fold Change | P-value |
|---|---|---|---|
| 64472_at | Cluster Incl. AA877900:nr12g08.s1 Homo sapiens cDNA, | 20.2668142 | 0.000019 |
| | 3 end /clone = IMAGE-1161662 /clone_end = 3′ | | |
| | /gb = AA877900 /gi = 2986865 /ug = Hs.5940 /len = 580′ | | |
| 60504_at | Cluster Incl. AI859849:wm21c04.x1 Homo sapiens cDNA, | 16.18781039 | 0.000015 |
| | 3 end /clone = IMAGE-2436582 /clone_end = 3′ | | |
| | /gb = AI859849 /gi = 5513454 /ug = Hs.184598 /len = 507′ | | |
| 48774_at | Cluster Incl. AA584310:nn79g01.s1 Homo sapiens cDNA, | 13.20890926 | 0.000632 |
| | 3 end /clone = IMAGE-1090128 /clone_end = 3′ | | |
| | /gb = AA584310 /gi = 2368919 /ug = Hs.99769 /len = 582′ | | |
| 65626_at | Cluster Incl. AA059458:zl96g05.r1 Homo sapiens cDNA, | 9.104795333 | 0.000345 |
| | 5 end /clone = IMAGE-512504 /clone_end = 5′ | | |
| | /gb = AA059458 /gi = 1553317 /ug = Hs.28792 /len = 572′ | | |
| 48797_at | Cluster Incl. AI346914:qp59c05.x1 Homo sapiens cDNA, | 8.180270082 | 0.001096 |
| | 3 end /clone = IMAGE-1927304 /clone_end = 3′ | | |
| | /gb = AI346914 /gi = 4084120 /ug = Hs.105484 /len = 725′ | | |
| 61671_at | Cluster Incl. AA194980:zr35f09.r1 Homo sapiens cDNA, | 7.888195657 | 0.000433 |
| | 5 end /clone = IMAGE-665417 /clone_end = 5′ | | |
| | /gb = AA194980 /gi = 1784901 /ug = Hs.30818 /len = 585′ | | |
| 61429_at | Cluster Incl. AI948490:wq06e01.x1 Homo sapiens cDNA, | 7.811425507 | 0.000148 |
| | 3 end /clone = IMAGE-2470488 /clone_end = 3′ | | |
| | /gb = AI948490 /gi = 5740800 /ug = Hs.98765 /len = 617′ | | |
| 57224_s_{—} | Cluster Incl. AI983033:wz30a12.x1 Homo sapiens cDNA, | 7.602018897 | 0.002796 |
| | 3 end /clone = IMAGE-2559550 /clone_end = 3′ | | |
| | /gb = AI983033 /gi = 5810252 /ug = Hs.233739 /len = 456′ | | |
| 61110_at | Cluster Incl. AA679297:zi28c04.s1 Homo sapiens cDNA, | 7.48284531 | 0.000022 |
| | 3 end /clone = IMAGE-432102 /clone_end = 3′ | | |
| | /gb = AA679297 /gi = 2659819 /ug = Hs.234505 /len = 564′ | | |
| 63382_at | Cluster Incl. AI073984:oy66c05.x1 Homo sapiens cDNA, | 7.482353067 | 0.000367 |
| | 3 end /clone = IMAGE-1670792 /clone_end = 3′ | | |
| | /gb = AI073984 /gi = 3400628 /ug = Hs.14453 /len = 585′ | | |
| 65744_at | Cluster Incl. W58252:zd25d01.r1 Homo sapiens cDNA, | 7.461285225 | 0.000186 |
| | 5 end /clone = IMAGE-341665 /clone_end = 5′ | | |
| | /gb = W58252 /gi = 1365194 /ug = Hs.182793 /len = 614′ | | |
| 60585_at | Cluster Incl. N63181:yz85d07.s1 Homo sapiens cDNA, | 7.363840433 | 0.000614 |
| | 3 end /clone = IMAGE-289837 /clone_end = 3′ | | |
| | /gb = N63181 /gi = 1211010 /ug = Hs.48729 /len = 479′ | | |
| 51129_s_{—} | Cluster Incl. AI436136:ti15h07.y1 Homo sapiens cDNA, | 7.144984025 | 0.001259 |
| | 5 end /clone = IMAGE-2130589 /clone_end = 5′ | | |
| | /gb = AI436136 /gi = 4308073 /ug = Hs.237278 /len = 525′ | | |
| 48777_at | Cluster Incl. N23796:yx36a10.r1 Homo sapiens cDNA, | 7.055961732 | 0.001113 |
| | 5 end /clone = IMAGE-263802 /clone_end = 5′ | | |
| | /gb = N23796 /gi = 1137946 /ug = Hs.100132 /len = 573′ | | |
| 52429_at | Cluster Incl. AA583350:nn45h04.s1 Homo sapiens cDNA, | 7.004425205 | 0.001398 |
| | 3 end /clone = IMAGE-1086871 /clone_end = 3′ | | |
| | /gb = AA583350 /gi = 2367959 /ug = Hs.30701 /len = 494′ | | |
| 65264_at | Cluster Incl. AI306471:qn13b01.x1 Homo sapiens cDNA, | 6.697303594 | 0.000258 |
| | 3 end /clone = IMAGE-1898089 /clone_end = 3′ | | |
| | /gb = AI306471 /gi = 3989542 /ug = Hs.151334 /len = 397′ | | |
| 64408_s_{—} | Cluster Incl. AW025529:wu97d12.x1 Homo sapiens cDNA, | 6.635144195 | 0.000009 |
| | 3 end /clone = IMAGE-990954 /clone_end = 3′ | | |
| | /gb = AW025529 /gi = 5879059 /ug = Hs.47536 /len = 661′ | | |
| 63335_at | Cluster Incl. AI285531:qu49b04.x1 Homo sapiens cDNA, | 6.550211507 | 0.000055 |
| | 3 end /clone = IMAGE-1968079 /clone_end = 3′ | | |
| | /gb = AI285531 /gi = 3923764 /ug = Hs.106260 /len = 598′ | | |
| 65963_at | Cluster Incl. W72043:zd66d01.s1 Homo sapiens cDNA, | 6.462494702 | 0.000108 |
| | 3 end /clone = IMAGE-345601 /clone_end = 3′ | | |
| | /gb = W72043 /gi = 1382313 /ug = Hs.84628 /len = 604′ | | |
| 50926_s_{—} | Cluster Incl. R54585:yg81f12.s1 Homo sapiens cDNA, | 6.26291301 | 0.001886 |
| | 3 end /clone = IMAGE-39892 /clone_end = 3′ | | |
| | /gb = R54585 /gi = 816487 /ug = Hs.233799 /len = 493′ | | |
| 49102_at | Cluster Incl. AA670193:af51d07.s1 Homo sapiens cDNA, | 6.242787256 | 0.002755 |
| | 3 end /clone = 1035181 /clone_end = 3′ | | |
| | /gb = AA670193 /gi = 2631692 /ug = Hs.8687 /len = 599′ | | |
| 57830_s_{—} | Cluster Incl. AW024474:wu76h03.x1 Homo sapiens cDNA, | 6.116453605 | 0.009079 |
| | 3 end /clone = IMAGE-990888 /clone_end = 3′ | | |
| | /gb = AW024474 /gi = 5878004 /ug = Hs.236884 /len = 229′ | | |
| 62959_at | Cluster Incl. AA204719:zq84c03.s1 Homo sapiens cDNA, | 5.981172544 | 0.000101 |
| | 3 end /clone = IMAGE-648292 /clone_end = 3′ | | |
| | /gb = AA204719 /gi = 1802700 /ug = Hs.86327 /len = 576′ | | |
| 62952_at | Cluster Incl. AA480075:zv42b06.r1 Homo sapiens cDNA, | 5.866713492 | 0.000011 |
| | 5 end /clone = IMAGE-756275 /clone_end = 5′ | | |
| | /gb = AA480075 /gi = 2208226 /ug = Hs.85015 /len = 588′ | | |
| 65792_at | Cluster Incl. AA526079:ni94d02.s1 Homo sapiens cDNA, | 5.682858552 | 0.002038 |
| | 3 end /clone = IMAGE-984483 /clone_end = 3′ | | |
| | /gb = AA526079 /gi = 2268148 /ug = Hs.234180 /len = 551′ | | |
| 65128_at | Cluster Incl. AI915649:wg74a06.x1 Homo sapiens cDNA, | 5.678364543 | 0.000005 |
| | 3 end /clone = IMAGE-2370802 /clone_end = 3′ | | |
| | /gb = AI915649 /gi = 5635558 /ug = Hs.11638 /len = 716′ | | |
| 57223_at | Cluster Incl. AI982768:wu16a03.x1 Homo sapiens cDNA, | 5.672386526 | 0.000027 |
| | 3 end /clone = IMAGE-2517100 /clone_end = 3′ | | |
| | /gb = AI982768 /gi = 5809987 /ug = Hs.233732 /len = 292′ | | |
| 55210_at | Cluster Incl. AI394117:tg21g11.x1 Homo sapiens cDNA, | 5.644425711 | 0.001741 |
| | 3 end /clone = IMAGE-2109476 /clone_end = 3′ | | |
| | /gb = AI394117 /gi = 4223664 /ug = Hs.156339 /len = 251′ | | |
| 56599_i_a | Cluster Incl. AI005676:ov59f10.s1 Homo sapiens cDNA, | 5.45726309 | 0.010029 |
| | 3 end /clone = IMAGE-1641643 /clone_end = 3′ | | |
| | /gb = AI005676 /gi = 3215186 /ug = Hs.237762 /len = 491′ | | |
| 57084_s_{—} | Cluster Incl. AW009817:ws87h04.x1 Homo sapiens cDNA, | 5.442500182 | 0.03874 |
| | 3 end /clone = IMAGE-2504983 /clone_end = 3′ | | |
| | /gb = AW009817 /gi = 5858595 /ug = Hs.236097 /len = 440′ | | |
| 65339_r_a | Cluster Incl:AA004528:zh91a09.s1 Homo sapiens cDNA, | 5.431448971 | 0.000043 |
| | 3 end /clone = IMAGE-428632 /clone_end = 3′ | | |
| | /gb = AA004528 /gi = 1448105 /ug = Hs.169444 /len = 415′ | | |
| 64246_at | Cluster Incl. AI660247:we68h08.x1 Homo sapiens cDNA, | 5.427125242 | 0.000386 |
| | 3 end /clone = IMAGE-2346303 /clone_end = 3′ | | |
| | /gb = AI660247 /gi = 4763817 /ug = Hs.32699 /len = 601′ | | |
| 65155_at | Cluster Incl. AA707308:zj27a11.s1 Homo sapiens cDNA, | 5.423294999 | 0.000008 |
| | 3 end /clone = 451484 /clone_end = 3′ | | |
| | /gb = AA707308 /gi = 2717226 /ug = Hs.12369 /len = 566′ | | |
| 63307_at | Cluster Incl. AL041566:DKFZp434l0217_s1 Homo sapiens cDNA, | 5.414527108 | 0.000272 |
| | 3 end /clone = DKFZp434I0217 /clone_end = 3′ | | |
| | /gb = AL041566 /gi = 5420916 /ug = Hs.100747 /len = 715′ | | |
| 51758_s_{—} | Cluster Incl. N21439:yx58f02.s1 Homo sapiens cDNA, | 5.412633228 | 0.000095 |
| | 3 end /clone = IMAGE-265947 /clone_end = 3′ | | |
| | /gb = N21439 /gi = 1126609 /ug = Hs.238941 /len = 510′ | | |
| 48744_g_{—} | Cluster Incl. AA612678:nq25e10.s1 Homo sapiens cDNA, | 5.282186627 | 0.000486 |
| | 3 end /clone = IMAGE-1144938 /clone_end = 3′ | | |
| | /gb = AA612678 /gi = 2463716 /ug = Hs.95583 /len = 565′ | | |
| 64740_at | Cluster Incl. AA470369:ne10a12.s1 Homo sapiens cDNA, | 5.272312377 | 0.00136 |
| | 3 end /clone = IMAGE-880798 /clone_end = 3′ | | |
| | /gb = AA470369 /gi = 2197678 /ug = Hs.72115 /len = 708′ | | |
| 65516_at | Cluster Incl. AA883422:am25a05.s1 Homo sapiens cDNA, | 5.212802857 | 0.005655 |
| | 3 end /clone = IMAGE-1467824 /clone_end = 3′ | | |
| | /gb = AA883422 /gi = 2992952 /ug = Hs.18791 /len = 714′ | | |
| 60071_s_{—} | Cluster Incl. AI961907:wt40f10.x1 Homo sapiens cDNA, | 5.197556431 | 0.000125 |
| | 3 end /clone = IMAGE-2509963 /clone_end = 3′ | | |
| | /gb = AI961907 /gi = 5754620 /ug = Hs.239230 /len = 448′ | | |
| 65883_at | Cluster Incl. AI954159:wx80e01.x1 Homo sapiens cDNA, | 5.022448143 | 0.003175 |
| | 3 end /clone = IMAGE-2549976 /clone_end = 3′ | | |
| | /gb = AI954159 /gi = 5746469 /ug = Hs.47622 /len = 534′ | | |
| 63927_f_a | Cluster Incl. AI963642:wr64b03.x1 Homo sapiens cDNA, | 4.776630548 | 0.000532 |
| | 3 end /clone = IMAGE-2492429 /clone_end = 3′ | | |
| | /gb = AI963642 /gi = 5756281 /ug = Hs.231969 /len = 577′ | | |
| 63673_at | Cluster Incl. AI635057:tz03g09.x1 Homo sapiens cDNA, | 4.699661783 | 0.016394 |
| | 3 end /clone = IMAGE-2287552 /clone_end = 3′ | | |
| | /gb = AI635057 /gi = 4686387 /ug = Hs.200030 /len = 703′ | | |
| 62565_at | Cluster Incl. W21872:57h7 Homo sapiens cDNA | 4.639766922 | 0.001581 |
| | /clone = (not-directional) /gb = W21872 /gi = 1298705 | | |
| | /ug = Hs.7907 /len = 773 | | |
| 65015_at | Cluster Incl. AI092131:qa59g12.s1 Homo sapiens cDNA, | 4.631614099 | 0.00018 |
| | 3 end /clone = IMAGE-1691110 /clone_end = 3′ | | |
| | /gb = AI092131 /gi = 3431125 /ug = Hs.11210 /len = 529′ | | |
| 61881_at | Cluster Incl. AA675917:e03503f Homo sapiens cDNA, | 4.514486314 | 0.000292 |
| | 3 end /clone = e03503 /clone_end = 3′ | | |
| | /gb = AA675917 /gi = 2775264 /ug = Hs.120912 /len = 787′ | | |
| 65645_at | Cluster Incl. AI675886:wc06f12.x1 Homo sapiens cDNA, | 4.50874656 | 0.000061 |
| | 3 end /clone = IMAGE-2314415 /clone_end = 3′ | | |
| | /gb = AI675886 /gi = 4876366 /ug = Hs.30299 /len = 590′ | | |
| 65578_at | Cluster Incl. AI817147:wg62c06.x1 Homo sapiens cDNA, | 4.446160162 | 0.000109 |
| | 3 end /clone = IMAGE-2369674 /clone_end = 3′ | | |
| | /gb = AI817147 /gi = 5436226 /ug = Hs.238105 /len = 763′ | | |
| 59510_at | Cluster Incl. AI832076:td12b02.x1 Homo sapiens cDNA, | 4.330867004 | 0.001152 |
| | 3 end /clone = IMAGE-2075403 /clone_end = 3′ | | |
| | /gb = AI832076 /gi = 5452747 /ug = Hs.127305 /len = 734′ | | |
| 54839_at | Cluster Incl. N51702:yy72d03.s1 Homo sapiens cDNA, | 4.278675286 | 0.040746 |
| | 3 end /clone = IMAGE-279077 /clone_end = 3′ | | |
| | /gb = N51702 /gi = 1192868 /ug = Hs.101392 /len = 514′ | | |
| 63596_f_a | Cluster Incl. AI983633:wt50b06.x1 Homo sapiens cDNA, | 4.226128442 | 0.000841 |
| | 3 end /clone = IMAGE-2510867 /clone_end = 3′ | | |
| | /gb = AI983633 /gi = 5810852 /ug = Hs.233757 /len = 610′ | | |
| 64504_r_a | Cluster Incl. AA868136:ak45g12.s1 Homo sapiens cDNA, | 4.175057561 | 0.001777 |
| | 3 end /clone = IMAGE-1408966 /clone_end = 3′ | | |
| | /gb = AA868136 /gi = 2963581 /ug = Hs.238884 /len = 306′ | | |
| 62240_at | Cluster Incl. AI096692:qb91b06.x1 Homo sapiens cDNA, | 4.173231366 | 0.000007 |
| | 3 end /clone = IMAGE-1707443 /clone_end = 3′ | | |
| | /gb = AI096692 /gi = 3446186 /ug = Hs.157461 /len = 447′ | | |
| 62467_at | Cluster Incl. W63576:zc55h09.r1 Homo sapiens cDNA, | 4.112846813 | 0.000093 |
| | 5 end /clone = IMAGE-326273 /clone_end = 5′ | | |
| | /gb = W63576 /gi = 1371149 /ug = Hs.58093 /len = 604′ | | |
| 54444_at | Cluster Incl. AA862350:og94c06.s1 Homo sapiens cDNA, | 4.090071897 | 0.004216 |
| | 3 end /clone = IMAGE-1455946 /clone_end = 3′ | | |
| | /gb = AA862350 /gi = 2954829 /ug = Hs.96151 /len = 590′ | | |
| 64454_at | Cluster Incl. AI805943:te52h02.x1 Homo sapiens cDNA, | 4.013492489 | |
| | 3 end /clone = IMAGE-2090355 /clone_end = 3′ | | |
| | /gb = AI805943 /gi = 5392509 /ug = Hs.5723 /len = 772′ | | |
| 64702_at | Cluster Incl. AI809005:wf67f06.x1 Homo sapiens cDNA, | 4.009878713 | 0.000142 |
| | 3 end /clone = IMAGE-2360675 /clone_end = 3′ | | |
| | /gb = AI809005 /gi = 5395571 /ug = Hs.50883 /len = 596′ | | |
| 61943_at | Cluster Incl. AI262104:qz28d10.y1 Homo sapiens cDNA, | 3.992422378 | 0.000194 |
| | 5 end /clone = IMAGE-2028211 /clone_end = 5′ | | |
| | /gb = AI262104 /gi = 3870307 /ug = Hs.140932 /len = 523′ | | |
| 62695_at | Cluster Incl. AA502331:ne26h06.s1 Homo sapiens cDNA, | 3.975578279 | 0.009789 |
| | 3 end /clone = IMAGE-898523 /clone_end = 3′ | | |
| | /gb = AA502331 /gi = 2237298 /ug = Hs.15951 /len = 534′ | | |
| 64514_at | Cluster Incl. AI811865:tw45f01.x1 Homo sapiens cDNA, | 3.900924095 | 0.003286 |
| | 3 end /clone = IMAGE-2262649 /clone_end = 3′ | | |
| | /gb = AI811865 /gi = 5398431 /ug = Hs.239080 /len = 401′ | | |
| 60860_f_a | Cluster Incl. AI086805:oz57e06.x1 Homo sapiens cDNA, | 3.889966281 | 0.003683 |
| | 3 end /clone = IMAGE-1679458 /clone_end = 3′ | | |
| | /gb = AI086805 /gi = 3425228 /ug = Hs.233789 /len = 420′ | | |
| 65690_at | Cluster Incl. AI091277:ow62b07.x1 Homo sapiens cDNA, | 3.881822704 | 0.002192 |
| | 3 end /clone = IMAGE-1651381 /clone_end = 3′ | | |
| | /gb = AI091277 /gi = 3430336 /ug = Hs.32659 /len = 692′ | | |
| 60082_s_{—} | Cluster Incl. AI991395:ws09f02.x1 Homo sapiens cDNA, | 3.80225973 | 0.008484 |
| | 3 end /clone = IMAGE-2496699 /clone_end = 3′ | | |
| | /gb = AI991395 /gi = 5838300 /ug = Hs.239944 /len = 300′ | | |
| 64735_at | Cluster Incl. AI684745:wa85f09.x1 Homo sapiens cDNA, | 3.799796162 | 0.031697 |
| | 3 end /clone = IMAGE-2302985 /clone_end = 3′ | | |
| | /gb = AI684745 /gi = 4896039 /ug = Hs.6952 /len = 652′ | | |
| 65139_at | Cluster Incl. AI925240:wn52d12.x1 Homo sapiens cDNA, | 3.79978649 | 0.00067 |
| | 3 end /clone = IMAGE-2449079 /clone_end = 3′ | | |
| | /gb = AI925240 /gi = 5661204 /ug = Hs.118983 /len = 494′ | | |
| 64742_at | Cluster Incl. AI638445:tt07a10.x1 Homo sapiens cDNA, | 3.799745386 | 0.038879 |
| | 3 end /clone = IMAGE-2240058 /clone_end = 3′ | | |
| | /gb = AI638445 /gi = 4690679 /ug = Hs.72484 /len = 718′ | | |
| 60797_at | Cluster Incl:AI076180:oz01f06.x1 Homo sapiens cDNA, | 3.77504304 | 0.005248 |
| | 3 end /clone = IMAGE-1674083 /clone_end = 3′ | | |
| | /gb = AI076180 /gi = 3405358 /ug = Hs.214410 /len = 603′ | | |
| 64258_f_a | Cluster Incl. AW016235:UI-H-BI0p-abl-a-06-0-UI.s1 Homo sapiens cDNA, | 3.774669144 | 0.002065 |
| | 3 end /clone = IMAGE-2712083 /clone_end = 3′ | | |
| | /gb = AW016235 /gi = 5864992 /ug = Hs.234283 /len = 438′ | | |
| 65572_at | Cluster Incl. AI635827:tz81d05.x1 Homo sapiens cDNA, | 3.740835788 | 0.016428 |
| | 3 end /clone = IMAGE-2294985 /clone_end = 3′ | | |
| | /gb = AI635827 /gi = 4687157 /ug = Hs.23723 /len = 709′ | | |
| 48664_at | Cluster Incl. AI762879:wi63d05.x1 Homo sapiens cDNA, | 3.728590312 | 0.00008 |
| | 3 end /clone = IMAGE-2397993 /clone_end = 3′ | | |
| | /gb = AI762879 /gi = 5178546 /ug = Hs.86437 /len = 604′ | | |
| 61126_at | Cluster Incl. N22262:yw36c04.s1 Homo sapiens cDNA, | 3.726525355 | 0.000113 |
| | 3 end /clone = IMAGE-254310 /clone_end = 3′ | | |
| | /gb = N22262 /gi = 1128396 /ug = Hs.30779 /len = 447′ | | |
| 63994_i_a | Cluster Incl. AW006898:ws15g04.x1 Homo sapiens cDNA, | 3.685571992 | 0.020562 |
| | 3 end /clone = IMAGE-2497302 /clone_end = 3′ | | |
| | /gb = AW006898 /gi = 5855676 /ug = Hs.234094 /len = 228′ | | |
| 62997_at | Cluster Incl. AI972895:wr44h09.x1 Homo sapiens cDNA, | 3.675466573 | 0.006908 |
| | 3 end /clone = IMAGE-2490593 /clone_end = 3′ | | |
| | /gb = AI972895 /gi = 5769721 /ug = Hs.91226 /len = 594′ | | |
| 51097_at | Cluster Incl. AW003232:wq64c12.x1 Homo sapiens cDNA, | 3.647036307 | 0.003288 |
| | 3 end /clone = IMAGE-2476054 /clone_end = 3′ | | |
| | /gb = AW003232 /gi = 5850148 /ug = Hs.236518 /len = 683′ | | |
| 58825_at | Cluster Incl. AI701911:tq19f05.x1 Homo sapiens cDNA, | 3.636855749 | 0.009978 |
| | 3 end /clone = IMAGE-2209281 /clone_end = 3′ | | |
| | /gb = AI701911 /gi = 4989811 /ug = Hs.236455 /len = 431′ | | |
| 50183_at | Cluster Incl. AI347165:tc04d10.x1 Homo sapiens cDNA, | 3.626154256 | 0.005991 |
| | 3 end /clone = IMAGE-2062867 /clone_end = 3′ | | |
| | /gb = AI347165 /gi = 4084371 /ug = Hs.71561 /len = 535′ | | |
| 65797_at | Cluster Incl. AA127736:zk88c12.r1 Homo sapiens cDNA, | 3.610269552 | 0.001966 |
| | 5 end /clone = IMAGE-489910 /clone_end = 5′ | | |
| | /gb = AA127736 /gi = 1687099 /ug = Hs.237523 /len = 616′ | | |
| 53261_at | Cluster Incl. AI337231:qx83h10.x1 Homo sapiens cDNA, | 3.583182611 | 0.000578 |
| | 3 end /clone = IMAGE-2009155 /clone_end = 3′ | | |
| | /gb = AI337231 /gi = 4074158 /ug = Hs.50115 /len = 484′ | | |
| 62536_at | Cluster Incl. AA527180:ni20b09.s1 Homo sapiens cDNA, | 3.56570974 | 0.001133 |
| | 3 end /clone = IMAGE-968537 /clone_end = 3′ | | |
| | /gb = AA527180 /gi = 2269249 /ug = Hs.67928 /len = 577′ | | |
| 56784_at | Cluster Incl. AI016894:ou31c06.x1 Homo sapiens cDNA, | 3.557237251 | 0.001861 |
| | 3 end /clone = IMAGE-1627882 /clone_end = 3′ | | |
| | /gb = AI016894 /gi = 3231230 /ug = Hs.45204 /len = 431′ | | |
| 57027_at | Cluster Incl. AI990405:ws20h10.x1 Homo sapiens cDNA, | 3.547382938 | 0.00167 |
| | 3 end /clone = IMAGE-2497795 /clone_end = 3′ | | |
| | /gb = AI990405 /gi = 5837286 /ug = Hs.233881 /len = 481′ | | |
| 61638_at | Cluster Incl. AA700160:zj36h03.s1 Homo sapiens cDNA, | 3.543445756 | 0.000324 |
| | 3 end /clone = 452405 /clone_end = 3′ | | |
| | /gb = AA700160 /gi = 2703123 /ug = Hs.28564 /len = 502′ | | |
| 60950_f_a | Cluster Incl. AA741117:nz04f09.s1 Homo sapiens cDNA | 3.543004523 | 0.000039 |
| | /clone = IMAGE-1286825 /gb = AA741117 /gi = 2779709 | | |
| | /ug = Hs.78684 /len = 466 | | |
| 62950_at | Cluster Incl. W84421:zd89g04.s1 Homo sapiens cDNA, | 3.487421091 | 0.000644 |
| | 3 end /clone = IMAGE-356694 /clone_end = 3′ | | |
| | /gb = W84421 /gi = 1395562 /ug = Hs.83992 /len = 648′ | | |
| 58848_at | Cluster Incl. AI809489:wf30b04.x1 Homo sapiens cDNA, | 3.471887883 | 0.007471 |
| | 3 end /clone = IMAGE-2357071 /clone_end = 3′ | | |
| | /gb = AI809489 /gi = 5396055 /ug = Hs.236580 /len = 787′ | | |
| 64274_at | Cluster Incl. AI766469:wi41a06.x1 Homo sapiens cDNA, | 3.471012959 | 0.020051 |
| | 3 end /clone = IMAGE-2392786 /clone_end = 3′ | | |
| | /gb = AI766469 /gi = 5232978 /ug = Hs.234483 /len = 337′ | | |
| 55678_at | Cluster Incl. W72885:zd59d05.s1 Homo sapiens cDNA, | 3.422525993 | 0.00455 |
| | 3 end /clone = IMAGE-344937 /clone_end = 3′ | | |
| | /gb = W72885 /gi = 1383020 /ug = Hs.237652 /len = 424′ | | |
| 62743_at | Cluster Incl. AA811088:oa85c12.s1 Homo sapiens cDNA | 3.399387119 | 0.011542 |
| | /clone = IMAGE-1319062 /gb = AA811088 /gi = 2880699 | | |
| | /ug = Hs.172581 /len = 571 | | |
| 65492_at | Cluster Incl. N56950:yy82g06.s1 Homo sapiens cDNA, | 3.365968673 | 0.00226 |
| | 3 end /clone = IMAGE-280090 /clone_end = 3′ | | |
| | /gb = N56950 /gi = 1200840 /ug = Hs.172241 /len = 602′ | | |
| 64144_at | Cluster Incl. AW026010:wv05e09.x1 Homo sapiens cDNA, | 3.358826512 | 0.000172 |
| | 3 end /clone = IMAGE-990875 /clone_end = 3′ | | |
| | /gb = AW026010 /gi = 5879540 /ug = Hs.239812 /len = 580′ | | |
| 58636_f_a | Cluster Incl. R33841:yh82a10.s1 Homo sapiens cDNA, | 3.356616113 | 0.00199 |
| | 3 end /clone = IMAGE-136218 /clone_end = 3′ | | |
| | /gb = R33841 /gi = 789699 /ug = Hs.175011 /len = 400′ | | |
| 49146_at | Cluster Incl. AA305101:EST176103 Homo sapiens cDNA, | 3.33565472 | 0.020805 |
| | 5 end /clone = ATCC-128591 /clone_end = 5′ | | |
| | /gb = AA305101 /gi = 1957429 /ug = Hs.107614 /len = 547′ | | |
| 60006_at | Cluster Incl. AI816780:wj34a09.x1 Homo sapiens cDNA, | 3.315740997 | 0.019201 |
| | 3 end /clone = IMAGE-2404696 /clone_end = 3′ | | |
| | /gb = AI816780 /gi = 5435859 /ug = Hs.15088 /len = 499′ | | |
| 48773_at | Cluster Incl. AA642981:nr60h11.s1 Homo sapiens cDNA, | 3.313599312 | 0.004231 |
| | 3 end /clone = IMAGE-1172421 /clone_end = 3′ | | |
| | /gb = AA642981 /gi = 2568199 /ug = Hs.9960 /len = 563′ | | |
| 53789_at | Cluster Incl. AI866583:tz51d04.x1 Homo sapiens cDNA, | 3.313016769 | 0.001358 |
| | 3 end /clone = IMAGE-2292103 /clone_end = 3′ | | |
| | /gb = AI866583 /gi = 5530690 /ug = Hs.74921 /len = 479′ | | |
| 66186_at | Cluster Incl. Z41019:HSC2MC092 Homo sapiens cDNA, | 3.303305427 | 0.000884 |
| | 3 end /clone = c-2mc09 /clone_end = 3′ | | |
| | /gb = Z41019 /gi = 566764 /ug = Hs.113530 /len = 317′ | | |
| 63800_at | Cluster Incl. AA195158:zr35f06.r1 Homo sapiens cDNA, | 3.293703299 | 0.000004 |
| | 5 end /clone = IMAGE-665411 /clone_end = 5′ | | |
| | /gb = AA1951587gi = 1784847 /ug = Hs.204154 /len = 723′ | | |
| 50202_s_{—} | Cluster Incl. AA917667:on46d02.s1 Homo sapiens cDNA, | 3.28918373 | 0.001767 |
| | 3 end /clone = IMAGE-1559715 /clone_end = 3′ | | |
| | /gb = AA917667 /gi = 3057557 /ug = Hs.77365 /len = 562′ | | |
| 66585_at | Cluster Incl. AA879466:oj91h03.s1 Homo sapiens cDNA, | 3.285845033 | 0.019475 |
| | 3 end /clone = IMAGE-1505717 /clone_end = 3′ | | |
| | /gb = AA879466 /gi = 2988577 /ug = Hs.125427 /len = 416′ | | |
| 61448_at | Cluster Incl. AA766126:oa27f10.s1 Homo sapiens cDNA | 3.275425186 | 0.000966 |
| | /clone = IMAGE-1306219 /gb = AA766126 /gi = 2817364 | | |
| | /ug = Hs.99338 /len = 398 | | |
| 63124_at | Cluster Incl. AA631399:np86e09.s1 Homo sapiens cDNA | 3.274156292 | 0.001603 |
| | /clone = IMAGE-1133224 /gb = AA631399 /gi = 2554010 | | |
| | /ug = Hs.191512 /len = 457 | | |
| 57034_at | Cluster Incl. AW003626:wx34e02.x1 Homo sapiens cDNA, | 3.255247904 | 0.020433 |
| | 3 end /clone = IMAGE-2545562 /clone end = 3′ | | |
| | /gb = AW003626 /gi = 5850542 /ug = Hs.234018 /len = 707′ | | |
| 49633_at | Cluster Incl. N21131:yx52g04.s1 Homo sapiens cDNA, | 3.253214384 | 0.014949 |
| | 3 end /clone = IMAGE-265398 /clone_end = 3′ | | |
| | /gb = N21131 /gi = 1126301 /ug = Hs.233612 /len = 571′ | | |
| 60314_at | Cluster Incl. AI014820:ot87f01.s1 Homo sapiens cDNA, | 3.249891116 | 0.001175 |
| | 3 end /clone = IMAGE-1623769 /clone_end = 3′ | | |
| | /gb = AI014820 /gi = 3229156 /ug = Hs.131756 /len = 311′ | | |
| 48746_at | Cluster Incl. AA527237:ng35e09.s1 Homo sapiens cDNA, | 3.24979003 | 0.016432 |
| | 3 end /clone = IMAGE-936808 /clone_end = 3′ | | |
| | /gb = AA527237 /gi = 2269306 /ug = Hs.95583 /len = 496′ | | |
| 61941_at | Cluster Incl. AI701037:we09h11.x1 Homo sapiens cDNA, | 3.247978145 | 0.004021 |
| | 3 end /clone = IMAGE-2340645 /clone_end = 3′ | | |
| | /gb = AI701037 /gi = 4988937 /ug = Hs.139784 /len = 586′ | | |
| 61664_r_a | Cluster Incl. AI859398:wm11a08.x1 Homo sapiens cDNA, | 3.236515692 | 0.008108 |
| | 3 end /clone = IMAGE-2435606 /clone_end = 3′ | | |
| | /gb = AI859398 /gi = 5513014 /ug = Hs.3046 /len = 518′ | | |
| 65443_at | Cluster Incl. AA085711:zl83c09.s1 Homo sapiens cDNA, | 3.236087458 | 0.018668 |
| | 3 end /clone = IMAGE-511216 /clone_end = 3′ | | |
| | /gb = AA085711 /gi = 1629467 /ug = Hs.15125 /len = 630′ | | |
| 59134_at | Cluster Incl. AA947258:od86c08.s1 Homo sapiens cDNA | 3.223980919 | 0.002204 |
| | /clone = IMAGE-1374830 /gb = AA947258 /gi = 3108511 | | |
| | /ug = Hs.127703 /len = 277 | | |
| 62214_at | Cluster Incl. AI583960:ts08g12.x1 Homo sapiens cDNA, | 3.15598437 | 0.021671 |
| | 3 end /clone = IMAGE-2228038 /clone_end = 3′ | | |
| | /gb = AI583960 /gi = 4569857 /ug = Hs.55918 /len = 1056′ | | |
| 48666_at | Cluster Incl. AA902949:ok43h08.s1 Homo sapiens cDNA, | 3.152741137 | 0.002793 |
| | 3 end /clone = IMAGE-1516767 /clone_end = 3′ | | |
| | /gb = AA902949 /gi = 3038072 /ug = Hs.86489 /len = 546′ | | |
| 64388_at | Cluster Incl. AI685200:wc70b09.x1 Homo sapiens cDNA, | 3.120427707 | 0.001253 |
| | 3 end /clone = IMAGE-2323961 /clone_end = 3′ | | |
| | /gb = AI685200 /gi = 4896494 /ug = Hs.44155 /len = 652′ | | |
| 62602_at | Cluster Incl. AA418402:zv92f02.s1 Homo sapiens cDNA, | 3.097490531 | 0.007758 |
| | 3 end /clone = IMAGE-767259 /clone_end = 3′ | | |
| | /gb = AA418402 /gi = 2080211 /ug = Hs.175034 /len = 543′ | | |
| 64668_at | Cluster Incl. AI983929:wz55g05.x1 Homo sapiens cDNA, | 3.095095738 | 0.001828 |
| | 3 end /clone = IMAGE-2562008 /clone end = 3′ | | |
| | /gb = AI983929 /gi = 5811148 /ug = Hs.44087 /len = 498′ | | |
| 60521_at | Cluster Incl. AL043931:DKFZp434F1328_s1 Homo sapiens cDNA, | 3.089245265 | 0.005927 |
| | 3 end /clone = DKFZp434F1328 /clone_end = 3′ | | |
| | /gb = AL043931 /gi = 5432159 /ug = Hs.194208 /len = 656′ | | |
| 64078_at | Cluster Incl. AA156269:zo50c04.s1 Homo sapiens cDNA, | 3.083000622 | 0.001219 |
| | 3 end /clone = IMAGE-590310 /clone_end = 3′ | | |
| | /gb = AA156269 /gi = 1727904 /ug = Hs.29383 /len = 640′ | | |
| 54843_at | Cluster Incl. AI807036:wf37d02.x1 Homo sapiens cDNA, | 3.064820883 | 0.000199 |
| | 3 end /clone = IMAGE-2357763 /clone_end = 3′ | | |
| | /gb = AI807036 /gi = 5393602 /ug = Hs.101619 /len = 593′ | | |
| 57704_at | Cluster Incl. H04885:yl74a03.r1 Homo sapiens cDNA, | 3.049533011 | 0.020401 |
| | 5 end /clone = IMAGE-43628 /clone_end = 5′ | | |
| | /gb = H04885 /gi = 868437 /ug = Hs.72363 /len = 517′ | | |
| 48799_at | Cluster Incl. AI569988:tr90f10.x1 Homo sapiens cDNA, | 3.034133542 | 0.004434 |
| | 3 end /clone = IMAGE-2226379 /clone_end = 3′ | | |
| | /gb = AI569988 /gi = 4533362 /ug = Hs.105547 /len = 744′ | | |
| 57697_at | Cluster Incl. AI445628:tj08c06.x1 Homo sapiens cDNA, | 3.03160246 | 0.005747 |
| | 3 end /clone = IMAGE-2140906 /clone_end = 3′ | | |
| | /gb = AI445628 /gi = 4289541 /ug = Hs.70980 /len = 486′ | | |
| 49728_s_{—} | Cluster Incl. AI056993:oz06b07.x1 Homo sapiens cDNA, | 3.030939775 | 0.038552 |
| | 3 end /clone = IMAGE-1674517 /clone_end = 3′ | | |
| | /gb = AI056993 /gi = 3330782 /ug = Hs.239310 /len = 536′ | | |
| 64103_at | Cluster Incl. N22751:yx65d07.s1 Homo sapiens cDNA, | 3.029370854 | 0.000031 |
| | 3 end /clone = IMAGE-266605 /clone_end = 3′ | | |
| | /gb = N22751 /gi = 1136901 /ug = Hs.34665 /len = 632′ | | |
| 64347_s_{—} | Cluster Incl. AW007256:wt54b12.x1 Homo sapiens cDNA, | 3.014120526 | 0.014055 |
| | 3 end /clone = IMAGE-2511263 /clone_end = 3′ | | |
| | /gb = AW007256 /gi = 5856034 /ug = Hs.237397 /len = 631′ | | |
| 54364_at | Cluster Incl. AI472136:tj86a07.x1 Homo sapiens cDNA, | 3.013549569 | 0.010051 |
| | 3 end /clone = IMAGE-2148372 /clone_end = 3′ | | |
| | /gb = AI472136 /gi = 4334226 /ug = Hs.88801 /len = 549′ | | |
| 61626_at | Cluster Incl. AA534969:nf72h05.s1 Homo sapiens cDNA, | 0.329753477 | 0.005999 |
| | 3 end /clone = IMAGE-925497 /clone_end = 3′ | | |
| | /gb = AA534969 /gi = 2279222 /ug = Hs.27536 /len = 552′ | | |
| 66390_at | Cluster Incl. AI302387:qn50g05.x1 Homo sapiens cDNA, | 0.329198527 | 0.000067 |
| | 3 end /clone = IMAGE-1901720 /clone_end = 3′ | | |
| | /gb = AI302387 /gi = 3961733 /ug = Hs.120568 /len = 396′ | | |
| 66860_at | Cluster Incl. AA994029:ou42e09.s1 Homo sapiens cDNA, | 0.328178816 | 0.002065 |
| | 3 end /clone = IMAGE-1628968 /clone_end = 3′ | | |
| | /gb = AA994029 /gi = 3180574 /ug = Hs.129464 /len = 471′ | | |
| 64285_at | Cluster Incl. AI050855:oy47a05.x1 Homo sapiens cDNA, | 0.327569476 | 0.000116 |
| | 3 end /clone = IMAGE-1668944 /clone_end = 3′ | | |
| | /gb = AI050855 /gi = 3307660 /ug = Hs.234984 /len = 427′ | | |
| 48866_at | Cluster Incl. AA176945:zp36c10.s1 Homo sapiens cDNA, | 0.327322228 | 0.044146 |
| | 3 end /clone = IMAGE-611538 /clone_end = 3′ | | |
| | /gb = AA176945 /gi = 1758103 /ug = Hs.111136 /len = 710′ | | |
| 54748_at | Cluster Incl. AI309007:qo90b04.x1 Homo sapiens cDNA, | 0.326909965 | 0.012793 |
| | 3 end /clone = IMAGE-1915759 /clone_end = 3′ | | |
| | /gb = AI309007 /gi = 4003878 /ug = Hs.152569 /len = 424′ | | |
| 65964_at | Cluster Incl. AA203147:zx57d05.r1 Homo sapiens cDNA, | 0.323997933 | 0.016279 |
| | 5 end /clone = IMAGE-446601 /clone_end = 5′ | | |
| | /gb = AA203147 /gi = 1799005 /ug = Hs.8518 /len = 889′ | | |
| 62824_at | Cluster Incl. AI095360:oy17f06.s1 Homo sapiens cDNA, | 0.32360218 | 0.009552 |
| | 3 end /clone = IMAGE-1666115 /clone_end = 3′ | | |
| | /gb = AI095360 /gi = 3434336 /ug = Hs.148696 /len = 428′ | | |
| 60491_at | Cluster Incl. AA767440:nz82c02.s1 Homo sapiens cDNA | 0.323586785 | 0.037968 |
| | /clone = IMAGE-1301954 /gb = AA767440 /gi = 2818455 | | |
| | /ug = Hs.182882 /len = 503 | | |
| 57255_f_a | Cluster Incl. AI991501:ws17d09.x1 Homo sapiens cDNA, | 0.323023022 | 0.016114 |
| | 3 end /clone = IMAGE-2497457 /clone_end = 3′ | | |
| | /gb = AI991501 /gi = 5838406 /ug = Hs.233904 /len = 290′ | | |
| 62246_at | Cluster Incl. T92947:ye27b10.s1 Homo sapiens cDNA, | 0.322335146 | 0.001962 |
| | 3 end /clone = IMAGE-118939 /clone_end = 3′ | | |
| | /gb = T92947 /gi = 724860 /ug = Hs.160999 /len = 458′ | | |
| 63972_at | Cluster Incl. AI985619:wr75e11.x1 Homo sapiens cDNA, | 0.321481527 | 0.012815 |
| | 3 end /clone = IMAGE-2493548 /clone_end = 3′ | | |
| | /gb = AI985619 /gi = 5812896 /ug = Hs.233813 /len = 645′ | | |
| 65471_at | Cluster Incl. AI744118:wc36e07.x1 Homo sapiens cDNA, | 0.319850405 | 0.03235 |
| | 3 end /clone = IMAGE-2317284 /clone_end = 3′ | | |
| | /gb = AI744118 /gi = 5112406 /ug = Hs.169575 /len = 588′ | | |
| 64684_at | Cluster Incl. AA528168:nh91g06.s1 Homo sapiens cDNA, | 0.318842345 | 0.006451 |
| | 3 end /clone = IMAGE-965914 /clone_end = 3′ | | |
| | /gb = AA528168 /gi = 2270237 /ug = Hs.48401 /len = 610′ | | |
| 49175_at | Cluster Incl. AI887986:wm27b05.x1 Homo sapiens cDNA, | 0.317648541 | 0.004512 |
| | 3 end /clone = IMAGE-2437137 /clone_end = 3′ | | |
| | /gb = AI887986 /gi = 5593150 /ug = Hs.110978 /len = 749′ | | |
| 55882_at | Cluster Incl. AI492376:ti27c10.x1 Homo sapiens cDNA, | 0.316649054 | 0.009055 |
| | 3 end /clone = IMAGE-2131698 /clone_end = 3′ | | |
| | /gb = AI492376 /gi = 4393379 /ug = Hs.17572 /len = 538′ | | |
| 64395_at | Cluster Incl. H10808:ym04c09.r1 Homo sapiens cDNA, | 0.316637145 | 0.004543 |
| | 5 end /clone = IMAGE-46861 /clone_end = 5′ | | |
| | /gb = H10808 /gi = 875628 /ug = Hs.44978 /len = 601′ | | |
| 62555_at | Cluster Incl. AI623176:ts78b04.x1 Homo sapiens cDNA, | 0.316473366 | 0.040377 |
| | 3 end /clone = IMAGE-2237359 /clone_end = 3′ | | |
| | /gb = AI623176 /gi = 4648101 /ug = Hs.7392 /len = 664′ | | |
| 64526_at | Cluster Incl. AW051272:wy83e11.x1 Homo sapiens cDNA, | 0.315849581 | 0.038785 |
| | 3 end /clone = IMAGE-2555180 /clone_end = 3′ | | |
| | /gb = AW051272 /gi = 5913542 /ug = Hs.25214 /len = 371′ | | |
| 65796_at | Cluster Incl. AI859366:wm10f01.x1 Homo sapiens cDNA, | 0.315525138 | 0.007601 |
| | 3 end /clone = IMAGE-2435545 /clone_end = 3′ | | |
| | /gb = AI859366 /gi = 5512982 /ug = Hs.237336 /len = 707′ | | |
| 61700_at | Cluster Incl. AI922821:wo14d06.x1 Homo sapiens cDNA, | 0.315495682 | 0.004301 |
| | 3 end /clone = IMAGE-2455307 /clone_end = 3′ | | |
| | /gb = AI922821 /gi = 5658785 /ug = Hs.32433 /len = 537′ | | |
| 61870_g_{—} | Cluster Incl. AI494617:qz17a11.x1 Homo sapiens cDNA, | 0.315484967 | 0.007413 |
| | 3 end /clone = IMAGE-2021756 /clone_end = 3′ | | |
| | /gb = AI494617 /gi = 4395620 /ug = Hs.118211 /len = 646′ | | |
| 60510_at | Cluster Incl. AA772381:ai44c03.s1 Homo sapiens cDNA, | 0.314791598 | 0.011959 |
| | 3 end /clone = 1359844 /clone_end = 3′ | | |
| | /gb = AA772381 /gi = 2824164 /ug = Hs.188429 /len = 570′ | | |
| 57738_at | Cluster Incl. AA195964:zp97c05.r1 Homo sapiens cDNA, | 0.314196784 | 0.007585 |
| | 5 end /clone = IMAGE-628136 /clone_end = 5′ | | |
| | /gb = AA195964 /gi = 1791589 /ug = Hs.87278 /len = 615′ | | |
| 62793_at | Cluster Incl. Al821806:nr33c08.x5 Homo sapiens cDNA, | 0.313701611 | 0.000016 |
| | 3 end /clone = IMAGE-1169774 /clone_ends = 3′ | | |
| | /gb = AI821806 /gi = 5440885 /ug = Hs.191846 /len = 679′ | | |
| 62707_at | Cluster Incl. N21031:yx46f05.s1 Homo sapiens cDNA, | 0.311461874 | 0.0078 |
| | 3 end /clone = IMAGE-264801 /clone_end = 3′ | | |
| | /gb = N21031 /gi = 1126201 /ug = Hs.164779 /len = 554′ | | |
| 63026_at | Cluster Incl. AA886976:oi14h01.s1 Homo sapiens cDNA, | 0.310368296 | 0.00234 |
| | 3 end /clone = IMAGE-1476529 /clone_end = 3′ | | |
| | /gb = AA886976 /gi = 3002084 /ug = Hs.95821 /len = 749′ | | |
| 57240_f_a | Cluster Incl. AI701742:we07g11.x1 Homo sapiens cDNA, | 0.308442897 | 0.002019 |
| | 3 end /clone = IMAGE-2340452 /clone_end = 3′ | | |
| | /gb = AI701742 /gi = 4989642 /ug = Hs.233791 /len = 344′ | | |
| 65185_g_{—} | Cluster Incl. AA015613:ze20f12.s1 Homo sapiens cDNA, | 0.306125434 | 0.000036 |
| | 3 end /clone = IMAGE-359567 /clone_end = 3′ | | |
| | /gb = AA015613 /gi = 1476661 /ug = Hs.13766 /len = 647′ | | |
| 65591_at | Cluster Incl. N64681:yz87g05.s1 Homo sapiens cDNA, | 0.303421174 | 0.022703 |
| | 3 end /clone = IMAGE-290072 /clone_end = 3′ | | |
| | /gb = N64681 /gi = 1212510 /ug = Hs.239120 /len = 606′ | | |
| 65543_at | Cluster Incl. AI621225:ts77a11.x1 Homo sapiens cDNA, | 0.302382221 | 0.003525 |
| | 3 end /clone = IMAGE-2237276 /clone_end = 3′ | | |
| | /gb = AI621225 /gi = 4630351 /ug = Hs.22452 /len = 582′ | | |
| 63952_at | Cluster Incl. AI970855:wr20f09.x1 Homo sapiens cDNA, | 0.302306661 | 0.000153 |
| | 3 end /clone = IMAGE-2488265 /clone_end = 3′ | | |
| | /gb = AI970855 /gi = 5767681 /ug = Hs.233633 /len = 590′ | | |
| 65514_at | Cluster Incl. AA166678:zq41e08.s1 Homo sapiens cDNA, | 0.302069999 | 0.000394 |
| | 3 end /clone = IMAGE-632294 /clone_end = 3′ | | |
| | /gb = AA166678 /gi = 1745133 /ug = Hs.18760 /len = 673′ | | |
| 62287_at | Cluster Incl. AW005044:wz94a11.x1 Homo sapiens cDNA, | 0.301869349 | 0.000105 |
| | 3 end /clone = IMAGE-2566460 /clone_end = 3′ | | |
| | /gb = AW005044 /gi = 5853822 /ug = Hs.167433 /len = 731′ | | |
| 65483_at | Cluster Incl. Al658928:tu07d08.x1 Homo sapiens cDNA, | 0.300258246 | 0.003195 |
| | 3 end /clone = IMAGE-2250351 /clone_end = 3′ | | |
| | /gb = AI658928 /gi = 4762498 /ug = Hs.17110 /len = 530′ | | |
| 64293_at | Cluster Incl. AI971000:wr22f07.x1 Homo sapiens cDNA, | 0.296907004 | 0.00001 |
| | 3 end /clone = IMAGE-2488453 /clone_end = 3′ | | |
| | /gb = AI971000 /gi = 5767826 /ug = Hs.235392 /len = 524′ | | |
| 56545_r_a | Cluster Incl. H77843:ys09f06.s1 Homo sapiens cDNA, | 0.296894418 | 0.000655 |
| | 3 end /clone = IMAGE-214307 /clone_end = 3′ | | |
| | /gb = H77843 /gi = 1055932 /ug = Hs.117886 /len = 331′ | | |
| 54887_at | Cluster Incl. AA772278:ai42d03.s1 Homo sapiens cDNA, | 0.295982417 | 0.003958 |
| | 3 end /clone = 1359653 /clone_end = 3′ | | |
| | /gb = AA772278 /gi = 2824061 /ug = Hs.104623 /len = 529′ | | |
| 56590_s_{—} | Cluster Incl. AI431800:ti25d06.x1 Homo sapiens cDNA, | 0.295067963 | 0.000049 |
| | 3 end /clone = IMAGE-2131499 /clone_end = 3′ | | |
| | /gb = AI431800 /gi = 4305717 /ug = Hs.237258 /len = 517′ | | |
| 60847_at | Cluster Incl. AA406400:zv12b07.r1 Homo sapiens cDNA, | 0.294009282 | 0.006324 |
| | 5 end /clone = IMAGE-753397 /clone_end = 5′ | | |
| | /gb = AA406400 /gi = 2064401 /ug = Hs.22851 /len = 509′ | | |
| 62066_at | Cluster Incl. AI919146:tt34d10.x1 Homo sapiens cDNA, | 0.292800813 | 0.001503 |
| | 3 end /clone = IMAGE-2242675 /clone_end = 3′ | | |
| | /gb = AI919146 /gi = 5639093 /ug = Hs.147293 /len = 174′ | | |
| 54215_at | Cluster Incl. AA742993:ny20a12.s1 Homo sapiens cDNA | 0.292648011 | 0.000982 |
| | /clone = IMAGE-1272286 /gb = AA742993 /gi = 2782499 | | |
| | /ug = Hs.190470 /len = 285 | | |
| 65934_at | Cluster Incl. AI546910:PN2.1_10_A10.r Homo sapiens cDNA, | 0.292566334 | 0.000017 |
| | 5 end /clone_end = 5′ /gb = AI546910 | | |
| | /gi = 4464398 /ug = Hs.7499 /len = 791′ | | |
| 49394_at | Cluster Incl. AA909687:om47d05.s1 Homo sapiens cDNA, | 0.291700552 | 0.00198 |
| | 3 end /clone = IMAGE-1544169 /clone_end = 3′ | | |
| | /gb = AA909687 /gi = 3049092 /ug = Hs.132222 /len = 530′ | | |
| 51763_at | Cluster Incl. AA621061:ag03g05.s1 Homo sapiens cDNA, | 0.29151259 | 0.001387 |
| | 3 end /clone = IMAGE-1056248 /clone_end = 3′ | | |
| | /gb = AA621061 /gi = 2525000 /ug = Hs.238944 /len = 473′ | | |
| 63998_at | Cluster Incl. AA587236:nn82c12.s1 Homo sapiens cDNA, | 0.291223086 | 0.000005 |
| | 3 end /clone = IMAGE-1090390 /clone_end = 3′ | | |
| | /gb = AA587236 /gi = 2398050 /ug = Hs.234146 /len = 630′ | | |
| 62502_at | Cluster Incl. C75510:C75510 Homo sapiens cDNA /clone = hbc7804 | 0.28932962 | 0.000001 |
| | /gb = C75510 /gi = 2366572 /ug = Hs.61933 /len = 543 | | |
| 64485_at | Cluster Incl. AA057445:zf57h11.s1 Homo sapiens cDNA, | 0.288246999 | 0.001207 |
| | 3 end /clone = IMAGE-381093 /clone_end = 3′ | | |
| | /gb = AA057445 /gi = 1550086 /ug = Hs.6176 /len = 576′ | | |
| 65460_at | Cluster Incl. AI669827:tu31g09.x1 Homo sapiens cDNA, | 0.28784121 | 0.001679 |
| | 3 end /clone = IMAGE-2252704 /clone_end = 3′ | | |
| | /gb = AI669827 /gi = 4834601 /ug = Hs.16577 /len = 593′ | | |
| 63782_at | Cluster Incl. AW051353:wy89g06.x1 Homo sapiens cDNA, | 0.287832225 | 0.000028 |
| | 3 end /clone = IMAGE-2555770 /clone_end = 3′ | | |
| | /gb = AW051353 /gi = 5913623 /ug = Hs.184668 /len = 577′ | | |
| 56234_r_a | Cluster Incl. AA053401:zl71b05.s1 Homo sapiens cDNA, | 0.287820353 | 0.00614 |
| | 3 end /clone = IMAGE-510033 /clone_end = 3′ | | |
| | /gb = AA053401 /gi = 1544038 /ug = Hs.177526 /len = 434′ | | |
| 61241_at | Cluster Incl. AI636422:tz79c01.x1 Homo sapiens cDNA, | 0.287143194 | 0.006593 |
| | 3 end /clone = IMAGE-2294784 /clone_end = 3′ | | |
| | /gb = AI636422 /gi = 4687752 /ug = Hs.140491 /len = 599′ | | |
| 49579_at | Cluster Incl. H45299:yn99g07.s1 Homo sapiens cDNA, | 0.2861687 | 0.000328 |
| | 3 end /clone = IMAGE-176604 /clone_end = 3′ | | |
| | /gb = H45299 /gi = 921351 /ug = Hs.19845 /len = 476′ | | |
| 65858_at | Cluster Incl. N49855:yz08h09.s1 Homo sapiens cDNA, | 0.284532394 | 0.000034 |
| | 3 end /clone = IMAGE-282497 /clone_end = 3′ | | |
| | /gb = N49855 /gi = 1191021 /ug = Hs.33054 /len = 633′ | | |
| 66525_at | Cluster Incl. AI860791:wl05h02.x1 Homo sapiens cDNA, | 0.283827138 | 0.00039 |
| | 3 end /clone = IMAGE-2424051 /clone_end = 3′ | | |
| | /gb = AI860791 /gi = 5514407 /ug = Hs.124359 /len = 379′ | | |
| 59962_at | Cluster Incl. AI703476:we24f08.x1 Homo sapiens cDNA, | 0.283690542 | 0.000172 |
| | 3 end /clone = IMAGE-2342055 /clone_end = 3′ | | |
| | /gb = AI703476 /gi = 4991376 /ug = Hs.13214 /len = 512′ | | |
| 62716_at | Cluster Incl. H98970:yx11c05.s1 Homo sapiens cDNA, | 0.282761935 | 0.020256 |
| | 3 end /clone = IMAGE-261416 /clone_end = 3′ | | |
| | /gb = H98970 /gi = 1123638 /ug = Hs.166659 /len = 623′ | | |
| 62115_at | Cluster Incl. H95265:yu20e07.s1 Homo sapiens cDNA, | 0.282482023 | 0.017318 |
| | 3 end /clone = IMAGE-234372 /clone_end = 3′ | | |
| | /gb = H95265 /gi = 1102898 /ug = Hs.42053 /len = 592′ | | |
| 49249_s_{—} | Cluster Incl. AA533079:nj19e12.s1 Homo sapiens cDNA, | 0.279331424 | 0.012289 |
| | 3 end /clone = IMAGE-992974 /clone end = 3′ | | |
| | /gb = AA533079 /gi = 2277175 /ug = Hs.136005 /len = 549′ | | |
| 49749_at | Cluster Incl. AA312110:EST182804 Homo sapiens cDNA, | 0.277737004 | 0.010242 |
| | 5 end /clone = ATCC-159298 /clone_end = 5′ | | |
| | /gb = AA312110 /gi = 1964438 /ug = Hs.239945 /len = 455′ | | |
| 48861_at | Cluster Incl. AA224174:zr15d10.r1 Homo sapiens cDNA, | 0.275987009 | 0.007743 |
| | 5 end /clone = IMAGE-663475 /clone_end = 5′ | | |
| | /gb = AA224174 /gi = 1844838 /ug = Hs.111099 /len = 452′ | | |
| 63674_r_a | Cluster Incl. AI653782:ty01d02.x1 Homo sapiens cDNA, | 0.274034829 | 0.012693 |
| | 3 end /clone = IMAGE-2277795 /clone_end = 3′ | | |
| | /gb = AI653782 /gi = 4737761 /ug = Hs.201337 /len = 548′ | | |
| 60426_at | Cluster Incl. AL047117:DKFZp586C2218_r1 Homo sapiens cDNA, | 0.271993302 | 0.003974 |
| | 5 end /clone = DKFZp586C2218 /clone end = 5′ | | |
| | /gb = AL047117 /gi = 5435160 /ug = Hs.169425 /len = 646′ | | |
| 64327_at | Cluster Incl. AW014889:UI-H-BIo-aaf-b-11-0-UI.s1 Homo sapiens cDNA, | 0.271558043 | 0.000001 |
| | 3 end /clone = IMAGE-2709069 /clone end = 3′ | | |
| | /gb = AW014889 /gi = 5863646 /ug = Hs.236762 /len = 565′ | | |
| 53582_at | Cluster Incl. AI141151:qe19e12.x1 Homo sapiens cDNA, | 0.271415126 | 0.000269 |
| | 3 end /clone = IMAGE-1739470 /clone_end = 3′ | | |
| | /gb = AI141151 /gi = 3648608 /ug = Hs.58330 /len = 426′ | | |
| 56147_f_a | Cluster Incl. T86696:yd86e02.s1 Homo sapiens cDNA, | 0.270730916 | 0.004461 |
| | 3 end /clone = IMAGE-115130 /clone_end = 3′ | | |
| | /gb = T86696 /gi = 715048 /ug = Hs.169968 /len = 329′ | | |
| 65577_at | Cluster Incl. AI870013:wl63g12.x1 Homo sapiens cDNA, | 0.270716848 | 0.016125 |
| | 3 end /clone = IMAGE-2429638 /clone_end = 3′ | | |
| | /gb = AI870013 /gi = 5543981 /ug = Hs.238049 /len = 536′ | | |
| 64451_at | Cluster Incl. AL048814:DKFZp434F1618_s1 Homo sapiens cDNA | 0.270363039 | 0.010424 |
| | /clone = DKFZp434F1618 /gb = AL048814 /gi = 4728123 | | |
| | /ug = Hs.5624 /len = 634 | | |
| 59694_at | Cluster Incl. AI971396:wr04d08.x1 Homo sapiens cDNA, | 0.26973595 | 0.00008 |
| | 3 end /clone = IMAGE-2480559 /clone_end = 3′ | | |
| | /gb = AI971396 /gi = 5768222 /ug = Hs.234838 /len = 624′ | | |
| 65734_at | Cluster Incl. AI744109:wc36d09.x1 Homo sapiens cDNA, | 0.268508824 | 0.025575 |
| | 3 end /clone = IMAGE-2317265 /clone_end = 3′ | | |
| | /gb = AI744109 /gi = 5112397 /ug = Hs.180141 /len = 828′ | | |
| 63892_at | Cluster Incl. AI056075:ox46e08.s1 Homo sapiens cDNA, | 0.267200526 | 0.016718 |
| | 3 end /clone = IMAGE-1659398 /clone_end = 3′ | | |
| | /gb = AI056075 /gi = 3329941 /ug = Hs.239678 /len = 490′ | | |
| AFFX-HU | M33197 Human glyceraldehyde-3-phosphate dehydrogenase (GAPDH) mRNA, | 0.266542141 | 0.018105 |
| | complete cds(_5, _M, _3 represent transcript | | |
| | regions 5 prime, Middle, and 3 prime respectively) | | |
| 54068_at | AA973035:op25g06.s1 Homo sapiens cDNA, | 0.265826141 | 0.002294 |
| | 3 end /clone = IMAGE-1577914 /clone_end = 3′ | | |
| | /gb = AA973035 /gi = 3148215 /ug = Hs.128271 /len = 494′ | | |
| 64094_at | Cluster Incl. AI123601:qa50d10.x1 Homo sapiens cDNA, | 0.26328184 | 0.003637 |
| | 3 end /clone = IMAGE-1690195 /clone end = 3′ | | |
| | /gb = AI123601 /gi = 3539367 /ug = Hs.32580 /len = 603′ | | |
| 50936_g_{—} | Cluster Incl. AA454184:zx48a10.s1 Homo sapiens cDNA, | 0.257098922 | 0.000135 |
| | 3 end /clone = IMAGE-795450 /clone_end = 3′ | | |
| | /gb = AA454184 /gi = 2167853 /ug = Hs.233805 /len = 559′ | | |
| 62972_at | Cluster Incl. W56118:zc56h04.r1 Homo sapiens cDNA, | 0.257076447 | 0.016006 |
| | 5 end /clone = IMAGE-326359 /clone_end = 5′ | | |
| | /gb = W56118 /gi = 1358007 /ug = Hs.87747 /len = 582′ | | |
| 62297_at | Cluster Incl. AL048338:DKFZp586C1424_s1 Homo sapiens cDNA | 0.253314399 | 0.002475 |
| | /clone = DKFZp586C1424 /gb = AL048338 | | |
| | /gi = 4727478 /ug = Hs.167956 /len = 805 | | |
| 48751_at | Cluster Incl. W61000:zc99g11.s1 Homo sapiens cDNA, | 0.252779772 | 0.007829 |
| | 3 end /clone = IMAGE-339332 /clone_end = 3′ | | |
| | /gb = W61000 /gi = 1367759 /ug = Hs.9578 /len = 607′ | | |
| 64095_at | Cluster Incl. AL046950:DKFZp586J1617_s1 Homo sapiens cDNA, | 0.252581936 | 0.026995 |
| | 3 end /clone = DKFZp586J1617 /clone end = 3′ | | |
| | /gb = AL046950 /gi = 5435008 /ug = Hs.32675 /len = 726′ | | |
| 64544_at | Cluster Incl. AA157506:zo55d01.s1 Homo sapiens cDNA, | 0.252105997 | 0.000747 |
| | 3 end /clone = IMAGE-590785 /clone_end = 3′ | | |
| | /gb = AA157506 /gi = 1729131 /ug = Hs.29003 /len = 427′ | | |
| 62942_at | Cluster Incl. AI346341:qp50b09.x1 Homo sapiens cDNA, | 0.251328324 | 0.000433 |
| | 3 end /clone = IMAGE-1926425 /clone_end = 3′ | | |
| | /gb = AI346341 /gi = 4083547 /ug = Hs.82669 /len = 549′ | | |
| 61639_at | Cluster Incl. AA863003:og99d06.s1 Homo sapiens cDNA, | 0.250029119 | 0.004024 |
| | 3 end /clone = IMAGE-1456427 /clone_end = 3′ | | |
| | /gb = AA863003 /gi = 2955482 /ug = Hs.28651 /len = 455′ | | |
| 63845_at | Cluster Incl. AI766309:wh71d09.x1 Homo sapiens cDNA, | 0.249268909 | 0.001144 |
| | 3 end /clone = IMAGE-2386193 /clone_end = 3′ | | |
| | /gb = AI766309 /gi = 5232818 /ug = Hs.23363 /len = 571′ | | |
| 66785_i_a | Cluster Incl. AI223376:qg43d08.x1 Homo sapiens cDNA, | 0.249160717 | 0.002653 |
| | 3 end /clone = IMAGE-1837935 /clone_end = 3′ | | |
| | /gb = AI223376 /gi = 3805579 /ug = Hs.128454 /len = 387′ | | |
| 52038_r_a | Cluster Incl. AI223016:qg52f04.x1 Homo sapiens cDNA, | 0.24791803 | 0.00541 |
| | 3 end /clone = IMAGE-1838815 /clone_end = 3′ | | |
| | /gb = AI223016 /gi = 3805219 /ug = Hs.144742 /len = 281′ | | |
| 56809_at | Cluster Incl. AI052524:oz27f07.x1 Homo sapiens cDNA, | 0.245992054 | 0.000385 |
| | 3 end /clone = IMAGE-1676581 /clone_end = 3′ | | |
| | /gb = AI052524 /gi = 3308515 /ug = Hs.4799 /len = 537′ | | |
| 65175_at | Cluster Incl. AI741026:wg25e11.x1 Homo sapiens cDNA, | 0.2449656 | 0.000409 |
| | 3 end /clone = IMAGE-2366156 /clone_end = 3′ | | |
| | /gb = AI741026 /gi = 5109314 /ug = Hs.13299 /len = 592′ | | |
| 65893_at | Cluster Incl. AI151271:qc88b06.x1 Homo sapiens cDNA, | 0.24384702 | 0.001358 |
| | 3 end /clone = IMAGE-1721267 /clone_end = 3′ | | |
| | /gb = AI151271 /gi = 3679740 /ug = Hs.5243 /len = 616′ | | |
| 62692_at | Cluster Incl. AA410788:zt35b11.r1 Homo sapiens cDNA, | 0.243344597 | 0.006983 |
| | 5 end /clone = IMAGE-724317 /clone_end = 5′ | | |
| | /gb = AA410788 /gi = 2069895 /ug = Hs.183673 /len = 484′ | | |
| 54730_at | Cluster Incl. AA553527:nk78a04.s1 Homo sapiens cDNA, | 0.242978961 | 0.000938 |
| | 3 end /clone = IMAGE-1019598 /clone_end = 3′ | | |
| | /gb = AA553527 /gi = 2324066 /ug = Hs.152432 /len = 510′ | | |
| 60481_i_a | Cluster Incl. AA932331:oo60a08.s1 Homo sapiens cDNA, | 0.241809966 | 0.006633 |
| | 3 end /clone = IMAGE-1570550 /clone_end = 3′ | | |
| | /gb = AA932331 /gi = 3086569 /ug = Hs.181174 /len = 508′ | | |
| 54556_at | Cluster Incl. AI554741:tn27g07.x1 Homo sapiens cDNA, | 0.241602147 | 0.003086 |
| | 3 end /clone = IMAGE-2168892 /clone_end = 3′ | | |
| | /gb = AI554741 /gi = 4487104 /ug = Hs.150657 /len = 466′ | | |
| 61247_f_a | Cluster Incl. R67332:yi32e02.s1 Homo sapiens cDNA, | 0.241131758 | 0.012114 |
| | 3 end /clone = IMAGE-140954 /clone_end = 3′ | | |
| | /gb = R67332 /gi = 839970 /ug = Hs.140987 /len = 423′ | | |
| 60029_i_a | Cluster Incl. AI589514:tm57a12.x1 Homo sapiens cDNA, | 0.23979923 | 0.005698 |
| | 3 end /clone = IMAGE-2162206 /clone_end = 3′ | | |
| | /gb = AI589514 /gi = 4598562 /ug = Hs.15688 /len = 493′ | | |
| 56675_at | Cluster Incl. H28004:yl59g07.s1 Homo sapiens cDNA, | 0.235365417 | 0.000016 |
| | 3 end /clone = IMAGE-162588 /clone_end = 3′ | | |
| | /gb = H28004 /gi = 898357 /ug = Hs.25699 /len = 462′ | | |
| 65113_at | Cluster Incl. AI952965:wq49d11.x1 Homo sapiens cDNA, | 0.234876957 | 0.001006 |
| | 3 end /clone = IMAGE-2474613 /clone_end = 3′ | | |
| | /gb = AI952965 /gi = 5745275 /ug = Hs.97837 /len = 472′ | | |
| 58434_at | Cluster Incl. AI268312:qm04b12.x1 Homo sapiens cDNA, | 0.233510248 | 0.000001 |
| | 3 end /clone = IMAGE-1880831 /clone_end = 3′ | | |
| | /gb = AI268312 /gi = 3887479 /ug = Hs.103009 /len = 609′ | | |
| 63311_at | Cluster Incl. AI742378:wg40c02.x1 Homo sapiens cDNA, | 0.232118688 | 0.004954 |
| | 3 end /clone = IMAGE-2367554 /clone_end = 3′ | | |
| | /gb = AI742378 /gi = 5110666 /ug = Hs.101514 /len = 672′ | | |
| 62573_at | Cluster Incl. H04627:yj49f04.r1 Homo sapiens cDNA, | 0.230460891 | 0.002567 |
| | 5 end /clone = IMAGE-152095 /clone_end = 5′ | | |
| | /gb = H04627 /gi = 867560 /ug = Hs.79793 /len = 576′ | | |
| 62351_at | Cluster Incl. AI217310:qh25d09.x1 Homo sapiens cDNA, | 0.230064353 | 0.000264 |
| | 3 end /clone = IMAGE-1845713 /clone_end = 3′ | | |
| | /gb = AI217310 /gi = 3797125 /ug = Hs.147568 /len = 390′ | | |
| 65173_at | Cluster Incl. H92987:yv07d04.s1 Homo sapiens cDNA, | 0.225358214 | 0.000567 |
| | 3 end /clone = IMAGE-242023 /clone_end = 3′ | | |
| | /gb = H92987 /gi = 1099315 /ug = Hs.13299 /len = 650′ | | |
| 60058_at | Cluster Incl. AA557237:nl75d07.s1 Homo sapiens cDNA, | 0.224424263 | 0.021308 |
| | 3 end /clone = IMAGE-1056493 /clone_end = 3′ | | |
| | /gb = AA557237 /gi = 2327714 /ug = Hs.168776 /len = 605′ | | |
| 56677_at | Cluster Incl. AA557324:nl81a02.s1 Homo sapiens cDNA, | 0.220198572 | 0.002338 |
| | 3 end /clone = IMAGE-1057034 /clone_end = 3′ | | |
| | /gb = AA557324 /gi = 2327801 /ug = Hs.26040 /len = 568′ | | |
| 64357_at | Cluster Incl. AI991817:wr32f11.x1 Homo sapiens cDNA, | 0.220156505 | 0.012518 |
| | 3 end /clone = IMAGE-2489421 /clone_end = 3′ | | |
| | /gb = AI991817 /gi = 5838722 /ug = Hs.237718 /len = 388′ | | |
| 60532_at | Cluster Incl. AA034289:zk18e07.r1 Homo sapiens cDNA, | 0.219583921 | 0.006195 |
| | 5 end /clone = IMAGE-470916 /clone_end = 5′ | | |
| | /gb = AA034289 /gi = 1506098 /ug = Hs.200499 /len = 588′ | | |
| 58504_at | Cluster Incl. AA149860:zl47e11.r1 Homo sapiens cDNA, | 0.218848321 | 0.000376 |
| | 5 end /clone = IMAGE-505100 /clone_end = 5′ | | |
| | /gb = AA149860 /gi = 1720940 /ug = Hs.107755 /len = 659′ | | |
| 63567_at | Cluster Incl. H50153:yo27e01.r1 Homo sapiens cDNA, | 0.21856838 | 0.000001 |
| | 5 end /clone = IMAGE-179160 /clone_end = 5′ | | |
| | /gb = H50153 /gi = 989994 /ug = Hs.21659 /len = 580′ | | |
| 64137_at | Cluster Incl. AI094860:qa09a11.x1 Homo sapiens cDNA, | 0.217551174 | 0.005466 |
| | 3 end /clone = IMAGE-1686236 /clone_end = 3′ | | |
| | /gb = AI094860 /gi = 3433836 /ug = Hs.239512 /len = 452′ | | |
| 58086_i_a | Cluster Incl. AA815371:ai64g06.s1 Homo sapiens cDNA, | 0.216986503 | 0.000395 |
| | 3 end /clone = 1375642 /clone_end = 3′ | | |
| | /gb = AA815371 /gi = 2884967 /ug = Hs.122965 /len = 426′ | | |
| 62562_at | Cluster Incl. AI674123:wd18d08.x1 Homo sapiens cDNA, | 0.215616168 | 0.000068 |
| | 3 end /clone = IMAGE-2328495 /clone_end = 3′ | | |
| | /gb = AI674123 /gi = 4874603 /ug = Hs.7615 /len = 547′ | | |
| 58404_at | Cluster Incl. AA487503:aa95c01.s1 Homo sapiens cDNA, | 0.214993271 | 0.000121 |
| | 3 end /clone = IMAGE-839040 /clone_end = 3′ | | |
| | /gb = AA487503 /gi = 2217667 /ug = Hs.100636 /len = 486′ | | |
| 57792_at | Cluster Incl. AA399630:zt93d07.s1 Homo sapiens cDNA, | 0.213130496 | 0.000122 |
| | 3 end /clone = IMAGE-729901 /clone_end = 3′ | | |
| | /gb = AA399630 /gi = 2052644 /ug = Hs.94396 /len = 553′ | | |
| 62599_r_a | Cluster Incl. AI742422:wg40f11.x1 Homo sapiens cDNA, | 0.209883294 | 0.000954 |
| | 3 end /clone = IMAGE-2367597 /clone_end = 3′ | | |
| | /gb = AI742422 /gi = 5110710 /ug = Hs.174363 /len = 577′ | | |
| 65165_at | Cluster Incl. AI453545:tj57c03.x1 Homo sapiens cDNA, | 0.20820513 | 0.011588 |
| | 3 end /clone = IMAGE-2145604 /clone_end = 3′ | | |
| | /gb = AI453545 /gi = 4282767 /ug = Hs.12842 /len = 636′ | | |
| 64709_at | Cluster Incl. AI810632:tu19b06.x1 Homo sapiens cDNA, | 0.20784757 | 0 |
| | 3 end /clone = IMAGE-2251475 /clone_end = 3′ | | |
| | /gb = AI810632 /gi = 5397198 /ug = Hs.55080 /len = 542′ | | |
| 61625_at | Cluster Incl. AI278995:qm22e01.x1 Homo sapiens cDNA, | 0.20742973 | 0.000095 |
| | 3 end /clone = IMAGE-1882584 /clone_end = 3′ | | |
| | /gb = AI278995 /gi = 3917229 /ug = Hs.27457 /len = 581′ | | |
| 60445_at | Cluster Incl. AA534542:nf77a03.s1 Homo sapiens cDNA, | 0.206277647 | 0.015576 |
| | 3 end /clone = IMAGE-925900 /clone_end = 3′ | | |
| | /gb = AA534542 /gi = 2278795 /ug = Hs.172849 /len = 489′ | | |
| 49068_at | Cluster Incl. AI031674:ow48g03.x1 Homo sapiens cDNA, | 0.204781876 | 0.000281 |
| | 3 end /clone = IMAGE-1650100 /clone_end = 3′ | | |
| | /gb = AI031674 /gi = 3249886 /ug = Hs.5850 /len = 804′ | | |
| 59911_f_a | Cluster Incl. AI124631:am59f08.x1 Homo sapiens cDNA, | 0.202857127 | 0.034818 |
| | 3 end /clone = IMAGE-1539879 /clone_end = 3′ | | |
| | /gb = AI124631 /gi = 3593145 /ug = Hs.130893 /len = 416′ | | |
| 53313_at | Cluster Incl. AA284232:zc39c01.T7 Homo sapiens cDNA, | 0.202265466 | 0.000233 |
| | 3 end /clone = IMAGE-324672 /clone_end = 3′ | | |
| | /gb = AA284232 /gi = 1928532 /ug = Hs.55983 /len = 546′ | | |
| 62763_at | Cluster Incl. AI978918:wr61h01.x1 Homo sapiens cDNA, | 0.200697429 | 0.000276 |
| | 3 end /clone = IMAGE-2492209 /clone_end = 3′ | | |
| | /gb = AI978918 /gi = 5803948 /ug = Hs.179608 /len = 588′ | | |
| 54326_at | Cluster Incl. AI928203:wo95d03.x1 Homo sapiens cDNA, | 0.196029076 | 0.000171 |
| | 3 end /clone = IMAGE-2463077 /clone_end = 3′ | | |
| | /gb = AI928203 /gi = 5664167 /ug = Hs.86379 /len = 554′ | | |
| 48857_at | Cluster Incl. AI924323:wn55d07.x1 Homo sapiens cDNA, | 0.194383948 | 0.000252 |
| | 3 end /clone = IMAGE-2449357 /clone_end = 3′ | | |
| | /gb = AI924323 /gi = 5660287 /ug = Hs.110024 /len = 525′ | | |
| 62593_at | Cluster Incl. AA425714:zv47e06:r1 Homo sapiens cDNA, | 0.194165766 | 0.000008 |
| | 5 end /clone = IMAGE-756802 /clone_end = 5′ | | |
| | /gb = AA425714 /gi = 2107364 /ug = Hs.173704 /len = 454′ | | |
| 55676_r_a | Cluster Incl. H40497:yn87f03.s1 Homo sapiens cDNA, | 0.193742511 | 0.000159 |
| | 3 end /clone = IMAGE-175421 /clone_end = 3′ | | |
| | /gb = H40497 /gi = 916549 /ug = Hs.237249 /len = 446′ | | |
| 65550_at | Cluster Incl. AI546943:PN2.1_10_E06.r Homo sapiens cDNA, | 0.19337696 | 0.000005 |
| | 5 end /clone_end = 5′ /gb = AI546943 | | |
| | /gi = 4464431 /ug = Hs.23136 /len = 706′ | | |
| 55581_at | Cluster Incl. AI051688:oy97e02.x1 Homo sapiens cDNA, | 0.192450405 | 0.035989 |
| | 3 end /clone = IMAGE-1673786 /clone_end = 3′ | | |
| | /gb = AI051688 /gi = 3307222 /ug = Hs.230273 /len = 449′ | | |
| 60401_at | Cluster Incl. AI769290:wg36b12.x1 Homo sapiens cDNA, | 0.191038508 | 0.000043 |
| | 3 end /clone = IMAGE-2367167 /clone_end = 3′ | | |
| | /gb = AI769290 /gi = 5235799 /ug = Hs.134190 /len = 486′ | | |
| 56691_at | Cluster Incl. AI935202:wp17a11.x1 Homo sapiens cDNA, | 0.181080399 | 0.002371 |
| | 3 end /clone = IMAGE-2465084 /clone end = 3′ | | |
| | /gb = AI935202 /gi = 5674072 /ug = Hs.31181 /len = 571′ | | |
| 63476_at | Cluster.Incl. AI796235:wh44c01.x1 Homo sapiens cDNA, | 0.179038438 | 0.000039 |
| | 3 end /clone = IMAGE-2383584 /clone end = 3′ | | |
| | /gb = AI796235 /gi = 5361687 /ug = Hs.21299 /len = 530′ | | |
| 53810_f_a | Cluster Incl. AA401302:zv66h05.s1 Homo sapiens cDNA, | 0.175285063 | 0.000001 |
| | 3 end /clone = IMAGE-758649 /clone_end = 3′ | | |
| | /gb = AA401302 /gi = 2055205 /ug = Hs.79881 /len = 543′ | | |
| 65876_at | Cluster Incl. R45447:yg43b01.s1 Homo sapiens cDNA, | 0.168282876 | 0.001526 |
| | 3 end /clone = IMAGE-35226 /clone_end = 3′ | | |
| | /gb = R45447 /gi = 820704 /ug = Hs.4213 /len = 537′ | | |
| 50938_at | Cluster Incl. AA778522:af85g10.s1 Homo sapiens cDNA, | 0.168035248 | 0.016005 |
| | 3 end /clone = 1048866 /clone_end = 3′ | | |
| | /gb = AA778522 /gi = 2837853 /ug = Hs.233824 /len = 499′ | | |
| 64151_r_a | Cluster Incl. H23482:ym57d12.s1 Homo sapiens cDNA, | 0.16558005 | 0.001158 |
| | 3 end /clone = IMAGE-52543 /clone_end = 3′ | | |
| | /gb = H23482 /gi = 892177 /ug = Hs.24088 /len = 560′ | | |
| 58065_at | Cluster Incl. AI765978:wj23a01.x1 Homo sapiens cDNA, | 0.165471752 | 0 |
| | 3 end /clone = IMAGE-2403624 /clone_end = 3′ | | |
| | /gb = AI765978 /gi = 5232487 /ug = Hs.122909 /len = 338′ | | |
| 62968_at | Cluster Incl. AI290476:ql98b08.x1 Homo sapiens cDNA, | 0.161962046 | 0.010789 |
| | 3 end /clone = IMAGE-1880343 /clone_end = 3′ | | |
| | /gb = AI290476 /gi = 3933250 /ug = Hs.87597 /len = 491′ | | |
| 61929_at | Cluster Incl. AW024884:wu92c03.x1 Homo sapiens cDNA, | 0.15832857 | 0.018434 |
| | 3 end /clone = IMAGE-990839 /clone_end = 3′ | | |
| | /gb = AW024884 /gi = 5878414 /ug = Hs.134478 /len = 472′ | | |
| 64925_at | Cluster Incl. AI307808:tb28d05.x1 Homo sapiens cDNA, | 0.158241319 | 0.001113 |
| | 3 end /clone = IMAGE-2055657 /clone_end = 3′ | | |
| | /gb = AI307808 /gi = 4002412 /ug = Hs.7402 /len = 622′ | | |
| 49042_at | Cluster Incl. AA402161:zu55f06.r1 Homo sapiens cDNA, | 0.156140952 | 0.000036 |
| | 5 end /clone = IMAGE-741923 /clone_end = 5′ | | |
| | /gb = AA402161 /gi = 2056126 /ug = Hs.239890 /len = 540′ | | |
| 61681_at | Cluster Incl. AA921922:om40h06.s1 Homo sapiens cDNA, | 0.147708822 | 0.012553 |
| | 3 end /clone = IMAGE-1543547 /clone_end = 3′ | | |
| | /gb = AA921922 /gi = 3069231 /ug = Hs.31412 /len = 488′ | | |
| 62723_r_a | Cluster Incl. AI590722:tw87h05.x1 Homo sapiens cDNA, | 0.137146209 | 0.014933 |
| | 3 end /clone = IMAGE-2266713 /clone_end = 3′ | | |
| | /gb = AI590722 /gi = 4599770 /ug = Hs.167569 /len = 691′ | | |
| 62817_at | Cluster Incl. AI201812:qi42c12.x1 Homo sapiens cDNA, | 0.13454914 | 0.007808 |
| | 3 end /clone = IMAGE-1859158 /clone_end = 3′ | | |
| | /gb = AI201812 /gi = 3754418 /ug = Hs.148587 /len = 464′ | | |
| 63131_i_a | Cluster Incl. R11248:yf41c02.r1 Homo sapiens cDNA, | 0.133193785 | 0.00248 |
| | 5 end /clone = IMAGE-129410 /clone_end = 5′ | | |
| | /gb = R11248 /gi = 763983 /ug = Hs.191935 /len = 456′ | | |
| 60231_at | Cluster Incl. W20054:zb40a08.r1 Homo sapiens cDNA, | 0.128912912 | 0.00004 |
| | 5 end /clone = IMAGE-306038 /clone_end = 5′ | | |
| | /gb = W20054 /gi = 1295925 /ug = Hs.102987 /len = 574′ | | |
| 63059_r_a | Cluster Incl. T79615:yd71e05.s1 Homo sapiens cDNA, | 0.109126683 | 0.000031 |
| | 3 end /clone = IMAGE-113696 /clone_end = 3′ | | |
| | /gb = T796.15 /gi = 698124 /ug = Hs.184260 /len = 569′ | | |
| 54320_at | Cluster Incl. AI962905:wt24f04.x1 Homo sapiens cDNA, | 0.103706856 | 0 |
| | 3 end /clone = IMAGE-2508415 /clone_end = 3′ | | |
| | /gb = AI962905 /gi = 5755618 /ug = Hs.86030 /len = 617′ | | |
| 61755_i_a | Cluster Incl. W67816:zd38a02.r1 Homo sapiens cDNA, | 0.098989494 | 0.012557 |
| | 5 end /clone = IMAGE-342890 /clone_end = 5′ | | |
| | /gb = W67816 /gi = 1376706 /ug = Hs.110196 /len = 599′ | | |
| 51794_at | Cluster Incl. AI971202:wr26d09.x1 Homo sapiens cDNA, | 0.082261318 | 0.000014 |
| | 3 end /clone = IMAGE-2488817 /clone_end = 3′ | | |
| | /gb = AI971202 /gi = 5768028 /ug = Hs.239290 /len = 587′ | | |
| 65618_at | Cluster Incl. AI672527:wb32c06.x1 Homo sapiens cDNA, | 0.078251936 | 0.000052 |
| | 3 end /clone = IMAGE-2307370 /clone_end = 3′ | | |
| | /gb = AI672527 /gi = 4852258 /ug = Hs.27836 /len = 529′ | | |
| 62998_at | Cluster Incl. AI831452:wj49b03.x1 Homo sapiens cDNA, | 0.07541159 | 0.004277 |
| | 3 end /clone = IMAGE-2406125 /clone_end = 3′ | | |
| | /gb = AI831452 /gi = 5452123 /ug = Hs.91539 /len = 597′ | | |
| 57186_at | Cluster Incl. AI979.134:wr70h09.x1 Homo sapiens cDNA, | 0.073273444 | 0.000306 |
| | 3 end /clone = IMAGE-2493089 /clone_end = 3′ | | |
| | /gb = AI979134 /gi = 5804164 /ug = Hs.230154 /len = 494′ | | |
| 60228_at | Cluster Incl. R24011:yh29e05.r1 Homo sapiens cDNA, | 0.059028357 | 0.000122 |
| | 5 end /clone = IMAGE-131168 /clone_end = 5′ | | |
| | /gb = R24011 /gi = 778899 /ug = Hs.100931 /len = 456′ | | |
| 54791_at | Cluster Incl. AI620463:tu56a09.x1 Homo sapiens cDNA, | 0.057352223 | 0.000062 |
| | 3 end /clone = IMAGE-2255032 /clone_end = 3′ | | |
| | /gb = AI620463 /gi = 4629589 /ug = Hs.99197 /len = 468′ | | |
| 60587_at | Cluster Incl. AI694073:wc82e02.x1 Homo sapiens cDNA, | 0.052183792 | 0.00012 |
| | 3 end /clone = IMAGE-2325146 /clone_end = 3′ | | |
| | /gb = AI694073 /gi = 4971413 /ug = Hs.48956 /len = 443′ | | |
| 54805_at | Cluster Incl. AI623978:ts25b06.x1 Homo sapiens cDNA, | 0.04331356 | 0.000001 |
| | 3 end /clone = IMAGE-2229587 /clone_end = 3′ | | |
| | /gb = AI623978 /gi = 4648909 /ug = Hs.99384 /len = 461′ | | |
| 62247_at | Cluster Incl. W68630:zd36c11.s1 Homo sapiens cDNA, | 0.035040967 | 0.000179 |
| | 3 end /clone = IMAGE-342740 /clone_end = 3′ | | |
| | /gb = W68630 /gi = 1377499 /ug = Hs.161566 /len = 510′ | | |
| 66443_at | Cluster Incl. AA775536:zf23h10.s1 Homo sapiens cDNA, | 0.032947258 | 0.000713 |
| | 3 end /clone = 377827 /clone_end = 3′ | | |
| | /gb = AA775536 /gi = 2834870 /ug = Hs.121824 /len = 368′ | | |
| 61756_r_a | Cluster Incl. W67816:zd38a02.r1 Homo sapiens cDNA, | 0.024870079 | 0.002205 |
| | 5 end /clone = IMAGE-342890 /clone_end = 5′ | | |
| | /gb = W67816 /gi = 1376706 /ug = Hs.110196 /len = 599′ | | |
| 66392_at | Cluster Incl. AA743820:ny29f11.s1 Homo sapiens cDNA | 0.020219012 | 0.000042 |
| | /clone = IMAGE-1273197 /gb = AA743820 /gi = 2783171 | | |
| | /ug = Hs.120606 /len = 466 | | |

TABLE 5
U95_D Fold Change Genes (>3 over-expressed in Barrett's associated
esophageal adenocarcinoma (BA), <0.33 under-expressed in BA)

| Affy ID | Gene Name | Fold Change | P-value |
|---|---|---|---|
| 72629_at | Cluster Incl. AI308063:tb22f06.x1 Homo sapiens cDNA, | 126.8832056 | 0.000003 |
| | 3 end /clone = IMAGE-2055107 /clone_end = 3′ | | |
| | /gb = AI308063 /gi = 4002698 /ug = Hs.222212 /len = 205′ | | |
| 89917_at | Cluster Incl. AW005911:wz90g01.x1 Homo sapiens cDNA, | 33.92289597 | 0.000005 |
| | 3 end /clone = IMAGE-2566128 /clone_end = 3′ | | |
| | /gb = AW005911 /gi = 5854701 /ug = Hs.234064 /len = 485′ | | |
| 86031_at | Cluster Incl. AI803868:tp36g05.x1 Homo sapiens cDNA, | 18.75245815 | 0.001902 |
| | 3 end /clone = IMAGE-2189912 /clone_end = 3′ | | |
| | /gb = AI803868 /gi = 5369329 /ug = Hs.177164 /len = 274′ | | |
| 89895_f_a | Cluster Incl. AW001128:wu24f06.x1 Homo sapiens cDNA, | 17.33803078 | 0.000302 |
| | 3 end /clone = IMAGE-990870 /clone_end = 3′ | | |
| | /gb = AW001128 /gi = 5848044 /ug = Hs.233945 /len = 239′ | | |
| 74815_at | Cluster Incl. AI864016:wj53h10.x1 Homo sapiens cDNA, | 17.16521833 | 0.002611 |
| | 3 end /clone = IMAGE-2406595 /clone_end = 3′ | | |
| | /gb = AI864016 /gi = 5528123 /ug = Hs.234375 /len = 511′ | | |
| 90481_at | Cluster Incl. AI125252:qd87h06.x1 Homo sapiens cDNA, | 15.90611723 | 0.000001 |
| | 3 end /clone = IMAGE-1736507 /clone_end = 3′ | | |
| | /gb = AI125252 /gi = 3593766 /ug = Hs.126419 /len = 466′ | | |
| 77946_at | Cluster Incl. AI744130:wc36f07.x1 Homo sapiens cDNA, | 14.72840034 | 0.00036 |
| | 3 end /clone = IMAGE-2317285 /clone_end = 3′ | | |
| | /gb = AI744130 /gi = 5112418 /ug = Hs.131201 /len = 483′ | | |
| 78668_r_a | Cluster Incl. AI673818:to73f12.x1 Homo sapiens cDNA, | 14.66402976 | 0.003391 |
| | 3 end /clone = IMAGE-2183951 /clone end = 3′ | | |
| | /gb = AI673818 /gi = 4853549 /ug = Hs.134665 /len = 201′ | | |
| 73337_at | Cluster Incl. AI355709:qt56h08.x1 Homo sapiens cDNA, | 12.24921906 | 0.001589 |
| | 3 end /clone = IMAGE-1952031 /clone_end = 3′ | | |
| | /gb = AI355709 /gi = 4095862 /ug = Hs.225823 /len = 460′ | | |
| 73859_at | Cluster Incl. AW008822:ws71h07.x1 Homo sapiens cDNA, | 12.03887849 | 0.000945 |
| | 3 end /clone = IMAGE-2502685 /clone_end = 3′ | | |
| | /gb = AW008822 /gi = 5857600 /ug = Hs.231934 /len = 369′ | | |
| 74904_at | Cluster Incl. AI984156:wu21d07.x1 Homo sapiens cDNA, | 11.93939119 | 0.000234 |
| | 3 end /clone = IMAGE-990944 /clone_end = 3′ | | |
| | /gb = AI984156 /gi = 5811375 /ug = Hs.235120 /len = 436′ | | |
| 85022_at | Cluster Incl. AI039701:ox32g01.s1 Homo sapiens cDNA, | 10.15512075 | 0.000008 |
| | 3 end /clone = IMAGE-1658064 /clone_end = 3′ | | |
| | /gb = AI039701 /gi = 3278895 /ug = Hs.167408 /len = 447′ | | |
| 74036_at | Cluster Incl. AW015189:UI-H-BI0p-aba-c-08-0-UI.s1 Homo sapiens cDNA, | 9.081911797 | 0.010398 |
| | 3 end /clone = IMAGE-2711054 /clone end = 3′ | | |
| | /gb = AW015189 /gi = 5863876 /ug = Hs.232065 /len = 407′ | | |
| 87776_at | Cluster Incl. AI023610:ov80c08.s1 Homo sapiens cDNA, | 8.799715822 | 0.000913 |
| | 3 end /clone = IMAGE-1643630 /clone_end = 3′ | | |
| | /gb = AI023610 /gi = 3238654 /ug = Hs.190586 /len = 436′ | | |
| 82342_at | Cluster Incl. AI675419:wb99b08.x1 Homo sapiens cDNA, | 7.772438703 | 0.014188 |
| | 3 end /clone = IMAGE-2313783 /clone_end = 3′ | | |
| | /gb = AI675419 /gi = 4875899 /ug = Hs.164464 /len = 504′ | | |
| 83665_at | Cluster Incl. AI375018:ta54d05.x1 Homo sapiens cDNA, | 7.219466585 | 0.005004 |
| | 3 end /clone = IMAGE-2047881 /clone_end = 3′ | | |
| | /gb = AI375018 /gi = 4175008 /ug = Hs.158717 /len = 474′ | | |
| 71502_at | Cluster Incl. AI910824:wi45g06.x1 Homo sapiens cDNA, | 7.213872979 | 0.000034 |
| | 3 end /clone = IMAGE-2393242 /clone_end = 3′ | | |
| | /gb = AI910824 /gi = 5630560 /ug = Hs.214202 /len = 235′ | | |
| 71136_at | Cluster Incl. AA631215:nq80g07.s1 Homo sapiens cDNA, | 7.099901302 | 0.001724 |
| | 3 end /clone = IMAGE-1158684 /clone_end = 3′ | | |
| | /gb = AA631215 /gi = 2553826 /ug = Hs.212640 /len = 491′ | | |
| 83222_at | Cluster Incl. AA911052:ok65e02.s1 Homo sapiens cDNA, | 7.077863313 | 0.005694 |
| | 3 end /clone = IMAGE-1518842 /clone_end = 3′ | | |
| | /gb = AA911052 /gi = 3050342 /ug = Hs.174950 /len = 451′ | | |
| 69494_at | Cluster Incl. AI703451:we24d06.x1 Homo sapiens cDNA, | 6.987691644 | 0.005066 |
| | 3 end /clone = IMAGE-2342027 /clone_end = 3′ | | |
| | /gb = AI703451 /gi = 4991351 /ug = Hs.202656 /len = 433′ | | |
| 70836_i_a | Cluster Incl. AI824055:wj35e12.x1 Homo sapiens cDNA, | 6.70015517 | 0.003057 |
| | 3 end /clone = IMAGE-2404846 /clone_end = 3′ | | |
| | /gb = AI824055 /gi = 5444726 /ug = Hs.211396 /len = 577′ | | |
| 74989_at | Cluster Incl. AW007442:wt55g06.x1 Homo sapiens cDNA, | 6.403445233 | 0.000249 |
| | 3 end /clone = IMAGE-2511418 /clone_end = 3′ | | |
| | /gb = AW007442 /gi = 5856220 /ug = Hs.235961 /len = 424′ | | |
| 74767_at | Cluster Incl AI869919:we75h03.x1 Homo sapiens cDNA, | 6.279662141 | 0.000435 |
| | 3 end /clone = IMAGE-2346965 /clone_end = 3′ | | |
| | /gb = AI869919 /gi = 5543887 /ug = Hs.234197 /len = 279′ | | |
| 76918_at | Cluster Incl. AI732969:oh70b01.x5 Homo sapiens cDNA, | 6.121303029 | 0.002211 |
| | 3 end /clone = IMAGE-1472329 /clone_end = 3′ | | |
| | /gb = AI732969 /gi = 5054082 /ug = Hs.126245 /len = 536′ | | |
| 74336_at | Cluster Incl. AW026718:wv44b03.x1 Homo sapiens cDNA, | 5.852600413 | 0.003442 |
| | 3 end /clone = IMAGE-990720 /clone_end = 3′ | | |
| | /gb = AW026718 /gi = 5880171 /ug = Hs.233303 /len = 693′ | | |
| 79398_at | Cluster Incl. AA580664:nd41b07.r1 Homo sapiens cDNA, | 5.528854288 | 0.013039 |
| | 5 end /clone = IMAGE-802837 /clone_end = 5′ | | |
| | /gb = AA580664 /gi = 2358321 /ug = Hs.142944 /len = 488′ | | |
| 81693_at | Cluster Incl. AI582751:tn17b08.x1 Homo sapiens cDNA, | 5.400844754 | 0.003535 |
| | 3 end /clone = IMAGE-2167863 /clone_end = 3′ | | |
| | /gb = AI582751 /gi = 4568648 /ug = Hs.159028 /len = 416′ | | |
| 76515_at | Cluster Incl. AA777011:zf28a12.s1 Homo sapiens cDNA, | 5.2764043 | 0.000093 |
| | 3 end /clone = 378238 /clone_end = 3′ | | |
| | /gb = AA777011 /gi = 2836342 /ug = Hs.122678 /len = 485′ | | |
| 74689_at | Cluster Incl. AW003135:wq63a10.x1 Homo sapiens cDNA, | 5.204740505 | 0.017468 |
| | 3 end /clone = IMAGE-2475930 /clone_end = 3′ | | |
| | /gb = AW003135 /gi = 5850051 /ug = Hs.234003 /len = 416′ | | |
| 89483_i_a | Cluster Incl. AI804066:tc60h05.x1 Homo sapiens cDNA, | 5.160756744 | 0.000671 |
| | 3 end /clone = IMAGE-2069049 /clone_end = 3′ | | |
| | /gb = AI804066 /gi = 5369538 /ug = Hs.210477 /len = 300′ | | |
| 80829_at | Cluster Incl. AA210905:zr90h02.r1 Homo sapiens cDNA, | 5.152266697 | 0.002582 |
| | 5 end /clone = IMAGE-682995 /clone_end = 5′ | | |
| | /gb = AA210905 /gi = 1809551 /ug = Hs.151875 /len = 588′ | | |
| 81411_at | Cluster Incl. AI339743:qq42g10.x1 Homo sapiens cDNA, | 5.081809717 | 0.000737 |
| | 3 end /clone = IMAGE-1935234 /clone_end = 3′ | | |
| | /gb = AI339743 /gi = 4076670 /ug = Hs.157177 /len = 476′ | | |
| 75224_s_{—} | Cluster Incl. AI992172:wt75e05.x1 Homo sapiens cDNA, | 5.038001155 | 0.000025 |
| | 3 end /clone = IMAGE-2513312 /clone_end = 3′ | | |
| | /gb = AI992172 /gi = 5839077 /ug = Hs.237455 /len = 762′ | | |
| 81496_at | Cluster Incl. AI870708:wl47h10.x1 Homo sapiens cDNA, | 5.031768797 | 0.000144 |
| | 3 end /clone = IMAGE-2428099 /clone_end = 3′ | | |
| | /gb = AI870708 /gi = 5544676 /ug = Hs.157601 /len = 398′ | | |
| 75258_f_a | Cluster Incl. AA224344:zr16d12.s1 Homo sapiens cDNA, | 5.018648158 | 0.002285 |
| | 3 end /clone = IMAGE-663575 /clone_end = 3′ | | |
| | /gb = AA224344 /gi = 1844967 /ug = Hs.237937 /len = 420′ | | |
| 75017_r_a | Cluster Incl. AW006583:wr25f01.x1 Homo sapiens cDNA, | 5.01502499 | 0.000541 |
| | 3 end /clone = IMAGE-2488729 /clone_end = 3′ | | |
| | /gb = AW006583 /gi = 5855361 /ug = Hs.236125 /len = 124′ | | |
| 73095_at | Cluster Incl. AI335277:tb29h06.x1 Homo sapiens cDNA, | 4.940911102 | 0.001852 |
| | 3 end /clone = IMAGE-2055803 /clone_end = 3′ | | |
| | /gb = AI335277 /gi = 4072204 /ug = Hs.224764 /len = 388′ | | |
| 77486_at | Cluster Incl. AA976712:oq06d04.s1 Homo sapiens cDNA, | 4.920359602 | 0.022545 |
| | 3 end /clone = IMAGE-1585543 /clone_end = 3′ | | |
| | /gb = AA976712 /gi = 3154158 /ug = Hs.128899 /len = 463′ | | |
| 87087_at | Cluster Incl. AI283643:qj67c11.x1 Homo sapiens cDNA, | 4.810346144 | 0.008358 |
| | 3 end /clone = IMAGE-1864532 /clone_end = 3′ | | |
| | /gb = AI283643 /gi = 3921876 /ug = Hs.188120 /len = 447′ | | |
| 73385_at | Cluster Incl. AA252082:zr63f09.s1 Homo sapiens cDNA, | 4.801518841 | 0.000861 |
| | 3 end /clone = IMAGE-668105 /clone_end = 3′ | | |
| | /gb = AA252082 /gi = 1887043 /ug = Hs.226391 /len = 399′ | | |
| 84678_i_a | Cluster Incl. AI056542:oy98c12.x1 Homo sapiens cDNA, | 4.720912558 | 0.002732 |
| | 3 end /clone = IMAGE-1673878 /clone_end = 3′ | | |
| | /gb = AI056542 /gi = 3330408 /ug = Hs.164276 /len = 361′ | | |
| 75737_g_{—} | Cluster Incl. AA625990:zu92h03.s1 Homo sapiens cDNA, | 4.719254539 | 0.004595 |
| | 3 end /clone = IMAGE-745493 /clone_end = 3′ | | |
| | /gb = AA625990 /gi = 2538377 /ug = Hs.116114 /len = 432′ | | |
| 77706_at | Cluster Incl. AA641972:ns18f01.r1 Homo sapiens cDNA, | 4.716780979 | 0.009315 |
| | 5 end /clone = IMAGE-1183993 /clone_end = 5′ | | |
| | /gb = AA641972 /gi = 2567190 /ug = Hs.130058 /len = 447′ | | |
| 75242_f_a | Cluster Incl. AW001140:wu24g09.x1 Homo sapiens cDNA, | 4.701250066 | 0.011085 |
| | 3 end /clone = IMAGE-990899 /clone_end = 3′ | | |
| | /gb = AW001140 /gi = 5848056 /ug = Hs.237782 /len = 415′ | | |
| 84996_at | Cluster Incl. AI379186:tc66d08.x1 Homo sapiens cDNA, | 4.6838985 | 0.001649 |
| | 3 end /clone = IMAGE-2069583 /clone_end = 3′ | | |
| | /gb = AI379186 /gi = 4189039 /ug = Hs.167183 /len = 473′ | | |
| 90289_at | Cluster Incl. AI827330:wl10d06.x1 Homo sapiens cDNA, | 4.653039196 | 0.014564 |
| | 3 end /clone = IMAGE-2424491 /clone_end = 3′ | | |
| | /gb = AI827330 /gi = 5448001 /ug = Hs.120750 /len = 435′ | | |
| 78672_at | Cluster Incl. AI276246:ql65e01.x1 Homo sapiens cDNA, | 4.623270914 | 0.000543 |
| | 3 end /clone = IMAGE-1877208 /clone_end = 3′ | | |
| | /gb = AI276246 /gi = 3898520 /ug = Hs.134702 /len = 473′ | | |
| 77739_i_a | Cluster Incl. AA910562:ok66g12.s1 Homo sapiens cDNA, | 4.589575228 | 0.00161 |
| | 3 end /clone = IMAGE-1518982 /clone_end = 3′ | | |
| | /gb = AA910562 /gi = 3049852 /ug = Hs.130207 /len = 416′ | | |
| 89820_at | Cluster Incl. AI828439:wk85b01.x1 Homo sapiens cDNA, | 4.574990842 | 0.016532 |
| | 3 end /clone = IMAGE-2422153 /clone_end = 3′ | | |
| | /gb = AI828439 /gi = 5449110 /ug = Hs.231580 /len = 388′ | | |
| 80876_at | Cluster Incl. AA513406:ne59g08.s1 Homo sapiens cDNA, | 4.547281772 | 0.019458 |
| | 3 end /clone = IMAGE-901694 /clone_end = 3′ | | |
| | /gb = AA513406 /gi = 2251818 /ug = Hs.152307 /len = 467′ | | |
| 83883_at | Cluster Incl. AA927837:on79e06.s1 Homo sapiens cDNA, | 4.503173747 | 0.011657 |
| | 3 end /clone = IMAGE-1562914 /clone_end = 3′ | | |
| | /gb = AA927837 /gi = 3077127 /ug = Hs.160112 /len = 348′ | | |
| 74077_at | Cluster Incl. AA888985:ak24f07.s1 Homo sapiens cDNA, | 4.464473172 | 0.004661 |
| | 3 end /clone = IMAGE-1406917 /clone_end = 3′ | | |
| | /gb = AA888985 /gi = 3015855 /ug = Hs.232115 /len = 369′ | | |
| 92121_r_a | Cluster Incl. AI022636:ox05f02.x1 Homo sapiens cDNA, | 4.380454753 | 0.045704 |
| | 3 end /clone = IMAGE-1655451 /clone_end = 3′ | | |
| | /gb = AI022636 /gi = 3237877 /ug = Hs.175531 /len = 495′ | | |
| 80193_at | Cluster Incl. AI130878:qc14d09.x1 Homo sapiens cDNA, | 4.312572045 | 0.005574 |
| | 3 end /clone = IMAGE-1709585 /clone_end = 3′ | | |
| | /gb = AI130878 /gi = 3600894 /ug = Hs.146517 /len = 432′ | | |
| 70825_at | Cluster Incl. AI301513:qn44h05.x1 Homo sapiens cDNA, | 4.257879375 | 0.000701 |
| | 3 end /clone = IMAGE-1901145 /clone_end = 3′ | | |
| | /gb = AI301513 /gi = 3960859 /ug = Hs.211386 /len = 427′ | | |
| 78478_r_a | Cluster Incl. H89231:yw25g04.r1 Homo sapiens cDNA, | 4.235271418 | 0.004183 |
| | 5 end /clone = IMAGE-253302 /clone_end = 5′ | | |
| | /gb = H89231/gi = 1071491/ug = Hs.133504 /len = 395′ | | |
| 90910_at | Cluster Incl. AI091388:oo26f02.x1 Homo sapiens cDNA, | 4.208360659 | 0.005383 |
| | 3 end /clone = IMAGE-1567323 /clone_end = 3′ | | |
| | /gb = AI091388 /gi = 3430447 /ug = Hs.134855 /len = 485′ | | |
| 81303._at | Cluster Incl. AI346656:qp52b03.x1 Homo sapiens cDNA, | 4.20318803 | 0.031305 |
| | 3 end /clone = IMAGE-1926605 /clone_end = 3′ | | |
| | /gb = AI346656 /gi = 4083862 /ug = Hs.156652 /len = 585′ | | |
| 82896_at | Cluster Incl. AI476732:tm23f10.x1 Homo sapiens cDNA, | 4.169388472 | 0.037823 |
| | 3 end /clone = IMAGE-2157451/clone_end = 3′ | | |
| | /gb = AI476732 /gi = 4329777 /ug = Hs.170624 /len = 446′ | | |
| 87624_at | Cluster Incl. AA668464:ab86a05.s1 Homo sapiens cDNA, | 4.167316157 | 0.002637 |
| | 3 end /clone = IMAGE-853808 /clone_end = 3′ | | |
| | /gb = AA668464 /gi = 2629963 /ug = Hs.190309 /len = 328′ | | |
| 91225_at | Cluster Incl. AW015211:UI-H-BI0p-aba-e-11-0-UI.s1 Homo sapiens cDNA, | 4.164345384 | 0.00008 |
| | 3 end /clone = IMAGE-2711156 /clone_end = 3′ | | |
| | /gb = AW015211/gi = 5863898 /ug = Hs.146181/len = 471′ | | |
| 81939_at | Cluster Incl. AI417773:th31c01.x1 Homo sapiens cDNA, | 4.138057591 | 0.004722 |
| | 3 end /clone = IMAGE-2119872 /clone end = 3′ | | |
| | /gb = AI417773 /gi = 4261277 /ug = Hs.161145 /len = 382′ | | |
| 69357_at | Cluster Incl. AI753726:cr14c07.x1 Homo sapiens cDNA, | 4.130344884 | 0.001956 |
| | 3 end /clone = HBMSC_cr14c07 /clone_end = 3′ | | |
| | /gb = AI753726 /gi = 5131990 /ug = Hs.202333 /len = 449′ | | |
| 77353_at | Cluster Incl. AI733279:oo46f12.x5 Homo sapiens cDNA, | 4.121422229 | 0.008861 |
| | 3 end /clone = IMAGE-1569263 /clone_end = 3′ | | |
| | /gb = AI733279 /gi = 5054392 /ug = Hs.128317 /len = 271′ | | |
| 79696_at | Cluster Incl. AA013440:ze26e09.s1 Homo sapiens cDNA, | 4.09631447 | 0.012229 |
| | 3 end /clone = IMAGE-360136 /clone_end = 3′ | | |
| | /gb = AA013440 /gi = 1474486 /ug = Hs.144252 /len = 463′ | | |
| 86510_at | Cluster Incl. AI076326:oy95d08.x1 Homo sapiens cDNA, | 4.086715666 | 0.025182 |
| | 3 end /clone = IMAGE-1673583 /clone end = 3′ | | |
| | /gb = AI076326 /gi = 3405504 /ug = Hs.185677 /len = 441′ | | |
| 79369_at | Cluster Incl. AA927001:om26d10.s1 Homo sapiens cDNA, | 4.051608302 | 0.000344 |
| | 3 end /clone = IMAGE-1542163 /clone_end = 3′ | | |
| | /gb = AA927001/gi = 3075898 /ug = Hs.142584 /len = 346′ | | |
| 88942_at | Cluster Incl. AI300447:qo13f06.x1 Homo sapiens cDNA, | 4.040588934 | 0.013394 |
| | 3 end /clone = IMAGE-1908419 /clone_end = 3′ | | |
| | /gb = AI300447 /gi = 3959793 /ug = Hs.202429 /len = 327′ | | |
| 84125_at | Cluster Incl. R86170:yp88e07.r1 Homo sapiens cDNA, | 4.014774285 | 0.00934 |
| | 5 end /clone = IMAGE-194532 /clone_end = 5′ | | |
| | /gb = R86170 /gi = 944576 /ug = Hs.161221 /len = 467′ | | |
| 73210_at | Cluster Incl. AI872472:ty42e07.x1 Homo sapiens cDNA, | 4.008599635 | 0.001324 |
| | 3 end /clone = IMAGE-2281764 /clone_end = 3′ | | |
| | /gb = AI872472 /gi = 5546521 /ug = Hs.225040 /len = 401′ | | |
| 78569_at | Cluster Incl. AI700684:we39c12.x1 Homo sapiens cDNA, | 4.002473204 | 0.000006 |
| | 3 end /clone = IMAGE-2343478 /clone_end = 3′ | | |
| | /gb = AI700684 /gi = 4988584 /ug = Hs.134166 /len = 571′ | | |
| 77117_at | Cluster Incl. AA938245:oo95b09.s1 Homo sapiens cDNA, | 3.972806977 | 0.012473 |
| | 3 end /clone = IMAGE-1573913 /clone_end = 3′ | | |
| | /gb = AA938245 /gi = 3096356 /ug = Hs.127160 /len = 421′ | | |
| 70838_r_a | Cluster Incl. AI824055:wj35e12.x1 Homo sapiens cDNA, | 3.946917752 | 0.023713 |
| | 3 end /clone = IMAGE-2404846 /clone_end = 3′ | | |
| | /gb = AI824055 /gi = 5444726 /ug = Hs.211396 /len = 577′ | | |
| 91676_at | Cluster Incl. AA534325:nf75e10.s1 Homo sapiens cDNA, | 3.926181357 | 0.019084 |
| | 3 end /clone = IMAGE-925770 /clone_end = 3′ | | |
| | /gb = AA534325 /gi = 2278578 /ug = Hs.162183 /len = 460′ | | |
| 68192_at | Cluster Incl. T92888:ye22e09.s1 Homo sapiens cDNA, | 3.901616313 | 0.014332 |
| | 3 end /clone = IMAGE-118504 /clone_end = 3′ | | |
| | /gb = T92888 /gi = 724801 /ug = Hs.191864 /len = 495′ | | |
| 84978_at | Cluster Incl. AI362920:qy81c11.x1 Homo sapiens cDNA, | 3.879227403 | 0.03697 |
| | 3 end /clone = IMAGE-2018420 /clone_end = 3′ | | |
| | /gb = AI362920 /gi = 4114541 /ug = Hs.167139 /len = 388′ | | |
| 82314_at | Cluster Incl. AI436813:th82h12.x1 Homo sapiens cDNA, | 3.836361251 | 0.02183 |
| | 3 end /clone = IMAGE-2125223 /clone_end = 3′ | | |
| | /gb = AI436813 /gi = 4284696 /ug = Hs.164217 /len = 508′ | | |
| 82070_at | Cluster Incl. AA564641:nj25e12.s1 Homo sapiens cDNA, | 3.77142292 | 0.003544 |
| | 3 end /clone = IMAGE-993550 /clone_end = 3′ | | |
| | /gb = AA564641 /gi = 2336280 /ug = Hs.162343 /len = 475′ | | |
| 79351_at | Cluster Incl. AA195514:zr33d12.s1 Homo sapiens cDNA, | 3.764326148 | 0.04421 |
| | 3 end /clone = IMAGE-665207 /clone end = 3′ | | |
| | /gb = AA195514 /gi = 1785400 /ug = Hs.142104 /len = 385′ | | |
| 84289_at | Cluster Incl. AI912393:tz05a01.x1 Homo sapiens cDNA, | 3.732611335 | 0.00756 |
| | 3 end /clone = IMAGE-2287656 /clone_end = 3′ | | |
| | /gb = AI912393 /gi = 5632248 /ug = Hs.162279 /len = 454′ | | |
| 70936_at | Cluster Incl. AI921300:wo23a09.x1 Homo sapiens cDNA, | 3.728217248 | 0.019033 |
| | 3 end /clone = IMAGE-2456152 /clone_end = 3′ | | |
| | /gb = AI921300 /gi = 5657264 /ug = Hs.211711 /len = 613′ | | |
| 74810_s_{—} | Cluster Incl. AW021074:df18e02.y1 Homo sapiens cDNA, | 3.722045457 | 0.00037 |
| | 5 end /clone = IMAGE-2483787 /clone_end = 5′ | | |
| | /gb = AW021074 /gi = 5874604 /ug = Hs.234355 /len = 351′ | | |
| 75014_i_a | Cluster Incl. AW006583:wr25f01.x1 Homo sapiens cDNA, | 3.681403454 | 0.000484 |
| | 3 end /clone = IMAGE-2488729 /clone_end = 3′ | | |
| | /gb = AW006583 /gi = 5855361 /ug = Hs.236125 /len = 124′ | | |
| 77741_r_a | Cluster Incl. AA910562:ok66g12.s1 Homo sapiens cDNA, | 3.68077061 | 0.004724 |
| | 3 end /clone = IMAGE-1518982 /clone_end = 3′ | | |
| | /gb = AA910562 /gi = 3049852 /ug = Hs.130207 /len = 416′ | | |
| 78304_at | Cluster Incl. AI056977:oy89d09.x1 Homo sapiens cDNA, | 3.617156362 | 0.013155 |
| | 3 end /clone = IMAGE-1673009 /clone_end = 3′ | | |
| | /gb = AI056977 /gi = 3330766 /ug = Hs.132900 /len = 459′ | | |
| 80124_at | Cluster Incl. AA643238:nr61f06.s1 Homo sapiens cDNA, | 3.580942752 | 0.001471 |
| | 3 end /clone = IMAGE-1172483 /clone_end = 3′ | | |
| | /gb = AA643238 /gi = 2568456 /ug = Hs.146144 /len = 476′ | | |
| 84304_at | Cluster Incl. AA521177:aa73f03.s1 Homo sapiens cDNA, | 3.57711283 | 0.002455 |
| | 3 end /clone = IMAGE-826589 /clone_end = 3′ | | |
| | /gb = AA521177 /gi = 2261720 /ug = Hs.162409 /len = 559′ | | |
| 89972_at | Cluster Incl. AI420969:te97b09.x1 Homo sapiens cDNA, | 3.575423765 | 0.007712 |
| | 3 end /clone = IMAGE-2094617 /clone_end = 3′ | | |
| | /gb = AI420969 /gi = 4266900 /ug = Hs.235870 /len = 383′ | | |
| 90581_at | Cluster Incl. AA974579:op28g10.s1 Homo sapiens cDNA, | 3.567570093 | 0.028452 |
| | 3 end /clone = IMAGE-1578210 /clone_end = 3′ | | |
| | /gb = AA974579 /gi = 3149759 /ug = Hs.128385 /len = 408′ | | |
| 74922_at | Cluster Incl. AW025554:wu97g07.x1 Homo sapiens cDNA, | 3.544362859 | 0.008025 |
| | 3 end /clone = IMAGE-990991 /clone_end = 3′ | | |
| | /gb = AW025554 /gi = 5879084 /ug = Hs.235239 /len = 443′ | | |
| 81319_at | Cluster Incl. AI686894:tp90h06.x1 Homo sapiens cDNA, | 3.538203629 | 0.01427 |
| | 3 end /clone = IMAGE-2206619 /clone_end = 3′ | | |
| | /gb = AI686894 /gi = 4898188 /ug = Hs.156704 /len = 476′ | | |
| 88620_at | Cluster Incl. AI970185:wr08c07.x1 Homo sapiens cDNA, | 3.533373724 | 0.00708 |
| | 3 end /clone = IMAGE-2480940 /clone_end = 3′ | | |
| | /gb = AI970185 /gi = 5767011 /ug = Hs.197641 /len = 514′ | | |
| 87615_r_a | Cluster Incl. AA626219:zv88a11.s1 Homo sapiens cDNA, | 3.515678463 | 0.030307 |
| | 3 end /clone = IMAGE-766844 /clone_end = 3′ | | |
| | /gb = AA626219 /gi = 2538606 /ug = Hs.190274 /len = 263′ | | |
| 77005_r_a | Cluster Incl. AA918616:ol67a12.s1 Homo sapiens cDNA, | 3.50824968 | 0.004026 |
| | 3 end /clone = IMAGE-1534654 /clone_end = 3′ | | |
| | /gb = AA918616 /gi = 3058506 /ug = Hs.126658 /len = 495′ | | |
| 81874_at | Cluster Incl. AI734974:as54e06.x1 Homo sapiens cDNA, | 3.495899557 | 0.042114 |
| | 3 end /clone = IMAGE-2332546 /clone_end = 3′ | | |
| | /gb = AI734974 /gi = 5056498 /ug = Hs.160604 /len = 506′ | | |
| 90190_at | Cluster Incl. AI819924:wj11c04.x1 Homo sapiens cDNA, | 3.492860427 | 0.000238 |
| | 3 end /clone = IMAGE-2402502 /clone_end = 3′ | | |
| | /gb = AI819924 /gi = 5439003 /ug = Hs.116559 /len = 534′ | | |
| 88949_at | Cluster Incl. AI744009:wc32f04.x1 Homo sapiens cDNA, | 3.484438351 | 0.000883 |
| | 3 end /clone = IMAGE-2316895 /clone_end = 3′ | | |
| | /gb = AI744009 /gi = 5112297 /ug = Hs.202451 /len = 568′ | | |
| 92039_at | Cluster Incl. AI955491:wt30e12.x1 Homo sapiens cDNA, | 3.436563997 | 0.006207 |
| | 3 end /clone = IMAGE-2509006 /clone_end = 3′ | | |
| | /gb = AI955491 /gi = 5747801 /ug = Hs.171372 /len = 671′ | | |
| 74868_at | Cluster Incl. AI991048:wu36c07.x1 Homo sapiens cDNA, | 3.42763578 | 0.000285 |
| | 3 end /clone = IMAGE-990847 /clone_end = 3′ | | |
| | /gb = AI991048 /gi = 5837945 /ug = Hs.234668 /len = 429′ | | |
| 85706_at | Cluster Incl. AI218358:qh21g07.x1 Homo sapiens cDNA, | 3.422351762 | 0.017153 |
| | 3 end /clone = IMAGE-1845372 /clone_end = 3′ | | |
| | /gb = AI218358 /gi = 3798173 /ug = Hs.175048 /len = 411′ | | |
| 71117_f_a | Cluster Incl. AI982832:wt44e02.x1 Homo sapiens cDNA, | 3.418516359 | 0.001844 |
| | 3 end /clone = IMAGE-2510330 /clone_end = 3′ | | |
| | /gb = AI982832 /gi = 5810051 /ug = Hs.212529 /len = 497′ | | |
| 68339_at | Cluster Incl. AI624028:ts25g07.x1 Homo sapiens cDNA, | 3.404452919 | 0.000617 |
| | 3 end /clone = IMAGE-2229660 /clone_end = 3′ | | |
| | /gb = AI624028 /gi = 4648959 /ug = Hs.192626 /len = 213′ | | |
| 78680_at | Cluster Incl. AA282171:zt02c06.s1 Homo sapiens cDNA, | 3.401834363 | 0.009019 |
| | 3 end /clone = IMAGE-711946 /clone_end = 3′ | | |
| | /gb = AA282171 /gi = 1925032 /ug = Hs.134740 /len = 427′ | | |
| 90436_at | Cluster Incl. W93113:zh45f06.s1 Homo sapiens cDNA, | 3.40090527 | 0.021093 |
| | 3 end /clone = IMAGE-415043 /clone_end = 3′ | | |
| | /gb = W93113 /gi = 1422275 /ug = Hs.125212 /len = 421′ | | |
| 81650_at | Cluster Incl. AA631047:nq77b01.s1 Homo sapiens cDNA, | 3.353588529 | 0.000357 |
| | 3 end /clone = IMAGE-1158313 /clone_end = 3′ | | |
| | /gb = AA631047 /gi = 2553658 /ug = Hs.158761 /len = 588′ | | |
| 73248_at | Cluster Incl. AI955755:wt59b10.x1 Homo sapiens cDNA, | 3.33387584 | 0.006207 |
| | 3 end /clone = IMAGE-2511739 /clone_end = 3′ | | |
| | /gb = AI955755 /gi = 5748065 /ug = Hs.225142 /len = 525′ | | |
| 73315_at | Cluster Incl. AI868289:tj44g02.x1 Homo sapiens cDNA, | 3.332728736 | 0.00066 |
| | 3 end /clone = IMAGE-2144402 /clone_end = 3′ | | |
| | /gb = AI868289 /gi = 5541305 /ug = Hs.225651 /len = 289′ | | |
| 67401_at | Cluster Incl. AI434675:ti35f10.x1 Homo sapiens cDNA, | 3.330352227 | 0.00106 |
| | 3 end /clone = IMAGE-2132491 /clone_end = 3′ | | |
| | /gb = AI434675 /gi = 4297838 /ug = Hs.164299 /len = 340′ | | |
| 76150_at | Cluster Incl. AA778773:zj41h06.s1 Homo sapiens cDNA, | 3.324537689 | 0.01164 |
| | 3 end /clone = 452891 /clone_end = 3′ | | |
| | /gb = AA778773 /gi = 2838104 /ug = Hs.119691 /len = 423′ | | |
| 75183_at | Cluster Incl. AI990633:ws22f07.x1 Homo sapiens cDNA, | 3.313399383 | 0.006284 |
| | 3 end /clone = IMAGE-2497957 /clone_end = 3′ | | |
| | /gb = AI990633 /gi = 5837514 /ug = Hs.237246 /len = 445′ | | |
| 80971_at | Cluster Incl. AI033377:ox03b10.s1 Homo sapiens cDNA, | 3.307979211 | 0.008879 |
| | 3 end /clone = IMAGE-1655227 /clone_end = 3′ | | |
| | /gb = AI033377 /gi = 3254330 /ug = Hs.153298 /len = 485′ | | |
| 86810_at | Cluster Incl. AI581415:to71f12.x1 Homo sapiens cDNA, | 3.286327472 | 0.027513 |
| | 3 end /clone = IMAGE-2183759 /clone_end = 3′ | | |
| | /gb = AI581415 /gi = 4565791 /ug = Hs.187260 /len = 392′ | | |
| 80261_s_{—} | Cluster Incl. AI961818:wt66g05.x1 Homo sapiens cDNA, | 3.248004767 | 0.00471 |
| | 3 end /clone = IMAGE-2512472 /clone_end = 3′ | | |
| | /gb = AI961818 /gi = 5754531 /ug = Hs.146916 /len = 643′ | | |
| 71177_at | Cluster Incl. AI921931:wn86g12.x1 Homo sapiens cDNA, | 3.241857044 | 0.020963 |
| | 3 end /clone = IMAGE-2452774 /clone_end = 3′ | | |
| | /gb = AI921931 /gi = 5657895 /ug = Hs.212815 /len = 516′ | | |
| 81927_at | Cluster Incl. AI417669:tg80g11.x1 Homo sapiens cDNA, | 3.235184899 | 0.038241 |
| | 3 end /clone = IMAGE-2115140 /clone_end = 3′ | | |
| | /gb = AI417669 /gi = 4261173 /ug = Hs.161134 /len = 391′ | | |
| 80623_s_{—} | Cluster Incl. AI640222:wa30f03.x1 Homo sapiens cDNA, | 3.218612679 | 0.008845 |
| | 3 end /clone = IMAGE-2299613 /clone_end = 3′ | | |
| | /gb = AI640222 /gi = 4703331 /ug = Hs.149993 /len = 451′ | | |
| 74696_r_a | Cluster Incl. AW003358:wq65g09.x1 Homo sapiens cDNA, | 3.216414783 | 0.000981 |
| | 3 end /clone = IMAGE-2476192 /clone_end = 3′ | | |
| | /gb = AW003358 /gi = 5850274 /ug = Hs.234010 /len = 406′ | | |
| 87133_at | Cluster Incl. AI275499:ql74d03.x1 Homo sapiens cDNA, | 3.215553093 | 0.029742 |
| | 3 end /clone = IMAGE-1878053 /clone_end = 3′ | | |
| | /gb = AI275499 /gi = 3897773 /ug = Hs.188350 /len = 472′ | | |
| 77363_at | Cluster Incl. AI651606:wb07c02.x1 Homo sapiens cDNA, | 3.205498008 | 0.0075 |
| | 3 end /clone = IMAGE-2304962 /clone_end = 3′ | | |
| | /gb = AI651606 /gi = 4735585 /ug = Hs.128395 /len = 588′ | | |
| 75791_f_a | Cluster Incl. AI821102:nr20h03.y5 Homo sapiens cDNA | 3.197729341 | 0.013912 |
| | /clone = IMAGE-1168565 /gb = AI821102 /gi = 5440181 | | |
| | /ug = Hs.116350 /len = 419 | | |
| 73261_at | Cluster Incl. AI692539:wd73e11.x1 Homo sapiens cDNA, | 3.194807353 | 0.000008 |
| | 3 end /clone = IMAGE-2337260 /clone_end = 3′ | | |
| | /gb = AI692539 /gi = 4969879 /ug = Hs.225186 /len = 534′ | | |
| 81717_at | Cluster Incl. AI375700:ta58g09.x1 Homo sapiens cDNA, | 3.189360956 | 0.024927 |
| | 3 end /clone = IMAGE-2048320 /clone_end = 3′ | | |
| | /gb = AI375700 /gi = 4175690 /ug = Hs.159185 /len = 407′ | | |
| 69102_at | Cluster Incl. AI950819:wx55f02.x1 Homo sapiens cDNA, | 3.182469138 | 0.037281 |
| | 3 end /clone = IMAGE-2547579 /clone_end = 3′ | | |
| | /gb = AI950819 /gi = 5743129 /ug = Hs.201680 /len = 484′ | | |
| 81452_at | Cluster Incl. AI421933:tf45a12.x1 Homo sapiens cDNA, | 3.159839766 | 0.024083 |
| | 3 end /clone = IMAGE-2099134 /clone_end = 3′ | | |
| | /gb = AI421933 /gi = 4267864 /ug = Hs.157383 /len = 465′ | | |
| 83115_at | Cluster Incl. AI346114:qp48f09.x1 Homo sapiens cDNA, | 3.143614459 | 0.003379 |
| | 3 end /clone = IMAGE-1926281 /clone_end = 3′ | | |
| | /gb = AI346114 /gi = 4083320 /ug = Hs.173495 /len = 458′ | | |
| 74630_r_a | Cluster Incl. AA995791:os29f07.s1 Homo sapiens cDNA, | 3.121958997 | 0.046206 |
| | 3 end /clone = IMAGE-1606789 /clone_end = 3′ | | |
| | /gb = AA995791 /gi = 3182280 /ug = Hs.233832 /len = 477′ | | |
| 80664_at | Cluster Incl. AI248671:qh66g01.x1 Homo sapiens cDNA, | 3.116104719 | 0.004129 |
| | 3 end /clone = IMAGE-1849680 /clone_end = 3′ | | |
| | /gb = AI248671 /gi = 3844068 /ug = Hs.150289 /len = 432′ | | |
| 78136_at | Cluster Incl. AI038623:ox39b12.s1 Homo sapiens cDNA, | 3.114649496 | 0.037841 |
| | 3 end /clone = IMAGE-1658687 /clone_end = 3′ | | |
| | /gb = AI038623 /gi = 3277817 /ug = Hs.132087 /len = 452′ | | |
| 68888_at | Cluster Incl. AI671829:wb34d12.x1 Homo sapiens cDNA, | 3.114197965 | 0.03414 |
| | 3 end /clone = IMAGE-2307575 /clone_end = 3′ | | |
| | /gb = AI671829 /gi = 4851560 /ug = Hs.200573 /len = 504′ | | |
| 79683_at | Cluster Incl. AI223034:qg52h05.x1 Homo sapiens cDNA, | 3.104715847 | 0.018458 |
| | 3 end /clone = IMAGE-1838841 /clone_end = 3′ | | |
| | /gb = AI223034 /gi = 3805237 /ug = Hs.144137 /len = 497′ | | |
| 75012_at | Cluster Incl. AI989698:ws35d09.x1 Homo sapiens cDNA, | 3.103880371 | 0.037238 |
| | 3 end /clone = IMAGE-2499185 /clone_end = 3′ | | |
| | /gb = AI989698 /gi = 5836579 /ug = Hs.236112 /len = 380′ | | |
| 67147_at | Cluster Incl. AI040048:ox28a08.x1 Homo sapiens cDNA, | 3.098224539 | 0.032493 |
| | 3 end /clone = IMAGE-1657622 /clone_end = 3′ | | |
| | /gb = AI040048 /gi = 3279242 /ug = Hs.157207 /len = 416′ | | |
| 72075_at | Cluster Incl. AA279019:zs83a10.s1 Homo sapiens cDNA, | 3.08870358 | 0.004529 |
| | 3 end /clone = IMAGE-704058 /clone_end = 3′ | | |
| | /gb = AA279019 /gi = 1920638 /ug = Hs.221039 /len = 445′ | | |
| 69524_at | Cluster Incl. AI739558:wi35a05.x1 Homo sapiens cDNA, | 3.079273337 | 0.033429 |
| | 3 end /clone = IMAGE-2392208 /clone_end = 3′ | | |
| | /gb = AI739558 /gi = 5101539 /ug = Hs.203481 /len = 335′ | | |
| 72512_at | Cluster Incl. AA653151:ag64c01.s1 Homo sapiens cDNA, | 3.065825864 | 0.041772 |
| | 3 end /clone = IMAGE-1127712 /clone_end = 3′ | | |
| | /gb = AA653151 /gi = 2589322 /ug = Hs.222057 /len = 408′ | | |
| 71211_at | Cluster Incl. AI050877:oy47c06.x1 Homo sapiens cDNA, | 3.041281957 | 0.002806 |
| | 3 end /clone = IMAGE-1668970 /clone_end = 3′ | | |
| | /gb = AI050877 /gi = 3307682 /ug = Hs.212948 /len = 357′ | | |
| 88788_i_a | Cluster Incl. AA969517:oo85a11.s1 Homo sapiens cDNA, | 3.032310778 | 0.005066 |
| | 3 end /clone = IMAGE-1572956 /clone_end = 3′ | | |
| | /gb = AA969517 /gi = 3144697 /ug = Hs.201079 /len = 377′ | | |
| 77052_at | Cluster Incl. AI914225:wd74g03.x1 Homo sapiens cDNA, | 3.023302076 | 0.004485 |
| | 3 end /clone = IMAGE-2337364 /clone_end = 3′ | | |
| | /gb = AI914225 /gi = 5634080 /ug = Hs.126865 /len = 447′ | | |
| 75551_at | Cluster Incl. AI566143:tn53h10.x1 Homo sapiens cDNA, | 3.017433674 | 0.021729 |
| | 3 end /clone = IMAGE-2172163 /clone_end = 3′ | | |
| | /gb = AI566143 /gi = 4524595 /ug = Hs.113010 /len = 420′ | | |
| 84740_at | Cluster Incl. N73628:yz78b01.s1 Homo sapiens cDNA, | 3.014935259 | 0.020032 |
| | 3 end /clone = IMAGE-289129 /clone_end = 3′ | | |
| | /gb = N73628 /gi = 1230913 /ug = Hs.165153 /len = 453′ | | |
| 74611_s_{—} | Cluster Incl. AI983615:wu19h07.x1 Homo sapiens cDNA, | 3.012380075 | 0.042251 |
| | 3 end /clone = IMAGE-990800 /clone_end = 3′ | | |
| | /gb = AI983615 /gi = 5810834 /ug = Hs.233756 /len = 482′ | | |
| 76464_g_{—} | Cluster Incl. AA781854:ai61h03.s1 Homo sapiens cDNA, | 3.006117339 | 0.0097 |
| | 3 end /clone = 1375349 /clone_end = 3′ | | |
| | /gb = AA781854 /gi = 2841185 /ug = Hs.122275 /len = 367′ | | |
| 90055_s_{—} | Cluster Incl. AA778124:zf46g09.s1 Homo sapiens cDNA, | 0.327601885 | 0.003177 |
| | 3 end /clone = 380032 /clone_end = 3′ | | |
| | /gb = AA778124 /gi = 2837525 /ug = Hs.238767 /len = 466′ | | |
| 89108_at | Cluster Incl. AA780912:ag98f07.s1 Homo sapiens cDNA, | 0.326532542 | 0.031272 |
| | 3 end /clone = 1155205 /clone_end = 3′ | | |
| | /gb = AA780912 /gi = 2840243 /ug = Hs.204249 /len = 489′ | | |
| 78594_at | Cluster Incl. AI080633:ox54b12.x1 Homo sapiens cDNA, | 0.326310174 | 0.04264 |
| | 3 end /clone = IMAGE-1660127 /clone_end = 3′ | | |
| | /gb = AI080633 /gi = 3416884 /ug = Hs.134225 /len = 519′ | | |
| 84816_at | Cluster Incl. AA995233:ou17a03.s1 Homo sapiens cDNA, | 0.326189959 | 0.000316 |
| | 3 end /clone = IMAGE-1626508 /clone_end = 3′ | | |
| | /gb = AA995233 /gi = 3181722 /ug = Hs.166309 /len = 497′ | | |
| 76410_at | Cluster Incl. H05391:yl80b11.r1 Homo sapiens cDNA, | 0.316419067 | 0.001262 |
| | 5 end /clone = IMAGE-44222 /clone_end = 5′ | | |
| | /gb = H05391 /gi = 868943 /ug = Hs.122008 /len = 443′ | | |
| 74367_at | Cluster Incl. AI989468:ws25b02.x1 Homo sapiens cDNA, | 0.315634857 | 0.005064 |
| | 3 end /clone = IMAGE-2498187 /clone_end = 3′ | | |
| | /gb = AI989468 /gi = 5836349 /ug = Hs.233354 /len = 407′ | | |
| 88668_at | Cluster Incl. AI631519:wa99a02.x1 Homo sapiens cDNA, | 0.314758398 | 0.028635 |
| | 3 end /clone = IMAGE-2304266 /clone_end = 3′ | | |
| | /gb = AI631519 /gi = 4682849 /ug = Hs.198000 /len = 494′ | | |
| 91172_at | Cluster Incl. AA772360:ai43d11.s1 Homo sapiens cDNA, | 0.31434908 | 0.013501 |
| | 3 end /clone = 1359765 /clone_end = 3′ | | |
| | /gb = AA772360 /gi = 2824143 /ug = Hs.145274 /len = 471′ | | |
| 80238_at | Cluster Incl. AA507036:ni02h04.s1 Homo sapiens cDNA, | 0.311633699 | 0.003808 |
| | 3 end /clone = IMAGE-966871 /clone_end = 3′ | | |
| | /gb = AA507036 /gi = 2243475 /ug = Hs.146822 /len = 466′ | | |
| 85907_at | Cluster Incl. AI276085:ql74f09.x1 Homo sapiens cDNA, | 0.31047302 | 0.004634 |
| | 3 end /clone = IMAGE-1878089 /clone_end = 3′ | | |
| | /gb = AI276085 /gi = 3898359 /ug = Hs.176222 /len = 465′ | | |
| 80485_i_a | Cluster Incl. AI808768:wf57h12.x1 Homo sapiens cDNA, | 0.308522904 | 0.016329 |
| | 3 end /clone = IMAGE-2359751 /clone_end = 3′ | | |
| | /gb = AI808768 /gi = 5395334 /ug = Hs.148867 /len = 481′ | | |
| 72026_g_{—} | Cluster Incl. AA427578:zw54b07.s1 Homo sapiens cDNA, | 0.308401991 | 0.001714 |
| | 3 end /clone = IMAGE-773845 /clone_end = 3′ | | |
| | /gb = AA427578 /gi = 2112006 /ug = Hs.220975 /len = 525′ | | |
| 72703_at | Cluster Incl. AA010008:zi07c10.s1 Homo sapiens cDNA, | 0.307232814 | 0.000017 |
| | 3 end /clone = IMAGE-430098 /clone_end = 3′ | | |
| | /gb = AA010008 /gi = 1471055 /ug = Hs.222505 /len = 436′ | | |
| 87230_at | Cluster Incl. AA521281:aa75g12.s1 Homo sapiens cDNA, | 0.305350897 | 0.049077 |
| | 3 end /clone = IMAGE-826822 /clone_end = 3′ | | |
| | /gb = AA521281 /gi = 2261824 /ug = Hs.188830 /len = 556′ | | |
| 77885_at | Cluster Incl. AI733673:ov09h03.x5 Homo sapiens cDNA, | 0.305290936 | 0.00235 |
| | 3 end /clone = IMAGE-1636853 /clone_end = 3′ | | |
| | /gb = AI733673 /gi = 5054786 /ug = Hs.131005 /len = 464′ | | |
| 87827_at | Cluster Incl. AI200630:qf87g12.x1 Homo sapiens cDNA, | 0.303877717 | 0.022954 |
| | 3 end /clone = IMAGE-1757062 /clone_end = 3′ | | |
| | /gb = AI200630 /gi = 3753236 /ug = Hs.190725 /len = 449′ | | |
| 73346_at | Cluster Incl. AA732736:ai28a09.s1 Homo sapiens cDNA, | 0.303166429 | 0.000992 |
| | 3 end /clone = 1344088 /clone_end = 3′ | | |
| | /gb = AA732736 /gi = 2754095 /ug = Hs.225853 /len = 464′ | | |
| 76154_i_a | Cluster Incl. AI634548:tx52g01.x1 Homo sapiens cDNA, | 0.302833962 | 0.000105 |
| | 3 end /clone = IMAGE-2273232 /clone_end = 3′ | | |
| | /gb = AI634548 /gi = 4685878 /ug = Hs.119748 /len = 523′ | | |
| 79735_at | Cluster Incl. AA037529:zk36c07.r1 Homo sapiens cDNA, | 0.302793001 | 0.000342 |
| | 5 end /clone = IMAGE-484908 /clone_end = 5′ | | |
| | /gb = AA037529 /gi = 1512629 /ug = Hs.144559 /len = 489′ | | |
| 84844_at | Cluster Incl. AI269474:qh47e02.x1 Homo sapiens cDNA, | 0.301592705 | 0.006328 |
| | 3 end /clone = IMAGE-1847834 /clone_end = 3′ | | |
| | /gb = AI269474 /gi = 3888641 /ug = Hs.166420 /len = 398′ | | |
| 80874_at | Cluster Incl. AI659898:tu01d09.x1 Homo sapiens cDNA, | 0.299388186 | 0.014565 |
| | 3 end /clone = IMAGE-2249777 /clone_end = 3′ | | |
| | /gb = AI659898 /gi = 4763468 /ug = Hs.152298 /len = 521′ | | |
| 84768_at | Cluster Incl. AI446224:tj25f11.x1 Homo sapiens cDNA, | 0.295386963 | 0.010618 |
| | 3 end /clone = IMAGE-2142573 /clone_end = 3′ | | |
| | /gb = AI446224 /gi = 4293712 /ug = Hs.165817 /len = 350′ | | |
| 77908_at | Cluster Incl. AI016841:ov33d08.x1 Homo sapiens cDNA, | 0.29124207 | 0.009569 |
| | 3 end /clone = IMAGE-1639119 /clone_end = 3′ | | |
| | /gb = AI016841 /gi = 3231177 /ug = Hs.131106 /len = 408′ | | |
| 76248_at | Cluster Incl. AI653732:wb36g02.x1 Homo sapiens cDNA, | 0.288672932 | 0.038981 |
| | 3 end /clone = IMAGE-2307794 /clone_end = 3′ | | |
| | /gb = AI653732 /gi = 4737711 /ug = Hs.120691 /len = 457′ | | |
| 75779_at | Cluster Incl. AA629059:zu84b05.s1 Homo sapiens cDNA, | 0.286202318 | 0.01828 |
| | 3 end /clone = IMAGE-744657 /clone_end = 3′ | | |
| | /gb = AA629059 /gi = 2541446 /ug = Hs.116298 /len = 570′ | | |
| 91145_at | Cluster Incl. AI759990:wh36g05.x1 Homo sapiens cDNA, | 0.285672403 | 0.004773 |
| | 3 end /clone = IMAGE-2382872 /clone_end = 3′ | | |
| | /gb = AI759990 /gi = 5175657 /ug = Hs.144824 /len = 351′ | | |
| 75293_s_{—} | Cluster Incl. AW009667:ws85g02.x1 Homo sapiens cDNA, | 0.284658559 | 0.003682 |
| | 3 end /clone = IMAGE-2504786 /clone_end = 3′ | | |
| | /gb = AW009667 /gi = 5858445 /ug = Hs.238353 /len = 601′ | | |
| 81973_r_a | Cluster Incl. AI421200:tf24f09.x1 Homo sapiens cDNA, | 0.284252973 | 0.010971 |
| | 3 end /clone = IMAGE-2097161 /clone_end = 3′ | | |
| | /gb = AI421200 /gi = 4267131 /ug = Hs.161312 /len = 422′ | | |
| AFFX-HU | M33197 Human glyceraldehyde-3-phosphate dehydrogenase (GAPDH) mRNA, | 0.284040649 | 0.015245 |
| | complete cds (_5, _M, _3 represent transcript regions | | |
| | 5 prime, Middle, and 3 prime respectively) | | |
| 91840_at | Cluster Incl. W39635:zc19c08.r1 Homo sapiens cDNA, | 0.282077522 | 0.038709 |
| | 5 end /clone = IMAGE-322766 /clone_end = 5′ | | |
| | /gb = W39635 /gi = 1321344 /ug = Hs.167828 /len = 435′ | | |
| 83720_at | Cluster Incl. AI380073:tf78f01.x1 Homo sapiens cDNA, | 0.280175516 | 0.000283 |
| | 3 end /clone = IMAGE-2105401 /clone_end = 3′ | | |
| | /gb = AI380073 /gi = 4189926 /ug = Hs.158949 /len = 443′ | | |
| 81035_i_a | Cluster Incl. AI573275:tn03f10.x1 Homo sapiens cDNA, | 0.278845944 | 0.042646 |
| | 3 end /clone = IMAGE-2166571 /clone_end = 3′ | | |
| | /gb = AI573275 /gi = 4536649 /ug = Hs.153892 /len = 503′ | | |
| 73217_at | Cluster Incl. AI950400:wq35a04.x1 Homo sapiens cDNA, | 0.278037315 | 0.023478 |
| | 3 end /clone = IMAGE-2473230 /clone_end = 3′ | | |
| | /gb = AI950400 /gi = 5742710 /ug = Hs.225073 /len = 684′ | | |
| 87996_at | Cluster Incl. AI215886:qm35g06.x1 Homo sapiens cDNA, | 0.271801622 | 0.027086 |
| | 3 end /clone = IMAGE-1883866 /clone_end = 3′ | | |
| | /gb = AI215886 /gi = 3784927 /ug = Hs.191824 /len = 483′ | | |
| 69515_at | Cluster Incl. AI738451:wi32a06.x1 Homo sapiens cDNA, | 0.26449689 | 0:003509 |
| | 3 end /clone = IMAGE-2391922 /clone_end = 3′ | | |
| | /gb = AI738451 /gi = 5100432 /ug = Hs.203304 /len = 534′ | | |
| 79472_at | Cluster Incl. N53442:yv41a09.s1 Homo sapiens cDNA, | 0.264444183 | 0.000068 |
| | 3 end /clone = IMAGE-245272 /clone_end = 3′ | | |
| | /gb = N53442 /gi = 1194608 /ug = Hs.143443 /len = 479′ | | |
| 81629_r_a | Cluster Incl. AI939503:qy19d04.x5 Homo sapiens cDNA, | 0.26339748 | 0.000103 |
| | 3 end /clone = IMAGE-2012455 /clone_end = 3′ | | |
| | /gb = AI939503 /gi = 5678292 /ug = Hs.158545 /len = 428′ | | |
| 92166_at | Cluster Incl. AI190864:qd55a08.x1 Homo sapiens cDNA, | 0.261339615 | 0.00325 |
| | 3 end /clone = IMAGE-1733366 /clone_end = 3′ | | |
| | /gb = AI190864 /gi = 3742073 /ug = Hs.178226 /len = 451′ | | |
| 81217_at | Cluster Incl. AI971594:wq87h01.x1 Homo sapiens cDNA, | 0.261004215 | 0.009305 |
| | 3 end /clone = IMAGE-2479057 /clone_end = 3′ | | |
| | /gb = AI971594 /gi = 5768420 /ug = Hs.156105 /len = 501′ | | |
| 89937_at | Cluster Incl. AA700625:zi43a06.s1 Homo sapiens cDNA, | 0.258257021 | 0.001392 |
| | 3 end /clone = 433522 /clone_end = 3′ | | |
| | /gb = AA700625 /gi = 2703588 /ug = Hs.234473 /len = 564′ | | |
| 81584_at | Cluster Incl. AI828498:wk30a12.x1 Homo sapiens cDNA, | 0.257194389 | 0.016235 |
| | 3 end /clone = IMAGE-2413822 /clone_end = 3′ | | |
| | /gb = AI828498 /gi = 5449169 /ug = Hs.158186 /len = 539′ | | |
| 70409_at | Cluster Incl. AI792564:qi73h09.y5 Homo sapiens cDNA, | 0.253124275 | 0.019626 |
| | 5 end /clone = IMAGE-1862177 /clone_end = 5′ | | |
| | /gb = AI792564 /gi = 5340280 /ug = Hs.209447 /len = 326′ | | |
| 88042_i_a | Cluster Incl. AA704465:zj19c02.s1 Homo sapiens cDNA, | 0.252686592 | 0.000395 |
| | 3 end /clone = 450722 /clone_end = 3′ | | |
| | /gb = AA704465 /gi = 2714383 /ug = Hs.191941 /len = 480′ | | |
| 74756_at | Cluster Incl. AI869008:wc17h01.x1 Homo sapiens cDNA, | 0.25104725 | 0.01521 |
| | 3 end /clone = IMAGE-2315473 /clone_end = 3′ | | |
| | /gb = AI869008 /gi = 5542976 /ug = Hs.234184 /len = 352′ | | |
| 80680_at | Cluster Incl. AI279514:ql51c11.x1 Homo sapiens cDNA, | 0.247710917 | 0.036068 |
| | 3 end /clone = IMAGE-1875860 /clone_end = 3′ | | |
| | /gb = AI279514 /gi = 3917748 /ug = Hs.150459 /len = 565′ | | |
| 71324_at | Cluster Incl. AI761935:wh50a05.x1 Homo sapiens cDNA, | 0.247617951 | 0.004563 |
| | 3 end /clone = IMAGE-2384144 /clone_end = 3′ | | |
| | /gb = AI761935 /gi = 5177602 /ug = Hs.213335 /len = 452′ | | |
| 69100_at | Cluster Incl. AI682297:wc51d01.x1 Homo sapiens cDNA, | 0.247438489 | 0.001126 |
| | 3 end /clone = IMAGE-2322145 /clone_end = 3′ | | |
| | /gb = AI682297 /gi = 4892479 /ug = Hs.201657 /len = 481′ | | |
| 82786_at | Cluster Incl. AW014764:UI-H-BI0-aae-f-08-0-UI.s1 Homo sapiens cDNA, | 0.245652038. | 0.017259 |
| | 3 end /clone = IMAGE-2709254 /clone_end = 3′ | | |
| | /gb = AW014764 /gi = 5863521 /ug = Hs.169169 /len = 383′ | | |
| 75211_s_{—} | Cluster Incl. H78699:yu29h0-1.s1 Homo sapiens cDNA, | 0.244829232 | 0.000058 |
| | 3 end /clone = IMAGE-235249 /clone_end = 3′ | | |
| | /gb = H78699 /gi = 1056788 /ug = Hs.237375 /len = 418′ | | |
| 78158_at | Cluster Incl. AI042180:oy37c12.x1 Homo sapiens cDNA, | 0.24479353 | 0.000431 |
| | 3 end /clone = IMAGE-1668022 /clone_end = 3′ | | |
| | /gb = AI042180 /gi = 3281374 /ug = Hs.132160 /len = 533′ | | |
| 79294_i_a | Cluster Incl. R95918:yq51f07.s1 Homo sapiens cDNA, | 0.240319896 | 0.003866 |
| | 3 end /clone = IMAGE-199333 /clone_end = 3′ | | |
| | /gb = R95918 /gi = 981578 /ug = Hs.140871 /len = 296′ | | |
| 68112_at | Cluster Incl. AA931562:oo56c11.s1 Homo sapiens cDNA, | 0.240105629 | 0.029475 |
| | 3 end /clone = IMAGE-1570196 /clone_end = 3′ | | |
| | /gb = AA931562 /gi = 3085948 /ug = Hs.190736 /len = 488′ | | |
| 79618_at | Cluster Incl. AI740621:wg23e12.x1 Homo sapiens cDNA, | 0.239308307 | 0.004818 |
| | 3 end /clone = IMAGE-2365966 /clone_end = 3′ | | |
| | /gb = AI740621 /gi = 5108909 /ug = Hs.143873 /len = 457′ | | |
| 75719_at | Cluster Incl. AI039291:ox33e10.s1 Homo sapiens cDNA, | 0.239047129 | 0.001528 |
| | 3 end /clone = IMAGE-1658154 /clone_end = 3′ | | |
| | /gb = AI039291 /gi = 3278485 /ug = Hs.116061 /len = 435′ | | |
| 78779_at | Cluster Incl. AI087874:ow60d03.x1 Homo sapiens cDNA, | 0.238520097 | 0.003141 |
| | 3 end /clone = IMAGE-1651205 /clone_end = 3′ | | |
| | /gb = AI087874 /gi = 3426907 /ug = Hs.135162 /len = 515′ | | |
| 71787_r_a | Cluster Incl. AI312358:ta78h12.x2 Homo sapiens cDNA, | 0.237885742 | 0.033666 |
| | 3 end /clone = IMAGE-2050247 /clone_end = 3′ | | |
| | /gb = AI312358 /gi = 4017963 /ug = Hs.220599 /len = 146′ | | |
| 71560_r_a | Cluster Incl. AI932356:wd27d09.x1 Homo sapiens cDNA, | 0.234298993 | 0.002953 |
| | 3 end /clone = IMAGE-2329361 /clone_end = 3′ | | |
| | /gb = AI932356 /gi = 5671093 /ug = Hs.216016 /len = 486′ | | |
| 78580_at | Cluster Incl. AI344345:tc03h05.x1 Homo sapiens cDNA, | 0.234044364 | 0.003148 |
| | 3 end /clone = IMAGE-2062809 /clone_end = 3′ | | |
| | /gb = AI344345 /gi = 4081551 /ug = Hs.134198 /len = 501′ | | |
| 78563_at | Cluster Incl. AI302188:qn58d06.x1 Homo sapiens cDNA, | 0.228535599 | 0.000468 |
| | 3 end /clone = IMAGE-1902443 /clone_end = 3′ | | |
| | /gb = AI302188 /gi = 3961534 /ug = Hs.134128 /len = 464′ | | |
| 87915_i_a | Cluster Incl. W93079:zh45a08.s1 Homo sapiens cDNA, | 0.223549654 | 0.000173 |
| | 3 end /clone = IMAGE-414998 /clone_end = 3′ | | |
| | /gb = W93079 /gi = 1422241 /ug = Hs.191396 /len = 352′ | | |
| 90914_at | Cluster Incl. AI088153:oz96a09.x1 Homo sapiens cDNA, | 0.219235587 | 0.040539 |
| | 3 end /clone = IMAGE-1683160 /clone_end = 3′ | | |
| | /gb = AI088153 /gi = 3427129 /ug = Hs.134902 /len = 485′ | | |
| 86791_at | Cluster Incl. AA461106:zx63g09.s1 Homo sapiens cDNA, | 0.218378808 | 0.000089 |
| | 3 end /clone = IMAGE-796192 /clone_end = 3′ | | |
| | /gb = AA461106 /gi = 2186226 /ug = Hs.187123 /len = 400′ | | |
| 89355_at | Cluster Incl. AI819564:wj91f12.x1 Homo sapiens cDNA, | 0.214671449 | 0.001157 |
| | 3 end /clone = IMAGE-2410223 /clone_end = 3′ | | |
| | /gb = AI819564 /gi = 5438643 /ug = Hs.208230 /len = 469′ | | |
| 88225_at | Cluster Incl. AA779891:af46d05.s1 Homo sapiens cDNA, | 0.213518745 | 0.005318 |
| | 3 end /clone = 1034697 /clone_end = 3′ | | |
| | /gb = AA779891 /gi = 2839222 /ug = Hs.193052 /len = 472′ | | |
| 73771_at | Cluster Incl. AI632232:tt20e05.x1 Homo sapiens cDNA, | 0.212420493 | 0.002735 |
| | 3 end /clone = IMAGE-2241344 /clone_end = 3′ | | |
| | /gb = AI632232 /gi = 4683562 /ug = Hs.231855 /len = 496′ | | |
| 82591_r_a | Cluster Incl. AI309223:qo68a10.x1 Homo sapiens cDNA, | 0.212211885 | 0.006857 |
| | 3 end /clone = IMAGE-1913658 /clone_end = 3′ | | |
| | /gb = AI309223 /gi = 4004094 /ug = Hs.167011 /len = 331′ | | |
| 80687_at | Cluster Incl. AA921994:om41a07.s1 Homo sapiens cDNA, | 0.210798369 | 0.010674 |
| | 3 end /clone = IMAGE-1543572 /clone_end = 3′ | | |
| | /gb = AA921994 /gi = 3069303 /ug = Hs.150493 /len = 411′ | | |
| 78370_at | Cluster Incl. AI053597:qi72e03.x1 Homo sapiens cDNA, | 0.209176341 | 0.001388 |
| | 3 end /clone = IMAGE-1862044 /clone_end = 3′ | | |
| | /gb = AI053597 /gi = 3321384 /ug = Hs.133138 /len = 546′ | | |
| 82231_at | Cluster Incl. AA367838:EST79039 Homo sapiens cDNA | 0.208973443 | 0.000022 |
| | /clone = ATCC-172567 /gb = AA367838 /gi = 2020198 | | |
| | /ug = Hs.163834 /len = 374 | | |
| 73517_at | Cluster Incl. AI865729:wk50e02.x1 Homo sapiens cDNA, | 0.208676712 | 0.001615 |
| | 3 end /clone = IMAGE-2418842 /clone_end = 3′ | | |
| | /gb = AI865729 /gi = 5529836 /ug = Hs.229800 /len = 283′ | | |
| 85305_at | Cluster Incl. AI440266:tj01e04.x1 Homo sapiens cDNA, | 0.207370287 | 0.000035 |
| | 3 end /clone = IMAGE-2140254 /clone_end = 3′ | | |
| | /gb = AI440266 /gi = 4281451 /ug = Hs.170673 /len = 461′ | | |
| 74891_at | Cluster Incl. AA928646:om75f04.s1 Homo sapiens cDNA, | 0.20599295 | 0.02766 |
| | 3 end /clone = IMAGE-1553023 /clone_end = 3′ | | |
| | /gb = AA928646 /gi = 3076937 /ug = Hs.234976 /len = 488′ | | |
| 78399_at | Cluster Incl. AI052543:oz27h04.x1 Homo sapiens cDNA, | 0.204116157 | 0.001194 |
| | 3 end /clone = IMAGE-1676599 /clone_end = 3′ | | |
| | /gb = AI052543 /gi = 3308534 /ug = Hs.133244 /len = 452′ | | |
| 77478_at | Cluster Incl. AA977896:oq62b04.s1 Homo sapiens cDNA, | 0.198979696 | 0.001194 |
| | 3 end /clone = IMAGE-1590895 /clone_end = 3′ | | |
| | /gb = AA977896 /gi = 3155342 /ug = Hs.128873 /len = 417′ | | |
| 72808_at | Cluster Incl. AI640523:wa29b01.x1 Homo sapiens cDNA, | 0.198366813 | 0.00004 |
| | 3 end /clone = IMAGE-2299465 /clone_end = 3′ | | |
| | /gb = AI640523 /gi = 4703632 /ug = Hs.223553 /len = 442′ | | |
| 84903_f_a | Cluster Incl. AI264299:qk20f06.x1 Homo sapiens cDNA, | 0.197923908 | 0.007339 |
| | 3 end /clone = IMAGE-1869539 /clone_end = 3′ | | |
| | /gb = AI264299 /gi = 3872502 /ug-Hs.166784 /ten = 396′ | | |
| 84077_at | Cluster Incl. AI379425:tc66g04x1 .Homo sapiens cDNA, | 0.195398279 | 0.002798 |
| | 3 end /clone = IMAGE-2069622 /clone_end = 3′ | | |
| | /gb = AI379425 /gi = 4189278 /ug = Hs.160942 /len = 472′ | | |
| 85747_at | Cluster Incl. AI610910:tt60a11.x1 Homo sapiens cDNA, | 0.193631412 | 0.000081 |
| | 3 end /clone = IMAGE-2245148 /clone_end = 3′ | | |
| | /gb = AI610910 /gi = 4620077 /ug = Hs.175357 /len = 122′ | | |
| 81913_at | Cluster Incl. AI371042:ta29f06.x1 Homo sapiens cDNA, | 0.193436596 | 0.001955 |
| | 3 end /clone = IMAGE-2045507 /clone_end = 3′ | | |
| | /gb = AI371042 /gi = 4149795 /ug = Hs.160911 /len = 484′ | | |
| 88656_at | Cluster Incl. AI650477:wa91d08.x1 Homo sapiens cDNA, | 0.190979429 | 0.037893 |
| | 3 end /clone = IMAGE-2303535 /clone_end = 3′ | | |
| | /gb = AI650477 /gi = 4734456 /ug = Hs.197758 /len = 478′ | | |
| 69365_at | Cluster Incl. AI703361:wd93d02.x1 Homo sapiens cDNA, | 0.184683765 | 0.000162 |
| | 3 end /clone = IMAGE-2339139 /clone_end = 3′ | | |
| | /gb = AI703361 /gi = 4991261 /ug = Hs.202354 /len = 530′ | | |
| 77598_at | Cluster Incl. AA994249:ou05b11.s1 Homo sapiens cDNA, | 0.182835726 | 0.0023 |
| | 3 end /clone = IMAGE-1625373 /clone_end = 3′ | | |
| | /gb = AA994249 /gi = 3180794 /ug = Hs.129479 /len = 414′ | | |
| 73971_at | Cluster Incl. AI375662:tc09c10.x1 Homo sapiens cDNA, | 0.181763269 | 0.001296 |
| | 3 end /clone = IMAGE-2063346 /clone_end = 3′ | | |
| | /gb = AI375662 /gi = 4175652 /ug = Hs.232023 /len = 435′ | | |
| 82063_at | Cluster Incl. AA552017:ng01g11.s1 Homo sapiens cDNA, | 0.17951496 | 0.002115 |
| | 3 end /clone = IMAGE-928196 /clone_end = 3′ | | |
| | /gb = AA552017 /gi = 2322269 /ug = Hs.162245 /len = 460′ | | |
| 90300_g_{—} | Cluster Incl. AA721234:nz72b08.s1 Homo sapiens cDNA | 0.17343166 | 0.010479 |
| | /clone = IMAGE-1300983 /gb = AA721234 /gi = 2737369 | | |
| | /ug = Hs.121121 /len = 345 | | |
| 91925_at | Cluster Incl. AI360231:qy84d11.x1 Homo sapiens cDNA, | 0.171324444 | 0.029254 |
| | 3 end /clone = IMAGE-2018709 /clone_end = 3′ | | |
| | /gb = AI360231 /gi = 4111852 /ug = Hs.170245 /len = 485′ | | |
| 79022_at | Cluster Incl. AI821803:nr20b05.x5 Homo sapiens cDNA | 0.170191727 | 0.000004 |
| | /clone = IMAGE-1168497 /gb = AI821803 /gi = 5440882 | | |
| | /ug = Hs.136580 /len = 307 | | |
| 82302_at | Cluster Incl. AI436290:th81c01.x1 Homo sapiens cDNA, | 0.169417008 | 0.000324 |
| | 3 end /clone = IMAGE-2125056 /clone_end = 3′ | | |
| | /gb = AI436290 /gi = 4309151 /ug = Hs.164162 /len = 497′ | | |
| 91591_at | Cluster Incl. AI469896:tj88c11.x1 Homo sapiens cDNA, | 0.164920143 | 0.002275 |
| | 3 end /clone = IMAGE-2148596 /clone_end = 3′ | | |
| | /gb = AI469896 /gi = 4331986 /ug = Hs.158866 /len = 459′ | | |
| 77589_at | Cluster Incl. AI377752:te56h12.x1 Homo sapiens cDNA, | 0.161975336 | 0.000178 |
| | 3 end /clone = IMAGE-2090759 /clone_end = 3′ | | |
| | /gb = AI377752 /gi = 4187605 /ug = Hs.129448 /len = 429′ | | |
| 84438_at | Cluster Incl. AA843562:aj54f01.s1 Homo sapiens cDNA, | 0.15021849 | 0.003363 |
| | 3 end /clone = IMAGE-1394137 /clone_end = 3′ | | |
| | /gb = AA843562 /gi = 2930080 /ug = Hs.163277 /len = 435′ | | |
| 74557_s_{—} | Cluster Incl. AI739473:wi14a05.xl Homo sapiens cDNA, | 0.142318725 | 0.000049 |
| | 3 end /clone = IMAGE-2390192 /clone_end = 3′ | | |
| | /gb = AI739473 /gi = 5101454 /ug = Hs.233630 /len = 463′ | | |
| 87274_at | Cluster Incl. AA700621;zi43a01.s1 Homo sapiens cDNA, | 0.138942719 | 0.000066 |
| | 3 end /clone = 433512 /clone_end = 3′ | | |
| | /gb = AA700621 /gi = 2703584 /ug = Hs.188964 /len = 519′ | | |
| 90647_at | Cluster Incl. AI701529:we36a05.x1 Homo sapiens cDNA, | 0.134163985 | 0.000207 |
| | 3 end /clone = IMAGE-2343152 /clone_end = 3′ | | |
| | /gb = AI701529 /gi = 4989429 /ug = Hs.129519 /len = 507′ | | |
| 91481_at | Cluster Incl. AW025687:wu07b01.x1 Homo sapiens cDNA, | 0.132765609 | 0.000971 |
| | 3 end /clone = IMAGE-2516233 /clone_end = 3′ | | |
| | /gb = AW025687 /gi = 5879217 /ug = Hs.156452 /len = 408′ | | |
| 89107_s_{—} | Cluster Incl. AA161147:zo74a11.s1 Homo sapiens cDNA, | 0.131705656 | 0.048733 |
| | 3 end /clone = IMAGE-592604 /clone end = 3′ | | |
| | /gb = AA161147 /gi = 1735384 /ug = Hs.204218 /len = 345′ | | |
| 77818_at | Cluster Incl. AA927633:om19g09.s1 Homo sapiens cDNA, | 0.129714004 | 0.000441 |
| | 3 end /clone = IMAGE-1541536 /clone_end = 3′ | | |
| | /gb = AA927633 /gi = 3077054 /ug = Hs.130685 /len = 654′ | | |
| 87657_at | Cluster Incl. AA699394:zi33d10.s1 Homo sapiens cDNA, | 0.128325924 | 0.010529 |
| | 3 end /clone = 432595 /clone_end = 3′ | | |
| | /gb = AA699394 /gi = 2702588 /ug = Hs.190348 /len = 445′ | | |
| 83947_at | Cluster Incl. AI221304:qg74f11.x1 Homo sapiens cDNA, | 0.127051245 | 0.004949 |
| | 3 end /clone = IMAGE-1840941 /clone_end = 3′ | | |
| | /gb = AI221304 /gi = 3803507 /ug = Hs.160372 /len = 410′ | | |
| 77148_at | Cluster Incl. AA939266:oq31a09.s1 Homo sapiens cDNA, | 0.126798542 | 0.01012 |
| | 3 end /clone = IMAGE-1587928 /clone_end = 3′ | | |
| | /gb = AA939266 /gi = 3099179 /ug = Hs.127309 /len = 513′ | | |
| 81632_i_a | Cluster Incl. AI369347:qy92a02.x1 Homo sapiens cDNA, | 0.126685823 | 0.000114 |
| | 3 end /clone = IMAGE-2019434 /clone_end = 3′ | | |
| | /gb = AI369347 /gi = 4148100 /ug = Hs.158592 /len = 215′ | | |
| 91500_at | Cluster Incl. AA069788:zf72h10.s1 Homo sapiens cDNA, | 0.126293774 | 0.006699 |
| | 3 end /clone = IMAGE-382531 /clone_end = 3′ | | |
| | /gb = AA069788 /gi = 1577156 /ug = Hs.156880 /len = 637′ | | |
| 67260_r_a | Cluster Incl. AI383679:tc45h10.x1 Homo sapiens cDNA, | 0.120913902 | 0.000002 |
| | 3 end /clone = IMAGE-2067619 /clone_end = 3′ | | |
| | /gb = AI383679 /gi = 4196460 /ug = Hs.159114 /len = 244′ | | |
| 72368_f_a | Cluster Incl. AI583312:ts31g07.x1 Homo sapiens cDNA, | 0.113556615 | 0.028878 |
| | 3 end /clone = IMAGE-2230236 /clone_end = 3′ | | |
| | /gb = AI583312 /gi = 4569209 /ug = Hs.221671 /len = 356′ | | |
| 80075_at | Cluster Incl. AI034221:ow09g01.x1 Homo sapiens cDNA, | 0.110154914 | 0.000049 |
| | 3 end /clone = IMAGE-1646352 /clone_end = 3′ | | |
| | /gb = AI034221 /gi = 3255174 /ug = Hs.146035 /len = 432′ | | |
| 91129_at | Cluster Incl. AI208356:qg35f06.x1 Homo sapiens cDNA, | 0.106889175 | 0.0002 |
| | 3 end /clone = IMAGE-1837187 /clone_end = 3′ | | |
| | /gb = AI208356 /gi = 3770298 /ug = Hs.144507 /len = 388′ | | |
| 69853_at | Cluster Incl. AI798028:wh80e11.x1 Homo sapiens cDNA, | 0.104569948 | 0.000275 |
| | 3 end /clone = IMAGE-2387084 /clone_end = 3′ | | |
| | /gb = AI798028 /gi = 5363500 /ug = Hs.207474 /len = 549′ | | |
| 74508_at | Cluster Incl. AW015038:UI-H-BI0-aal-a-03-0-UI.s1 Homo sapiens cDNA, | 0.104558745 | 0.004341 |
| | 3 end /clone = IMAGE-2709413 /clone_end = 3′ | | |
| | /gb = AW015038 /gi = 5863795 /ug = Hs.233530 /len = 423′ | | |
| 72462_f_a | Cluster Incl. H40456:yp60d12.s1 Homo sapiens cDNA, | 0.101016546 | 0.000992 |
| | 3 end /clone = IMAGE-191831 /clone_end = 3′ | | |
| | /gb = H40456 /gi = 916508 /ug = Hs.221895 /len = 418′ | | |
| 84947_at | Cluster Incl. AI286188:qi02a08.x1 Homo sapiens cDNA, | 0.092519177 | 0.001823 |
| | 3 end /clone = IMAGE-1855286 /clone_end = 3′ | | |
| | /gb = AI286188 /gi = 3924421 /ug = Hs.166980 /len = 417′ | | |
| 70855_at | Cluster Incl. AI829652:wf02h07.x1 Homo sapiens cDNA, | 0.083077091 | 0.000164 |
| | 3 end /clone = IMAGE-2349469 /clone_end = 3′ | | |
| | /gb = AI829652 /gi = 5450323 /ug = Hs.211443 /len = 494′ | | |
| 90484_at | Cluster Incl. AI809578:wf31a09.x1 Homo sapiens cDNA, | 0.079933078 | 0.000211 |
| | 3 end /clone = IMAGE-2357176 /clone_end = 3′ | | |
| | /gb = AI809578 /gi = 5396144 /ug = Hs.126434 /len = 496′ | | |
| 69162_at | Cluster Incl. AI692566:wd73h03.x1 Homo sapiens cDNA, | 0.079613815 | 0.000067 |
| | 3 end /clone = IMAGE-2337269 /clone_end = 3′ | | |
| | /gb = AI692566 /gi = 4969906 /ug = Hs.201888 /len = 455′ | | |
| 69526_at | Cluster Incl. AA158249:zo76c07.s1 Homo sapiens cDNA, | 0.075582558 | 0.000006 |
| | 3 end /clone = IMAGE-592812 /clone_end = 3′ | | |
| | /gb = AA158249 /gi = 1733044 /ug = Hs.203537 /len = 580′ | | |
| 91579_at | Cluster Incl. AI939511:qy28e02.x5 Homo sapiens cDNA, | 0.06757725 | 0.00086 |
| | 3 end /clone = IMAGE-2013338 /clone_end = 3′ | | |
| | /gb = AI939511 /gi = 5678300 /ug = Hs.158678 /len = 547′ | | |
| 86283_r_a | Cluster Incl. T47055:yb12g05.s1 Homo sapiens cDNA, | 0.067022858 | 0.005615 |
| | 3 end /clone = IMAGE-71000 /clone_end = 3′ | | |
| | /gb = T47055 /gi = 649038 /ug = Hs.182019 /len = 348′ | | |
| 84082_at | Cluster Incl. AI566771:tq67e02.x1 Homo sapiens cDNA, | 0.059933336 | 0.00077 |
| | 3 end /clone = IMAGE-2213882 /clone_end = 3′ | | |
| | /gb = AI566771 /gi = 4525223 /ug = Hs.160953 /len = 461′ | | |
| 83466_at | Cluster Incl. AA468767:ne09e12.s1 Homo sapiens cDNA, | 0.057826652 | 0 |
| | 3 end /clone = IMAGE-880750 /clone_end = 3′ | | |
| | /gb = AA468767 /gi = 2195301 /ug = Hs.178337 /len = 442′ | | |
| 73402_at | Cluster Incl. AA781220:ai46a03.s1 Homo sapiens cDNA, | 0.052624187 | 0.000058 |
| | 3 end /clone = 1360012 /clone_end = 3′ | | |
| | /gb = AA781220 /gi = 2840551 /ug = Hs.226722 /len = 426′ | | |
| 84869_at | Cluster Incl. AI123917:qa37e01.x1 Homo sapiens cDNA, | 0.049170602 | 0.000018 |
| | 3 end /clone = IMAGE-1688952. /clone end = 3′ | | |
| | /gb = AI123917 /gi = 3539683 /ug = Hs.166597 /len = 495′ | | |
| 73243_at | Cluster Incl. AI199897:qf84a05.x1 Homo sapiens cDNA, | 0.045513684 | 0 |
| | 3 end /clone = IMAGE-1756688 /clone_end = 3′ | | |
| | /gb = AI199897 /gi = 3752503 /ug = Hs.225137 /len = 563′ | | |
| 73485_at | Cluster Incl. T71258:yd35b05.s1 Homo sapiens cDNA, | 0.041793009 | 0.000017 |
| | 3 end /clone = IMAGE-110193 /clone_end = 3′ | | |
| | /gb = T71258 /gi = 685779 /ug = Hs.228790 /len = 517′ | | |
| 75857_at | Cluster Incl. W80832:zd90g02.s1 Homo sapiens cDNA, | 0.035564271 | 0.000051 |
| | 3 end /clone = IMAGE-356786 /clone_end = 3′ | | |
| | /gb = W80832 /gi = 1391875 /ug = Hs.116773 /len = 467′ | | |
| 90122_f_a | Cluster Incl. AW025309:wu73c09.x1 Homo sapiens cDNA, | 0.024715815 | 0 |
| | 3 end /clone = IMAGE-990947 /clone_end = 3′ | | |
| | /gb = AW025309 /gi = 5878839 /ug = Hs.113165 /len = 497′ | | |
| 91361_at | Cluster Incl. AI265958:qq90h05.x1 Homo sapiens cDNA, | 0.017169688 | 0.00001 |
| | 3 end /clone = IMAGE-1938681 /clone_end = 3′ | | |
| | /gb = AI265958 /gi = 3884116 /ug = Hs.150821 /len = 490′ | | |
| 84270_at | Cluster Incl. AI829641:wf02g08.x1 Homo sapiens cDNA, | 0.014034927 | 0.003989 |
| | 3 end /clone = IMAGE-2349470 /clone_end = 3′ | | |
| | /gb = AI829641 /gi = 5450312 /ug = Hs.162204 /len = 348′ | | |
| 69149_at | Cluster Incl. AI282714:qt65b06.x1 Homo sapiens cDNA, | 0.012878608 | 0.00022 |
| | 3 end /clone-IMAGE-1960115 /clone_end = 3′ | | |
| | /gb = AI282714 /gi = 3920947 /ug = Hs.201877 /len = 412′ | | |

TABLE 6
U95_E Fold Change Genes (>3 over-expressed in Barrett's associated
esophageal adenocarcinoma (BA), <0.33 under-expressed in BA)

| Affy ID | Gene Name | Fold Change | P-value |
|---|---|---|---|
| 88567_s_{—} | Cluster Incl. AI344053:tc01h08.x1 Homo sapiens cDNA, | 33.12163404 | 0.000003 |
| | 3 end /clone = IMAGE-2062623 /clone_end = 3′ | | |
| | /gb = AI344053 /gi = 4081259 /ug = Hs.115838 /len = 449′ | | |
| 76739_at | Cluster Incl. AI337014:qx87g02.x1 Homo sapiens cDNA, | 24.46364831 | 0 |
| | 3 end /clone = IMAGE-2009522 /clone_end = 3′ | | |
| | /gb = AI337014 /gi = 4073941 /ug = Hs.132989 /len = 679′ | | |
| 83365_at | Cluster Incl. AI341602:qx92g05.x1 Homo sapiens cDNA, | 21.6308226 | 0.001328 |
| | 3 end /clone = IMAGE-2010008 /clone_end = 3′ | | |
| | /gb = AI341602 /gi = 4078529 /ug = Hs.152932 /len = 461′ | | |
| 81562_at | Cluster Incl. AI733811:qk04h06.x5 Homo sapiens cDNA, | 20.13979542 | 0.000018 |
| | 3 end /clone = IMAGE-1868027 /clone_end = 3′ | | |
| | /gb = AI733811 /gi = 5054924 /ug = Hs.121194 /len = 609′ | | |
| 73111_s_{—} | Cluster Incl. AI859854:wm21c10.x1 Homo sapiens cDNA, | 19.38056138 | 0.000001 |
| | 3 end /clone = IMAGE-2436594 /clone_end = 3′ | | |
| | /gb = AI859854 /gi = 5513459 /ug = Hs.234479 /len = 455′ | | |
| 90421_at | Cluster Incl. AA633203:nq57b02.s1 Homo sapiens cDNA, | 19.2499917 | 0.000051 |
| | 3 end /clone = IMAGE-1147947 /clone_end = 3′ | | |
| | /gb = AA633203 /gi = 2556617 /ug = Hs.14258 /len = 570′ | | |
| 88580_at | Cluster Incl. AI275140:ql70h02.x1 Homo sapiens cDNA, | 18.08284727 | 0.000109 |
| | 3 end /clone = IMAGE-1877715 /clone_end = 3′ | | |
| | /gb = AI275140 /gi = 3897414 /ug = Hs.116104 /len = 444′ | | |
| 78328_at | Cluster Incl. AI691066:wf22b05.x1 Homo sapiens cDNA, | 15.78241233 | 0.000067 |
| | 3 end /clone = IMAGE-2351313 /clone_end = 3′ | | |
| | /gb = AI691066 /gi = 4902368 /ug = Hs.165619 /len = 448′ | | |
| 89808_at | Cluster Incl. AA628967:af28h06.s1 Homo sapiens cDNA, | 15.29942334 | 0.00001 |
| | 3 end /clone = IMAGE-1033019 /clone_end = 3′ | | |
| | /gb = AA628967 /gi = 2541354 /ug = Hs.115274 /len = 753′ | | |
| 90582_at | Cluster Incl. AI688604:wc90f10.x1 Homo sapiens cDNA, | 15.09542862 | 0.000754 |
| | 3 end /clone = IMAGE-2325931 /clone_end = 3′ | | |
| | /gb = AI688604 /gi = 4899898 /ug = Hs.154903 /len = 454′ | | |
| 87884_at | Cluster Incl. AI922828:wo14e03.x1 Homo sapiens cDNA, | 14.54781052 | 0.00033 |
| | 3 end /clone = IMAGE-2455324 /clone_end = 3′ | | |
| | /gb = AI922828 /gi = 5658792 /ug = Hs.236102 /len = 548′ | | |
| 68693_at | Cluster Incl. T69015:yc31f04.s1 Homo sapiens cDNA, | 14.08619278 | 0.000013 |
| | 3 end /clone = IMAGE-82303 /clone_end = 3′ | | |
| | /gb = T69015 /gi = 680163 /ug = Hs.192728 /len = 478′ | | |
| 73054_at | Cluster Incl. C06075:C06075 Homo sapiens cDNA | 11.3375198 | 0.012822 |
| | /clone = hbc5496 /gb = C06075 /gi = 1502851 | | |
| | /ug = Hs.234147 /len = 454 | | |
| 81774_at | Cluster Incl. AI683036:tw48f06.x1 Homo sapiens cDNA, | 10.36686915 | 0.004902 |
| | 3 end /clone = IMAGE-2262947 /clone_end = 3′ | | |
| | /gb = AI683036 /gi = 4893218 /ug = Hs.124135 /len = 482′ | | |
| 82840_at | Cluster Incl. AI799626:to74b03.x1 Homo sapiens cDNA, | 10.18825096 | 0.000015 |
| | 3 end /clone = IMAGE-2183981 /clone_end = 3′ | | |
| | /gb = AI799626 /gi = 5365098 /ug = Hs.145807 /len = 558′ | | |
| 88504_at | Cluster Incl. AI955239:wx31f07.x1 Homo sapiens cDNA, | 9.83796577 | 0.002999 |
| | 3 end /clone = IMAGE-2545285 /clone_end = 3′ | | |
| | /gb = AI955239 /gi = 5747549 /ug = Hs.112460 /len = 467′ | | |
| 77510_at | Cluster Incl. AA088446:zl89f04.s1 Homo sapiens cDNA, | 9.77410756 | 0.000003 |
| | 3 end /clone = IMAGE-511807 /clone_end = 3′ | | |
| | /gb = AA088446 /gi = 1633958 /ug = Hs.170298 /len = 590′ | | |
| 78379_at | Cluster Incl. AI684746:wa85f10.x1 Homo sapiens cDNA, | 9.652586214 | 0.000012 |
| | 3 end /clone = IMAGE-2302987 /clone_end = 3′ | | |
| | /gb = AI684746 /gi = 4896040 /ug = Hs.119274 /len = 768′ | | |
| 73464_r_a | Cluster Incl. AA648038:HGBBT038 Homo sapiens cDNA | 9.485196734 | 0.002348 |
| | /gb = AA648038 /gi = 2574467 /ug = Hs.95614 /len = 180 | | |
| 90136_at | Cluster Incl. AI638295:tt09b11.x1 Homo sapiens cDNA, | 9.024823944 | 0.001058 |
| | 3 end /clone = IMAGE-2240253 /clone_end = 3′ | | |
| | /gb = AI638295 /gi = 4690529 /ug = Hs.128685 /len = 547′ | | |
| 73826_at | Cluster Incl. AI971242:wr27a01.x1 Homo sapiens cDNA, | 8.515076242 | 0.000011 |
| | 3 end /clone = IMAGE-2488872 /clone_end = 3′ | | |
| | /gb = AI971242 /gi = 5768068 /ug = Hs.124854 /len = 453′ | | |
| 84271_r_a | Cluster Incl. AW025560:wu97h02.x1 Homo sapiens cDNA, | 7.818636431 | 0.002861 |
| | 3 end /clone = IMAGE-990982 /clone_end = 3′ | | |
| | /gb = AW025560 /gi = 5879090 /ug = Hs.166398 /len = 458′ | | |
| 73493_at | Cluster Incl. AA701983:zi70a04.s1 Homo sapiens cDNA, | 7.302601124 | 0.00004 |
| | 3 end /clone = 436110 /clone_end = 3′ | | |
| | /gb = AA701983 /gi = 2705096 /ug = Hs.114085 /len = 522′ | | |
| 76364_at | Cluster Incl. AA643507:nq86f06.s1 Homo sapiens cDNA, | 6.913074606 | 0.001015 |
| | 3 end /clone = IMAGE-1159235 /clone_end = 3′ | | |
| | /gb = AA643507 /gi = 2568725 /ug = Hs.124840 /len = 509′ | | |
| 91095_s_{—} | Cluster Incl. AA535819:nj79e01.s1 Homo sapiens cDNA | 6.873901823 | 0.002754 |
| | /clone = IMAGE-998712 /gb = AA535819 /gi = 2280072 | | |
| | /ug = Hs.238355 /len = 522 | | |
| 81623_at | Cluster Incl. AI208954:qg29e05.x1 Homo sapiens cDNA, | 6.825834554 | 0.001662 |
| | 3 end /clone = IMAGE-1836608 /clone_end = 3′ | | |
| | /gb = AI208954 /gi = 3770896 /ug = Hs.121876 /len = 418′ | | |
| 81690_at | Cluster Incl. AI093231:qb04b09.x1 Homo sapiens cDNA, | 6.794338221 | 0.000834 |
| | 3 end /clone = IMAGE-1695257 /clone_end = 3′ | | |
| | /gb = AI093231 /gi = 3432207 /ug = Hs.122910 /len = 451′ | | |
| 81932_at | Cluster Incl. AI659966:we64e02.x1 Homo sapiens cDNA, | 6.724516081 | 0.00524 |
| | 3 end /clone = IMAGE-2345882 /clone_end = 3′ | | |
| | /gb = AI659966 /gi = 4763536 /ug = Hs.126478 /len = 490′ | | |
| 77540_at | Cluster Incl. AA007367:zh98b03.s1 Homo sapiens cDNA, | 6.57607309 | 0.003018 |
| | 3 end /clone = IMAGE-429293 /clone_end = 3′ | | |
| | /gb = AA007367 /gi = 1463371 /ug = Hs.173088 /len = 462′ | | |
| 73238_at | Cluster Incl. AI457905:tj48b10.x1 Homo sapiens cDNA, | 6.48771467 | 0.006137 |
| | 3 end /clone = IMAGE-2144731 /clone_end = 3′ | | |
| | /gb = AI457905 /gi = 4310786 /ug = Hs.235943 /len = 546′ | | |
| 85141_at | Cluster Incl. AI192838:qe63c04.x1 Homo sapiens cDNA, | 6.18069379 | 0.002186 |
| | 3 end /clone = IMAGE-1743654 /clone_end = 3′ | | |
| | /gb = AI192838 /gi = 3744047 /ug = Hs.183053 /len = 444′ | | |
| 85126_at | Cluster Incl. AI751438:cn10a03.y1 Homo sapiens cDNA | 6.087887372 | 0.00056 |
| | /clone = NHTBC_cn10a03-(random) /gb = AI751438 | | |
| | /gi = 5129702 /ug = Hs.182827 /len = 513 | | |
| 81668_at | Cluster Incl. AI346446:qp45a07.x1 Homo sapiens cDNA, | 6.04859158 | 0.000438 |
| | 3 end /clone = IMAGE-1925940 /clone_end = 3′ | | |
| | /gb = AI346446 /gi = 4083652 /ug = Hs.122587 /len = 470′ | | |
| 88155_at | Cluster Incl. AI125923:qc41f07.x1 Homo sapiens cDNA, | 5.967007568 | 0.020546 |
| | 3 end /clone = IMAGE-1712197 /clone_end = 3′ | | |
| | /gb = AI125923 /gi = 3594437 /ug = Hs.239324 /len = 512′ | | |
| 88222_at | Cluster Incl. AW013949:UI-H-BI0-aad-g-11-0-UI.s1 Homo sapiens cDNA, | 5.934357544 | 0.000747 |
| | 3 end /clone = IMAGE-2708949 /clone_end = 3′ | | |
| | /gb = AW013949 /gi = 5862706 /ug = Hs.239673 /len = 361′ | | |
| 80604_at | Cluster Incl. AW007566:wt02e03.x1 Homo sapiens cDNA, | 5.811542288 | 0.015477 |
| | 3 end /clone = IMAGE-2506300 /clone_end = 3′ | | |
| | /gb = AW007566 /gi = 5856429 /ug = Hs.239193 /len = 624′ | | |
| 67197_r_a | Cluster Incl. AI187329:qe26c02.x1 Homo sapiens cDNA, | 5.794363406 | 0.031677 |
| | 3 end /clone = IMAGE-1740098 /clone_end = 3′ | | |
| | /gb = AI187329 /gi = 3737967 /ug = Hs.182051 /len = 483′ | | |
| 77572_at | Cluster Incl. AI740961:wg18g09.x1 Homo sapiens cDNA, | 5.69131656 | 0.000179 |
| | 3 end /clone = IMAGE-2365504 /clone_end = 3′ | | |
| | /gb = AI740961 /gi = 5109249 /ug = Hs.179791 /len = 538′ | | |
| 88041_at | Cluster Incl. AI984261:wz57h06.x1 Homo sapiens cDNA, | 5.620260813 | 0.000122 |
| | 3 end /clone = IMAGE-2562203 /clone_end = 3′ | | |
| | /gb = AI984261 /gi = 5811480 /ug = Hs.238551 /len = 486′ | | |
| 85197_at | Cluster Incl. H53229:yq84e08.r1 Homo sapiens cDNA, | 5.587699119 | 0.001786 |
| | 5 end /clone = IMAGE-202502 /clone_end = 5′ | | |
| | /gb = H53229 /gi = 993376 /ug = Hs.183775 /len = 529′ | | |
| 90566_at | Cluster Incl. AI831961:wh90b03.x1 Homo sapiens cDNA, | 5.56730113 | 0.023455 |
| | 3 end /clone = IMAGE-2387981 /clone_end = 3′ | | |
| | /gb = AI831961 /gi = 5452632 /ug = Hs.153850 /len = 496′ | | |
| 84480_at | Cluster Incl. AI821052:nr06c07.y5 Homo sapiens cDNA, | 5.510588082 | 0.003876 |
| | 5 end /clone = IMAGE-1161036 /clone_end = 5′ | | |
| | /gb = AI821052 /gi = 5440131 /ug = Hs.168974 /len = 579′ | | |
| 87888_s_{—} | Cluster Incl. AI611162:tw39g10.x1 Homo sapiens cDNA, | 5.340707416 | 0.001304 |
| | 3 end /clone = IMAGE-2262114 /clone_end = 3′ | | |
| | /gb = AI611162 /gi = 4620329 /ug = Hs.236173 /len = 513′ | | |
| 91873_s_{—} | Cluster Incl. AI690773:tx99c07.x1 Homo sapiens cDNA, | 5.339491382 | 0.016636 |
| | 3 end /clone = IMAGE-2277708 /clone_end = 3′ | | |
| | /gb = AI690773 /gi = 4902075 /ug = Hs.133294 /len = 368′ | | |
| 73036_r_a | Cluster Incl. AA602984:np31e11.s1 Homo sapiens cDNA, | 5.273912907 | 0.0033 |
| | 3 end /clone = IMAGE-1117964 /clone_end = 3′ | | |
| | /gb = AA602984 /gi = 2436845 /ug = Hs.234035 /len = 487′ | | |
| 76463_at | Cluster Incl. AW007116:wt09d07.x1 Homo sapiens cDNA, | 5.263622629 | 0.000009 |
| | 3 end /clone = IMAGE-2506957 /clone_end = 3′ | | |
| | /gb = AW007116 /gi = 5855894 /ug = Hs.127007 /len = 521′ | | |
| 82385_at | Cluster Incl. AA534591:nf81b04.s1 Homo sapiens cDNA, | 5.252002344 | 0.011367 |
| | 3 end /clone = IMAGE-926287 /clone_end = 3′ | | |
| | /gb = AA534591 /gi = 2278844 /ug = Hs.135657 /len = 492′ | | |
| 82482_r_a | Cluster Incl. H07125:yl82e05.s1 Homo sapiens cDNA, | 5.144291508 | 0.005336 |
| | 3 end /clone = IMAGE-44497 /clone_end = 3′ | | |
| | /gb = H07125 /gi = 870657 /ug = Hs.138418 /len = 526′ | | |
| 69197_r_a | Cluster Incl. AA508552:nh67b08.s1 Homo sapiens cDNA | 5.137532156 | 0.001736 |
| | /clone = IMAGE-957399 /gb = AA508552 /gi = 2246055 | | |
| | /ug = Hs.195839 /len = 315 | | |
| 67549_at | Cluster Incl. AA001232:zh82g06.r1 Homo sapiens cDNA, | 5.065760716 | 0.00041 |
| | 5 end /clone = IMAGE-427834 /clone_end = 5′ | | |
| | /gb = AA001232 /gi = 1437355 /ug = Hs.186593 /len = 473′ | | |
| 70873_at | Cluster Incl. AI807627:wf49b09.x1 Homo sapiens cDNA, | 5.04405858 | 0.000025 |
| | 3 end /clone = IMAGE-2358905 /clone_end = 3′ | | |
| | /gb = AI807627 /gi = 5394193 /ug = Hs.209978 /len = 530′ | | |
| 73800_at | Cluster Incl. AI890347:wm84b05.x1 Homo sapiens cDNA, | 4.884088199 | 0.000153 |
| | 3 end /clone = IMAGE-2442609 /clone_end = 3′ | | |
| | /gb = AI890347 /gi = 5595511 /ug = Hs.124380 /len = 620′ | | |
| 70170_f_a | Cluster Incl. AI251913:qu76a04.x1 Homo sapiens cDNA, | 4.811398521 | 0.002454 |
| | 3 end /clone = IMAGE-1977966 /clone_end = 3′ | | |
| | /gb = AI251913 /gi = 3848442 /ug = Hs.203582 /len = 289′ | | |
| 69170_at | Cluster Incl. AI990825:ws32c05.x1 Homo sapiens cDNA, | 4.779712151 | 0.033749 |
| | 3 end /clone = IMAGE-2498888 /clone_end = 3′ | | |
| | /gb = AI990825 /gi = 5837706 /ug = Hs.195398 /len = 549′ | | |
| 74042_g_{—} | Cluster Incl. AI732208:ne53h01.x5 Homo sapiens cDNA, | 4.765311344 | 0.006798 |
| | 3 end /clone = IMAGE-901105 /clone_end = 3′ | | |
| | /gb = AI732208 /gi = 5053321 /ug = Hs.126783 /len = 536′ | | |
| 88923_at | Cluster Incl. AW025183:wu72c05.x1 Homo sapiens cDNA, | 4.616589303 | 0.000839 |
| | 3 end /clone = IMAGE-990843 /clone_end = 3′ | | |
| | /gb = AW025183 /gi = 5878713 /ug = Hs.127680 /len = 504′ | | |
| 89983_at | Cluster Incl. AI762857:wi63b04.x1 Homo sapiens cDNA, | 4.571238045 | 0.004145 |
| | 3 end /clone = IMAGE-2397967 /clone_end = 3′ | | |
| | /gb = AI762857 /gi = 5178524 /ug = Hs.123177 /len = 554′ | | |
| 88043_g_{—} | Cluster Incl. AI984261:wz57h06.x1 Homo sapiens cDNA, | 4.56014179 | 0.001077 |
| | 3 end /clone = IMAGE-2562203 /clone_end = 3′ | | |
| | /gb = AI984261 /gi = 5811480 /ug = Hs.238551 /len = 486′ | | |
| 82985_at | Cluster Incl. AI817781:wk40b08.x1 Homo sapiens cDNA, | 4.501873026 | 0.038048 |
| | 3 end /clone = IMAGE-2417847 /clone_end = 3′ | | |
| | /gb = AI817781 /gi = 5436860 /ug = Hs.146905 /len = 470′ | | |
| 67667_at | Cluster Incl. R02826:ye80g11.s1 Homo sapiens cDNA, | 4.458109865 | 0.008755 |
| | 3 end /clone = IMAGE-124100 /clone_end = 3′ | | |
| | /gb = R02826 /gi = 752562 /ug = Hs.187456 /len = 371′ | | |
| 74965_at | Cluster Incl. AI075710:oz24f10.x1 Homo sapiens cDNA, | 4.433933192 | 0.000294 |
| | 3 end /clone = IMAGE-1676299 /clone_end = 3′ | | |
| | /gb = AI075710 /gi = 3404888 /ug = Hs.133988 /len = 458′ | | |
| 81447_at | Cluster Incl. AI540087:td09b08.x1 Homo sapiens cDNA, | 4.336850383 | 0.034037 |
| | 3 end /clone = IMAGE-2075127 /clone_end = 3′ | | |
| | /gb = AI540087 /gi = 4457460 /ug = Hs.120243 /len = 489′ | | |
| 85757_at | Cluster Incl. AI635504:ts95a01.x1 Homo sapiens cDNA, | 4.307200411 | 0.001228 |
| | 3 end /clone = IMAGE-2238984 /clone_end = 3′ | | |
| | /gb = AI635504 /gi = 4686834 /ug = Hs.191229 /len = 496′ | | |
| 86946_s_{—} | Cluster Incl. AL044106:DKFZp434M0528_s1 Homo sapiens cDNA, | 4.274201098 | 0.000009 |
| | 3 end /clone = DKFZp434M0528 /clone_end = 3′ | | |
| | /gb = AL044106 /gi = 5432332 /ug = Hs.209359 /len = 574′ | | |
| 78442_at | Cluster Incl. AI819340:wg61a01.x1 Homo sapiens cDNA, | 4.255225315 | 0.03273 |
| | 3 end /clone = IMAGE-2369544 /clone_end = 3′ | | |
| | /gb = AI819340 /gi = 5438419 /ug = Hs.13561 /len = 539′ | | |
| 77354_at | Cluster Incl. AI935827:wo51c06.x1 Homo sapiens cDNA, | 4.221724462 | 0.000009 |
| | 3 end /clone = IMAGE-2458858 /clone_end = 3′ | | |
| | /gb = AI935827 /gi = 5674697 /ug = Hs.161791 /len = 533′ | | |
| 73264_f_a | Cluster Incl. AW025939:wu09c06.x1 Homo sapiens cDNA, | 4.184011102 | 0.012722 |
| | 3 end /clone = IMAGE-2516458 /clone_end = 3′ | | |
| | /gb = AW025939 /gi = 5879469 /ug = Hs.236356 /len = 326′ | | |
| 82129_at | Cluster Incl. AI914323:wd77d01.x1 Homo sapiens cDNA, | 4.163620114 | 0.007416 |
| | 3 end /clone = IMAGE-2337601 /clone_end = 3′ | | |
| | /gb = AI914323 /gi = 5634178 /ug = Hs.131175 /len = 543′ | | |
| 77848_f_a | Cluster Incl. AA018663:ze53f11.s1 Homo sapiens cDNA, | 4.141843067 | 0.001411 |
| | 3 end /clone = IMAGE-362733 /clone_end = 3′ | | |
| | /gb = AA018663 /gi = 1481928 /ug = Hs.204007 /len = 496′ | | |
| 82858_at | Cluster Incl. AI754693:cr28h02.x1 Homo sapiens cDNA, | 4.138901583 | 0.000443 |
| | 3 end /clone = HBMSC_cr28h02 /clone_end = 3′ | | |
| | /gb = AI754693 /gi = 5132957 /ug = Hs.145968 /len = 515′ | | |
| 87558_f_a | Cluster Incl. W85857:zh52f11.s1 Homo sapiens cDNA, | 4.078971064 | 0.015864 |
| | 3 end /clone = IMAGE-415725 /clone_end = 3′ | | |
| | /gb = W85857 /gi = 1398355 /ug = Hs.226604 /len = 446′ | | |
| 76571_at | Cluster Incl. AI820585:os03a03.x5 Homo sapiens cDNA, | 4.07737079 | 0.00185 |
| | 3 end /clone = IMAGE-1604236 /clone end = 3′ | | |
| | /gb = AI820585 /gi = 5439664 /ug = Hs.129107 /len = 538′ | | |
| AFFX-Bio | J04423 E coli bioB gene biotin synthetase (-5, -M, -3 represent | 4.074822599 | 0.006154 |
| | transcript regions 5 prime, Middle, and 3 prime respectively) | | |
| 77364_at | Cluster Incl. AI676059:wc04g08.x1 Homo sapiens cDNA, | 4.032646341 | 0.027423 |
| | 3 end /clone = IMAGE-2314238 /clone_end = 3′ | | |
| | /gb = AI676059 /gi = 4876539 /ug = Hs.163900 /len = 562′ | | |
| 79405_at | Cluster Incl. AA740146:ob26a07.s1 Homo sapiens cDNA | 4.01839873 | 0.013599 |
| | /clone = IMAGE-1324788 /gb = AA740146 /gi = 2778738 | | |
| | /ug = Hs.192397 /len = 460 | | |
| 90224_at | Cluster Incl. AI276259:ql65f03.x1 Homo sapiens cDNA, | 4.004273997 | 0.008055 |
| | 3 end /clone = IMAGE-1877213 /clone_end = 3′ | | |
| | /gb = AI276259 /gi = 3898533 /ug = Hs.13337 /len = 505′ | | |
| 87802_at | Cluster Incl. AA613291:no39d04.s1 Homo sapiens cDNA, | 3.985917634 | 0.004647 |
| | 3 end /clone = IMAGE-1103047 /clone_end = 3′ | | |
| | /gb = AA613291 /gi = 2464329 /ug = Hs.234346 /len = 482′ | | |
| 87684_at | Cluster Incl. AW007058:ws49f05.x1 Homo sapiens cDNA, | 3.965226206 | 0.017504 |
| | 3 end /clone = IMAGE-2500545 /clone_end = 3′ | | |
| | /gb = AW007058 /gi = 5855836 /ug = Hs.232261 /len = 345′ | | |
| 82496_at | Cluster Incl. AA832417:oc99d03.s1 Homo sapiens cDNA, | 3.933719194 | 0.022181 |
| | 3 end /clone = IMAGE-1357829 /clone_end = 3′ | | |
| | /gb = AA832417 /gi = 2905516 /ug = Hs.139650 /len = 402′ | | |
| 89253_at | Cluster Incl:AI653050:wb42h02.x1 Homo sapiens cDNA, | 3.928266882 | 0.000754 |
| | 3 end /clone = IMAGE-2308371 /clone_end = 3′ | | |
| | /gb = AI653050 /gi = 4737029 /ug = Hs.162717 /len = 698′ | | |
| 78846_at | Cluster Incl. AA931876:oo58f04.s1 Homo sapiens cDNA, | 3.910353677 | 0.005377 |
| | 3 end /clone = IMAGE-1570399 /clone_end = 3′ | | |
| | /gb = AA931876 /gi = 3086262 /ug = Hs.181245 /len = 489′ | | |
| 88840_at | Cluster Incl. AI393205:tg14b07.x1 Homo sapiens cDNA, | 3.900166459 | 0.01782 |
| | 3 end /clone = IMAGE-2108725 /clone_end = 3′ | | |
| | /gb = AI393205 /gi = 4222752 /ug = Hs.126265 /len = 614′ | | |
| 86478_at | Cluster Incl. AI741566:wg21g12.x1 Homo sapiens cDNA, | 3.89585652 | 0.000249 |
| | 3 end /clone = IMAGE-2365798 /clone_end = 3′ | | |
| | /gb = AI741566 /gi = 5109854 /ug = Hs.200099 /len = 614′ | | |
| 89973_at | Cluster Incl. AI656161:tt43g10.x1 Homo sapiens cDNA, | 3.853888055 | 0.003549 |
| | 3 end /clone = IMAGE-2243586 /clone_end = 3′ | | |
| | /gb = AI656161 /gi = 4740140 /ug = Hs.122810 /len = 542′ | | |
| 69247_at | Cluster Incl. AI984074:wz56c02.x1 Homo sapiens cDNA, | 3.846943175 | 0.002703 |
| | 3 end /clone = IMAGE-2562050 /clone_end = 3′ | | |
| | /gb = AI984074 /gi = 5811293 /ug = Hs.196398 /len = 614′ | | |
| 77225_at | Cluster Incl. AI740516:wg16a11.x1 Homo sapiens cDNA, | 3.845535109 | 0.003211 |
| | 3 end /clone = IMAGE-2365244 /clone_end = 3′ | | |
| | /gb = AI740516 /gi = 5108804 /ug = Hs.152812 /len = 591′ | | |
| 90524_at | Cluster Incl. AI889361:wm98b08.x1 Homo sapiens cDNA, | 3.806709116 | 0.032956 |
| | 3 end /clone = IMAGE-2443959 /clone_end = 3′ | | |
| | /gb = AI889361 /gi = 5594525 /ug = Hs.148289 /len = 486′ | | |
| 72388_at | Cluster Incl. AA608808:af04e10.s1 Homo sapiens cDNA, | 3.793457244 | 0.000011 |
| | 3 end /clone = IMAGE-1030698 /clone_end = 3′ | | |
| | /gb = AA608808 /gi = 2457236 /ug = Hs.225118 /len = 393′ | | |
| 84407_r_a | Cluster Incl. AI032906:ox19e08.s1 Homo sapiens cDNA, | 3.767444971 | 0.00167 |
| | 3 end /clone = IMAGE-1656806 /clone_end = 3′ | | |
| | /gb = AI032906 /gi = 3253859 /ug = Hs.167800 /len = 471′ | | |
| 75621_at | Cluster Incl. AI935035:wd17g08.x1 Homo sapiens cDNA, | 3.764029378 | 0.048154 |
| | 3 end /clone = IMAGE-2328446 /clone_end = 3′ | | |
| | /gb = AI935035 /gi = 5673905 /ug = Hs.145656 /len = 483′ | | |
| 82539_at | Cluster Incl. AI935586:wo98c05.x1 Homo sapiens cDNA, | 3.761588053 | 0.004131 |
| | 3 end /clone = IMAGE-2463368 /clone_end = 3′ | | |
| | /gb = AI935586 /gi = 5674456 /ug = Hs.142869 /len = 516′ | | |
| 90265_at | Cluster Incl. AW050627:wz19e03.x1 Homo sapiens cDNA, | 3.751652632 | 0.000052 |
| | 3 end /clone = IMAGE-2558524 /clone_end = 3′ | | |
| | /gb = AW050627 /gi = 5912897 /ug = Hs.135183 /len = 477′ | | |
| 81787_i_a | Cluster Incl. AI425019:tg50c11.x1 Homo sapiens cDNA, | 3.729431315 | 0.020585 |
| | 3 end /clone = IMAGE-2112212 /clone_end = 3′ | | |
| | /gb = AI425019 /gi = 4270950 /ug = Hs.124280 /len = 509′ | | |
| 81953_at | Cluster Incl. AI733253:oo60c07.x5 Homo sapiens cDNA, | 3.673677735 | 0.001218 |
| | 3 end /clone = IMAGE-1570572 /clone_end = 3′ | | |
| | /gb = AI733253 /gi = 5054366 /ug = Hs.126860 /len = 531′ | | |
| 89731_at | Cluster Incl. AI017165:ou28a11.x1 Homo sapiens cDNA, | 3.660093991 | 0.026076 |
| | 3 end /clone = IMAGE-1627580 /clone_end = 3′ | | |
| | /gb = AI017165 /gi = 3231501 /ug = Hs.104573 /len = 461′ | | |
| 88214_at | Cluster Incl. AI473909:ti68d07.x1 Homo sapiens cDNA, | 3.657078333 | 0.001554 |
| | 3 end /clone = IMAGE-2137165 /clone_end = 3′ | | |
| | /gb = AI473909 /gi = 4326954 /ug = Hs.239658 /len = 402′ | | |
| 71534_at | Cluster Incl. AA584378:nn77b04.s1 Homo sapiens cDNA, | 3.64418462 | 0.000698 |
| | 3 end /clone = IMAGE-1089871 /clone_end = 3′ | | |
| | /gb = AA584378 /gi = 2368987 /ug = Hs.219473 /len = 404′ | | |
| 91313_at | Cluster Incl. AA131041:zo16e03.s1 Homo sapiens cDNA, | 3.641675284 | 0.003603 |
| | 3 end /clone = IMAGE-587068 /clone_end = 3′ | | |
| | /gb = AA131041 /gi = 1692593 /ug = Hs.82554 /len = 539′ | | |
| 74698_at | Cluster Incl. AW003102:wr03e03.x1 Homo sapiens cDNA, | 3.636444818 | 0.004914 |
| | 3 end /clone = IMAGE-2480476 /clone_end = 3′ | | |
| | /gb = AW003102 /gi = 5849940 /ug = Hs.131886 /len = 512′ | | |
| 76293_at | Cluster Incl. AI632750:wa33g09.x1 Homo sapiens cDNA, | 3.614166118 | 0.001198 |
| | 3 end /clone = IMAGE-2299936 /clone_end = 3′ | | |
| | /gb = AI632750 /gi = 4684080 /ug = Hs.121233 /len = 519′ | | |
| 67246_at | Cluster Incl. AI040381:ox16c09.x1 Homo-sapienscDNA, | 3.607391355 | 0.010336 |
| | 3 end /clone = IMAGE-1656496 /clone_end = 3′ | | |
| | /gb = AI040381 /gi = 3279575 /ug = Hs.182430 /len = 535′ | | |
| 76041_g_{—} | Cluster Incl. AI492574:ti29f08.x1 Homo sapiens cDNA, | 3.554944957 | 0.001981 |
| | 3 end /clone = IMAGE-2131911 /clone_end = 3′ | | |
| | /gb = AI492574 /gi = 4393577 /ug = Hs.234190 /len = 535′ | | |
| 91762_at | Cluster Incl. AA496243:zx70g12.s1 Homo sapiens cDNA, | 3.524153823 | 0.004236 |
| | 3 end /clone = IMAGE-796870 /clone_end = 3′ | | |
| | /gb = AA496243 /gi = 2229564 /ug = Hs.84640 /len = 579′ | | |
| 76137_at | Cluster Incl. AA639753:nq82c12.s1 Homo sapiens cDNA, | 3.523452759 | 0.005364 |
| | 3 end /clone = IMAGE-1158838 /clone_end = 3′ | | |
| | /gb = AA639753 /gi = 2563532 /ug = Hs.116462 /len = 645′ | | |
| 87456_at | Cluster Incl. AI990471:ws40a05.x1 Homo sapiens cDNA, | 3.511142884 | 0.000815 |
| | 3 end /clone = IMAGE-2499632 /clone_end = 3′ | | |
| | /gb = AI990471 /gi = 5837352 /ug = Hs.224630 /len = 497′ | | |
| 80760_i_a | Cluster Incl. AI888428:wn20h08.x1 Homo sapiens cDNA, | 3.509284285 | 0.024008 |
| | 3 end /clone = IMAGE-2446047 /clone_end = 3′ | | |
| | /gb = AI888428 /gi = 5593515 /ug = Hs.5402 /len = 395′ | | |
| 88678_at | Cluster Incl. AF121164:AF121164 Homo sapiens cDNA | 3.494772543 | 0.001841 |
| | /clone = PFGTG1-2 /gb = AF121164 /gi = 4886240 | | |
| | /ug = Hs.120879 /len = 654 | | |
| 90509_at | Cluster Incl. AI968197:wu14c07.x1 Homo sapiens cDNA, | 3.477306776 | 0.010461 |
| | 3 end /clone = IMAGE-2516940 /clone_end = 3′ | | |
| | /gb = AI968197 /gi = 5765015 /ug = Hs.14587 /len = 541′ | | |
| 73188_s_{—} | Cluster Incl. AA948682:oq49b11.s1 Homo sapiens cDNA, | 3.462332464 | 0.003352 |
| | 3 end /clone = IMAGE-1589661 /clone_end = 3′ | | |
| | /gb = AA948682 /gi = 3109935 /ug = Hs.235393 /len = 411′ | | |
| 74964_at | Cluster Incl. AA045735:zk81g12.s1 Homo sapiens cDNA, | 3.461482296 | 0.000176 |
| | 3 end /clone = IMAGE-489286 /clone_end = 3′ | | |
| | /gb = AA045735 /gi = 1525629 /ug = Hs.133961 /len = 399′ | | |
| 89166_at | Cluster Incl. AI953998:wx78c12.x1 Homo sapiens cDNA, | 3.455539647 | 0.000616 |
| | 3 end /clone = IMAGE-2549782 /clone_end = 3′ | | |
| | /gb = AI953998 /gi = 5746308 /ug = Hs.152510 /len = 679′ | | |
| 90732_at | Cluster Incl. W45137:zc81g05.s1 Homo sapiens cDNA, | 3.437671708 | 0.001008 |
| | 3 end /clone = IMAGE-328760 /clone_end = 3′ | | |
| | /gb = W45137 /gi = 1329442 /ug = Hs.183576 /len = 481′ | | |
| 69037_r_a | Cluster Incl. AA730798:nw49b12.s1 Homo sapiens cDNA | 3.434365508 | 0.02404 |
| | /clone = IMAGE-1249919 /gb = AA730798 /gi = 2752002 | | |
| | /ug = Hs.194135 /len = 310 | | |
| 84983_at | Cluster Incl. AI741880:wg48b12.x1 Homo sapiens cDNA, | 3.429078419 | 0.022269 |
| | 3 end /clone = IMAGE-2368319 /clone_end = 3′ | | |
| | /gb = AI741880 /gi = 5110255 /ug = Hs.179891 /len = 454′ | | |
| 92067_s_{—} | Cluster Incl. AI348900:ta98e05.x1 Homo sapiens cDNA, | 3.420352947 | 0.031666 |
| | 3 end /clone = IMAGE-2052128 /clone_end = 3′ | | |
| | /gb = AI348900 /gi = 4086106 /ug = Hs.25199 /len = 420′ | | |
| 80859_at | Cluster Incl. AI684877:wa86f08.x1 Homo sapiens cDNA, | 3.414213425 | 0.000763 |
| | 3 end /clone = IMAGE-2303079 /clone_end = 3′ | | |
| | /gb = AI684877 /gi = 4896171 /ug = Hs.105489 /len = 768′ | | |
| 75954_at | Cluster Incl. AI978606:wr57b07.x1 Homo sapiens cDNA, | 3.410569117 | 0.019697 |
| | 3 end /clone = IMAGE-2491765 /clone_end = 3′ | | |
| | /gb = AI978606 /gi = 5803636 /ug = Hs.201615 /len = 585′ | | |
| 74159_at | Cluster Incl. AA534390:nf79h07.s1 Homo sapiens cDNA, | 3.401564012 | 0.029962 |
| | 3 end /clone = IMAGE-926173 /clone_end = 3′ | | |
| | /gb = AA534390 /gi = 2278643 /ug = Hs.127762 /len = 588′ | | |
| 76075_at | Cluster Incl. AW014657:UI-H-BI0p-aaw-b-11-0-UI.s1 Homo sapiens cDNA, | 3.375380748 | 0.006718 |
| | 3 end /clone = IMAGE-2710628 /clone_end = 3′ | | |
| | /gb = AW014557 /gi = 5863314 /ug = Hs.112420 /len = 618′ | | |
| 90203_at | Cluster Incl. AI819204:wj32f08.x1 Homo sapiens cDNA, | 3.345450186 | 0.000782 |
| | 3 end /clone = IMAGE-2404551 /clone_end = 3′ | | |
| | /gb = AI819204 /gi = 5438283 /ug = Hs.132743 /len = 514′ | | |
| 89795_at | Cluster Incl. AL040892:DKFZp434I1715_s1 Homo sapiens cDNA, | 3.343086565 | 0.01438 |
| | 3 end /clone = DKFZp434I1715 /clone end = 3′ | | |
| | /gb = AL040892 /gi = 5409837 /ug = Hs.114173 /len = 717′ | | |
| 86866_r_a | Cluster Incl. AI765629:wi82e01.x1 Homo sapiens cDNA, | 3.315297451 | 0.010622 |
| | 3 end /clone = IMAGE-2399832 /clone_end = 3′ | | |
| | /gb = AI765629 /gi = 5232138 /ug = Hs.207278 /len = 332′ | | |
| 87205_at | Cluster Incl. AA419233:zv35f05.s1 Homo sapiens cDNA, | 3.312326275 | 0.004096 |
| | 3 end /clone = IMAGE-755649 /clone_end = 3′ | | |
| | /gb = AA419233 /gi = 2078997 /ug = Hs.215682 /len = 423′ | | |
| 78393_at | Cluster Incl. AA725820:ai25b05.s1 Homo sapiens cDNA, | 3.311882393 | 0.021412 |
| | 3 end /clone = 1343793 /clone_end = 3′ | | |
| | /gb = AA725820 /gi = 2743527 /ug = Hs.123426 /len = 472′ | | |
| 74179_at | Cluster Incl. AI889959:wm65f08.x1 Homo sapiens cDNA, | 3.305411403 | 0.004734 |
| | 3 end /clone = IMAGE-2440839 /clone_end = 3′ | | |
| | /gb = AI889959 /gi = 5595123 /ug = Hs.127797 /len = 479′ | | |
| 89306_at | Cluster Incl. AI934965:wd17a03.x1 Homo sapiens cDNA, | 3.300123522 | 0.020207 |
| | 3 end /clone = IMAGE-2328364 /clone_end = 3′ | | |
| | /gb = AI934965 /gi = 5673835 /ug = Hs.181261 /len = 588′ | | |
| 69031_at | Cluster Incl. AI686521:tu34g02.x1 Homo sapiens cDNA, | 3.296015126 | 0.011834 |
| | 3 end /clone = IMAGE-2252978 /clone_end = 3′ | | |
| | /gb = AI686521 /gi = 4897815 /ug = Hs.194118 /len = 597′ | | |
| 88302_at | Cluster Incl. AI082708:ox59f01.s1 Homo sapiens cDNA, | 3.287284047 | 0.009528 |
| | 3 end /clone = IMAGE-1660633 /clone_end = 3′ | | |
| | /gb = AI082708 /gi = 3419500 /ug = Hs.31588 /len = 369′ | | |
| 78861_f_a | Cluster Incl. H18887:yn52d11.s1 Homo sapiens cDNA, | 3.283264496 | 0.000041 |
| | 3 end /clone = IMAGE-172053 /clone end = 3′ | | |
| | /gb = H18887 /gi = 885127 /ug = Hs.181836 /len = 401′ | | |
| 81733_at | Cluster Incl. AI632972:tx55h08.x1 Homo sapiens cDNA, | 3.270101441 | 0.001223 |
| | 3 end /clone = IMAGE-2273535 /clone_end = 3′ | | |
| | /gb = AI632972 /gi = 4684302 /ug = Hs.123370 /len = 562′ | | |
| 90603_at | Cluster Incl. AI916544:wa26h03.x1 Homo sapiens cDNA, | 3.268418769 | 0.013059 |
| | 3 end /clone = IMAGE-2299253 /clone_end = 3′ | | |
| | /gb = AI916544 /gi = 5636399 /ug = Hs.158549 /len = 475′ | | |
| 71228_at | Cluster Incl. AI919493:tp22a03.x1 Homo sapiens cDNA, | 3.264815022 | 0.016554 |
| | 3 end /clone = IMAGE-2188492 /clone_end = 3′ | | |
| | /gb = AI919493 /gi = 5639348 /ug = Hs.212925 /len = 320′ | | |
| 68060_at | Cluster Incl. AI681180:tx44h02.x1 Homo sapiens cDNA, | 3.258903714 | 0.003186 |
| | 3 end /clone = IMAGE-2272467 /clone_end = 3′ | | |
| | /gb = AI681180 /gi = 4891362 /ug = Hs.189394 /len = 489′ | | |
| 81731_at | Cluster Incl. AA811371:6b82b10.s1 Homo sapiens cDNA | 3.254465131 | 0.008956 |
| | /clone = IMAGE-1337851 /gb = AA811371 | | |
| | /gi = 2880982 /ug = Hs.123362 /len = 482 | | |
| 80538_s_{—} | Cluster Incl. AW020375:df08h01.y1 Homo sapiens cDNA, | 3.238426465 | 0.000033 |
| | 5 end /clone = IMAGE-2483185 /clone_end = 5′ | | |
| | /gb = AW020375 /gi = 5873905 /ug = Hs.238653 /len = 339′ | | |
| 83130_at | Cluster Incl. AI683864:tw54a08.x1 Homo sapiens cDNA, | 3.237963905 | 0.002982 |
| | 3 end /clone = IMAGE-2263478 /clone_end = 3′ | | |
| | /gb = AI683864 /gi = 4894046 /ug = Hs.149264 /len = 489′ | | |
| 86750_at | Cluster Incl. AI888991:wj16b04.x1 Homo sapiens cDNA, | 3.22255218 | 0.002743 |
| | 3 end /clone = IMAGE-2402959 /clone_end = 3′ | | |
| | /gb = AI888991 /gi = 5594155 /ug = Hs.204044 /len = 528′ | | |
| 82094_i_a | Cluster Incl. AI810266:wb86h07.x1 Homo sapiens cDNA, | 3.218521352 | 0.031896 |
| | 3 end /clone = IMAGE-2312605 /clone_end = 3′ | | |
| | /gb = AI810266 /gi = 5396832 /ug = Hs.130853 /len = 553′ | | |
| 80080_at | Cluster Incl. AI916889:wb46g08.x1 Homo sapiens cDNA, | 3.213809716 | 0.003436 |
| | 3 end /clone = IMAGE-2308766 /clone_end = 3′ | | |
| | /gb = AI916889 /gi = 5636744 /ug = Hs.213436 /len = 480′ | | |
| 76779_at | Cluster Incl. AW014285:UI-H-BI0-aak-h-03-0-UI.s1 Homo sapiens cDNA, | 3.210119895 | 0.012957 |
| | 3 end /clone = IMAGE-2709748 /clone_end = 3′ | | |
| | /gb = AW014285 /gi = 5863042 /ug = Hs.133925 /len = 493′ | | |
| 88103_s_{—} | Cluster Incl. AW007018:ws49b10.x1 Homo sapiens cDNA, | 3.190608857 | 0.030944 |
| | 3 end /clone = IMAGE-2500507 /clone_end = 3′ | | |
| | /gb = AW007018 /gi = 5855796 /ug = Hs.239125 /len = 524′ | | |
| 88452_at | Cluster Incl. AI625821:ty65b01.x1 Homo sapiens cDNA, | 3.188760476 | 0.004402 |
| | 3 end /clone = IMAGE-2283913 /clone_end = 3′ | | |
| | /gb = AI625821 /gi = 4650752 /ug = Hs.101320 /len = 647′ | | |
| 78788_at | Cluster Incl. AI674088:wd18a08.x1 Homo sapiens cDNA, | 3.185705139 | 0.013029 |
| | 3 end /clone = IMAGE-2328470 /clone_end = 3′ | | |
| | /gb = AI674088 /gi = 4874568 /ug = Hs.8122 /len = 576′ | | |
| 75722_at | Cluster Incl. AA766775:oa35d08.s1 Homo sapiens cDNA | 3.176732451 | 0.004027 |
| | /clone = IMAGE-1306959 /gb = AA766775 | | |
| | /gi = 2818013 /ug = Hs.163195 /len = 440 | | |
| 85325_at | Cluster Incl. W89075:zh73a10.s1 Homo sapiens cDNA, | 3.174084701 | 0.001733 |
| | 3 end /clone = IMAGE-417690 /clone_end = 3′ | | |
| | /gb = W89075 /gi = 1404168 /ug = Hs.186182 /len = 456′ | | |
| 72349_at | Cluster Incl. D29047:HUMNK041 Homo sapiens cDNA | 3.169517307 | 0.000389 |
| | /clone = 041 /gb = D29047 /gi = 598980 | | |
| | /ug = Hs.224812 /len = 418 | | |
| 85939_at | Cluster Incl. AI694139:wd89c05.x1 Homo sapiens cDNA, | 3.161612049 | 0.02562 |
| | 3 end /clone = IMAGE-2338760 /clone_end = 3′ | | |
| | /gb = AI694139 /gi = 4971479 /ug = Hs.192855 /len = 330′ | | |
| 79188_r_a | Cluster Incl. T72642:yd19d02.s1 Homo sapiens cDNA, | 3.154189137 | 0.009489 |
| | 3 end /clone = IMAGE-108675 /clone_end = 3′ | | |
| | /gb = T72642 /gi = 689317 /ug = Hs.191118 /len = 428′ | | |
| 87790_at | Cluster Incl. AW015585:UI-H-BI0p-aau-h-09-0-UI.s1 Homo sapiens cDNA, | 3.153789914 | 0.000844 |
| | 3 end /clone = IMAGE-2710888 /clone_end = 3′ | | |
| | /gb = AW015585 /gi = 5864342 /ug = Hs.234252 /len = 630′ | | |
| 89603_at | Cluster Incl. R77227:yi72c10.s1 Homo sapiens cDNA, | 3.137562053 | 0.004856 |
| | 3 end /clone = IMAGE-144786 /clone_end = 3′ | | |
| | /gb = R77227 /gi = 851859 /ug = Hs.237156 /len = 449′ | | |
| 84981_r_a | Cluster Incl. T54146:ya92h02.s1 Homo sapiens cDNA, | 3.123046617 | 0.031758 |
| | 3 end /clone = IMAGE-69171 /clone_end = 3′ | | |
| | /gb = T54146 /gi = 656007 /ug = Hs.179847 /len = 559′ | | |
| 81355_at | Cluster Incl. AA946617:oq38e04.s1 Homo sapiens cDNA, | 3.109445352 | 0.008631 |
| | 3 end /clone = IMAGE-1588638 /clone_end = 3′ | | |
| | /gb = AA946617 /gi = 3110012 /ug = Hs.119183 /len = 525′ | | |
| 87837_at | Cluster Incl. AI656113:tt38c07.x1 Homo sapiens cDNA, | 3.097465077 | 0.0051 |
| | 3 end /clone = IMAGE-2243052 /clone_end = 3′ | | |
| | /gb = AI656113 /gi = 4740092 /ug = Hs,235408 /len = 436′ | | |
| 91875_s_{—} | Cluster Incl. AI053741:qi69h07.x1 Homo sapiens cDNA, | 3.095993734 | 0.017489 |
| | 3 end /clone = IMAGE-1861789 /clone_end = 3′ | | |
| | /gb = AI053741 /gi = 3321528 /ug = Hs.133294 /len = 337′ | | |
| 83569_at | Cluster Incl. AI823960:wj21d05.x1 Homo sapiens cDNA, | 3.088550292 | 0.044324 |
| | 3 end /clone = IMAGE-2403465 /clone_end = 3′ | | |
| | /gb = AI823960 /gi = 5444631 /ug = Hs.155718 /len = 420′ | | |
| 83331_at | Cluster Incl. AA568924:nm23b06.s1 Homo sapiens cDNA, | 3.075127644 | 0.007888 |
| | 3 end /clone = IMAGE-1060979 /clone_end = 3′ | | |
| | /gb = AA568924 /gi = 2341978 /ug = Hs.152484 /len = 475′ | | |
| 84009_at | Cluster Incl. AA218643:zq14c10.s1 Homo sapiens cDNA, | 3.073579917 | 0.000394 |
| | 3 end /clone = IMAGE-629682 /clone_end = 3′ | | |
| | /gb = AA218643 /gi = 1832735 /ug = Hs.161690 /len = 460′ | | |
| 86916_at | Cluster Incl. R24723:yg43c04.r1 Homo sapiens cDNA, | 3.069508621 | 0.001131 |
| | 5 end /clone = IMAGE-35336 /clone_end = 5′ | | |
| | /gb = R24723 /gi = 779611 /ug = Hs.208543 /len = 430′ | | |
| 84893_at | Cluster Incl. AI446168:tj07h07.x1 Homo sapiens cDNA, | 3.066866421 | 0.023278 |
| | 3 end /clone = IMAGE-2140861 /clone_end = 3′ | | |
| | /gb = AI446168 /gi = 4293320 /ug = Hs.176708 /len = 435′ | | |
| 84612_at | Cluster Incl. AI864428:wl53a09.x1 Homo sapiens cDNA, | 3.065443135 | 0.005335 |
| | 3 end /clone = IMAGE-2428600 /clone_end = 3′ | | |
| | /gb = AI864428 /gi = 5528535 /ug = Hs.170880 /len = 343′ | | |
| 73780_at | Cluster Incl. AA838748:ak03c10.s1 Homo sapiens cDNA, | 3.06404637 | 0.009987 |
| | 3 end /clone = IMAGE-1404882 /clone_end = 3′ | | |
| | /gb = AA838748 /gi = 2914860 /ug = Hs.123910 /len = 543′ | | |
| 75705_at | Cluster Incl. AI634652:wa07e10.x1 Homo sapiens cDNA, | 3.058453349 | 0.001904 |
| | 3 end /clone = IMAGE-2297418 /clone_end = 3′ | | |
| | /gb = AI634652 /gi = 4685982 /ug = Hs.160795 /len = 563′ | | |
| 70150_at | Cluster Incl. AI791593:oj57h03.y5 Homo sapiens cDNA, | 3.050814176 | 0.000397 |
| | 5 end /clone = IMAGE-1502453 /clone_end = 5′ | | |
| | /gb = AI791593 /gi = 5339309 /ug = Hs.203472 /len = 472′ | | |
| 72125_at | Cluster Incl. N73802:yz80f05.s1 Homo sapiens cDNA, | 3.037955969 | 0.009677 |
| | 3 end /clone = IMAGE-289377 /clone_end = 3′ | | |
| | /gb = N73802 /gi = 1231087 /ug = Hs.222824 /len = 385′ | | |
| 75212_at | Cluster Incl. AI365208:qx97f11.x1 Homo sapiens cDNA, | 3.027135566 | 0.003503 |
| | 3 end /clone = IMAGE-2010477 /clone_end = 3′ | | |
| | /gb = AI365208 /gi = 4124897 /ug = Hs.138480 /len = 291′ | | |
| 91678_at | Cluster Incl. AI814761:wk65d07.x1 Homo sapiens cDNA, | 3.012896674 | 0.018287 |
| | 3 end /clone = IMAGE-2420269 /clone_end = 3′ | | |
| | /gb = AI814761 /gi = 5425976 /ug = Hs.9204 /len = 497′ | | |
| 90016_at | Cluster Incl. AI961177:wq59b09.x1 Homo sapiens cDNA, | 3.00787812 | 0.013132 |
| | 3 end /clone = IMAGE-2475545 /clone_end = 3′ | | |
| | /gb = AI961177 /gi = 5753890 /ug = Hs.124832 /len = 835′ | | |
| 73002_at | Cluster Incl. AI052526:oz27f09.x1 Homo sapiens cDNA, | 2.999958702 | 0.009676 |
| | 3 end /clone = IMAGE-1676585 /clone_end = 3′ | | |
| | /gb = AI052526 /gi = 3308517 /ug = Hs.233871 /len = 290′ | | |
| 90757_at | Cluster Incl. AI985612:wr75d05.x1 Homo sapiens cDNA, | 0.329081864 | 0.008916 |
| | 3 end /clone = IMAGE-2493513 /clone_end = 3′ | | |
| | /gb = AI985612 /gi = 5812889 /ug = Hs.184484 /len = 486′ | | |
| 78336_at | Cluster Incl. AI434477:ti37d01.x1 Homo sapiens cDNA, | 0.326191217 | 0.0046 |
| | 3 end /clone = IMAGE-2132641 /clone_end = 3′ | | |
| | /gb = AI434477 /gi = 4296452 /ug = Hs.210531 /len = 421′ | | |
| 73721_at | Cluster Incl. AA813527:ai67f11.s1 Homo sapiens cDNA, | 0.325630713 | 0.0109 |
| | 3 end /clone = 1375917 /clone_end = 3′ | | |
| | /gb = AA813527 /gi = 2882212 /ug = Hs.122814 /len = 459′ | | |
| 72371_at | Cluster Incl. AI168188:oo09g11.x1 Homo sapiens cDNA, | 0.324150837 | 0.000589 |
| | 3 end /clone = IMAGE-1565732 /clone_end = 3′ | | |
| | /gb = AI168188 /gi = 3701358 /ug = Hs.225023 /len = 448′ | | |
| 71418_at | Cluster Incl. AI093188:qa98b05.x1 Homo sapiens cDNA, | 0.323684024 | 0.003636 |
| | 3 end /clone = IMAGE-1694769 /clone_end = 3′ | | |
| | /gb = AI093188 /gi = 3432164 /ug = Hs.215319 /len = 471′ | | |
| 86567_at | Cluster Incl. R19892:yg38f12.r1 Homo sapiens cDNA, | 0.320952495 | 0.00589 |
| | 5 end /clone = IMAGE-34798 /clone_end = 5′ | | |
| | /gb = R19892 /gi = 774526 /ug = Hs.201458 /len = 503′ | | |
| AFFX-HS | X00351 Human mRNA for beta-actin (_5, _M, _3 represent transcript | 0.320408458 | 0.004505 |
| | regions 5 prime, Middle, and 3 prime respectively) | | |
| 72290_r_a | Cluster Incl. AA814901:oa75g08.s1 Homo sapiens cDNA | 0.320177197 | 0.042106 |
| | /clone = IMAGE-1318142 /gb = AA814901 | | |
| | /gi = 2884497 /ug = Hs.224495 /len = 459 | | |
| 87950_at | Cluster Incl. AA313781:EST185644 Homo sapiens cDNA, | 0.320113508 | 0.001602 |
| | 5 end /clone = ATCC-109963 /clone_end = 5′ | | |
| | /gb = AA313781 /gi = 1966110 /ug = Hs.236903 /len = 599′ | | |
| 91014_at | Cluster Incl. N23781:yx35e09.r1 Homo sapiens cDNA, | 0.31964593 | 0.028338 |
| | 5 end /clone = IMAGE-263752 /clone_end = 5′ | | |
| | /gb = N23781 /gi = 1137931 /ug = Hs.226614 /len = 592′ | | |
| 70650_at | Cluster Incl. AA779712:af43h05.s1 Homo sapiens cDNA, | 0.318766299 | 0.010363 |
| | 3 end /clone = 1034457 /clone_end = 3′ | | |
| | /gb = AA779712 /gi = 2839043 /ug = Hs.208718 /len = 606′ | | |
| 69829_at | Cluster Incl. AI684559:wa84a03.x1 Homo sapiens cDNA, | 0.315917401 | 0.020149 |
| | 3 end /clone = IMAGE-2302828 /clone_end = 3′ | | |
| | /gb = AI684559 /gi = 4895853 /ug = Hs.201637 /len = 507′ | | |
| 91462_at | Cluster Incl. W26201:22c1 Homo sapiens cDNA | 0.315659043 | 0.00959 |
| | /gb = W26201 /gi = 1306612 /ug = Hs.183793 /len = 470 | | |
| 84412_at | Cluster Incl. AI525044:promrna-5.C09.r Homo sapiens cDNA, | 0.313783504 | 0.009654 |
| | 5 end /clone_end = 5′ /gb = AI525044 | | |
| | /gi = 4439179 /ug = Hs.168007 /len = 639′ | | |
| 78228_s_{—} | Cluster Incl. AI561042:tq29e02.x1 Homo sapiens cDNA, | 0.312957775 | 0.008401 |
| | 3 end /clone = IMAGE-2210234 /clone_end = 3′ | | |
| | /gb = AI561042 /gi = 4511383 /ug = Hs.239771 /len = 635′ | | |
| 67280_at | Cluster Incl. AI243125:qh26h01.x1 Homo sapiens cDNA, | 0.312853457 | 0.03165 |
| | 3 end /clone = IMAGE-1845841 /clone_end = 3′ | | |
| | /gb = AI243125 /gi = 3838522 /ug = Hs.182947 /len = 388′ | | |
| 72558_at | Cluster Incl. AI858718:wl41f12.x1 Homo sapiens cDNA, | 0.312511549 | 0.014896 |
| | 3 end /clone = IMAGE-2427503 /clone_end = 3′ | | |
| | /gb = AI858718 /gi = 5512334 /ug = Hs.226562 /len = 654′ | | |
| 76043_at | Cluster Incl. AW015534:UI-H-BI0p-aau-b-12-0-UI.s1 Homo sapiens cDNA, | 0.312460844 | 0.007489 |
| | 3 end /clone = IMAGE-2710606 /clone_end = 3′ | | |
| | /gb = AW015534 /gi = 5864281 /ug = Hs.234248 /len = 519′ | | |
| 84991_at | Cluster Incl. AI732539:ni01f09.x5 Homo sapiens cDNA, | 0.31169073 | 0.040979 |
| | 3 end /clone = IMAGE-966761 /clone_end = 3′ | | |
| | /gb = AI732539 /gi = 5053652 /ug = Hs.180142 /len = 508′ | | |
| 71283_at | Cluster Incl. R35259:yg61b10.r1 Homo sapiens cDNA, | 0.311634623 | 0.000087 |
| | 5 end /clone = IMAGE-37315 /clone_end = 5′ | | |
| | /gb = R35259 /gi = 792160 /ug = Hs.213548 /len = 513′ | | |
| 79909_i_a | Cluster Incl. AI808615:wf56f01.x1 Homo sapiens cDNA, | 0.310115615 | 0.000765 |
| | 3 end /clone = IMAGE-2359609 /clone_end = 3′ | | |
| | /gb = AI808615 /gi = 5395181 /ug = Hs.202625 /len = 443′ | | |
| 84081_at | Cluster Incl. Al420234:te98b01.x1 Homo sapiens cDNA, | 0.309511353 | 0.000025 |
| | 3 end /clone = IMAGE-2094697 /clone_end = 3′ | | |
| | /gb = AI420234 /gi = 4266165 /ug = Hs.163645 /len = 501′ | | |
| 83406_at | Cluster Incl. AI935292:wp16e07.x1 Homo sapiens cDNA, | 0.307974308 | 0.006092 |
| | 3 end /clone = IMAGE-2465028 /clone_end = 3′ | | |
| | /gb = AI935292 /gi = 5674162 /ug = Hs.153408 /len = 573′ | | |
| 68268_at | Cluster Incl. AA702143:zi85h05.s1 Homo sapiens cDNA, | 0.30729455 | 0.027087 |
| | 3 end /clone = 447609 /clone_end = 3′ | | |
| | /gb = AA702143 /gi = 2705256 /ug = Hs.190365 /len = 447′ | | |
| 84950_at | Cluster Incl. AI681868:tx50a12.x1 Homo sapiens cDNA, | 0.304600093 | 0.000743 |
| | 3 end /clone = IMAGE-2272990 /clone_end = 3′ | | |
| | /gb = AI681868 /gi = 4892050 /ug = Hs.178784 /len = 562′ | | |
| 71416_at | Cluster Incl. AI023259:ov64g12.s1 Homo sapiens cDNA, | 0.303909365 | 0.000025 |
| | 3 end /clone = IMAGE-1642150 /clone_end = 3′ | | |
| | /gb = AI023259 /gi = 3238500 /ug = Hs.215260 /len = 501′ | | |
| 79484_r_a | Cluster Incl. N73382:EST55b03 Homo sapiens cDNA | 0.303863976 | 0.033101 |
| | /clone = 55b03 /gb = N73382 /gi = 1230490 /ug = Hs.193146 | | |
| | /len = 398 | | |
| 76752_at | Cluster Incl. AI653380:wb45d09.x1 Homo sapiens cDNA, | 0.30383599 | 0.00007 |
| | 3 end /clone = IMAGE-2308625 /clone_end = 3′ | | |
| | /gb = AI653380 /gi = 4737359 /ug = Hs.133081 /len = 517′ | | |
| 88480_at | Cluster Incl. AI076929:ov20f07.x1 Homo sapiens cDNA, | 0.303580242 | 0.04562 |
| | 3 end /clone = IMAGE-1637893 /clone_end = 3′ | | |
| | /gb = AI076929 /gi = 3404758 /ug = Hs.105887 /len = 589′ | | |
| 76056_at | Cluster Incl. AA161405:zo72g08.r1 Homo sapiens cDNA, | 0.301518123 | 0.016888 |
| | 5 end /clone = IMAGE-592478 /clone_end = 5′ | | |
| | /gb = AA161405 /gi = 1735835 /ug = Hs.237871 /len = 666′ | | |
| 84827_at | Cluster Incl. H45983:yo13b04.s1 Homo sapiens cDNA, | 0.299353312 | 0.004482 |
| | 3 end /clone = IMAGE-177775 /clone_end = 3′ | | |
| | /gb = H45983 /gi = 922035 /ug = Hs.174198 /len = 485′ | | |
| 91207_at | Cluster Incl. AA923142:on18e11.s1 Homo sapiens cDNA, | 0.294579992 | 0.002012 |
| | 3 end /clone = IMAGE-1557068 /clone_end = 3′ | | |
| | /gb = AA923142 /gi = 3070451 /ug = Hs.24884 /len = 653′ | | |
| 81631_at | Cluster Incl. AW007764:wt68h01.x1 Homo sapiens cDNA, | 0.294066245 | 0.024164 |
| | 3 end /clone = IMAGE-2512657 /clone_end = 3′ | | |
| | /gb = AW007764 /gi = 5856542 /ug = Hs.121915 /len = 516′ | | |
| 90059_at | Cluster Incl. AI921788:wn65c05.x1 Homo sapiens cDNA, | 0.292038446 | 0.000325 |
| | 3 end /clone = IMAGE-2450312 /clone_end = 3′ | | |
| | /gb = AI921788 /gi = 5657752 /ug = Hs.126082 /len = 550′ | | |
| 71426_f_a | Cluster Incl. AI277946:qm51b03.x1 Homo sapiens cDNA, | 0.291075799 | 0.005808 |
| | 3 end /clone = IMAGE-1892237 /clone_end = 3′ | | |
| | /gb = AI277946 /gi = 3900214 /ug = Hs.215571 /len = 334′ | | |
| 81951_at | Cluster Incl. AI640180:wa30a12.x1 Homo sapiens cDNA, | 0.291050583 | 0.003823 |
| | 3 end /clone = IMAGE-2299582 /clone_end = 3′ | | |
| | /gb = AI640180 /gi = 4703289 /ug = Hs.126855 /len = 463′ | | |
| 70677_at | Cluster Incl. AI783611:tz99c01.x1 Homo sapiens cDNA, | 0.290005448 | 0.027407 |
| | 3 end /clone = IMAGE-2296704 /clone_end = 3′ | | |
| | /gb = AI783611 /gi = 5325420 /ug = Hs.208907 /len = 325′ | | |
| 79507_r_a | Cluster Incl. AA180065:zp40h07.s1 Homo sapiens cDNA, | 0.289433499 | 0.01687 |
| | 3 end /clone = IMAGE-611965 /clone_end = 3′ | | |
| | /gb = AA180065 /gi = 1761331 /ug = Hs.193579 /len = 438′ | | |
| 86556_at | Cluster Incl. AI769511:wh57h04.x1 Homo sapiens cDNA, | 0.288562177 | 0.00028 |
| | 3 end /clone = IMAGE-2384887 /clone_end = 3′ | | |
| | /gb = AI769511 /gi = 5236020 /ug = Hs.201259 /len = 550′ | | |
| 75652_at | Cluster Incl. AI983437:wt49b04.x1 Homo sapiens cDNA, | 0.28652751 | 0.006637 |
| | 3 end /clone = IMAGE-2510767 /clone_end = 3′ | | |
| | /gb = AI983437 /gi = 5810656 /ug = Hs.155145 /len = 504′ | | |
| 84975_r_a | Cluster Incl. AA649608:ns46b02.s1 Homo sapiens cDNA | 0.28641313 | 0.000038 |
| | /clone = IMAGE-1186635 /gb = AA649608 /gi = 2576936 | | |
| | /ug = Hs.179821 /len = 316 | | |
| 80632_at | Cluster Incl. AA846471:aj56d07.s1 Homo sapiens cDNA, | 0.286244603 | 0.007418 |
| | 3 end /clone = IMAGE-1394317 /clone_end = 3′ | | |
| | /gb = AA846471 /gi = 2932611 /ug = Hs.239354 /len = 643′ | | |
| 70813_at | Cluster Incl. AI797429:we54c10.x1 Homo sapiens cDNA, | 0.285905069 | 0.001632 |
| | 3 end /clone = IMAGE-2344914 /clone_end = 3′ | | |
| | /gb = AI797429 /gi = 5362901 /ug = Hs.209518 /len = 357′ | | |
| 76199_at | Cluster Incl. AA057583:zf62e12.s1 Homo sapiens cDNA, | 0.285315234 | 0.000117 |
| | 3 end /clone = IMAGE-381550 /clone_end = 3′ | | |
| | /gb = AA057583 /gi = 1550223 /ug = Hs.118553 /len = 524′ | | |
| 72405_at | Cluster Incl. AI948717:wq25a02.x1 Homo sapiens cDNA, | 0.28501066 | 0.003409 |
| | 3 end /clone = IMAGE-2472266 /clone_end = 3′ | | |
| | /gb = AI948717 /gi = 5741027 /ug = Hs.225155 /len = 505′ | | |
| 74580_at | Cluster Incl. AA417643:zv04a08.r1 Homo sapiens cDNA, | 0.284080797 | 0.006142 |
| | 5 end /clone = IMAGE-752630 /clone_end = 5′ | | |
| | /gb = AA417643 /gi = 2079462 /ug = Hs.130546 /len = 490′ | | |
| 77399_at | Cluster Incl. AI361798:qz24f08.x1 Homo sapiens cDNA, | 0.282876221 | 0.009253 |
| | 3 end /clone = IMAGE-2027847 /clone_end = 3′ | | |
| | /gb = AI361798 /gi = 4113419 /ug = Hs.164675 /len = 541′ | | |
| 78543_at | Cluster Incl. AA877572:nr05g06.s1 Homo sapiens cDNA, | 0.282804511 | 0.002194 |
| | 3 end /clone = IMAGE-1160986 /clone_end = 3′ | | |
| | /gb = AA877572 /gi = 2986537 /ug = Hs.155512 /len = 429′ | | |
| 69157_at | Cluster Incl. AI651803:wb55f04.x1 Homo sapiens cDNA, | 0.277835487 | 0.001605 |
| | 3 end /clone = IMAGE-2309599 /clone_end = 3′ | | |
| | /gb = AI651803 /gi = 4735794 /ug = Hs.195264 /len = 359′ | | |
| 86353_at | Cluster Incl. AI767724:wh39a07.x1 Homo sapiens cDNA, | 0.276458086 | 0.000703 |
| | 3 end /clone = IMAGE-2383092 /clone_end = 3′ | | |
| | /gb = AI767724 /gi = 5234233 /ug = Hs.197287 /len = 556′ | | |
| 89837_r_a | Cluster Incl. T89638:ye11d12.s1 Homo sapiens cDNA, | 0.275506295 | 0.007145 |
| | 3 end /clone = IMAGE-117431 /clone_end = 3′ | | |
| | /gb = T89638 /gi = 718151 /ug = Hs.116792 /len = 517′ | | |
| 86050_at | Cluster Incl. AA213912:zn57h11.s1 Homo sapiens cDNA, | 0.274761317 | 0.000129 |
| | 3 end /clone = IMAGE-562341 /clone_end = 3′ | | |
| | /gb = AA213912 /gi = 1812714 /ug = Hs.193564 /len = 544′ | | |
| 91748_at | Cluster Incl. W68223:zd35c12.r1 Homo sapiens cDNA, | 0.273530639 | 0.043603 |
| | 5 end /clone = IMAGE-342646 /clone_end = 5′ | | |
| | /gb = W68223 /gi = 1377161 /ug = Hs.238901 /len = 609′ | | |
| 86004_at | Cluster Incl. AA767895:oa40a11.s1 Homo sapiens cDNA | 0.273401061 | 0.000134 |
| | /clone = IMAGE-1307420 /gb = AA767895 /gi = 2818910 | | |
| | /ug = Hs.193247 /len = 538 | | |
| 87972_s_{—} | Cluster Incl. AW023444:df54h10.y1 Homo sapiens cDNA, | 0.271780784 | 0.000549 |
| | 5 end /clone = IMAGE-2487403 /clone_end = 5′ | | |
| | /gb = AW023444 /gi = 5876974 /ug = Hs.237396 /len = 465′ | | |
| 82088_at | Cluster Incl. H24953:yl39f08.s1 Homo sapiens cDNA, | 0.271229201 | 0.006574 |
| | 3 end /clone = IMAGE-160647 /clone_end = 3′ | | |
| | /gb = H24953 /gi = 893852 /ug = Hs.130815 /len = 529′ | | |
| 86204_at | Cluster Incl. AI827476:wf29g07.x1 Homo sapiens cDNA, | 0.269758658 | 0.019109 |
| | 3 end /clone = IMAGE-2357052 /clone_end = 3′ | | |
| | /gb = AI827476 /gi = 5448147 /ug = Hs.194421 /len = 520′ | | |
| 90088_at | Cluster Incl. AI807902:wf52b01.x1 Homo sapiens cDNA, | 0.269431153 | 0.001195 |
| | 3 end /clone = IMAGE-2359177 /clone_end = 3′ | | |
| | /gb = AI807902 /gi = 5394468 /ug = Hs.127301 /len = 554′ | | |
| 73728_at | Cluster Incl. AI073865:oo12a05.x1 Homo sapiens cDNA, | 0.269254487 | 0.004875 |
| | 3 end /clone = IMAGE-1565936 /clone_end = 3′ | | |
| | /gb = AI073865 /gi = 3400509 /ug = Hs.122821 /len = 458′ | | |
| 89656_at | Cluster Incl. R53734:yi02g05.r1 Homo sapiens cDNA, | 0.268646481 | 0.005615 |
| | 5 end /clone = IMAGE-138104 /clone_end = 5′ | | |
| | /gb = R53734 /gi = 815636 /ug = Hs.25978 /len = 430′ | | |
| 71466_at | Cluster Incl. AI453103:tj61f03.x1 Homo sapiens cDNA, | 0.267307121 | 0.034897 |
| | 3 end /clone = IMAGE-2146013 /clone_end = 3′ | | |
| | /gb = AI453103 /gi = 4308016 /ug = Hs.216699 /len = 481′ | | |
| 81596_at | Cluster Incl. AI694593:wd88f10.x1 Homo sapiens cDNA, | 0.266846366 | 0.009889 |
| | 3 end /clone = IMAGE-2338699 /clone_end = 3′ | | |
| | /gb = AI694593 /gi = 4971933 /ug = Hs.121574 /len = 538′ | | |
| 87152_at | Cluster Incl. AA554814:ni34c04.s1 Homo sapiens cDNA, | 0.265915547 | 0.017712 |
| | 3 end /clone = IMAGE-978726 /clone_end = 3′ | | |
| | /gb = AA554814 /gi = 2325353 /ug = Hs.213551 /len = 443′ | | |
| 83897_at | Cluster Incl. AI458439:tj82c11.x1 Homo sapiens cDNA, | 0.26411577 | 0.000037 |
| | 3 end /clone = IMAGE-2148020 /clone_end = 3′ | | |
| | /gb = AI458439 /gi = 4311018 /ug = Hs.159115 /len = 557′ | | |
| 77613_at | Cluster Incl. R54310:yg74a09.r1 Homo sapiens cDNA, | 0.262617018 | 0.014534 |
| | 5 end /clone = IMAGE-38941 /clone_end = 5′ | | |
| | /gb = R54310 /gi = 816212 /ug = Hs.18685 /len = 522′ | | |
| 81862_at | Cluster Incl. AI138884:qd99c07.x1 Homo sapiens cDNA, | 0.262322708 | 0.048252 |
| | 3 end /clone = IMAGE-1737612 /clone_end = 3′ | | |
| | /gb = AI138884 /gi = 3644856 /ug = Hs.125490 /len = 482′ | | |
| 87927_at | Cluster Incl. AW003850:ws61b04.x1 Homo sapiens cDNA, | 0.26115592 | 0.002756 |
| | 3 end /clone = IMAGE-2501647 /clone_end = 3′ | | |
| | /gb = AW003850 /gi = 5850766 /ug = Hs.236664 /len = 562′ | | |
| 86211_at | Cluster Incl. AI621034:ts76b11.x1 Homo sapiens cDNA, | 0.261152746 | 0.033787 |
| | 3 end /clone = IMAGE-2237181 /clone_end = 3′ | | |
| | /gb = AI621034 /gi = 4630160 /ug = Hs.194449 /len = 556′ | | |
| 88019_f_a | Cluster Incl. AI968388:wu02e09.x1 Homo sapiens cDNA, | 0.259572265 | 0.049172 |
| | 3 end /clone = IMAGE-2515816 /clone_end = 3′ | | |
| | /gb = AI968388 /gi = 5765206 /ug = Hs.238114 /len = 301′ | | |
| AFFX-HU | M33197 Human glyceraldehyde-3-phosphate dehydrogenase (GAPDH) mRNA, | 0.257980182 | 0.015423 |
| | complete cds (_5, _M, _3 represent transcript regions | | |
| | 5 prime, Middle, and 3 prime respectively) | | |
| 77773_at | Cluster Incl. AA593528:nn28a06.s1 Homo sapiens cDNA, | 0.2562359 | 0.028242 |
| | 3 end /clone = IMAGE-1085170 /clone_end = 3′ | | |
| | /gb = AA593528 /gi = 2409290 /ug = Hs.198976 /len = 433′ | | |
| 85360_at | Cluster Incl. AA255965:zs28g07.s1 Homo sapiens cDNA, | 0.255474175 | 0.006366 |
| | 3 end /clone = IMAGE-686556 /clone_end = 3′ | | |
| | /gb = AA255965 /gi = 1891523 /ug = Hs.186853 /len = 406′ | | |
| 76212_at | Cluster Incl. W26941:16f10 Homo sapiens cDNA | 0.255231274 | 0.000598 |
| | /gb = W26941 /gi = 1306169 /ug = Hs.118727 /len = 547 | | |
| 90585_at | Cluster Incl. AI368415:qy08b11.x1 Homo sapiens cDNA, | 0.25397395 | 0.006918 |
| | 3 end /clone = IMAGE-2011389 /clone_end = 3′ | | |
| | /gb = AI368415 /gi = 4147168 /ug = Hs.155074 /len = 759′ | | |
| 67405_at | Cluster Incl. AI610167:tp13h07.x1 Homo sapiens cDNA, | 0.253224559 | 0.005829 |
| | 3 end /clone = IMAGE-2187709 /clone_end = 3′ | | |
| | /gb = AI610167 /gi = 4619334 /ug = Hs.185056 /len = 567′ | | |
| 86842_at | Cluster Incl. W06950:za91a12.r1 Homo sapiens cDNA, | 0.252673195 | 0.001281 |
| | 5 end /clone = IMAGE-299902 /clone_end = 5′ | | |
| | /gb = W06950 /gi = 1280952 /ug = Hs.206454 /len = 523′ | | |
| 82083_at | Cluster Incl. AA903403:ok62c08.s1 Homo sapiens cDNA, | 0.252499158 | 0.002692 |
| | 3 end /clone = IMAGE-1518542 /clone_end = 3′ | | |
| | /gb = AA903403 /gi = 3038526 /ug = Hs.130761 /len = 551′ | | |
| 67461_at | Cluster Incl. AA702220:zi84e01.s1 Homo sapiens cDNA, | 0.252109936 | 0.002038 |
| | 3 end /clone = 447480 /clone_end = 3′ | | |
| | /gb = AA702220 /gi = 2705333 /ug = Hs.185783 /len = 462′ | | |
| 80797_at | Cluster Incl. AL044128:DKFZp434N0628_s1 Homo sapiens cDNA, | 0.251370696 | 0.000508 |
| | 3 end /clone = DKFZp434N0628 /clone end = 3′ | | |
| | /gb = AL044128 /gi = 5432354 /ug = Hs.94195 /len = 983′ | | |
| 81053_at | Cluster Incl. AI273624:ql56c06.x1 Homo sapiens cDNA, | 0.249890723 | 0.000955 |
| | 3 end /clone = IMAGE-1876330 /clone_end = 3′ | | |
| | /gb = AI273624 /gi = 3895892 /ug = Hs.115192 /len = 414′ | | |
| 77218_at | Cluster Incl. AA634799:ab28b06.r1 Homo sapiens cDNA, | 0.249121821 | 0.000134 |
| | 5 end /clone = IMAGE-842099 /clone_end = 5′ | | |
| | /gb = AA634799 /gi = 2558013 /ug = Hs.151888 /len = 538′ | | |
| 79663_at | Cluster Incl. AI566193:tq69f02.x1 Homo sapiens cDNA, | 0.249088172 | 0.035959 |
| | 3 end /clone = IMAGE-2214075 /clone_end = 3′ | | |
| | /gb = AI566193 /gi = 4524645 /ug = Hs.196927 /len = 393′ | | |
| 85962_at | Cluster Incl. AI923659:wn59b05.x1 Homo sapiens cDNA, | 0.247682275 | 0.00136 |
| | 3 end /clone = IMAGE-2449713 /clone_end = 3′ | | |
| | /gb = AI923659 /gi = 5659623 /ug = Hs.192972 /len = 577′ | | |
| 83951_r_a | Cluster Incl. AA812024:ob39g08.s1 Homo sapiens cDNA | 0.243142897 | 0.009508 |
| | /clone = IMAGE-1333790 /gb = AA812024 /gi = 2881635 | | |
| | /ug = Hs.159669 /len = 410 | | |
| 78463_at | Cluster Incl. AI160540:qc87a04.x1 Homo sapiens cDNA, | 0.242819085 | 0.017392 |
| | 3 end /clone = IMAGE-1721166 /clone_end = 3′ | | |
| | /gb = AI160540 /gi = 3693920 /ug = Hs.13781 /len = 579′ | | |
| 84535_at | Cluster Incl. N50065:yz10h03.s1 Homo sapiens cDNA, | 0.242621323 | 0.040681 |
| | 3 end /clone = IMAGE-282677 /clone_end = 3′ | | |
| | /gb = N50065 /gi = 1191231 /ug = Hs.169732 /len = 550′ | | |
| 91147_at | Cluster Incl. AI929144:au65f04.y1 Homo sapiens cDNA | 0.241886973 | 0.000589 |
| | /clone = IMAGE /gb = AI929144 /gi = 5665108 | | |
| | /ug = Hs.239175 /len = 724 | | |
| 80851_at | Cluster Incl. AI733027:oj14a06.x5 Homo sapiens cDNA, | 0.241842162 | 0.000183 |
| | 3 end /clone = IMAGE-1492114 /clone_end = 3′ | | |
| | /gb = AI733027 /gi = 5054140 /ug = Hs.105206 /len = 590′ | | |
| 91750_at | Cluster Incl. AI921883:wp07f05.x1 Homo sapiens cDNA, | 0.234701355 | 0.000625 |
| | 3 end /clone = IMAGE-2464161 /clone_end = 3′ | | |
| | /gb = AI921883 /gi = 5657847 /ug = Hs.23921 /len = 582′ | | |
| 74161_at | Cluster Incl. AA960997:oo51f06.s1 Homo sapiens cDNA, | 0.234459062 | 0.005177 |
| | 3 end /clone = IMAGE-1569731 /clone_end = 3′ | | |
| | /gb = AA960997 /gi = 3127551 /ug = Hs.127765 /len = 434′ | | |
| 90373_r_a | Cluster Incl. N54525:yv37d07.s1 Homo sapiens cDNA, | 0.2338597 | 0 |
| | 3 end /clone = IMAGE-244909 /clone_end = 3′ | | |
| | /gb = N54525 /gi = 1195845 /ug = Hs.141278 /len = 495′ | | |
| 86687_i_a | Cluster Incl. AI885491:wm24f11.x1 Homo sapiens cDNA, | 0.233577442 | 0.000091 |
| | 3 end /clone = IMAGE-2436909 /clone_end = 3′ | | |
| | /gb = AI885491 /gi = 5590655 /ug = Hs.203196 /len = 464′ | | |
| 76769_at | Cluster Incl. AI758223:ty66b04.x1 Homo sapiens cDNA, | 0.233395219 | 0.000097 |
| | 3 end /clone = IMAGE-2284015 /clone_end = 3′ | | |
| | /gb = AI758223 /gi = 5151946 /ug = Hs.133471 /len = 535′ | | |
| 73026_s_{—} | Cluster Incl. AI420118:tf05b01.x1 Homo sapiens cDNA, | 0.22622915 | 0.035572 |
| | 3 end /clone = IMAGE-2095273 /clone_end = 3′ | | |
| | /gb = AI420118 /gi = 4266049 /ug = Hs.234008 /len = 429′ | | |
| 91887_f_a | Cluster Incl. AI860139:wh39f08.x1 Homo sapiens cDNA, | 0.225862338 | 0.001895 |
| | 3 end /clone = IMAGE-2383143 /clone_end = 3′ | | |
| | /gb = AI860139 /gi = 5513755 /ug = Hs.134556 /len = 528′ | | |
| 81686_at | Cluster Incl. AA807273:oc33e01.s1 Homo sapiens cDNA | 0.221895364 | 0.030677 |
| | /clone = IMAGE-1351512 /gb = AA807273 /gi = 2876849 | | |
| | /ug = Hs.122832 /len = 469 | | |
| 78622_r_a | Cluster Incl. W26589:33d9 Homo sapiens cDNA | 0.221208606 | 0.003353 |
| | /gb = W26589 /gi = 1307432 /ug = Hs.22954 /len = 594 | | |
| 89844_at | Cluster Incl. AI806507:wf08h01.x1 Homo sapiens cDNA, | 0.219504698 | 0.001214 |
| | 3 end /clone = IMAGE-2350033 /clone_end = 3′ | | |
| | /gb = AI806507 /gi = 5393073 /ug = Hs.117772 /len = 565′ | | |
| 76942_i_a | Cluster Incl. AI821447:yp24d10.x5 Homo sapiens cDNA, | 0.218832459 | 0.00729 |
| | 3 end /clone = IMAGE-188371 /clone_end = 3′ | | |
| | /gb = AI821447 /gi = 5440526 /ug = Hs.141120 /len = 542′ | | |
| 82865_at | Cluster Incl. AI829717:wf09f05.x1 Homo sapiens cDNA, | 0.218261577 | 0.006778 |
| | 3 end /clone = IMAGE-2350113 /clone_end = 3′ | | |
| | /gb = AI829717 /gi = 5450388 /ug = Hs.146016 /len = 541′ | | |
| 90629_at | Cluster Incl. X84721:HSEST222 Homo sapiens cDNA | 0.21759462 | 0.018709 |
| | /clone = MEC-222 /gb = X84721 /gi = 673398 | | |
| | /ug = Hs.164866 /len = 558 | | |
| 78209_at | Cluster Incl. T79705:yd71e10.r1 Homo sapiens cDNA, | 0.217378721 | 0.0002 |
| | 5 end /clone = IMAGE-113706 /clone_end = 5′ | | |
| | /gb = T79705 /gi = 698214 /ug = Hs.239664 /len = 453′ | | |
| 89338_at | Cluster Incl. AA102335:zl91b05.s1 Homo sapiens cDNA, | 0.216876929 | 0.011804 |
| | 3 end /clone = IMAGE-511953 /clone_end = 3′ | | |
| | /gb = AA102335 /gi = 1647042 /ug = Hs.183620 /len = 540′ | | |
| 87935_r_a | Cluster Incl. AI817659:td15b09.x1 Homo sapiens cDNA, | 0.216095926 | 0.001085 |
| | 3 end /clone = IMAGE-2075705 /clone_end = 3′ | | |
| | /gb = AI817659 /gi = 5436738 /ug = Hs.236720 /len = 457′ | | |
| 90111_at | Cluster Incl. AI417537:tg79c03.x1 Homo sapiens cDNA, | 0.214818394 | 0.03032 |
| | 3 end /clone = IMAGE-2114980 /clone end = 3′ | | |
| | /gb = AI417537 /gi = 4261041 /ug = Hs.127808 /len = 512′ | | |
| 88754_at | Cluster Incl. AA775833:ad17c09.s1 Homo sapiens cDNA, | 0.210857155 | 0.040755 |
| | 3 end /clone = 878512 /clone_end = 3′ | | |
| | /gb = AA775833 /gi = 2835167 /ug = Hs.122655 /len = 501′ | | |
| 71736_at | Cluster Incl. AA937952:oc08c08.s1 Homo sapiens cDNA, | 0.206013142 | 0.000046 |
| | 3 end /clone = IMAGE-1340270 /clone_end = 3′ | | |
| | /gb = AA937952 /gi = 3096063 /ug = Hs.221237 /len = 373′ | | |
| 79489_at | Cluster Incl. AI620433:tu47b05.x1 Homo sapiens cDNA, | 0.205729323 | 0.001955 |
| | 3 end /clone = IMAGE-2254161 /clone_end = 3′ | | |
| | /gb = AI620433 /gi = 4629559 /ug = Hs.193201 /len = 486′ | | |
| 89360_at | Cluster Incl. AA630327:ac08g11.s1 Homo sapiens cDNA, | 0.20325397 | 0.003 |
| | 3 end /clone = IMAGE-855908 /clone_end = 3′ | | |
| | /gb = AA630327 /gi = 2552938 /ug = Hs.185158 /len = 637′ | | |
| 91460_at | Cluster Incl. AI342616:qt33e02.x1 Homo sapiens cDNA, | 0.200209939 | 0.037457 |
| | 3 end /clone = IMAGE-1949786 /clone_end = 3′ | | |
| | /gb = AI342616 /gi = 4079543 /ug = Hs.183793 /len = 471′ | | |
| 76759_r_a | Cluster Incl. AI224653:qw97g08.x1 Homo sapiens cDNA, | 0.196540489 | 0.000067 |
| | 3 end /clone = IMAGE-1999070 /clone_end = 3′ | | |
| | /gb = AI224653 /gi = 3807366 /ug = Hs.133286 /len = 448′ | | |
| 84240_at | Cluster Incl. W93868:zd97a07.r1 Homo sapiens cDNA, | 0.196285806 | 0.003331 |
| | 5 end /clone = IMAGE-357396 /clone_end = 5′ | | |
| | /gb = W93868 /gi = 1423010 /ug = Hs.166241 /len = 426′ | | |
| 71069_at | Cluster Incl. AI832201:td14b10.x1 Homo sapiens cDNA, | 0.195317324 | 0.002561 |
| | 3 end /clone = IMAGE-2075611 /clone_end = 3′ | | |
| | /gb = AI832201 /gi = 5452872 /ug = Hs.211469 /len = 475′ | | |
| 85168_at | Cluster Incl. AI653441:tq94b09.x1 Homo sapiens cDNA, | 0.193174439 | 0.000276 |
| | 3 end /clone = IMAGE-2216441 /clone_end = 3′ | | |
| | /gb = AI653441 /gi = 4737420 /ug = Hs.183409 /len = 448′ | | |
| 73285_i_a | Cluster Incl. AI971748:wr07f08.x1 Homo sapiens cDNA, | 0.185052334 | 0.002302 |
| | 3 end /clone = IMAGE-2480871 /clone end = 3′ | | |
| | /gb = AI971748 /gi = 5768574 /ug = Hs.236704 /len = 841′ | | |
| 91306_s_{—} | Cluster Incl. AA613715:nq25b01.s1 Homo sapiens cDNA, | 0.184479141 | 0.000085 |
| | 3 end /clone = IMAGE-1144873 /clone_end = 3′ | | |
| | /gb = AA613715 /gi = 2463685 /ug = Hs.81795 /len = 607′ | | |
| 91309_r_a | Cluster Incl. AA613715:nq25b01.s1 Homo sapiens cDNA, | 0.180034385 | 0.000184 |
| | 3 end /clone = IMAGE-1144873 /clone_end = 3′ | | |
| | /gb = AA613715 /gi = 2463685 /ug = Hs.81795 /len = 607′ | | |
| 77736_at | Cluster Incl. AA604618:no84b05.s1 Homo sapiens cDNA, | 0.178791804 | 0.000087 |
| | 3 end /clone = IMAGE-1113489 /clone_end = 3′ | | |
| | /gb = AA604618 /gi = 2445482 /ug = Hs.194090 /len = 437′ | | |
| 67440_r_a | Cluster Incl. AA419260:zv35c03.s1 Homo sapiens cDNA, | 0.176702997 | 0.015013 |
| | 3 end /clone = IMAGE-755620 /clone_end = 3′ | | |
| | /gb = AA419260 /gi = 2078973 /ug = Hs.185665 /len = 456′ | | |
| 82862_at | Cluster Incl. AI817698:wk25b08.x1 Homo sapiens cDNA, | 0.176441557 | 0.036843 |
| | 3 end /clone = IMAGE-2413335 /clone_end = 3′ | | |
| | /gb = AI817698 /gi = 5436777 /ug = Hs.145994 /len = 548′ | | |
| 72186_at | Cluster Incl. AI580176:tk17g08.x1 Homo sapiens cDNA, | 0.175060418 | 0.005973 |
| | 3 end /clone = IMAGE-2151326 /clone_end = 3′ | | |
| | /gb = AI580176 /gi = 4564552 /ug = Hs.223394 /len = 513′ | | |
| 87011_at | Cluster Incl. AI040273:ox92d10.x1 Homo sapiens cDNA, | 0.174753613 | 0.016883 |
| | 3 end /clone = IMAGE-1663795 /clone_end = 3′ | | |
| | /gb = AI040273 /gi = 3279467 /ug = Hs.210508 /len = 450′ | | |
| 67117_at | Cluster Incl. AA026238:zj99f11.s1 Homo sapiens cDNA, | 0.174410782 | 0.003724 |
| | 3 end /clone = IMAGE-469197 /clone_end = 3′ | | |
| | /gb = AA026238 /gi = 1492777 /ug = Hs.181272 /len = 395′ | | |
| 72418_r_a | Cluster Incl. AI990292:ws30h05.x1 Homo sapiens cDNA, | 0.172944529 | 0.001836 |
| | 3 end /clone = IMAGE-2498745 /clone_end = 3′ | | |
| | /gb = AI990292 /gi = 5837173 /ug = Hs.225457 /len = 445′ | | |
| 76618_at | Cluster Incl. AI806805:wf15e07.x1 Homo sapiens cDNA, | 0.171672809 | 0.004788 |
| | 3 end /clone = IMAGE-2350692 /clone_end = 3′ | | |
| | /gb = AI806805 /gi = 5393371 /ug = Hs.129965 /len = 487′ | | |
| 74321_at | Cluster Incl. AI702482:tz94e07.x1 Homo sapiens cDNA, | 0.165834292 | 0.003435 |
| | 3 end /clone = IMAGE-2296260 /clone_end = 3′ | | |
| | /gb = AI702482 /gi = 4990382 /ug = Hs.128672 /len = 494′ | | |
| 71946_at | Cluster Incl. AI005278:ou07g04.x1 Homo sapiens cDNA, | 0.165484982 | 0.002113 |
| | 3 end /clone = IMAGE-1625622 /clone_end = 3′ | | |
| | /gb = AI005278 /gi = 3214788 /ug = Hs.222140 /len = 481′ | | |
| 91356_at | Cluster Incl. AI392846:tg10d02.x1 Homo sapiens cDNA, | 0.164854129 | 0.005954 |
| | 3 end /clone = IMAGE-2108355 /clone_end = 3′ | | |
| | /gb = AI392846 /gi = 4222393 /ug = Hs.95744 /len = 542′ | | |
| 85092_g_{—} | Cluster Incl. AI554809:tp73f01.x1 Homo sapiens cDNA, | 0.164014819 | 0.000531 |
| | 3 end /clone = IMAGE-2204953 /clone_end = 3′ | | |
| | /gb = AI554809 /gi = 4487172 /ug = Hs.182339 /len = 1035′ | | |
| 91414_at | Cluster Incl. N72573:za46c03.s1 Homo sapiens cDNA, | 0.16120887 | 0.005273 |
| | 3 end /clone = IMAGE-295588 /clone_end = 3′ | | |
| | /gb = N72573 /gi = 1229677 /ug = Hs.118183 /len = 482′ | | |
| 86418_at | Cluster Incl. AI978581:wq72d08.x1 Homo sapiens cDNA, | 0.160027369 | 0.000057 |
| | 3 end /clone = IMAGE-2476815 /clone_end = 3′ | | |
| | /gb = AI978581 /gi = 5803611 /ug = Hs.198694 /len = 475′ | | |
| 86370_at | Cluster Incl. AW003775:ws16h04.x1 Homo sapiens cDNA, | 0.159738807 | 0.015431 |
| | 3 end /clone = IMAGE-2497399 /clone_end = 3′ | | |
| | /gb = AW003775 /gi = 5850691 /ug = Hs.197737 /len = 647′ | | |
| 83047_at | Cluster Incl. AI627358:ty75d11.x1 Homo sapiens cDNA, | 0.158131021 | 0.016115 |
| | 3 end /clone = IMAGE-2284917 /clone_end = 3′ | | |
| | /gb = AI627358 /gi = 4664158 /ug = Hs.148367 /len = 552′ | | |
| 89805_at | Cluster Incl. AI568633:tn41f11.x1 Homo sapiens cDNA, | 0.157868321 | 0.000963 |
| | 3 end /clone = IMAGE-2170221 /clone_end = 3′ | | |
| | /gb = AI568633 /gi = 4532007 /ug = Hs.114670 /len = 481′ | | |
| 68787_at | Cluster Incl. AA682328:zj98d05.s1 Homo sapiens cDNA, | 0.157814866 | 0.016057 |
| | 3 end /clone = IMAGE-462921 /clone_end = 3′ | | |
| | /gb = AA682328 /gi = 2669645 /ug = Hs.193084 /len = 484′ | | |
| 75096_at | Cluster Incl. AA769588:nz42b06.s1 Homo sapiens cDNA | 0.156801675 | 0.003643 |
| | /clone = IMAGE-1290419 /gb = AA769588 /gi = 2820826 | | |
| | /ug = Hs.135723 /len = 607 | | |
| 86085_r_a | Cluster Incl. AA419311:zv35c03.r1 Homo sapiens cDNA, | 0.154848601 | 0.002758 |
| | 5 end /clone = IMAGE-755620 /clone_end = 5′ | | |
| | /gb = AA419311 /gi = 2079024 /ug = Hs.193745 /len = 690′ | | |
| 77356_at | Cluster Incl. AL110434:DKFZp434N1031_r1 Homo sapiens cDNA, | 0.153135161 | 0.002875 |
| | 5 end /clone = DKFZp434N1031 /clone end = 5′ | | |
| | /gb = AL110434 /gi = 5866042 /ug = Hs.162685 /len = 638′ | | |
| 86396_at | Cluster Incl. AW001450:wu31f11.x1 Homo sapiens cDNA, | 0.153122052 | 0.002676 |
| | 3 end /clone = IMAGE-990784 /clone_end = 3′ | | |
| | /gb = AW001450 /gi = 5848366 /ug = Hs.198312 /len = 517′ | | |
| 73425_at | Cluster Incl. AI971458:wq86e05.x1 Homo sapiens cDNA, | 0.152644343 | 0.005367 |
| | 3 end /clone = IMAGE-2478944 /clone_end = 3′ | | |
| | /gb = AI971458 /gi = 5768284 /ug = Hs.239145 /len = 513′ | | |
| 67222_at | Cluster Incl. AI219734:qg88c02.x1 Homo sapiens cDNA, | 0.147483257 | 0.000055 |
| | 3 end /clone = IMAGE-1842242 /clone end = 3′ | | |
| | /gb = AI219734 /gi = 3801937 /ug = Hs.182221 /len = 443′ | | |
| 81926_at | Cluster Incl. AI685069:wc67b06.x1 Homo sapiens cDNA, | 0.143799679 | 0.001664 |
| | 3 end /clone = IMAGE-2323667 /clone_end = 3′ | | |
| | /gb = AI685069 /gi = 4896374 /ug = Hs.126383 /len = 504′ | | |
| 79554_at | Cluster Incl. H97871:yw05a04.s1 Homo sapiens cDNA, | 0.141155538 | 0.000118 |
| | 3 end /clone = IMAGE-251310 /clone_end = 3′ | | |
| | /gb = H97871 /gi = 1118756 /ug = Hs.193907 /len = 445′ | | |
| 70209_at | Cluster Incl. AI761629:wg66g02.x1 Homo sapiens cDNA, | 0.140186121 | 0.000424 |
| | 3 end /clone = IMAGE-2370098 /clone_end = 3′ | | |
| | /gb = AI761629 /gi = 5177296 /ug = Hs.203696 /len = 535′ | | |
| 80602_at | Cluster Incl. AW003595:wx34a07.x1 Homo sapiens cDNA, | 0.131039201 | 0.004556 |
| | 3 end /clone = IMAGE-2545524 /clone_end = 3′ | | |
| | /gb = AW003595 /gi = 5850511 /ug = Hs.239177 /len = 586′ | | |
| 91072_at | Cluster Incl. R78708:yi74a09.r1 Homo sapiens cDNA, | 0.126447658 | 0.000068 |
| | 5 end /clone = IMAGE-144952 /clone_end = 5′ | | |
| | /gb = R78708 /gi = 854989 /ug = Hs.237163 /len = 588′ | | |
| 91826_at | Cluster Incl. AI219073:qg16e08.x1 Homo sapiens cDNA, | 0.123573477 | 0.02356 |
| | 3 end /clone = IMAGE-1759718 /clone_end = 3′ | | |
| | /gb = AI219073 /gi = 3801276 /ug = Hs.126062 /len = 555′ | | |
| 88725_at | Cluster Incl. AA622392:no45c05.s1 Homo sapiens cDNA, | 0.12192783 | 0.007314 |
| | 3 end /clone = IMAGE-1103624 /clone_end = 3′ | | |
| | /gb = AA622392 /gi = 2526268 /ug = Hs.122244 /len = 503′ | | |
| 77982_at | Cluster Incl. AI820925:zu53f08.y5 Homo sapiens cDNA, | 0.120125995 | 0.000268 |
| | 5 end /clone = IMAGE-741735 /clone_end = 5′ | | |
| | /gb = AI820925 /gi = 5440004 /ug = Hs.227048 /len = 600′ | | |
| 78295_r_a | Cluster Incl. AI733324:op02a10.x5 Homo sapiens cDNA, | 0.11926614 | 0.009 |
| | 3 end /clone = IMAGE-1574490 /clone_end = 3′ | | |
| | /gb = AI733324 /gi = 5054437 /ug = Hs.57138 /len = 500′ | | |
| 84128_at | Cluster Incl. AI434862:ti13c03.x1 Homo sapiens cDNA, | 0.11733946 | 0.001254 |
| | 3 end /clone = IMAGE-2130340 /clone_end = 3′ | | |
| | /gb = AI434862 /gi = 4299148 /ug = Hs.164224 /len = 391′ | | |
| 76068_at | Cluster Incl. AI819863:wj45h05.x1 Homo sapiens cDNA, | 0.109228797 | 0.001154 |
| | 3 end /clone = IMAGE-2405817 /clone_end = 3′ | | |
| | /gb = AI819863 /gi = 5438942 /ug = Hs.106243 /len = 612′ | | |
| 76847_at | Cluster Incl. AI951161:wx64c08.x1 Homo sapiens cDNA, | 0.106139272 | 0.000562 |
| | 3 end /clone = IMAGE-2548430 /clone_end = 3′ | | |
| | /gb = AI951161 /gi = 5743471 /ug = Hs.135184 /len = 451′ | | |
| 86083_i_a | Cluster Incl. AA419311:zv35c03.r1 Homo sapiens cDNA, | 0.105672634 | 0.00005 |
| | 5 end /clone = IMAGE-755620 /clone end = 5′ | | |
| | /gb = AA419311 /gi = 2079024 /ug = Hs.193745 /len = 690′ | | |
| 89807_at | Cluster Incl. AI418405:tg36e10.x1 Homo sapiens cDNA, | 0.105567733 | 0.002877 |
| | 3 end /clone = IMAGE-2110890 /clone_end = 3′ | | |
| | /gb = AI418405 /gi = 4264336 /ug = Hs.115173 /len = 472′ | | |
| 69815_at | Cluster Incl. AI684761:wa85h04.x1 Homo sapiens cDNA, | 0.105063837 | 0.002168 |
| | 3 end /clone = IMAGE-2302999 /clone end = 3′ | | |
| | /gb = AI684761 /gi = 4896055 /ug = Hs.201552 /len = 678′ | | |
| 79447_at | Cluster Incl. AI452552:tj62h11.x1 Homo sapiens cDNA, | 0.102930746 | 0 |
| | 3 end /clone = IMAGE-2146149 /clone_end = 3′ | | |
| | /gb = AI452552 /gi = 4286151 /ug = Hs.192825 /len = 398′ | | |
| 74723_at | Cluster Incl. AI375142:tc09h05.x1 Homo sapiens cDNA, | 0.10188118 | 0.000018 |
| | 3 end /clone = IMAGE-2063385 /clone_end = 3′ | | |
| | /gb = AI375142 /gi = 4175132 /ug = Hs.132127 /len = 458′ | | |
| 87275_at | Cluster Incl. AA190398:zp86g11.s1 Homo sapiens cDNA, | 0.098256541 | 0.000886 |
| | 3 end /clone = IMAGE-627140 /clone_end = 3′ | | |
| | /gb = AA190398 /gi = 1779348 /ug = Hs.220905 /len = 570′ | | |
| 81445_at | Cluster Incl. AA830533:oc52c05.s1 Homo sapiens cDNA | 0.095034843 | 0.000324 |
| | /clone = IMAGE-1353320 /gb = AA830533 /gi = 2903632 | | |
| | /ug = Hs.120219 /len = 426 | | |
| 85093_at | Cluster Incl. AI991109:wu38c03.x1 Homo sapiens cDNA, | 0.090173646 | 0.001181 |
| | 3 end /clone = IMAGE-991031 /clone_end = 3′ | | |
| | /gb = AI991109 /gi = 5838012 /ug = Hs.182339 /len = 832′ | | |
| 80411_g_{—} | Cluster Incl. AA961504:op44f07.s1 Homo sapiens cDNA, | 0.082703596 | 0.000614 |
| | 3 end /clone = IMAGE-1579717 /clone_end = 3′ | | |
| | /gb = AA961504 /gi = 3133668 /ug = Hs.234808 /len = 473′ | | |
| 86688_f_a | Cluster Incl. AI885491:wm24f11.x1 Homo sapiens cDNA, | 0.080705969 | 0.000529 |
| | 3 end /clone = IMAGE-2436909 /clone_end = 3′ | | |
| | /gb = AI885491 /gi = 5590655 /ug = Hs.203196 /len = 464′ | | |
| 83638_at | Cluster Incl. AI570023:tr91b11.x1 Homo sapiens cDNA, | 0.076248907 | 0.001755 |
| | 3 end /clone = IMAGE-2226429 /clone_end = 3′ | | |
| | /gb = AI570023 /gi = 4533397 /ug = Hs.156457 /len = 401′ | | |
| 84121_at | Cluster Incl. AI480137:tm33f01.x1 Homo sapiens cDNA, | 0.070707403 | 0.000394 |
| | 3 end /clone = IMAGE-2159929 /clone_end = 3′ | | |
| | /gb = AI480137 /gi = 4373305 /ug = Hs.164198 /len = 558′ | | |
| 72187_g_{—} | Cluster Incl. AI580176:tk17g08.x1 Homo sapiens cDNA, | 0.07033885 | 0.000099 |
| | 3 end /clone = IMAGE-2151326 /clone_end = 3′ | | |
| | /gb = AI580176 /gi = 4564552 /ug = Hs.223394 /len = 513′ | | |
| 74335_at | Cluster Incl. AI950844:wx55h06.x1 Homo sapiens cDNA, | 0.062942854 | 0.000823 |
| | 3 end /clone = IMAGE-2547611 /clone_end = 3′ | | |
| | /gb = AI950844 /gi = 5743154 /ug = Hs.128738 /len = 426′ | | |
| 74168_at | Cluster Incl. AI859619:wm14d07.x1 Homo sapiens cDNA, | 0.062714223 | 0.000097 |
| | 3 end /clone = IMAGE-2435917 /clone_end = 3′ | | |
| | /gb = AI859619 /gi = 5513235 /ug = Hs.127780 /len = 525′ | | |
| 75414_at | Cluster Incl. AA897501:aj62a04.s1 Homo sapiens cDNA, | 0.061379361 | 0 |
| | 3 end /clone = IMAGE-1394862 /clone_end = 3′ | | |
| | /gb = AA897501 /gi = 3034121 /ug = Hs.143529 /len = 504′ | | |
| 81956_at | Cluster Incl. AI459244:tk11d02.x1 Homo sapiens cDNA, | 0.060318825 | 0.000092 |
| | 3 end /clone = IMAGE-2150691 /clone_end = 3′ | | |
| | /gb = AI459244 /gi = 4311823 /ug = Hs.126897 /len = 489′ | | |
| 80408_at | Cluster Incl. AA961504:op44f07.s1 Homo sapiens cDNA, | 0.058236213 | 0.002633 |
| | 3 end /clone = IMAGE-1579717 /clone_end = 3′ | | |
| | /gb = AA961504 /gi = 3133668 /ug = Hs.234808 /len = 473′ | | |
| 91978_r_a | Cluster Incl. AI457596:tj63b09.x1 Homo sapiens cDNA, | 0.057105024 | 0 |
| | 3 end /clone = IMAGE-2146169 /clone_end = 3′ | | |
| | /gb = AI457596 /gi = 4310465 /ug = Hs.204169 /len = 513′ | | |
| 88237_s_{—} | Cluster Incl. AA490688:aa45b06.r1 Homo sapiens cDNA, | 0.056521395 | 0.000735 |
| | 5 end /clone = IMAGE-823859 /clone_end = 5′ | | |
| | /gb = AA490688 /gi = 2219861 /ug = Hs.239723 /len = 561′ | | |
| 83425_at | Cluster Incl. AI739630:wi37d03.x1 Homo sapiens cDNA, | 0.050662272 | 0 |
| | 3 end /clone = IMAGE-2392421 /clone_end = 3′ | | |
| | /gb = AI739630 /gi = 5101611 /ug = Hs.153590 /len = 461′ | | |
| 89468_at | Cluster Incl. AA614254:np09h02.s1 Homo sapiens cDNA | 0.048326422 | 0.000573 |
| | /clone = IMAGE-1115859 /gb = AA614254 /gi = 2466388 | | |
| | /ug = Hs.200539 /len = 468 | | |
| 70495_at | Cluster Incl. AI453095:tj52e10.x1 Homo sapiens cDNA, | 0.047278322 | 0.000031 |
| | 3 end /clone = IMAGE-2145162 /clone_end = 3′ | | |
| | /gb = AI453095 /gi = 4307387 /ug = Hs.207393 /len = 555′ | | |
| 67146_i_a | Cluster Incl. AI349119:ta51g03.x2 Homo sapiens cDNA, | 0.04516733 | 0 |
| | 3 end /clone = IMAGE-2047636 /clone_end = 3′ | | |
| | /gb = AI349119 /gi = 4086325 /ug = Hs.181625 /len = 114′ | | |
| 82818_at | Cluster Incl. AI733800:qk36c08.x5 Homo sapiens cDNA, | 0.042193449 | 0.000056 |
| | 3 end /clone = IMAGE-1871054 /clone_end = 3′ | | |
| | /gb = AI733800 /gi = 5054913 /ug = Hs.145582 /len = 489′ | | |
| 76345_r_a | Cluster Incl. AA843931:ai90g08.s1 Homo sapiens cDNA, | 0.037828358 | 0.000112 |
| | 3 end /clone = IMAGE-1388126 /clone_end = 3′ | | |
| | /gb = AA843931 /gi = 2930382 /ug = Hs.124221 /len = 493′ | | |
| 78159_s_{—} | Cluster Incl. AI582193:tq63e12.x1 Homo sapiens cDNA, | 0.028462315 | 0.000001 |
| | 3 end /clone = IMAGE-2213518 /clone_end = 3′ | | |
| | /gb = AI582193 /gi = 4568090 /ug = Hs.239207 /len = 461′ | | |
| 75060_at | Cluster Incl. AI814274:wj71a06.x1 Homo sapiens cDNA, | 0.008079595 | 0.000572 |
| | 3 end /clone = IMAGE-2408242 /clone_end = 3′ | | |
| | /gb = AI814274 /gi = 5425489 /ug = Hs.135188 /len = 499′ | | |

TABLE 7
Genes present in the U95_A cluster which are not in the Fold Change list (>1 over-expressed
in Barrett's-asssociated esophageal denocarcinoma (BA), <1 under-expressed in BA)

| Affy ID | Gene Name | Fold Change | P-value |
|---|---|---|---|
| 760_at | Y09216/FEATURE=/DEFINITION=HSDYRK2 H.sapiens mRNA for protein kinase, Dyrk2 | 1.776710327 | 0.029598 |
| 39260_at | Cluster Incl U59185:Human putative monocarboxylate transporter (MCT) mRNA, complete cds | 1.853453277 | 0.020819 |
| | /cds=(182,1645)/gb=U59185/gi=2463627/ug=Hs.23590/len=2529 | | |
| 35219_at | Cluster Incl AL050202:Homo sapiens mRNA; cDNA DKFZp586E2023 (from clone DKFZp586E2023) | 1.960259397 | 0.00553 |
| | /cds=UNKNOWN/gb=AL050202/gi=4884441/ug=Hs.29005/len=1169 | | |
| 34778_at | Cluster Incl AA418080:zv97h07.s1 Homo sapiens cDNA, 3 end/clone=IMAGE-767773/clone_end=3“ | 1.974272775 | 0.015166 |
| | /gb=AA418080/gi=2079881/ug=Hs.3972/len=543” | | |
| 37147_at | Cluster Incl AF020044:Homo sapiens lymphocyte secreted C-type lectin precursor, mRNA, complete cds | 2.009944208 | 0.007262 |
| | /cds=(179,1150)/gb=AF020044/gi=2828595/ug=Hs.105927/len=1391 | | |
| 41405_at | Cluster Incl AF026692:Homo sapiens frizzled related protein frpHE mRNA, complete cds/cds=(257,1297) | 2.176388814 | 0.011092 |
| | /gb=AF026692/gi=2920803/ug=Hs.105700/len=2840 | | |
| 38637_at | Cluster Incl L16895:Human lysyl oxidase (LOX) gene, exon 7/cds=(0,6)/gb=L16895/gi=292923 | 2.180657934 | 0.034442 |
| | /ug=Hs.102267/len=2052 | | |
| 1114_at | M22490/FEATURE=/DEFINITION=HUMBMP2B Human bone morphogenetic protein-2B (BMP-2B) mRNA | 2.23232076 | 0.013214 |
| 671_at | J03040/FEATURE=/DEFINITION=HUMSPARC Human SPARC/osteonectin mRNA, complete cds | 2.271250535 | 0.007548 |
| 652_g_at | L07493/FEATURE=/DEFINITION=HUM14RPA Homo sapiens replication protein A 14 kDa subunit (RPA) | 2.271349484 | 0.001554 |
| | mRNA, complete cds | | |
| 41169_at | Cluster Incl X74039:H.sapiens mRNA for urokinase plasminogen activator receptor/cds=(46,891) | 2.278539606 | 0.023709 |
| | /gb=X74039/gi=456192/ug=Hs.179657/len=1070 | | |
| 39710_at | Cluster Incl U30521:Human P311 HUM (3.1) mRNA, complete cds/cds=(202,408)/gb=U30521/gi=963091 | 2.338829553 | 0.004316 |
| | /ug=Hs.142827/len=2036 | | |
| 38466_at | Cluster Incl X82153:H.sapiens mRNA for cathepsin O/cds=(129,1118)/gb=X82153/gi=562756 | 2.362505408 | 0.002649 |
| | /ug=Hs.83942/len=1669 | | |
| 36007_at | Cluster Incl AL050137:Homo sapiens mRNA; cDNA DKFZp586L151 (from clone DKFZp586L151) | 2.433041892 | 0.013328 |
| | /cds=(0,1300)/gb=AL050137/gi=4884149/ug=Hs.43658/len=1810 | | |
| 41870_at | Cluster Incl AF030428:Homo sapiens lung type-I cell membrane-associated protein hT1a-2 (hT1a-2) mRNA, | 2.540477039 | 0.017889 |
| | complete cds/cds=(180,668)/gb=AF030428/gi=4103968/ug=Hs.135150/len=865 | | |
| 39271_at | Cluster Incl AA461365:zx70e07.r1 Homo sapiens cDNA, 5 end/clone=IMAGE-796836/clone_end=5“ | 2.588708014 | 0.020462 |
| | /gb=AA461365/gi=2186485/ug=Hs.237742/len=591” | | |
| 37678_at | Cluster Incl U23070:Human putative transmembrane protein (nma) mRNA, complete cds/cds=(372,1154) | 2.591572193 | 0.005203 |
| | /gb=U23070/gi=1262172/ug=Hs.78776/len=1521 | | |
| 37906_at | Cluster Incl Z37976:H.sapiens mRNA for latent transforming growth factor-beta binding protein (LTBP-2) | 2.599655226 | 0.001094 |
| | /cds=(388,5853)/gb=Z37976/gi=1272663/ug=Hs.83337/len=7000 | | |
| 40681_at | Cluster Incl AB008375:Homo sapiens mRNA for osteoblast specific cysteine-rich protein, complete cds | 2.647250803 | 0.006876 |
| | /cds=(378,1589)/gb=AB008375/gi=2570151/ug=Hs.82582/len=2375 | | |
| 34862_at | Cluster Incl AA005018:zh96a09.r1 Homo sapiens cDNA, 5 end/clone=IMAGE-429112/clone end=5“ | 2.87530968 | 0.002292 |
| | /gb=AA005018/gi=1448859/ug=Hs.4963/len=637” | | |
| 38112_g_ | Cluster Incl X15998:H.sapiens mRNA for the chondroitin sulphate proteoglycan versican, V1 splice-variant; | 2.907786251 | 0.015823 |
| | precursor peptide/cds=(266,7495)/gb=X15998/gi=37662/ug=Hs.81800/len=8224 | | |
| 32617_at | Cluster Incl W74442:zd75e09.s1 Homo sapiens cDNA, 3 end/clone=IMAGE-346504/clone_end=3“ | 1.863661777 | 0.014302 |
| | /gb=W74442/gi=1384777/ug=Hs.80961/len=580” | | |
| 35479_at | Cluster Incl AJ242015:Homo sapiens mRNA for eMDC II protein/cds=(47,2374)/gb=AJ242015/gi=4757043 | 2.399523898 | 0.010738 |
| | /ug=Hs.174030/len=3056 | | |
| 33168_at | Cluster Incl H24861:y142e11.r1 Homo sapiens cDNA, 5 end/clone=IMAGE-160940/clone_end=5“ | 2.450031618 | 0.000099 |
| | /gb=H24861/gi=893760/ug=Hs.90145/len=517” | | |
| 37821_at | Cluster Incl AF041260:Homo sapiens AIBC1 (AIBC1) mRNA, complete cds/cds=(117,1871)/gb=AF041260 | 2.641191847 | 0.03309 |
| | /gi=3335398/ug=Hs.129057/len=2813 | | |
| 34830_at | Cluster Incl W25986:17e7 Homo sapiens cDNA/gb=W25986/gi=1306253/ug=Hs.4750/len=769 | 2.743466262 | 0.000057 |
| 35724_at | Cluster Incl Y07867:H.sapiens mRNA for Pirin, isolate 1/cds=(204,1076)/gb=Y07867/gi=1907075 | 0.390677117 | 0.000031 |
| | /ug=Hs.38842/len=1277 | | |
| 1779_s_at | M16750/FEATURE=/DEFINITION=HUMPIM1 Human pim-1 oncogene mRNA, complete cds | 0.361169226 | 0.000229 |
| 37533_r_a | Cluster Incl D86980:Human mRNA for KIAA0227 gene, partial cds/cds=(0,1011)/gb=D86980/gi=1504033 | 0.358908632 | 0.000665 |
| | /ug=Hs.79170/len=5217 | | |
| 38553_r_a | Cluster Incl A1984786:wr85c06.x1 Homo sapiens cDNA, 3 end/clone=IMAGE-2494474/clone_end=3“ | 0.354417702 | 0.000137 |
| | /gb=Al984786/gi=5812063/ug=Hs.175941/len=814” | | |
| 38349_at | Cluster Incl AF038564:Homo sapiens atrophin-1 interacting protein 4 (AIP4) mRNA, partial cds/cds=(0,2219) | 0.351660122 | 0.000684 |
| | /gb=AF038564/gi=2708328/ug=Hs.98074/len=2358 | | |
| 37596_at | Cluster Incl U09117:Human phospholipase c delta 1 mRNA, complete cds/cds=(94,2364)/gb=U09117 | 0.351047496 | 0.001325 |
| | /gi=483919/ug=Hs.80776/len=2627 | | |
| 34931_at | Cluster Incl AB023157:Homo sapiens mRNA for KIAA0940 protein, complete cds/cds=(89,2143) | 0.350176398 | 0.000066 |
| | /gb=AB023157/gi=4589523/ug=Hs.131945/len=5715 | | |
| 36116_at | Cluster Incl AJ000414:Homo sapiens mRNA for Cdc42-interacting protein 4 (CIP4)/cds=(39,1676) | 0.346256072 | 0.00077 |
| | /gb=AJ000414/gi=2274965/ug=Hs.73999/len=2001 | | |
| 37217_at | Cluster Incl AI208485:qg36f11.x1 Homo sapiens cDNA, 3 end/clone=IMAGE-1837293/clone_end=3“ | 0.337140887 | 0.000001 |
| | /gb=AI208485/gi=3770427/ug=Hs.77253/len=537” | | |

TABLE 8
Genes identified by heirarchical clustering of
the full Human Genome U95 set showing fold change between
normal and diseased sample sets.

| | FOLD | |
|---|---|---|
| ACCESSION | CHANGE | NAME |
| AI199897 | −22.0 | EST |
| W69365 | −8.2 | EST |
| AI081571 | −8.9 | EST |
| AI962905 | −9.6 | CGI-119 PROTEIN |
| AI885390 | −8.6 | EST |
| X94323 | −11.2 | SPECIFIC GRANULE PROTEIN |
| M62982 | −11.4 | ARACHIDONATE 12-LIPOXYGENASE |
| X87159 | −6.5 | SODIUM CHANNEL (SCNNIB) |
| M32402 | −9.7 | PLACENTA PROTEIN 11 |
| AI739630 | −19.7 | EST |
| AW025309 | −40.5 | EST |
| AB001325 | −20.2 | AQUAPORIN 3 |
| AI582193 | −35.1 | EST |
| AB002134 | −12.1 | AIRWAY TRYPSIN-LIKE PROTEASE |
| AL050220 | −17.6 | KALLIKREIN 13 |
| AI971202 | −12.2 | EST |
| AI669212 | −14.3 | EST |
| AI916261 | −9.2 | EST |
| M24902 | −1.0 | ACID PHOSPHATASE PROSTATE |
| U83115 | −8.1 | AIM1 (ABSENT IN MELANOMA) |
| Y09538 | −17.3 | ZINC FINGER PROTEIN 185 |
| AI142832 | −15.4 | EST |
| Y16961 | −12.6 | TUMOR PROTEIN P63 |
| AA130221 | −22.4 | EST |
| AA781220 | −19.0 | PAIRED BOX GENE 9 (PAX9) |
| AI282714 | −77.6 | DESCI PROTEIN |
| M98477 | −22.2 | TRANSGLUTAMINASE 1 |
| AI540870 | −27.6 | EST |
| AF045941 | −24.5 | SCIELLIN |
| R37637 | −44.5 | EST |
| AA743820 | −49.5 | EST |
| AI623978 | −03.1 | EST |
| AI859619 | −15.9 | EST |
| AJ223693 | −10.5 | GPI-ANCHORED HOMOLOG |
| AI378979 | −37.1 | KATANIN P60 SUBUNIT A1 |
| S66896 | −14.2 | SCCAI |
| W68630 | −28.5 | EST |
| AA401397 | −16.4 | KATANIN 13 |
| AA010777 | −35.7 | GALECTIN 7 |
| L10386 | −25.1 | TRANSGLUTAMINASE 3 |
| AI814274 | −123.8 | EST |
| X76342 | −20.7 | ALCOHOL DEHYDROGENASE 7 |
| AI692575 | −46.0 | EST |
| T71258 | −23.9 | ? |
| AI265958 | −58.2 | EST |
| M13903 | −15.7 | INVOLUCRIN |
| M60047 | −20.7 | HBP17 |
| X99977 | −35.3 | ARS GENE, COMPONENT B |
| AI369347 | −7.9 | EST |
| A1052020 | −7.4 | EST |
| AI818579 | 2.5 | EST |
| AI557210 | 4.7 | EST |
| AI754693 | 4.1 | EST |
| AB029000 | 8.3 | MRNA FOR KIAA1077 PROTEIN |
| U09278 | 7.9 | FIBROBLAST ACTIVATION PROTEIN, |
| | | ALPHA |
| AA044844 | 3.4 | SOLUTE CARRIER FAMILY 11, |
| | | MEMBER 3 |
| J04162 | 3.1 | Fc FRAGMENT OF IgC, LOW AFFINITY |
| | | IIIA |
| AA147884 | 5.0 | EST |
| AA584310 | 13.2 | CGI-101 PROTEIN |
| D21254 | 3.8 | CADHERIN-11 |
| D21255 | 3.9 | CADHERIN-11 |
| X82153 | 2.4 | CATHEPSIN K |
| AA127736 | 3.6 | COLLAGEN, TYPE V, ALPHA 2 |
| AW007442 | 6.4 | BIGLYCAN |
| Z37976 | 2.6 | LATENT TGFB BINDING PROTEIN 2 |
| AI686894 | 3.5 | EST |
| AA007367 | 6.6 | EST |
| AF052124 | 9.5 | SPPI (OSTEOPONTIN) |
| AA088177 | 4.3 | EST |
| J04765 | 5.4 | SPPI (OSTEOPONTIN) |
| AA447232 | 4.3 | CATHEPSIN B |
| X15998 | 4.3 | CHONDROITIN SULFATE |
| | | PROTEOGLYCAN 2 |
| AA426499 | 3.8 | CHONDROITIN SULFATE |
| | | PROTEOGLYCAN 2 |
| X15998 | 2.9 | CHONDROITIN SULFATE |
| | | PROTEOGLYCAN 2 |
| AA704137 | 5.4 | THY-1 CELL SURFACE ANTIGEN |
| AI740961 | 5.7 | EST |
| AI864014 | 17.2 | SPPI (OSTEOPONTIN) |
| D13666 | 5.0 | OSTEOBLAST SPECIFIC FACTOR 2 |
| AL050137 | 3.1 | EST |
| AI970896 | 3.1 | EST |
| AF020044 | 2.0 | STEM CELL GROWTH FACTOR |
| AI333224 | 3.0 | EST |
| AI091277 | 3.9 | EST |
| W74476 | 4.7 | EST |
| AA056278 | 5.3 | EHM2 GENE |
| AA877900 | 20.3 | HYPOTHETICAL PROTEIN FLJ20063 |
| AI799626 | 10.2 | EST |
| M35252 | 33.9 | TRANSMEMBRANE, 4, SUPERFAMILY |
| | | MEMBER 3 |
| AI961220 | 30.1 | SERINE PROTEASE INHIBITOR |
| | | (SPINK1) |
| AI148745 | 9.6 | EST |
| AI982768 | 5.7 | EST |
| H30385 | 4.8 | EST |
| AB023171 | 10.6 | MRNA FOR KIAA0954 PROTEIN |
| AW007803 | 7.4 | EST |
| AA458524 | 7.5 | EST |
| AI301060 | 9.0 | EST |
| AI859849 | 16.2 | EST |
| AA156240 | 5.9 | SERINE PROTEASE, UMBLICAL |
| | | ENDOTHELIUM |
| AI691066 | 15.8 | EST |
| AB006781 | 20.9 | GALECTIN 4 |
| AA535447 | 18.6 | EST |
| AI125252 | 15.9 | EST |
| AI308063 | 126.9 | EST |
| U73167 | 4.3 | COSMID CLONE |
| N30008 | 3.8 | EST |
| AI392817 | 5.6 | HEPATOCYTE NUCLEAR FACTOR 3 |
| | | GAMMA |
| AB018335 | 5.2 | MRNA FOR KIAA0792 PROTEIN |
| AF065388 | 5.3 | TETRASPAN-1 |

What is claimed is:

1. A method of diagnosing esophageal cancer in a patient, comprising:
(a) detecting the level of expression in a tissue sample of two or more genes from Tables 2-8; wherein differential expression of the genes in Tables 2-8 is indicative of esophageal cancer.

2. A method of detecting the progression of esophageal cancer in a patient, comprising:
(a) detecting the level of expression in a tissue sample of two or more genes from Tables 2-8; wherein differential expression of the genes in Tables 2-8 is indicative of esophageal cancer progression.

3. A method according to claim 2, wherein the progression is the progression of Barrett's esophagus to adenocarcinoma.

4. A method of monitoring the treatment of a patient with esophageal cancer, comprising:
(a) administering a pharmaceutical composition to the patient;
(b) preparing a gene expression profile from a cell or tissue sample from the patient; and
(c) comparing the patient gene expression profile to a gene expression from a cell population selected from the group consisting of normal esophageal cells, cells from Barrett's esophagus and esophageal adenocarcinoma cells.

5. A method of treating a patient with esophageal cancer, comprising:
(a) administering to the patient a pharmaceutical composition, wherein the composition alters the expression of at least one gene in Tables 2-8;
(b) preparing a gene expression profile from a cell or tissue sample from the patient comprising esophageal cancer cells; and
(c) comparing the patient expression profile to a gene expression profile selected from the group consisting of normal esophageal cells, cells from Barrett's esophagus and esophageal adenocarcinoma cells.

6. A method of diagnosing esophageal adenocarcinoma in a patient, comprising:
(a) detecting the level of expression in a tissue sample of two or more genes from Tables 2-8; wherein differential expression of the genes in Tables 2-8 is indicative of esophageal adenocarcinoma.

7. A method of detecting the progression of esophageal adenocarcinoma in a patient, comprising:
(a) detecting the level of expression in a tissue sample of two or more genes from Tables 2-8; wherein differential expression of the genes in Tables 2-8 is indicative of esophageal adenocarcinoma progression.

8. A method of monitoring the treatment of a patient with esophageal adenocarcinoma, comprising:
(a) administering a pharmaceutical composition to the patient;
(b) preparing a gene expression profile from a cell or tissue sample from the patient; and
(c) comparing the patient gene expression profile to a gene expression from a cell population comprising normal esophagealo cells or to a gene expression profile from a cell population comprising esophageal adenocarcinoma cells or to both.

9. A method of treating a patient with esophageal adenocarcinoma, comprising:
(a) administering to the patient a pharmaceutical composition, wherein the composition alters the expression of at least one gene in Tables 2-8;
(b) preparing a gene expression profile from a cell or tissue sample from the patient comprising esophageal adenocarcinoma cells; and
(c) comparing the patient expression profile to a gene expression profile from an untreated cell population comprising esophageal adenocarcinoma cells.

10. A method of screening for an agent capable of modulating the onset or progression of esophageal cancer, comprising:
(a) preparing a first gene expression profile of a cell population comprising esophageal cancer cells, wherein the expression profile determines the expression level of one or more genes from Tables 2-8;
(b) exposing the cell population to the agent;
(c) preparing second gene expression profile of the agent-exposed cell population; and
(d) comparing the first and second gene expression profiles.

11. The method of claim 10, wherein the esophageal cancer is a esophageal adenocarcinoma.

12. A composition comprising at least two oligonucleotides, wherein each of the oligonucleotides comprises a sequence that specifically hybridizes to a gene in Tables 2-8.

13. A composition according to claim 12, wherein the composition comprises at least 3 oligonucleotides.

14. A composition according to claim 12, wherein the composition comprises at least 5 oligonucleotides.

15. A composition according to claim 12, wherein the composition comprises at least 7 oligonucleotides.

16. A composition according to claim 12, wherein the composition comprises at least 10 oligonucleotides.

17. A composition according to any one of claims 12-16, wherein the oligonucleotides are attached to a solid support.

18. A composition according to claim 17, wherein the solid support is selected from a group consisting of a membrane, a glass support, a filter, a tissue culture dish, a polymeric material, a bead and a silica support.

19. A solid support comprising at least two oligonucleotides, wherein each of the oligonucleotides comprises a sequence that specifically hybridizes to a gene in Tables 2-8.

20. A solid support according to claim 19, wherein the oligonucleotides are covalently attached to the solid support.

21. A solid support according to claim 19, wherein the oligonucleotides are non-covalently attached to the solid support.

22. A solid support according to claim 19, wherein the support comprises at least about 10 different oligonucleotides in discrete locations per square centimeter.

23. A solid support according to claim 19, wherein the support comprises at least about 100 different oligonucleotides in discrete locations per square centimeter.

24. A solid support according to claim 19, wherein the support comprises at least about 1000 different oligonucleotides in discrete locations per square centimeter.

25. A solid support according to claim 19, wherein the support comprises at least about 10,000 different oligonucleotides in discrete locations per square centimeter.

26. A computer system comprising:
(a) a database containing information identifying the expression level in esophageal tissue of a set of genes comprising at least two genes in Tables 2-8; and
(b) a user interface to view the information.

27. A computer system of claim 26, wherein the database her comprises sequence information for the genes.

28. A computer system of claim 26, wherein the database further comprises information identifying the expression level for the set of genes in normal esophageal tissue.

29. A computer system of claim 26, wherein the database further comprises information identifying the expression level of the set of genes in esophageal cancer tissue.

30. A computer system of claim 29, wherein the esophageal cancer tissue comprises esophageal adenocarcinoma cells.

31. A computer system of claim 26-30, further comprising records including descriptive information from an external database, which information correlates said genes to records in the external database.

32. A computer system of claim 31, wherein the external database is GenBank.

33. A method of using a computer system of any one of claims 26-30 to present information identifying the expression level in a tissue or cell of at least one gene in Tables 2-8, comprising:
(a) comparing the expression level of at least one gene in Tables 2-8 in the tissue or cell to the level of expression of the gene in the database.

34. A method of claim 33, wherein the expression level of at least two genes are compared.

35. A method of claim 33, wherein the expression level of at least five genes are compared.

36. A method of claim 33, wherein the expression level of at least ten genes are compared.

37. A method of claim 33, further comprising displaying the level of expression of at least one gene in the tissue or cell sample compared to the expression lev l in esophageal cancer.
